SEQUENCE LISTING (190 sequences)

SEQ ID NO: 1 - protein, 184 aa, Homo sapiens
MLQMAGQCSQNEYFDSLLHACIPCQLRCSSNTPPLTCQRYCNASVTNSVKGTNAILWTCLGLSLIISLAVFVLMFLLRKINSEPLKDEFKNTGSGLLGMANIDLEKSRTGDEIILPRGLEYTVEECTCEDCIKSKPKVDSDHCFPLPAMEEGATILVTTKTNDYCKSLPAALSATEIEKSISAR

SEQ ID NO: 2 - protein, 117 aa, Artificial Sequence
  Synthesized
QIQLVQSGPELKKPGETVKISCKASGYTFTHYSINWVKRAPGKGLKWMGWINTESGEPTYAYDFKGRFAFSLETSASTAYLQINNLKYEDTATYFCALDYESAMDYWGQGTSVTVSS

SEQ ID NO: 3 - DNA, 351 bp, Artificial Sequence
  Synthesized
caaattcagctggtccagagcggacctgagctgaaaaaacccggcgagactgttaagatcagttgtaaagcatctggctataccttcacccactacagcataaattgggtgaaacgggcccctggaaagggcctcaaatggatgggttggatcaataccgaatctggggagcctacttatgcatatgacttcaaagggagattcgccttttcactcgagacatctgcctctactgcttacctccaaataaacaacctcaagtatgaagatacagccacttacttttgcgccctcgactatgagagtgccatggactactggggacagggaacctccgttaccgtcagttcc

SEQ ID NO: 4 - protein, 111 aa, Artificial Sequence
  Synthesized
DIVLTQSPPSLAMSLGKRATISCRASESVTIPGQHLINWYQQKPGQPPKLLIQRASNVESGVPARFSGSGSRTDFTLTIDPVEEDDVAVYYCLQTRGIPRTFGGGTKLEIK

SEQ ID NO: 5 - DNA, 333 bp, Artificial Sequence
  Synthesized
gacattgtgctcactcagtcacctcccagcctggccatgagcctgggaaaaagggccaccatctcctgtagagccagtgagtccgtcacaatccctgggcagcatctcatcaactggtatcagcagaagcccgggcagcctccaaagcttcttattcagcgtgcgtcaaacgtcgagtcgggtgtacctgccagattttctggtagcgggtcccgcactgattttacactgaccatagatccagtggaagaagacgatgtggccgtgtattattgtctgcagactagaggtattcctcggacatttggtgggggtactaagctggagattaag

SEQ ID NO: 6 - protein, 117 aa, Artificial Sequence
  Synthesized
QIQLVQSGPELKKPGETVKISCKASGYTFTHYSINWVKRAPGKGLKWMGWINTETRESTYAYDFKGRFAFSLETSASTAYLQINNLKYEDTATYFCALDYKQAMDYWGQGTSVTVSS

SEQ ID NO: 7 - DNA, 351 bp, Artificial Sequence
  Synthesized
caaattcagctggtccagagcggacctgagctgaaaaaacccggcgagactgttaagatcagttgtaaagcatctggctataccttcacccactacagcataaattgggtgaaacgggcccctggaaagggcctcaaatggatgggttggatcaataccgaaactagggagtctacttatgcatatgacttcaaagggagattcgccttttcactcgagacatctgcctctactgcttacctccaaataaacaacctcaagtatgaagatacagccacttacttttgcgccctcgactataagcaggccatggactactggggacagggaacctccgttaccgtcagttcc

SEQ ID NO: 8 - protein, 111 aa, Artificial Sequence
  Synthesized
DIVLTQSPPSLAMSLGKRATISCRASESVTIPGQHLIHWYQQRPGQPPKLLIQRASNLESGVPARFSGSGSRTDFTLTIDPVEEDDVAVYYCLQTRKIPRTFGGGTKLEIK

SEQ ID NO: 9 - DNA, 333 bp, Artificial Sequence
  Synthesized
gacattgtgctcactcagtcacctcccagcctggccatgagcctgggaaaaagggccaccatctcctgtagagccagtgagtccgtcacaatcccggggcagcatttaatccactggtatcagcagaggcccgggcagcctccaaagcttcttattcagcgtgcgtcaaacctcgagtcgggtgtacctgccagattttctggtagcgggtcccgcactgattttacactgaccatagatccagtggaagaagacgatgtggccgtgtattattgtctgcagactagaaagattcctaggacatttggtgggggtactaagctggagattaag

SEQ ID NO: 10 - protein, 117 aa, Artificial Sequence
  Synthesized
QIQLVQSGPELKKPGETVKISCKASGYTFTHYSINWVKRAPGKGLKWMGWINTETRESTYAYDFKGRFAFSLETSASTAYLQINNLKYEDTATYFCALDYWSAMDYWGQGTSVTVSS

SEQ ID NO: 11 - DNA, 351 bp, Artificial Sequence
  Synthesized
caaattcagctggtccagagcggacctgagctgaaaaaacccggcgagactgttaagatcagttgtaaagcatctggctataccttcacccactacagcattaattgggtgaaacgggcccctggaaagggcctcaaatggatgggttggatcaataccgaaactcgggagtctacttatgcatatgacttcaaagggagattcgccttttcactcgagacatctgcctctactgcttacctccaaataaacaacctcaagtatgaagatacagccacttacttttgcgccctcgactattggagtgccatggactactggggacagggaacctccgttaccgtcagttcc

SEQ ID NO: 12 - protein, 111 aa, Artificial Sequence
  Synthesized
DIVLTQSPPSLAMSLGKRATISCRASESVTIQGLHLIHWYQQKPGQPPKLLIQRASNVQSGVPARFSGSGSRTDFTLTIDPVEEDDVAVYYCQQTRRIPRTFGGGTKLEIK

SEQ ID NO: 13 - DNA, 333 bp, Artificial Sequence
  Synthesized
gacattgtgctcactcagtcacctcccagcctggccatgagcctgggaaaaagggccaccatctcctgtagagccagtgagtccgtcacaatccaggggttgcatttaattcactggtatcagcagaagcccgggcagcctccaaagcttcttattcagagagcgtcaaacgttcagtcgggtgtacctgccagattttctggtagcgggtcccgcactgattttacactgaccatagatccagtggaagaagacgatgtggccgtgtattattgtcagcagactagacgtattcctcgtacatttggtgggggtactaagctggagattaag

SEQ ID NO: 14 - protein, 5 aa, Artificial Sequence
  Synthesized
HYSIN

SEQ ID NO: 15 - protein, 17 aa, Artificial Sequence
  Synthesized
WINTESGEPTYAYDFKG

SEQ ID NO: 16 - protein, 8 aa, Artificial Sequence
  Synthesized
DYESAMDY

SEQ ID NO: 17 - protein, 15 aa, Artificial Sequence
  Synthesized
RASESVTIPGQHLIN

SEQ ID NO: 18 - protein, 7 aa, Artificial Sequence
  Synthesized
RASNVES

SEQ ID NO: 19 - protein, 9 aa, Artificial Sequence
  Synthesized
LQTRGIPRT

SEQ ID NO: 20 - protein, 5 aa, Artificial Sequence
  Synthesized
HYSIN

SEQ ID NO: 21 - protein, 17 aa, Artificial Sequence
  Synthesized
WINTETRESTYAYDFKG

SEQ ID NO: 22 - protein, 8 aa, Artificial Sequence
  Synthesized
DYKQAMDY

SEQ ID NO: 23 - protein, 15 aa, Artificial Sequence
  Synthesized
RASESVTIPGQHLIH

SEQ ID NO: 24 - protein, 7 aa, Artificial Sequence
  Synthesized
RASNLES

SEQ ID NO: 25 - protein, 9 aa, Artificial Sequence
  Synthesized
LQTRKIPRT

SEQ ID NO: 26 - protein, 5 aa, Artificial Sequence
  Synthesized
HYSIN

SEQ ID NO: 27 - protein, 17 aa, Artificial Sequence
  Synthesized
WINTETRESTYAYDFKG

SEQ ID NO: 28 - protein, 8 aa, Artificial Sequence
  Synthesized
DYWSAMDY

SEQ ID NO: 29 - protein, 15 aa, Artificial Sequence
  Synthesized
RASESVTIQGLHLIH

SEQ ID NO: 30 - protein, 7 aa, Artificial Sequence
  Synthesized
RASNVQS

SEQ ID NO: 31 - protein, 9 aa, Artificial Sequence
  Synthesized
QQTRRIPRT

SEQ ID NO: 32 - DNA, 54 bp, Artificial Sequence
  Synthesized
ggaagcacgtccggctcagggaagccgggctccggcgagggaagcacgaagggg

SEQ ID NO: 33 - DNA, 54 bp, Artificial Sequence
  Synthesized
ggcagtactagcggtggtggctccgggggcggttccggtgggggcggcagcagc

SEQ ID NO: 34 - protein, 18 aa, Artificial Sequence
  Synthesized
GSTSGSGKPGSGEGSTKG

SEQ ID NO: 35 - protein, 18 aa, Artificial Sequence
  Synthesized
GSTSGGGSGGGSGGGGSS

SEQ ID NO: 36 - protein, 15 aa, Artificial Sequence
  Synthesized
GGGGSGGGGSGGGGS

SEQ ID NO: 37 - protein, 21 aa, Artificial Sequence
  Synthesized
SRGGGGSGGGGSGGGGSLEMA

SEQ ID NO: 38 - protein, 21 aa, Artificial Sequence
  Synthesized
SRGGGGSGGGGSGGGGSLEMA

SEQ ID NO: 39 - protein, 5 aa, Artificial Sequence
  Synthesized
GGGGS

SEQ ID NO: 40 - protein, 9 aa, Artificial Sequence
  Synthesized
GGGGSGGGS

SEQ ID NO: 41 - protein, 10 aa, Artificial Sequence
  Synthesized
GGGGSGGGGS

SEQ ID NO: 42 - protein, 18 aa, Artificial Sequence
  Synthesized
GGGGSGGGGSGGGGGGGS

SEQ ID NO: 43 - protein, 20 aa, Artificial Sequence
  Synthesized
GGGGSGGGGSGGGGSGGGGS

SEQ ID NO: 44 - protein, 25 aa, Artificial Sequence
  Synthesized
GGGGSGGGGSGGGGSGGGGSGGGGS

SEQ ID NO: 45 - protein, 30 aa, Artificial Sequence
  Synthesized
GGGGSGGGGSGGGGSGGGGSGGGGSGGGGS

SEQ ID NO: 46 - protein, 35 aa, Artificial Sequence
  Synthesized
GGGGSGGGGSGGGGSGGGGSGGGGSGGGGSGGGGS

SEQ ID NO: 47 - protein, 15 aa, Artificial Sequence
  Synthesized
EPKSCDKTHTCPPCP

SEQ ID NO: 48 - protein, 24 aa, Artificial Sequence
  Synthesized
GGGGSGGGSEPKSCDKTHTCPPCP

SEQ ID NO: 49 - protein, 62 aa, Artificial Sequence
  Synthesized
ELKTPLGDTTHTCPRCPEPKSCDTPPPCPRCPEPKSCDTPPPCPRCPEPKSCDTPPPCPRCP

SEQ ID NO: 50 - protein, 6 aa, Artificial Sequence
  Synthesized
GSGSGS

SEQ ID NO: 51 - protein, 3 aa, Artificial Sequence
  Synthesized
AAA

SEQ ID NO: 52 - protein, 5 aa, Artificial Sequence
  Synthesized
GLSGL

SEQ ID NO: 53 - DNA, 15 bp, Artificial Sequence
  Synthesized
ggcctgagcggcctg

SEQ ID NO: 54 - protein, 45 aa, Artificial Sequence
  Synthesized
TTTPAPRPPTPAPTIASQPLSLRPEACRPAAGGAVHTRGLDFACD

SEQ ID NO: 55 - DNA, 135 bp, Artificial Sequence
  Synthesized
actactaccccagctccacgtcctcccacgccagctccaacgatagcaagtcagcccttatctcttcgccctgaggcttgcaggcccgcggcgggcggcgccgttcacacgcgaggactagacttcgcctgcgac

SEQ ID NO: 56 - protein, 24 aa, Artificial Sequence
  Synthesized
IYIWAPLAGTCGVLLLSLVITLYC

SEQ ID NO: 57 - DNA, 72 bp, Artificial Sequence
  Synthesized
atctacatctgggcaccactagccgggacttgcggagtgttgttgttgagcttggtaataacgctctactgc

SEQ ID NO: 58 - protein, 42 aa, Artificial Sequence
  Synthesized
KHSRKKFVHLLKRPFIKTTGAAQMEDASSCRCPQEEEGECDL

SEQ ID NO: 59 - DNA, 126 bp, Artificial Sequence
  Synthesized
aaacatagccgcaaaaaatttgtgcatctgctgaaacgcccgtttattaaaaccaccggcgcggcgcagatggaagatgcgagcagctgccgctgcccgcaggaagaagaaggcgaatgcgatctg

SEQ ID NO: 60 - protein, 42 aa, Artificial Sequence
  Synthesized
KASRKKAAAAAKSPFASPASSAQEEDASSCRAPSEEEGSCEL

SEQ ID NO: 61 - DNA, 126 bp, Artificial Sequence
  Synthesized
aaagcgagccgcaaaaaagcggcggcggcggcgaaaagcccgtttgcgagcccggcgagcagcgcgcaggaagaagatgcgagcagctgccgcgcgccgagcgaagaagaaggcagctgcgaactg

SEQ ID NO: 62 - protein, 42 aa, Artificial Sequence
  Synthesized
KRGRKKLLYIFKQPFMRPVQTTQEEDGCSCRFPEEEEGGCEL

SEQ ID NO: 63 - DNA, 126 bp, Artificial Sequence
  Synthesized
aagcgtgggagaaagaagctcttgtacattttcaagcagccattcatgcgtcccgttcagacgactcaggaggaggacggctgctcgtgccgattcccggaggaggaggagggcggttgcgaactc

SEQ ID NO: 64 - protein, 41 aa, Artificial Sequence
  Synthesized
RSKRSRLLHSDYMNMTPRRPGPTRKHYQPYAPPRDFAAYRS

SEQ ID NO: 65 - DNA, 123 bp, Artificial Sequence
  Synthesized
aggagtaagaggagcaggctcctgcacagtgactacatgaacatgactccccgccgcccagggcccacccgcaagcattaccagccctatgccccaccacgcgacttcgcagcctatcgctcc

SEQ ID NO: 66 - protein, 112 aa, Artificial Sequence
  Synthesized
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 67 - DNA, 336 bp, Artificial Sequence
  Synthesized
agagtgaagttctctcgctccgcggacgcacccgcttaccagcagggtcagaaccagctatacaacgagttaaacctggggcgccgggaggagtacgacgtgttagacaagcgtagaggtagggacccggagatgggaggcaagcctcggagaaagaacccccaggagggcctgtacaacgaactccagaaggacaagatggctgaggcgtactcggagattggtatgaagggcgagagacgtcgcggaaagggacacgacggcttataccaggggctttccaccgcgaccaaggacacatacgacgcgctgcacatgcaagccttaccacctcga

SEQ ID NO: 68 - protein, 21 aa, Artificial Sequence
  Synthesized
MALPVTALLLPLALLLHAAQP

SEQ ID NO: 69 - DNA, 63 bp, Artificial Sequence
  Synthesized
atggcgctcccagtgacagccttacttttacctctggcgttattattgcatgcggcccagccg

SEQ ID NO: 70 - protein, 22 aa, Artificial Sequence
  Synthesized
MALPVTALLLPLALLLHAAQPA

SEQ ID NO: 71 - DNA, 66 bp, Artificial Sequence
  Synthesized
atggcgctcccagtgacagccttacttttacctctggcgttattattgcatgcggcccagccggcc

SEQ ID NO: 72 - DNA, 164 bp, Artificial Sequence
  Synthesized
gggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgt

SEQ ID NO: 73 - DNA, 212 bp, Artificial Sequence
  Synthesized
gggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacag

SEQ ID NO: 74 - DNA, 22 bp, Homo Sapiens
tggcctggagcaacaaatctga

SEQ ID NO: 75 - DNA, 22 bp, Homo Sapiens
accggacctcgttgtttagact

SEQ ID NO: 76 - protein, 354 aa, Artificial Sequence
  Synthesized
MNTKYNKEFLLYLAGFVDGDGSIYAVIYPHQRAKFKHFLKLLFTVSQSTKRRWFLDKLVDEIGVGYVYDLPRTSEYRLSEIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSIYACIRPRQGSKFKHRLTLGFAVGQKTQRRWFLDKLVDEIGVGYVYDRGSVSEYVLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 77 - protein, 327 aa, Artificial Sequence
  Synthesized
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 78 - DNA, 981 bp, Artificial Sequence
  Synthesized
gctagcaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagagcctctccctgtctccgggtaaa

SEQ ID NO: 79 - protein, 107 aa, Artificial Sequence [sequence omitted]

SEQ ID NO: 80 - DNA, 321 bp, Artificial Sequence
  Synthesized
cgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 81 - protein, 246 aa, Artificial Sequence
  Synthesized
QIQLVQSGPELKKPGETVKISCKASGYTFTHYSINWVKRAPGKGLKWMGWINTESGEPTYAYDFKGRFAFSLETSASTAYLQINNLKYEDTATYFCALDYESAMDYWGQGTSVTVSSGSTSGSGKPGSGEGSTKGDIVLTQSPPSLAMSLGKRATISCRASESVTIPGQHLINWYQQKPGQPPKLLIQRASNVESGVPARFSGSGSRTDFTLTIDPVEEDDVAVYYCLQTRGIPRTFGGGTKLEIK

SEQ ID NO: 82 - protein, 246 aa, Artificial Sequence
  Synthesized
DIVLTQSPPSLAMSLGKRATISCRASESVTIPGQHLINWYQQKPGQPPKLLIQRASNVESGVPARFSGSGSRTDFTLTIDPVEEDDVAVYYCLQTRGIPRTFGGGTKLEIKGSTSGSGKPGSGEGSTKGQIQLVQSGPELKKPGETVKISCKASGYTFTHYSINWVKRAPGKGLKWMGWINTESGEPTYAYDFKGRFAFSLETSASTAYLQINNLKYEDTATYFCALDYESAMDYWGQGTSVTVSS

SEQ ID NO: 83 - protein, 246 aa, Artificial Sequence
  Synthesized
QIQLVQSGPELKKPGETVKISCKASGYTFTHYSINWVKRAPGKGLKWMGWINTETRESTYAYDFKGRFAFSLETSASTAYLQINNLKYEDTATYFCALDYKQAMDYWGQGTSVTVSSGSTSGSGKPGSGEGSTKGDIVLTQSPPSLAMSLGKRATISCRASESVTIPGQHLIHWYQQRPGQPPKLLIQRASNLESGVPARFSGSGSRTDFTLTIDPVEEDDVAVYYCLQTRKIPRTFGGGTKLEIK

SEQ ID NO: 84 - protein, 246 aa, Artificial Sequence
  Synthesized
DIVLTQSPPSLAMSLGKRATISCRASESVTIPGQHLIHWYQQRPGQPPKLLIQRASNLESGVPARFSGSGSRTDFTLTIDPVEEDDVAVYYCLQTRKIPRTFGGGTKLEIKGSTSGSGKPGSGEGSTKGQIQLVQSGPELKKPGETVKISCKASGYTFTHYSINWVKRAPGKGLKWMGWINTETRESTYAYDFKGRFAFSLETSASTAYLQINNLKYEDTATYFCALDYKQAMDYWGQGTSVTVSS

SEQ ID NO: 85 - protein, 246 aa, Artificial Sequence
  Synthesized
QIQLVQSGPELKKPGETVKISCKASGYTFTHYSINWVKRAPGKGLKWMGWINTETRESTYAYDFKGRFAFSLETSASTAYLQINNLKYEDTATYFCALDYWSAMDYWGQGTSVTVSSGSTSGSGKPGSGEGSTKGDIVLTQSPPSLAMSLGKRATISCRASESVTIQGLHLIHWYQQKPGQPPKLLIQRASNVQSGVPARFSGSGSRTDFTLTIDPVEEDDVAVYYCQQTRRIPRTFGGGTKLEIK

SEQ ID NO: 86 - protein, 246 aa, Artificial Sequence
  Synthesized
DIVLTQSPPSLAMSLGKRATISCRASESVTIQGLHLIHWYQQKPGQPPKLLIQRASNVQSGVPARFSGSGSRTDFTLTIDPVEEDDVAVYYCQQTRRIPRTFGGGTKLEIKGSTSGSGKPGSGEGSTKGQIQLVQSGPELKKPGETVKISCKASGYTFTHYSINWVKRAPGKGLKWMGWINTETRESTYAYDFKGRFAFSLETSASTAYLQINNLKYEDTATYFCALDYWSAMDYWGQGTSVTVSS

SEQ ID NO: 87 - protein, 246 aa, Artificial Sequence
  Synthesized
QIQLVQSGPELKKPGETVKISCKASGYTFTHYSINWVKRAPGKGLKWMGWINTESGEPTYAYDFKGRFAFSLETSASTAYLQINNLKYEDTATYFCALDYESAMDYWGQGTSVTVSSGSTSGSGKPGSGEGSTKGDIVLTQSPPSLAMSLGKRATISCRASESVTIPGQHLIHWYQQRPGQPPKLLIQRASNLESGVPARFSGSGSRTDFTLTIDPVEEDDVAVYYCLQTRKIPRTFGGGTKLEIK

SEQ ID NO: 88 - protein, 246 aa, Artificial Sequence
  Synthesized
DIVLTQSPPSLAMSLGKRATISCRASESVTIPGQHLINWYQQKPGQPPKLLIQRASNVESGVPARFSGSGSRTDFTLTIDPVEEDDVAVYYCLQTRGIPRTFGGGTKLEIKGSTSGSGKPGSGEGSTKGQIQLVQSGPELKKPGETVKISCKASGYTFTHYSINWVKRAPGKGLKWMGWINTETRESTYAYDFKGRFAFSLETSASTAYLQINNLKYEDTATYFCALDYKQAMDYWGQGTSVTVSS

SEQ ID NO: 89 - protein, 246 aa, Artificial Sequence
  Synthesized
QIQLVQSGPELKKPGETVKISCKASGYTFTHYSINWVKRAPGKGLKWMGWINTESGEPTYAYDFKGRFAFSLETSASTAYLQINNLKYEDTATYFCALDYESAMDYWGQGTSVTVSSGSTSGSGKPGSGEGSTKGDIVLTQSPPSLAMSLGKRATISCRASESVTIQGLHLIHWYQQKPGQPPKLLIQRASNVQSGVPARFSGSGSRTDFTLTIDPVEEDDVAVYYCQQTRRIPRTFGGGTKLEIK

SEQ ID NO: 90 - protein, 246 aa, Artificial Sequence
  Synthesized
DIVLTQSPPSLAMSLGKRATISCRASESVTIPGQHLINWYQQKPGQPPKLLIQRASNVESGVPARFSGSGSRTDFTLTIDPVEEDDVAVYYCLQTRGIPRTFGGGTKLEIKGSTSGSGKPGSGEGSTKGQIQLVQSGPELKKPGETVKISCKASGYTFTHYSINWVKRAPGKGLKWMGWINTETRESTYAYDFKGRFAFSLETSASTAYLQINNLKYEDTATYFCALDYWSAMDYWGQGTSVTVSS

SEQ ID NO: 91 - protein, 246 aa, Artificial Sequence
  Synthesized
QIQLVQSGPELKKPGETVKISCKASGYTFTHYSINWVKRAPGKGLKWMGWINTETRESTYAYDFKGRFAFSLETSASTAYLQINNLKYEDTATYFCALDYKQAMDYWGQGTSVTVSSGSTSGSGKPGSGEGSTKGDIVLTQSPPSLAMSLGKRATISCRASESVTIPGQHLINWYQQKPGQPPKLLIQRASNVESGVPARFSGSGSRTDFTLTIDPVEEDDVAVYYCLQTRGIPRTFGGGTKLEIK

SEQ ID NO: 92 - protein, 246 aa, Artificial Sequence
  Synthesized
DIVLTQSPPSLAMSLGKRATISCRASESVTIPGQHLIHWYQQRPGQPPKLLIQRASNLESGVPARFSGSGSRTDFTLTIDPVEEDDVAVYYCLQTRKIPRTFGGGTKLEIKGSTSGSGKPGSGEGSTKGQIQLVQSGPELKKPGETVKISCKASGYTFTHYSINWVKRAPGKGLKWMGWINTESGEPTYAYDFKGRFAFSLETSASTAYLQINNLKYEDTATYFCALDYESAMDYWGQGTSVTVSS

SEQ ID NO: 93 - protein, 246 aa, Artificial Sequence
  Synthesized
QIQLVQSGPELKKPGETVKISCKASGYTFTHYSINWVKRAPGKGLKWMGWINTETRESTYAYDFKGRFAFSLETSASTAYLQINNLKYEDTATYFCALDYKQAMDYWGQGTSVTVSSGSTSGSGKPGSGEGSTKGDIVLTQSPPSLAMSLGKRATISCRASESVTIQGLHLIHWYQQKPGQPPKLLIQRASNVQSGVPARFSGSGSRTDFTLTIDPVEEDDVAVYYCQQTRRIPRTFGGGTKLEIK

SEQ ID NO: 94 - protein, 246 aa, Artificial Sequence
  Synthesized
DIVLTQSPPSLAMSLGKRATISCRASESVTIPGQHLIHWYQQRPGQPPKLLIQRASNLESGVPARFSGSGSRTDFTLTIDPVEEDDVAVYYCLQTRKIPRTFGGGTKLEIKGSTSGSGKPGSGEGSTKGQIQLVQSGPELKKPGETVKISCKASGYTFTHYSINWVKRAPGKGLKWMGWINTETRESTYAYDFKGRFAFSLETSASTAYLQINNLKYEDTATYFCALDYWSAMDYWGQGTSVTVSS

SEQ ID NO: 95 - protein, 246 aa, Artificial Sequence
  Synthesized
QIQLVQSGPELKKPGETVKISCKASGYTFTHYSINWVKRAPGKGLKWMGWINTETRESTYAYDFKGRFAFSLETSASTAYLQINNLKYEDTATYFCALDYWSAMDYWGQGTSVTVSSGSTSGSGKPGSGEGSTKGDIVLTQSPPSLAMSLGKRATISCRASESVTIPGQHLINWYQQKPGQPPKLLIQRASNVESGVPARFSGSGSRTDFTLTIDPVEEDDVAVYYCLQTRGIPRTFGGGTKLEIK

SEQ ID NO: 96 - protein, 246 aa, Artificial Sequence
  Synthesized
DIVLTQSPPSLAMSLGKRATISCRASESVTIQGLHLIHWYQQKPGQPPKLLIQRASNVQSGVPARFSGSGSRTDFTLTIDPVEEDDVAVYYCQQTRRIPRTFGGGTKLEIKGSTSGSGKPGSGEGSTKGQIQLVQSGPELKKPGETVKISCKASGYTFTHYSINWVKRAPGKGLKWMGWINTESGEPTYAYDFKGRFAFSLETSASTAYLQINNLKYEDTATYFCALDYESAMDYWGQGTSVTVSS

SEQ ID NO: 97 - protein, 246 aa, Artificial Sequence
  Synthesized
QIQLVQSGPELKKPGETVKISCKASGYTFTHYSINWVKRAPGKGLKWMGWINTETRESTYAYDFKGRFAFSLETSASTAYLQINNLKYEDTATYFCALDYWSAMDYWGQGTSVTVSSGSTSGSGKPGSGEGSTKGDIVLTQSPPSLAMSLGKRATISCRASESVTIPGQHLIHWYQQRPGQPPKLLIQRASNLESGVPARFSGSGSRTDFTLTIDPVEEDDVAVYYCLQTRKIPRTFGGGTKLEIK

SEQ ID NO: 98 - protein, 246 aa, Artificial Sequence
  Synthesized
DIVLTQSPPSLAMSLGKRATISCRASESVTIQGLHLIHWYQQKPGQPPKLLIQRASNVQSGVPARFSGSGSRTDFTLTIDPVEEDDVAVYYCQQTRRIPRTFGGGTKLEIKGSTSGSGKPGSGEGSTKGQIQLVQSGPELKKPGETVKISCKASGYTFTHYSINWVKRAPGKGLKWMGWINTETRESTYAYDFKGRFAFSLETSASTAYLQINNLKYEDTATYFCALDYKQAMDYWGQGTSVTVSS

SEQ ID NO: 99 - DNA, 738 bp, Artificial Sequence
  Synthesized
caaattcagctggtccagagcggacctgagctgaaaaaacccggcgagactgttaagatcagttgtaaagcatctggctataccttcacccactacagcataaattgggtgaaacgggcccctggaaagggcctcaaatggatgggttggatcaataccgaatctggggagcctacttatgcatatgacttcaaagggagattcgccttttcactcgagacatctgcctctactgcttacctccaaataaacaacctcaagtatgaagatacagccacttacttttgcgccctcgactatgagagtgccatggactactggggacagggaacctccgttaccgtcagttccggaagcacgtccggctcagggaagccgggctccggcgagggaagcacgaagggggacattgtgctcactcagtcacctcccagcctggccatgagcctgggaaaaagggccaccatctcctgtagagccagtgagtccgtcacaatccctgggcagcatctcatcaactggtatcagcagaagcccgggcagcctccaaagcttcttattcagcgtgcgtcaaacgtcgagtcgggtgtacctgccagattttctggtagcgggtcccgcactgattttacactgaccatagatccagtggaagaagacgatgtggccgtgtattattgtctgcagactagaggtattcctcggacatttggtgggggtactaagctggagattaag

SEQ ID NO: 100 - DNA, 738 bp, Artificial Sequence
  Synthesized
gacattgtgctcactcagtcacctcccagcctggccatgagcctgggaaaaagggccaccatctcctgtagagccagtgagtccgtcacaatccctgggcagcatctcatcaactggtatcagcagaagcccgggcagcctccaaagcttcttattcagcgtgcgtcaaacgtcgagtcgggtgtacctgccagattttctggtagcgggtcccgcactgattttacactgaccatagatccagtggaagaagacgatgtggccgtgtattattgtctgcagactagaggtattcctcggacatttggtgggggtactaagctggagattaagggaagcacgtccggctcagggaagccgggctccggcgagggaagcacgaaggggcaaattcagctggtccagagcggacctgagctgaaaaaacccggcgagactgttaagatcagttgtaaagcatctggctataccttcacccactacagcataaattgggtgaaacgggcccctggaaagggcctcaaatggatgggttggatcaataccgaatctggggagcctacttatgcatatgacttcaaagggagattcgccttttcactcgagacatctgcctctactgcttacctccaaataaacaacctcaagtatgaagatacagccacttacttttgcgccctcgactatgagagtgccatggactactggggacagggaacctccgttaccgtcagttcc

SEQ ID NO: 101 - DNA, 738 bp, Artificial Sequence
  Synthesized
caaattcagctggtccagagcggacctgagctgaaaaaacccggcgagactgttaagatcagttgtaaagcatctggctataccttcacccactacagcataaattgggtgaaacgggcccctggaaagggcctcaaatggatgggttggatcaataccgaaactagggagtctacttatgcatatgacttcaaagggagattcgccttttcactcgagacatctgcctctactgcttacctccaaataaacaacctcaagtatgaagatacagccacttacttttgcgccctcgactataagcaggccatggactactggggacagggaacctccgttaccgtcagttccggaagcacgtccggctcagggaagccgggctccggcgagggaagcacgaagggggacattgtgctcactcagtcacctcccagcctggccatgagcctgggaaaaagggccaccatctcctgtagagccagtgagtccgtcacaatcccggggcagcatttaatccactggtatcagcagaggcccgggcagcctccaaagcttcttattcagcgtgcgtcaaacctcgagtcgggtgtacctgccagattttctggtagcgggtcccgcactgattttacactgaccatagatccagtggaagaagacgatgtggccgtgtattattgtctgcagactagaaagattcctaggacatttggtgggggtactaagctggagattaag

SEQ ID NO: 102 - DNA, 738 bp, Artificial Sequence
  Synthesized
gacattgtgctcactcagtcacctcccagcctggccatgagcctgggaaaaagggccaccatctcctgtagagccagtgagtccgtcacaatcccggggcagcatttaatccactggtatcagcagaggcccgggcagcctccaaagcttcttattcagcgtgcgtcaaacctcgagtcgggtgtacctgccagattttctggtagcgggtcccgcactgattttacactgaccatagatccagtggaagaagacgatgtggccgtgtattattgtctgcagactagaaagattcctaggacatttggtgggggtactaagctggagattaagggaagcacgtccggctcagggaagccgggctccggcgagggaagcacgaaggggcaaattcagctggtccagagcggacctgagctgaaaaaacccggcgagactgttaagatcagttgtaaagcatctggctataccttcacccactacagcataaattgggtgaaacgggcccctggaaagggcctcaaatggatgggttggatcaataccgaaactagggagtctacttatgcatatgacttcaaagggagattcgccttttcactcgagacatctgcctctactgcttacctccaaataaacaacctcaagtatgaagatacagccacttacttttgcgccctcgactataagcaggccatggactactggggacagggaacctccgttaccgtcagttcc

SEQ ID NO: 103 - DNA, 738 bp, Artificial Sequence
  Synthesized
caaattcagctggtccagagcggacctgagctgaaaaaacccggcgagactgttaagatcagttgtaaagcatctggctataccttcacccactacagcattaattgggtgaaacgggcccctggaaagggcctcaaatggatgggttggatcaataccgaaactcgggagtctacttatgcatatgacttcaaagggagattcgccttttcactcgagacatctgcctctactgcttacctccaaataaacaacctcaagtatgaagatacagccacttacttttgcgccctcgactattggagtgccatggactactggggacagggaacctccgttaccgtcagttccggaagcacgtccggctcagggaagccgggctccggcgagggaagcacgaagggggacattgtgctcactcagtcacctcccagcctggccatgagcctgggaaaaagggccaccatctcctgtagagccagtgagtccgtcacaatccaggggttgcatttaattcactggtatcagcagaagcccgggcagcctccaaagcttcttattcagagagcgtcaaacgttcagtcgggtgtacctgccagattttctggtagcgggtcccgcactgattttacactgaccatagatccagtggaagaagacgatgtggccgtgtattattgtcagcagactagacgtattcctcgtacatttggtgggggtactaagctggagattaag

SEQ ID NO: 104 - DNA, 738 bp, Artificial Sequence
  Synthesized
gacattgtgctcactcagtcacctcccagcctggccatgagcctgggaaaaagggccaccatctcctgtagagccagtgagtccgtcacaatccaggggttgcatttaattcactggtatcagcagaagcccgggcagcctccaaagcttcttattcagagagcgtcaaacgttcagtcgggtgtacctgccagattttctggtagcgggtcccgcactgattttacactgaccatagatccagtggaagaagacgatgtggccgtgtattattgtcagcagactagacgtattcctcgtacatttggtgggggtactaagctggagattaagggaagcacgtccggctcagggaagccgggctccggcgagggaagcacgaaggggcaaattcagctggtccagagcggacctgagctgaaaaaacccggcgagactgttaagatcagttgtaaagcatctggctataccttcacccactacagcattaattgggtgaaacgggcccctggaaagggcctcaaatggatgggttggatcaataccgaaactcgggagtctacttatgcatatgacttcaaagggagattcgccttttcactcgagacatctgcctctactgcttacctccaaataaacaacctcaagtatgaagatacagccacttacttttgcgccctcgactattggagtgccatggactactggggacagggaacctccgttaccgtcagttcc

SEQ ID NO: 105 - DNA, 738 bp, Artificial Sequence
  Synthesized
caaattcagctggtccagagcggacctgagctgaaaaaacccggcgagactgttaagatcagttgtaaagcatctggctataccttcacccactacagcataaattgggtgaaacgggcccctggaaagggcctcaaatggatgggttggatcaataccgaatctggggagcctacttatgcatatgacttcaaagggagattcgccttttcactcgagacatctgcctctactgcttacctccaaataaacaacctcaagtatgaagatacagccacttacttttgcgccctcgactatgagagtgccatggactactggggacagggaacctccgttaccgtcagttccggaagcacgtccggctcagggaagccgggctccggcgagggaagcacgaagggggacattgtgctcactcagtcacctcccagcctggccatgagcctgggaaaaagggccaccatctcctgtagagccagtgagtccgtcacaatcccggggcagcatttaatccactggtatcagcagaggcccgggcagcctccaaagcttcttattcagcgtgcgtcaaacctcgagtcgggtgtacctgccagattttctggtagcgggtcccgcactgattttacactgaccatagatccagtggaagaagacgatgtggccgtgtattattgtctgcagactagaaagattcctaggacatttggtgggggtactaagctggagattaag

SEQ ID NO: 106 - DNA, 738 bp, Artificial Sequence
  Synthesized
gacattgtgctcactcagtcacctcccagcctggccatgagcctgggaaaaagggccaccatctcctgtagagccagtgagtccgtcacaatccctgggcagcatctcatcaactggtatcagcagaagcccgggcagcctccaaagcttcttattcagcgtgcgtcaaacgtcgagtcgggtgtacctgccagattttctggtagcgggtcccgcactgattttacactgaccatagatccagtggaagaagacgatgtggccgtgtattattgtctgcagactagaggtattcctcggacatttggtgggggtactaagctggagattaagggaagcacgtccggctcagggaagccgggctccggcgagggaagcacgaaggggcaaattcagctggtccagagcggacctgagctgaaaaaacccggcgagactgttaagatcagttgtaaagcatctggctataccttcacccactacagcataaattgggtgaaacgggcccctggaaagggcctcaaatggatgggttggatcaataccgaaactagggagtctacttatgcatatgacttcaaagggagattcgccttttcactcgagacatctgcctctactgcttacctccaaataaacaacctcaagtatgaagatacagccacttacttttgcgccctcgactataagcaggccatggactactggggacagggaacctccgttaccgtcagttcc

SEQ ID NO: 107 - DNA, 738 bp, Artificial Sequence
  Synthesized
caaattcagctggtccagagcggacctgagctgaaaaaacccggcgagactgttaagatcagttgtaaagcatctggctataccttcacccactacagcataaattgggtgaaacgggcccctggaaagggcctcaaatggatgggttggatcaataccgaatctggggagcctacttatgcatatgacttcaaagggagattcgccttttcactcgagacatctgcctctactgcttacctccaaataaacaacctcaagtatgaagatacagccacttacttttgcgccctcgactatgagagtgccatggactactggggacagggaacctccgttaccgtcagttccggaagcacgtccggctcagggaagccgggctccggcgagggaagcacgaagggggacattgtgctcactcagtcacctcccagcctggccatgagcctgggaaaaagggccaccatctcctgtagagccagtgagtccgtcacaatccaggggttgcatttaattcactggtatcagcagaagcccgggcagcctccaaagcttcttattcagagagcgtcaaacgttcagtcgggtgtacctgccagattttctggtagcgggtcccgcactgattttacactgaccatagatccagtggaagaagacgatgtggccgtgtattattgtcagcagactagacgtattcctcgtacatttggtgggggtactaagctggagattaag

SEQ ID NO: 108 - DNA, 738 bp, Artificial Sequence
  Synthesized
gacattgtgctcactcagtcacctcccagcctggccatgagcctgggaaaaagggccaccatctcctgtagagccagtgagtccgtcacaatccctgggcagcatctcatcaactggtatcagcagaagcccgggcagcctccaaagcttcttattcagcgtgcgtcaaacgtcgagtcgggtgtacctgccagattttctggtagcgggtcccgcactgattttacactgaccatagatccagtggaagaagacgatgtggccgtgtattattgtctgcagactagaggtattcctcggacatttggtgggggtactaagctggagattaagggaagcacgtccggctcagggaagccgggctccggcgagggaagcacgaaggggcaaattcagctggtccagagcggacctgagctgaaaaaacccggcgagactgttaagatcagttgtaaagcatctggctataccttcacccactacagcattaattgggtgaaacgggcccctggaaagggcctcaaatggatgggttggatcaataccgaaactcgggagtctacttatgcatatgacttcaaagggagattcgccttttcactcgagacatctgcctctactgcttacctccaaataaacaacctcaagtatgaagatacagccacttacttttgcgccctcgactattggagtgccatggactactggggacagggaacctccgttaccgtcagttcc

SEQ ID NO: 109 - DNA, 738 bp, Artificial Sequence
  Synthesized
caaattcagctggtccagagcggacctgagctgaaaaaacccggcgagactgttaagatcagttgtaaagcatctggctataccttcacccactacagcataaattgggtgaaacgggcccctggaaagggcctcaaatggatgggttggatcaataccgaaactagggagtctacttatgcatatgacttcaaagggagattcgccttttcactcgagacatctgcctctactgcttacctccaaataaacaacctcaagtatgaagatacagccacttacttttgcgccctcgactataagcaggccatggactactggggacagggaacctccgttaccgtcagttccggaagcacgtccggctcagggaagccgggctccggcgagggaagcacgaagggggacattgtgctcactcagtcacctcccagcctggccatgagcctgggaaaaagggccaccatctcctgtagagccagtgagtccgtcacaatccctgggcagcatctcatcaactggtatcagcagaagcccgggcagcctccaaagcttcttattcagcgtgcgtcaaacgtcgagtcgggtgtacctgccagattttctggtagcgggtcccgcactgattttacactgaccatagatccagtggaagaagacgatgtggccgtgtattattgtctgcagactagaggtattcctcggacatttggtgggggtactaagctggagattaag

SEQ ID NO: 110 - DNA, 738 bp, Artificial Sequence
  Synthesized
gacattgtgctcactcagtcacctcccagcctggccatgagcctgggaaaaagggccaccatctcctgtagagccagtgagtccgtcacaatcccggggcagcatttaatccactggtatcagcagaggcccgggcagcctccaaagcttcttattcagcgtgcgtcaaacctcgagtcgggtgtacctgccagattttctggtagcgggtcccgcactgattttacactgaccatagatccagtggaagaagacgatgtggccgtgtattattgtctgcagactagaaagattcctaggacatttggtgggggtactaagctggagattaagggaagcacgtccggctcagggaagccgggctccggcgagggaagcacgaaggggcaaattcagctggtccagagcggacctgagctgaaaaaacccggcgagactgttaagatcagttgtaaagcatctggctataccttcacccactacagcataaattgggtgaaacgggcccctggaaagggcctcaaatggatgggttggatcaataccgaatctggggagcctacttatgcatatgacttcaaagggagattcgccttttcactcgagacatctgcctctactgcttacctccaaataaacaacctcaagtatgaagatacagccacttacttttgcgccctcgactatgagagtgccatggactactggggacagggaacctccgttaccgtcagttcc

SEQ ID NO: 111 - DNA, 738 bp, Artificial Sequence
  Synthesized
caaattcagctggtccagagcggacctgagctgaaaaaacccggcgagactgttaagatcagttgtaaagcatctggctataccttcacccactacagcataaattgggtgaaacgggcccctggaaagggcctcaaatggatgggttggatcaataccgaaactagggagtctacttatgcatatgacttcaaagggagattcgccttttcactcgagacatctgcctctactgcttacctccaaataaacaacctcaagtatgaagatacagccacttacttttgcgccctcgactataagcaggccatggactactggggacagggaacctccgttaccgtcagttccggaagcacgtccggctcagggaagccgggctccggcgagggaagcacgaagggggacattgtgctcactcagtcacctcccagcctggccatgagcctgggaaaaagggccaccatctcctgtagagccagtgagtccgtcacaatccaggggttgcatttaattcactggtatcagcagaagcccgggcagcctccaaagcttcttattcagagagcgtcaaacgttcagtcgggtgtacctgccagattttctggtagcgggtcccgcactgattttacactgaccatagatccagtggaagaagacgatgtggccgtgtattattgtcagcagactagacgtattcctcgtacatttggtgggggtactaagctggagattaag

SEQ ID NO: 112 - DNA, 738 bp, Artificial Sequence
  Synthesized
gacattgtgctcactcagtcacctcccagcctggccatgagcctgggaaaaagggccaccatctcctgtagagccagtgagtccgtcacaatcccggggcagcatttaatccactggtatcagcagaggcccgggcagcctccaaagcttcttattcagcgtgcgtcaaacctcgagtcgggtgtacctgccagattttctggtagcgggtcccgcactgattttacactgaccatagatccagtggaagaagacgatgtggccgtgtattattgtctgcagactagaaagattcctaggacatttggtgggggtactaagctggagattaagggaagcacgtccggctcagggaagccgggctccggcgagggaagcacgaaggggcaaattcagctggtccagagcggacctgagctgaaaaaacccggcgagactgttaagatcagttgtaaagcatctggctataccttcacccactacagcattaattgggtgaaacgggcccctggaaagggcctcaaatggatgggttggatcaataccgaaactcgggagtctacttatgcatatgacttcaaagggagattcgccttttcactcgagacatctgcctctactgcttacctccaaataaacaacctcaagtatgaagatacagccacttacttttgcgccctcgactattggagtgccatggactactggggacagggaacctccgttaccgtcagttcc

SEQ ID NO: 113 - DNA, 738 bp, Artificial Sequence
  Synthesized
caaattcagctggtccagagcggacctgagctgaaaaaacccggcgagactgttaagatcagttgtaaagcatctggctataccttcacccactacagcattaattgggtgaaacgggcccctggaaagggcctcaaatggatgggttggatcaataccgaaactcgggagtctacttatgcatatgacttcaaagggagattcgccttttcactcgagacatctgcctctactgcttacctccaaataaacaacctcaagtatgaagatacagccacttacttttgcgccctcgactattggagtgccatggactactggggacagggaacctccgttaccgtcagttccggaagcacgtccggctcagggaagccgggctccggcgagggaagcacgaagggggacattgtgctcactcagtcacctcccagcctggccatgagcctgggaaaaagggccaccatctcctgtagagccagtgagtccgtcacaatccctgggcagcatctcatcaactggtatcagcagaagcccgggcagcctccaaagcttcttattcagcgtgcgtcaaacgtcgagtcgggtgtacctgccagattttctggtagcgggtcccgcactgattttacactgaccatagatccagtggaagaagacgatgtggccgtgtattattgtctgcagactagaggtattcctcggacatttggtgggggtactaagctggagattaag

SEQ ID NO: 114 - DNA, 738 bp, Artificial Sequence
  Synthesized
gacattgtgctcactcagtcacctcccagcctggccatgagcctgggaaaaagggccaccatctcctgtagagccagtgagtccgtcacaatccaggggttgcatttaattcactggtatcagcagaagcccgggcagcctccaaagcttcttattcagagagcgtcaaacgttcagtcgggtgtacctgccagattttctggtagcgggtcccgcactgattttacactgaccatagatccagtggaagaagacgatgtggccgtgtattattgtcagcagactagacgtattcctcgtacatttggtgggggtactaagctggagattaagggaagcacgtccggctcagggaagccgggctccggcgagggaagcacgaaggggcaaattcagctggtccagagcggacctgagctgaaaaaacccggcgagactgttaagatcagttgtaaagcatctggctataccttcacccactacagcataaattgggtgaaacgggcccctggaaagggcctcaaatggatgggttggatcaataccgaatctggggagcctacttatgcatatgacttcaaagggagattcgccttttcactcgagacatctgcctctactgcttacctccaaataaacaacctcaagtatgaagatacagccacttacttttgcgccctcgactatgagagtgccatggactactggggacagggaacctccgttaccgtcagttcc

SEQ ID NO: 115 - DNA, 738 bp, Artificial Sequence
  Synthesized
caaattcagctggtccagagcggacctgagctgaaaaaacccggcgagactgttaagatcagttgtaaagcatctggctataccttcacccactacagcattaattgggtgaaacgggcccctggaaagggcctcaaatggatgggttggatcaataccgaaactcgggagtctacttatgcatatgacttcaaagggagattcgccttttcactcgagacatctgcctctactgcttacctccaaataaacaacctcaagtatgaagatacagccacttacttttgcgccctcgactattggagtgccatggactactggggacagggaacctccgttaccgtcagttccggaagcacgtccggctcagggaagccgggctccggcgagggaagcacgaagggggacattgtgctcactcagtcacctcccagcctggccatgagcctgggaaaaagggccaccatctcctgtagagccagtgagtccgtcacaatcccggggcagcatttaatccactggtatcagcagaggcccgggcagcctccaaagcttcttattcagcgtgcgtcaaacctcgagtcgggtgtacctgccagattttctggtagcgggtcccgcactgattttacactgaccatagatccagtggaagaagacgatgtggccgtgtattattgtctgcagactagaaagattcctaggacatttggtgggggtactaagctggagattaag

SEQ ID NO: 116 - DNA, 738 bp, Artificial Sequence
  Synthesized
gacattgtgctcactcagtcacctcccagcctggccatgagcctgggaaaaagggccaccatctcctgtagagccagtgagtccgtcacaatccaggggttgcatttaattcactggtatcagcagaagcccgggcagcctccaaagcttcttattcagagagcgtcaaacgttcagtcgggtgtacctgccagattttctggtagcgggtcccgcactgattttacactgaccatagatccagtggaagaagacgatgtggccgtgtattattgtcagcagactagacgtattcctcgtacatttggtgggggtactaagctggagattaagggaagcacgtccggctcagggaagccgggctccggcgagggaagcacgaaggggcaaattcagctggtccagagcggacctgagctgaaaaaacccggcgagactgttaagatcagttgtaaagcatctggctataccttcacccactacagcataaattgggtgaaacgggcccctggaaagggcctcaaatggatgggttggatcaataccgaaactagggagtctacttatgcatatgacttcaaagggagattcgccttttcactcgagacatctgcctctactgcttacctccaaataaacaacctcaagtatgaagatacagccacttacttttgcgccctcgactataagcaggccatggactactggggacagggaacctccgttaccgtcagttcc

SEQ ID NO: 117 - protein, 474 aa, Artificial Sequence
  Synthesized
QIQLVQSGPELKKPGETVKISCKASGYTFTHYSINWVKRAPGKGLKWMGWINTESGEPTYAYDFKGRFAFSLETSASTAYLQINNLKYEDTATYFCALDYESAMDYWGQGTSVTVSSGSTSGSGKPGSGEGSTKGDIVLTQSPPSLAMSLGKRATISCRASESVTIPGQHLINWYQQKPGQPPKLLIQRASNVESGVPARFSGSGSRTDFTLTIDPVEEDDVAVYYCLQTRGIPRTFGGGTKLEIKGLSGLTTTPAPRPPTPAPTIASQPLSLRPEACRPAAGGAVHTRGLDFACDIYIWAPLAGTCGVLLLSLVITLYCKASRKKAAAAAKSPFASPASSAQEEDASSCRAPSEEEGSCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 118 - protein, 474 aa, Artificial Sequence
  Synthesized
DIVLTQSPPSLAMSLGKRATISCRASESVTIPGQHLINWYQQKPGQPPKLLIQRASNVESGVPARFSGSGSRTDFTLTIDPVEEDDVAVYYCLQTRGIPRTFGGGTKLEIKGSTSGSGKPGSGEGSTKGQIQLVQSGPELKKPGETVKISCKASGYTFTHYSINWVKRAPGKGLKWMGWINTESGEPTYAYDFKGRFAFSLETSASTAYLQINNLKYEDTATYFCALDYESAMDYWGQGTSVTVSSGLSGLTTTPAPRPPTPAPTIASQPLSLRPEACRPAAGGAVHTRGLDFACDIYIWAPLAGTCGVLLLSLVITLYCKASRKKAAAAAKSPFASPASSAQEEDASSCRAPSEEEGSCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 119 - protein, 474 aa, Artificial Sequence
  Synthesized
QIQLVQSGPELKKPGETVKISCKASGYTFTHYSINWVKRAPGKGLKWMGWINTETRESTYAYDFKGRFAFSLETSASTAYLQINNLKYEDTATYFCALDYKQAMDYWGQGTSVTVSSGSTSGSGKPGSGEGSTKGDIVLTQSPPSLAMSLGKRATISCRASESVTIPGQHLIHWYQQRPGQPPKLLIQRASNLESGVPARFSGSGSRTDFTLTIDPVEEDDVAVYYCLQTRKIPRTFGGGTKLEIKGLSGLTTTPAPRPPTPAPTIASQPLSLRPEACRPAAGGAVHTRGLDFACDIYIWAPLAGTCGVLLLSLVITLYCKASRKKAAAAAKSPFASPASSAQEEDASSCRAPSEEEGSCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 120 - protein, 474 aa, Artificial Sequence
  Synthesized
DIVLTQSPPSLAMSLGKRATISCRASESVTIPGQHLIHWYQQRPGQPPKLLIQRASNLESGVPARFSGSGSRTDFTLTIDPVEEDDVAVYYCLQTRKIPRTFGGGTKLEIKGSTSGSGKPGSGEGSTKGQIQLVQSGPELKKPGETVKISCKASGYTFTHYSINWVKRAPGKGLKWMGWINTETRESTYAYDFKGRFAFSLETSASTAYLQINNLKYEDTATYFCALDYKQAMDYWGQGTSVTVSSGLSGLTTTPAPRPPTPAPTIASQPLSLRPEACRPAAGGAVHTRGLDFACDIYIWAPLAGTCGVLLLSLVITLYCKASRKKAAAAAKSPFASPASSAQEEDASSCRAPSEEEGSCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 121 - protein, 474 aa, Artificial Sequence
  Synthesized
QIQLVQSGPELKKPGETVKISCKASGYTFTHYSINWVKRAPGKGLKWMGWINTETRESTYAYDFKGRFAFSLETSASTAYLQINNLKYEDTATYFCALDYWSAMDYWGQGTSVTVSSGSTSGSGKPGSGEGSTKGDIVLTQSPPSLAMSLGKRATISCRASESVTIQGLHLIHWYQQKPGQPPKLLIQRASNVQSGVPARFSGSGSRTDFTLTIDPVEEDDVAVYYCQQTRRIPRTFGGGTKLEIKGLSGLTTTPAPRPPTPAPTIASQPLSLRPEACRPAAGGAVHTRGLDFACDIYIWAPLAGTCGVLLLSLVITLYCKASRKKAAAAAKSPFASPASSAQEEDASSCRAPSEEEGSCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 122 - protein, 474 aa, Artificial Sequence
  Synthesized
DIVLTQSPPSLAMSLGKRATISCRASESVTIQGLHLIHWYQQKPGQPPKLLIQRASNVQSGVPARFSGSGSRTDFTLTIDPVEEDDVAVYYCQQTRRIPRTFGGGTKLEIKGSTSGSGKPGSGEGSTKGQIQLVQSGPELKKPGETVKISCKASGYTFTHYSINWVKRAPGKGLKWMGWINTETRESTYAYDFKGRFAFSLETSASTAYLQINNLKYEDTATYFCALDYWSAMDYWGQGTSVTVSSGLSGLTTTPAPRPPTPAPTIASQPLSLRPEACRPAAGGAVHTRGLDFACDIYIWAPLAGTCGVLLLSLVITLYCKASRKKAAAAAKSPFASPASSAQEEDASSCRAPSEEEGSCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 123 - protein, 474 aa, Artificial Sequence
  Synthesized
QIQLVQSGPELKKPGETVKISCKASGYTFTHYSINWVKRAPGKGLKWMGWINTESGEPTYAYDFKGRFAFSLETSASTAYLQINNLKYEDTATYFCALDYESAMDYWGQGTSVTVSSGSTSGSGKPGSGEGSTKGDIVLTQSPPSLAMSLGKRATISCRASESVTIPGQHLIHWYQQRPGQPPKLLIQRASNLESGVPARFSGSGSRTDFTLTIDPVEEDDVAVYYCLQTRKIPRTFGGGTKLEIKGLSGLTTTPAPRPPTPAPTIASQPLSLRPEACRPAAGGAVHTRGLDFACDIYIWAPLAGTCGVLLLSLVITLYCKASRKKAAAAAKSPFASPASSAQEEDASSCRAPSEEEGSCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 124 - protein, 474 aa, Artificial Sequence
  Synthesized
DIVLTQSPPSLAMSLGKRATISCRASESVTIPGQHLINWYQQKPGQPPKLLIQRASNVESGVPARFSGSGSRTDFTLTIDPVEEDDVAVYYCLQTRGIPRTFGGGTKLEIKGSTSGSGKPGSGEGSTKGQIQLVQSGPELKKPGETVKISCKASGYTFTHYSINWVKRAPGKGLKWMGWINTETRESTYAYDFKGRFAFSLETSASTAYLQINNLKYEDTATYFCALDYKQAMDYWGQGTSVTVSSGLSGLTTTPAPRPPTPAPTIASQPLSLRPEACRPAAGGAVHTRGLDFACDIYIWAPLAGTCGVLLLSLVITLYCKASRKKAAAAAKSPFASPASSAQEEDASSCRAPSEEEGSCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 125 - protein, 474 aa, Artificial Sequence
  Synthesized
QIQLVQSGPELKKPGETVKISCKASGYTFTHYSINWVKRAPGKGLKWMGWINTESGEPTYAYDFKGRFAFSLETSASTAYLQINNLKYEDTATYFCALDYESAMDYWGQGTSVTVSSGSTSGSGKPGSGEGSTKGDIVLTQSPPSLAMSLGKRATISCRASESVTIQGLHLIHWYQQKPGQPPKLLIQRASNVQSGVPARFSGSGSRTDFTLTIDPVEEDDVAVYYCQQTRRIPRTFGGGTKLEIKGLSGLTTTPAPRPPTPAPTIASQPLSLRPEACRPAAGGAVHTRGLDFACDIYIWAPLAGTCGVLLLSLVITLYCKASRKKAAAAAKSPFASPASSAQEEDASSCRAPSEEEGSCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 126 - protein, 474 aa, Artificial Sequence
  Synthesized
DIVLTQSPPSLAMSLGKRATISCRASESVTIPGQHLINWYQQKPGQPPKLLIQRASNVESGVPARFSGSGSRTDFTLTIDPVEEDDVAVYYCLQTRGIPRTFGGGTKLEIKGSTSGSGKPGSGEGSTKGQIQLVQSGPELKKPGETVKISCKASGYTFTHYSINWVKRAPGKGLKWMGWINTETRESTYAYDFKGRFAFSLETSASTAYLQINNLKYEDTATYFCALDYWSAMDYWGQGTSVTVSSGLSGLTTTPAPRPPTPAPTIASQPLSLRPEACRPAAGGAVHTRGLDFACDIYIWAPLAGTCGVLLLSLVITLYCKASRKKAAAAAKSPFASPASSAQEEDASSCRAPSEEEGSCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 127 - protein, 474 aa, Artificial Sequence
  Synthesized
QIQLVQSGPELKKPGETVKISCKASGYTFTHYSINWVKRAPGKGLKWMGWINTETRESTYAYDFKGRFAFSLETSASTAYLQINNLKYEDTATYFCALDYKQAMDYWGQGTSVTVSSGSTSGSGKPGSGEGSTKGDIVLTQSPPSLAMSLGKRATISCRASESVTIPGQHLINWYQQKPGQPPKLLIQRASNVESGVPARFSGSGSRTDFTLTIDPVEEDDVAVYYCLQTRGIPRTFGGGTKLEIKGLSGLTTTPAPRPPTPAPTIASQPLSLRPEACRPAAGGAVHTRGLDFACDIYIWAPLAGTCGVLLLSLVITLYCKASRKKAAAAAKSPFASPASSAQEEDASSCRAPSEEEGSCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 128 - protein, 474 aa, Artificial Sequence
  Synthesized
DIVLTQSPPSLAMSLGKRATISCRASESVTIPGQHLIHWYQQRPGQPPKLLIQRASNLESGVPARFSGSGSRTDFTLTIDPVEEDDVAVYYCLQTRKIPRTFGGGTKLEIKGSTSGSGKPGSGEGSTKGQIQLVQSGPELKKPGETVKISCKASGYTFTHYSINWVKRAPGKGLKWMGWINTESGEPTYAYDFKGRFAFSLETSASTAYLQINNLKYEDTATYFCALDYESAMDYWGQGTSVTVSSGLSGLTTTPAPRPPTPAPTIASQPLSLRPEACRPAAGGAVHTRGLDFACDIYIWAPLAGTCGVLLLSLVITLYCKASRKKAAAAAKSPFASPASSAQEEDASSCRAPSEEEGSCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 129 - protein, 474 aa, Artificial Sequence
  Synthesized
QIQLVQSGPELKKPGETVKISCKASGYTFTHYSINWVKRAPGKGLKWMGWINTETRESTYAYDFKGRFAFSLETSASTAYLQINNLKYEDTATYFCALDYKQAMDYWGQGTSVTVSSGSTSGSGKPGSGEGSTKGDIVLTQSPPSLAMSLGKRATISCRASESVTIQGLHLIHWYQQKPGQPPKLLIQRASNVQSGVPARFSGSGSRTDFTLTIDPVEEDDVAVYYCQQTRRIPRTFGGGTKLEIKGLSGLTTTPAPRPPTPAPTIASQPLSLRPEACRPAAGGAVHTRGLDFACDIYIWAPLAGTCGVLLLSLVITLYCKASRKKAAAAAKSPFASPASSAQEEDASSCRAPSEEEGSCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 130 - protein, 474 aa, Artificial Sequence
  Synthesized
DIVLTQSPPSLAMSLGKRATISCRASESVTIPGQHLIHWYQQRPGQPPKLLIQRASNLESGVPARFSGSGSRTDFTLTIDPVEEDDVAVYYCLQTRKIPRTFGGGTKLEIKGSTSGSGKPGSGEGSTKGQIQLVQSGPELKKPGETVKISCKASGYTFTHYSINWVKRAPGKGLKWMGWINTETRESTYAYDFKGRFAFSLETSASTAYLQINNLKYEDTATYFCALDYWSAMDYWGQGTSVTVSSGLSGLTTTPAPRPPTPAPTIASQPLSLRPEACRPAAGGAVHTRGLDFACDIYIWAPLAGTCGVLLLSLVITLYCKASRKKAAAAAKSPFASPASSAQEEDASSCRAPSEEEGSCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 131 - protein, 474 aa, Artificial Sequence
  Synthesized
QIQLVQSGPELKKPGETVKISCKASGYTFTHYSINWVKRAPGKGLKWMGWINTETRESTYAYDFKGRFAFSLETSASTAYLQINNLKYEDTATYFCALDYWSAMDYWGQGTSVTVSSGSTSGSGKPGSGEGSTKGDIVLTQSPPSLAMSLGKRATISCRASESVTIPGQHLINWYQQKPGQPPKLLIQRASNVESGVPARFSGSGSRTDFTLTIDPVEEDDVAVYYCLQTRGIPRTFGGGTKLEIKGLSGLTTTPAPRPPTPAPTIASQPLSLRPEACRPAAGGAVHTRGLDFACDIYIWAPLAGTCGVLLLSLVITLYCKASRKKAAAAAKSPFASPASSAQEEDASSCRAPSEEEGSCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 132 - protein, 474 aa, Artificial Sequence
  Synthesized
DIVLTQSPPSLAMSLGKRATISCRASESVTIQGLHLIHWYQQKPGQPPKLLIQRASNVQSGVPARFSGSGSRTDFTLTIDPVEEDDVAVYYCQQTRRIPRTFGGGTKLEIKGSTSGSGKPGSGEGSTKGQIQLVQSGPELKKPGETVKISCKASGYTFTHYSINWVKRAPGKGLKWMGWINTESGEPTYAYDFKGRFAFSLETSASTAYLQINNLKYEDTATYFCALDYESAMDYWGQGTSVTVSSGLSGLTTTPAPRPPTPAPTIASQPLSLRPEACRPAAGGAVHTRGLDFACDIYIWAPLAGTCGVLLLSLVITLYCKASRKKAAAAAKSPFASPASSAQEEDASSCRAPSEEEGSCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 133 - protein, 474 aa, Artificial Sequence
  Synthesized
QIQLVQSGPELKKPGETVKISCKASGYTFTHYSINWVKRAPGKGLKWMGWINTETRESTYAYDFKGRFAFSLETSASTAYLQINNLKYEDTATYFCALDYWSAMDYWGQGTSVTVSSGSTSGSGKPGSGEGSTKGDIVLTQSPPSLAMSLGKRATISCRASESVTIPGQHLIHWYQQRPGQPPKLLIQRASNLESGVPARFSGSGSRTDFTLTIDPVEEDDVAVYYCLQTRKIPRTFGGGTKLEIKGLSGLTTTPAPRPPTPAPTIASQPLSLRPEACRPAAGGAVHTRGLDFACDIYIWAPLAGTCGVLLLSLVITLYCKASRKKAAAAAKSPFASPASSAQEEDASSCRAPSEEEGSCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 134 - protein, 474 aa, Artificial Sequence
  Synthesized
DIVLTQSPPSLAMSLGKRATISCRASESVTIQGLHLIHWYQQKPGQPPKLLIQRASNVQSGVPARFSGSGSRTDFTLTIDPVEEDDVAVYYCQQTRRIPRTFGGGTKLEIKGSTSGSGKPGSGEGSTKGQIQLVQSGPELKKPGETVKISCKASGYTFTHYSINWVKRAPGKGLKWMGWINTETRESTYAYDFKGRFAFSLETSASTAYLQINNLKYEDTATYFCALDYKQAMDYWGQGTSVTVSSGLSGLTTTPAPRPPTPAPTIASQPLSLRPEACRPAAGGAVHTRGLDFACDIYIWAPLAGTCGVLLLSLVITLYCKASRKKAAAAAKSPFASPASSAQEEDASSCRAPSEEEGSCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 135 - DNA, 1422 bp, Artificial Sequence
  Synthesized
caaattcagctggtccagagcggacctgagctgaaaaaacccggcgagactgttaagatcagttgtaaagcatctggctataccttcacccactacagcataaattgggtgaaacgggcccctggaaagggcctcaaatggatgggttggatcaataccgaatctggggagcctacttatgcatatgacttcaaagggagattcgccttttcactcgagacatctgcctctactgcttacctccaaataaacaacctcaagtatgaagatacagccacttacttttgcgccctcgactatgagagtgccatggactactggggacagggaacctccgttaccgtcagttccggaagcacgtccggctcagggaagccgggctccggcgagggaagcacgaagggggacattgtgctcactcagtcacctcccagcctggccatgagcctgggaaaaagggccaccatctcctgtagagccagtgagtccgtcacaatccctgggcagcatctcatcaactggtatcagcagaagcccgggcagcctccaaagcttcttattcagcgtgcgtcaaacgtcgagtcgggtgtacctgccagattttctggtagcgggtcccgcactgattttacactgaccatagatccagtggaagaagacgatgtggccgtgtattattgtctgcagactagaggtattcctcggacatttggtgggggtactaagctggagattaagggcctgagcggcctgactactaccccagctccacgtcctcccacgccagctccaacgatagcaagtcagcccttatctcttcgccctgaggcttgcaggcccgcggcgggcggcgccgttcacacgcgaggactagacttcgcctgcgacatctacatctgggcaccactagccgggacttgcggagtgttgttgttgagcttggtaataacgctctactgcaaagcgagccgcaaaaaagcggcggcggcggcgaaaagcccgtttgcgagcccggcgagcagcgcgcaggaagaagatgcgagcagctgccgcgcgccgagcgaagaagaaggcagctgcgaactgagagtgaagttctctcgctccgcggacgcacccgcttaccagcagggtcagaaccagctatacaacgagttaaacctggggcgccgggaggagtacgacgtgttagacaagcgtagaggtagggacccggagatgggaggcaagcctcggagaaagaacccccaggagggcctgtacaacgaactccagaaggacaagatggctgaggcgtactcggagattggtatgaagggcgagagacgtcgcggaaagggacacgacggcttataccaggggctttccaccgcgaccaaggacacatacgacgcgctgcacatgcaagccttaccacctcga

SEQ ID NO: 136 - DNA, 1422 bp, Artificial Sequence
  Synthesized
gacattgtgctcactcagtcacctcccagcctggccatgagcctgggaaaaagggccaccatctcctgtagagccagtgagtccgtcacaatccctgggcagcatctcatcaactggtatcagcagaagcccgggcagcctccaaagcttcttattcagcgtgcgtcaaacgtcgagtcgggtgtacctgccagattttctggtagcgggtcccgcactgattttacactgaccatagatccagtggaagaagacgatgtggccgtgtattattgtctgcagactagaggtattcctcggacatttggtgggggtactaagctggagattaagggaagcacgtccggctcagggaagccgggctccggcgagggaagcacgaaggggcaaattcagctggtccagagcggacctgagctgaaaaaacccggcgagactgttaagatcagttgtaaagcatctggctataccttcacccactacagcataaattgggtgaaacgggcccctggaaagggcctcaaatggatgggttggatcaataccgaatctggggagcctacttatgcatatgacttcaaagggagattcgccttttcactcgagacatctgcctctactgcttacctccaaataaacaacctcaagtatgaagatacagccacttacttttgcgccctcgactatgagagtgccatggactactggggacagggaacctccgttaccgtcagttccggcctgagcggcctgactactaccccagctccacgtcctcccacgccagctccaacgatagcaagtcagcccttatctcttcgccctgaggcttgcaggcccgcggcgggcggcgccgttcacacgcgaggactagacttcgcctgcgacatctacatctgggcaccactagccgggacttgcggagtgttgttgttgagcttggtaataacgctctactgcaaagcgagccgcaaaaaagcggcggcggcggcgaaaagcccgtttgcgagcccggcgagcagcgcgcaggaagaagatgcgagcagctgccgcgcgccgagcgaagaagaaggcagctgcgaactgagagtgaagttctctcgctccgcggacgcacccgcttaccagcagggtcagaaccagctatacaacgagttaaacctggggcgccgggaggagtacgacgtgttagacaagcgtagaggtagggacccggagatgggaggcaagcctcggagaaagaacccccaggagggcctgtacaacgaactccagaaggacaagatggctgaggcgtactcggagattggtatgaagggcgagagacgtcgcggaaagggacacgacggcttataccaggggctttccaccgcgaccaaggacacatacgacgcgctgcacatgcaagccttaccacctcga

SEQ ID NO: 137 - DNA, 1422 bp, Artificial Sequence
  Synthesized
caaattcagctggtccagagcggacctgagctgaaaaaacccggcgagactgttaagatcagttgtaaagcatctggctataccttcacccactacagcataaattgggtgaaacgggcccctggaaagggcctcaaatggatgggttggatcaataccgaaactagggagtctacttatgcatatgacttcaaagggagattcgccttttcactcgagacatctgcctctactgcttacctccaaataaacaacctcaagtatgaagatacagccacttacttttgcgccctcgactataagcaggccatggactactggggacagggaacctccgttaccgtcagttccggaagcacgtccggctcagggaagccgggctccggcgagggaagcacgaagggggacattgtgctcactcagtcacctcccagcctggccatgagcctgggaaaaagggccaccatctcctgtagagccagtgagtccgtcacaatcccggggcagcatttaatccactggtatcagcagaggcccgggcagcctccaaagcttcttattcagcgtgcgtcaaacctcgagtcgggtgtacctgccagattttctggtagcgggtcccgcactgattttacactgaccatagatccagtggaagaagacgatgtggccgtgtattattgtctgcagactagaaagattcctaggacatttggtgggggtactaagctggagattaagggcctgagcggcctgactactaccccagctccacgtcctcccacgccagctccaacgatagcaagtcagcccttatctcttcgccctgaggcttgcaggcccgcggcgggcggcgccgttcacacgcgaggactagacttcgcctgcgacatctacatctgggcaccactagccgggacttgcggagtgttgttgttgagcttggtaataacgctctactgcaaagcgagccgcaaaaaagcggcggcggcggcgaaaagcccgtttgcgagcccggcgagcagcgcgcaggaagaagatgcgagcagctgccgcgcgccgagcgaagaagaaggcagctgcgaactgagagtgaagttctctcgctccgcggacgcacccgcttaccagcagggtcagaaccagctatacaacgagttaaacctggggcgccgggaggagtacgacgtgttagacaagcgtagaggtagggacccggagatgggaggcaagcctcggagaaagaacccccaggagggcctgtacaacgaactccagaaggacaagatggctgaggcgtactcggagattggtatgaagggcgagagacgtcgcggaaagggacacgacggcttataccaggggctttccaccgcgaccaaggacacatacgacgcgctgcacatgcaagccttaccacctcga

SEQ ID NO: 138 - DNA, 1422 bp, Artificial Sequence
  Synthesized
gacattgtgctcactcagtcacctcccagcctggccatgagcctgggaaaaagggccaccatctcctgtagagccagtgagtccgtcacaatcccggggcagcatttaatccactggtatcagcagaggcccgggcagcctccaaagcttcttattcagcgtgcgtcaaacctcgagtcgggtgtacctgccagattttctggtagcgggtcccgcactgattttacactgaccatagatccagtggaagaagacgatgtggccgtgtattattgtctgcagactagaaagattcctaggacatttggtgggggtactaagctggagattaagggaagcacgtccggctcagggaagccgggctccggcgagggaagcacgaaggggcaaattcagctggtccagagcggacctgagctgaaaaaacccggcgagactgttaagatcagttgtaaagcatctggctataccttcacccactacagcataaattgggtgaaacgggcccctggaaagggcctcaaatggatgggttggatcaataccgaaactagggagtctacttatgcatatgacttcaaagggagattcgccttttcactcgagacatctgcctctactgcttacctccaaataaacaacctcaagtatgaagatacagccacttacttttgcgccctcgactataagcaggccatggactactggggacagggaacctccgttaccgtcagttccggcctgagcggcctgactactaccccagctccacgtcctcccacgccagctccaacgatagcaagtcagcccttatctcttcgccctgaggcttgcaggcccgcggcgggcggcgccgttcacacgcgaggactagacttcgcctgcgacatctacatctgggcaccactagccgggacttgcggagtgttgttgttgagcttggtaataacgctctactgcaaagcgagccgcaaaaaagcggcggcggcggcgaaaagcccgtttgcgagcccggcgagcagcgcgcaggaagaagatgcgagcagctgccgcgcgccgagcgaagaagaaggcagctgcgaactgagagtgaagttctctcgctccgcggacgcacccgcttaccagcagggtcagaaccagctatacaacgagttaaacctggggcgccgggaggagtacgacgtgttagacaagcgtagaggtagggacccggagatgggaggcaagcctcggagaaagaacccccaggagggcctgtacaacgaactccagaaggacaagatggctgaggcgtactcggagattggtatgaagggcgagagacgtcgcggaaagggacacgacggcttataccaggggctttccaccgcgaccaaggacacatacgacgcgctgcacatgcaagccttaccacctcga

SEQ ID NO: 139 - DNA, 1422 bp, Artificial Sequence
  Synthesized
caaattcagctggtccagagcggacctgagctgaaaaaacccggcgagactgttaagatcagttgtaaagcatctggctataccttcacccactacagcattaattgggtgaaacgggcccctggaaagggcctcaaatggatgggttggatcaataccgaaactcgggagtctacttatgcatatgacttcaaagggagattcgccttttcactcgagacatctgcctctactgcttacctccaaataaacaacctcaagtatgaagatacagccacttacttttgcgccctcgactattggagtgccatggactactggggacagggaacctccgttaccgtcagttccggaagcacgtccggctcagggaagccgggctccggcgagggaagcacgaagggggacattgtgctcactcagtcacctcccagcctggccatgagcctgggaaaaagggccaccatctcctgtagagccagtgagtccgtcacaatccaggggttgcatttaattcactggtatcagcagaagcccgggcagcctccaaagcttcttattcagagagcgtcaaacgttcagtcgggtgtacctgccagattttctggtagcgggtcccgcactgattttacactgaccatagatccagtggaagaagacgatgtggccgtgtattattgtcagcagactagacgtattcctcgtacatttggtgggggtactaagctggagattaagggcctgagcggcctgactactaccccagctccacgtcctcccacgccagctccaacgatagcaagtcagcccttatctcttcgccctgaggcttgcaggcccgcggcgggcggcgccgttcacacgcgaggactagacttcgcctgcgacatctacatctgggcaccactagccgggacttgcggagtgttgttgttgagcttggtaataacgctctactgcaaagcgagccgcaaaaaagcggcggcggcggcgaaaagcccgtttgcgagcccggcgagcagcgcgcaggaagaagatgcgagcagctgccgcgcgccgagcgaagaagaaggcagctgcgaactgagagtgaagttctctcgctccgcggacgcacccgcttaccagcagggtcagaaccagctatacaacgagttaaacctggggcgccgggaggagtacgacgtgttagacaagcgtagaggtagggacccggagatgggaggcaagcctcggagaaagaacccccaggagggcctgtacaacgaactccagaaggacaagatggctgaggcgtactcggagattggtatgaagggcgagagacgtcgcggaaagggacacgacggcttataccaggggctttccaccgcgaccaaggacacatacgacgcgctgcacatgcaagccttaccacctcga

SEQ ID NO: 140 - DNA, 1422 bp, Artificial Sequence
  Synthesized
gacattgtgctcactcagtcacctcccagcctggccatgagcctgggaaaaagggccaccatctcctgtagagccagtgagtccgtcacaatccaggggttgcatttaattcactggtatcagcagaagcccgggcagcctccaaagcttcttattcagagagcgtcaaacgttcagtcgggtgtacctgccagattttctggtagcgggtcccgcactgattttacactgaccatagatccagtggaagaagacgatgtggccgtgtattattgtcagcagactagacgtattcctcgtacatttggtgggggtactaagctggagattaagggaagcacgtccggctcagggaagccgggctccggcgagggaagcacgaaggggcaaattcagctggtccagagcggacctgagctgaaaaaacccggcgagactgttaagatcagttgtaaagcatctggctataccttcacccactacagcattaattgggtgaaacgggcccctggaaagggcctcaaatggatgggttggatcaataccgaaactcgggagtctacttatgcatatgacttcaaagggagattcgccttttcactcgagacatctgcctctactgcttacctccaaataaacaacctcaagtatgaagatacagccacttacttttgcgccctcgactattggagtgccatggactactggggacagggaacctccgttaccgtcagttccggcctgagcggcctgactactaccccagctccacgtcctcccacgccagctccaacgatagcaagtcagcccttatctcttcgccctgaggcttgcaggcccgcggcgggcggcgccgttcacacgcgaggactagacttcgcctgcgacatctacatctgggcaccactagccgggacttgcggagtgttgttgttgagcttggtaataacgctctactgcaaagcgagccgcaaaaaagcggcggcggcggcgaaaagcccgtttgcgagcccggcgagcagcgcgcaggaagaagatgcgagcagctgccgcgcgccgagcgaagaagaaggcagctgcgaactgagagtgaagttctctcgctccgcggacgcacccgcttaccagcagggtcagaaccagctatacaacgagttaaacctggggcgccgggaggagtacgacgtgttagacaagcgtagaggtagggacccggagatgggaggcaagcctcggagaaagaacccccaggagggcctgtacaacgaactccagaaggacaagatggctgaggcgtactcggagattggtatgaagggcgagagacgtcgcggaaagggacacgacggcttataccaggggctttccaccgcgaccaaggacacatacgacgcgctgcacatgcaagccttaccacctcga

SEQ ID NO: 141 - DNA, 1422 bp, Artificial Sequence
  Synthesized
caaattcagctggtccagagcggacctgagctgaaaaaacccggcgagactgttaagatcagttgtaaagcatctggctataccttcacccactacagcataaattgggtgaaacgggcccctggaaagggcctcaaatggatgggttggatcaataccgaatctggggagcctacttatgcatatgacttcaaagggagattcgccttttcactcgagacatctgcctctactgcttacctccaaataaacaacctcaagtatgaagatacagccacttacttttgcgccctcgactatgagagtgccatggactactggggacagggaacctccgttaccgtcagttccggaagcacgtccggctcagggaagccgggctccggcgagggaagcacgaagggggacattgtgctcactcagtcacctcccagcctggccatgagcctgggaaaaagggccaccatctcctgtagagccagtgagtccgtcacaatcccggggcagcatttaatccactggtatcagcagaggcccgggcagcctccaaagcttcttattcagcgtgcgtcaaacctcgagtcgggtgtacctgccagattttctggtagcgggtcccgcactgattttacactgaccatagatccagtggaagaagacgatgtggccgtgtattattgtctgcagactagaaagattcctaggacatttggtgggggtactaagctggagattaagggcctgagcggcctgactactaccccagctccacgtcctcccacgccagctccaacgatagcaagtcagcccttatctcttcgccctgaggcttgcaggcccgcggcgggcggcgccgttcacacgcgaggactagacttcgcctgcgacatctacatctgggcaccactagccgggacttgcggagtgttgttgttgagcttggtaataacgctctactgcaaagcgagccgcaaaaaagcggcggcggcggcgaaaagcccgtttgcgagcccggcgagcagcgcgcaggaagaagatgcgagcagctgccgcgcgccgagcgaagaagaaggcagctgcgaactgagagtgaagttctctcgctccgcggacgcacccgcttaccagcagggtcagaaccagctatacaacgagttaaacctggggcgccgggaggagtacgacgtgttagacaagcgtagaggtagggacccggagatgggaggcaagcctcggagaaagaacccccaggagggcctgtacaacgaactccagaaggacaagatggctgaggcgtactcggagattggtatgaagggcgagagacgtcgcggaaagggacacgacggcttataccaggggctttccaccgcgaccaaggacacatacgacgcgctgcacatgcaagccttaccacctcga

SEQ ID NO: 142 - DNA, 1422 bp, Artificial Sequence
  Synthesized
gacattgtgctcactcagtcacctcccagcctggccatgagcctgggaaaaagggccaccatctcctgtagagccagtgagtccgtcacaatccctgggcagcatctcatcaactggtatcagcagaagcccgggcagcctccaaagcttcttattcagcgtgcgtcaaacgtcgagtcgggtgtacctgccagattttctggtagcgggtcccgcactgattttacactgaccatagatccagtggaagaagacgatgtggccgtgtattattgtctgcagactagaggtattcctcggacatttggtgggggtactaagctggagattaagggaagcacgtccggctcagggaagccgggctccggcgagggaagcacgaaggggcaaattcagctggtccagagcggacctgagctgaaaaaacccggcgagactgttaagatcagttgtaaagcatctggctataccttcacccactacagcataaattgggtgaaacgggcccctggaaagggcctcaaatggatgggttggatcaataccgaaactagggagtctacttatgcatatgacttcaaagggagattcgccttttcactcgagacatctgcctctactgcttacctccaaataaacaacctcaagtatgaagatacagccacttacttttgcgccctcgactataagcaggccatggactactggggacagggaacctccgttaccgtcagttccggcctgagcggcctgactactaccccagctccacgtcctcccacgccagctccaacgatagcaagtcagcccttatctcttcgccctgaggcttgcaggcccgcggcgggcggcgccgttcacacgcgaggactagacttcgcctgcgacatctacatctgggcaccactagccgggacttgcggagtgttgttgttgagcttggtaataacgctctactgcaaagcgagccgcaaaaaagcggcggcggcggcgaaaagcccgtttgcgagcccggcgagcagcgcgcaggaagaagatgcgagcagctgccgcgcgccgagcgaagaagaaggcagctgcgaactgagagtgaagttctctcgctccgcggacgcacccgcttaccagcagggtcagaaccagctatacaacgagttaaacctggggcgccgggaggagtacgacgtgttagacaagcgtagaggtagggacccggagatgggaggcaagcctcggagaaagaacccccaggagggcctgtacaacgaactccagaaggacaagatggctgaggcgtactcggagattggtatgaagggcgagagacgtcgcggaaagggacacgacggcttataccaggggctttccaccgcgaccaaggacacatacgacgcgctgcacatgcaagccttaccacctcga

SEQ ID NO: 143 - DNA, 1422 bp, Artificial Sequence
  Synthesized
caaattcagctggtccagagcggacctgagctgaaaaaacccggcgagactgttaagatcagttgtaaagcatctggctataccttcacccactacagcataaattgggtgaaacgggcccctggaaagggcctcaaatggatgggttggatcaataccgaatctggggagcctacttatgcatatgacttcaaagggagattcgccttttcactcgagacatctgcctctactgcttacctccaaataaacaacctcaagtatgaagatacagccacttacttttgcgccctcgactatgagagtgccatggactactggggacagggaacctccgttaccgtcagttccggaagcacgtccggctcagggaagccgggctccggcgagggaagcacgaagggggacattgtgctcactcagtcacctcccagcctggccatgagcctgggaaaaagggccaccatctcctgtagagccagtgagtccgtcacaatccaggggttgcatttaattcactggtatcagcagaagcccgggcagcctccaaagcttcttattcagagagcgtcaaacgttcagtcgggtgtacctgccagattttctggtagcgggtcccgcactgattttacactgaccatagatccagtggaagaagacgatgtggccgtgtattattgtcagcagactagacgtattcctcgtacatttggtgggggtactaagctggagattaagggcctgagcggcctgactactaccccagctccacgtcctcccacgccagctccaacgatagcaagtcagcccttatctcttcgccctgaggcttgcaggcccgcggcgggcggcgccgttcacacgcgaggactagacttcgcctgcgacatctacatctgggcaccactagccgggacttgcggagtgttgttgttgagcttggtaataacgctctactgcaaagcgagccgcaaaaaagcggcggcggcggcgaaaagcccgtttgcgagcccggcgagcagcgcgcaggaagaagatgcgagcagctgccgcgcgccgagcgaagaagaaggcagctgcgaactgagagtgaagttctctcgctccgcggacgcacccgcttaccagcagggtcagaaccagctatacaacgagttaaacctggggcgccgggaggagtacgacgtgttagacaagcgtagaggtagggacccggagatgggaggcaagcctcggagaaagaacccccaggagggcctgtacaacgaactccagaaggacaagatggctgaggcgtactcggagattggtatgaagggcgagagacgtcgcggaaagggacacgacggcttataccaggggctttccaccgcgaccaaggacacatacgacgcgctgcacatgcaagccttaccacctcga

SEQ ID NO: 144 - DNA, 1422 bp, Artificial Sequence
  Synthesized
gacattgtgctcactcagtcacctcccagcctggccatgagcctgggaaaaagggccaccatctcctgtagagccagtgagtccgtcacaatccctgggcagcatctcatcaactggtatcagcagaagcccgggcagcctccaaagcttcttattcagcgtgcgtcaaacgtcgagtcgggtgtacctgccagattttctggtagcgggtcccgcactgattttacactgaccatagatccagtggaagaagacgatgtggccgtgtattattgtctgcagactagaggtattcctcggacatttggtgggggtactaagctggagattaagggaagcacgtccggctcagggaagccgggctccggcgagggaagcacgaaggggcaaattcagctggtccagagcggacctgagctgaaaaaacccggcgagactgttaagatcagttgtaaagcatctggctataccttcacccactacagcattaattgggtgaaacgggcccctggaaagggcctcaaatggatgggttggatcaataccgaaactcgggagtctacttatgcatatgacttcaaagggagattcgccttttcactcgagacatctgcctctactgcttacctccaaataaacaacctcaagtatgaagatacagccacttacttttgcgccctcgactattggagtgccatggactactggggacagggaacctccgttaccgtcagttccggcctgagcggcctgactactaccccagctccacgtcctcccacgccagctccaacgatagcaagtcagcccttatctcttcgccctgaggcttgcaggcccgcggcgggcggcgccgttcacacgcgaggactagacttcgcctgcgacatctacatctgggcaccactagccgggacttgcggagtgttgttgttgagcttggtaataacgctctactgcaaagcgagccgcaaaaaagcggcggcggcggcgaaaagcccgtttgcgagcccggcgagcagcgcgcaggaagaagatgcgagcagctgccgcgcgccgagcgaagaagaaggcagctgcgaactgagagtgaagttctctcgctccgcggacgcacccgcttaccagcagggtcagaaccagctatacaacgagttaaacctggggcgccgggaggagtacgacgtgttagacaagcgtagaggtagggacccggagatgggaggcaagcctcggagaaagaacccccaggagggcctgtacaacgaactccagaaggacaagatggctgaggcgtactcggagattggtatgaagggcgagagacgtcgcggaaagggacacgacggcttataccaggggctttccaccgcgaccaaggacacatacgacgcgctgcacatgcaagccttaccacctcga

SEQ ID NO: 145 - DNA, 1422 bp, Artificial Sequence
  Synthesized
caaattcagctggtccagagcggacctgagctgaaaaaacccggcgagactgttaagatcagttgtaaagcatctggctataccttcacccactacagcataaattgggtgaaacgggcccctggaaagggcctcaaatggatgggttggatcaataccgaaactagggagtctacttatgcatatgacttcaaagggagattcgccttttcactcgagacatctgcctctactgcttacctccaaataaacaacctcaagtatgaagatacagccacttacttttgcgccctcgactataagcaggccatggactactggggacagggaacctccgttaccgtcagttccggaagcacgtccggctcagggaagccgggctccggcgagggaagcacgaagggggacattgtgctcactcagtcacctcccagcctggccatgagcctgggaaaaagggccaccatctcctgtagagccagtgagtccgtcacaatccctgggcagcatctcatcaactggtatcagcagaagcccgggcagcctccaaagcttcttattcagcgtgcgtcaaacgtcgagtcgggtgtacctgccagattttctggtagcgggtcccgcactgattttacactgaccatagatccagtggaagaagacgatgtggccgtgtattattgtctgcagactagaggtattcctcggacatttggtgggggtactaagctggagattaagggcctgagcggcctgactactaccccagctccacgtcctcccacgccagctccaacgatagcaagtcagcccttatctcttcgccctgaggcttgcaggcccgcggcgggcggcgccgttcacacgcgaggactagacttcgcctgcgacatctacatctgggcaccactagccgggacttgcggagtgttgttgttgagcttggtaataacgctctactgcaaagcgagccgcaaaaaagcggcggcggcggcgaaaagcccgtttgcgagcccggcgagcagcgcgcaggaagaagatgcgagcagctgccgcgcgccgagcgaagaagaaggcagctgcgaactgagagtgaagttctctcgctccgcggacgcacccgcttaccagcagggtcagaaccagctatacaacgagttaaacctggggcgccgggaggagtacgacgtgttagacaagcgtagaggtagggacccggagatgggaggcaagcctcggagaaagaacccccaggagggcctgtacaacgaactccagaaggacaagatggctgaggcgtactcggagattggtatgaagggcgagagacgtcgcggaaagggacacgacggcttataccaggggctttccaccgcgaccaaggacacatacgacgcgctgcacatgcaagccttaccacctcga

SEQ ID NO: 146 - DNA, 1422 bp, Artificial Sequence
  Synthesized
gacattgtgctcactcagtcacctcccagcctggccatgagcctgggaaaaagggccaccatctcctgtagagccagtgagtccgtcacaatcccggggcagcatttaatccactggtatcagcagaggcccgggcagcctccaaagcttcttattcagcgtgcgtcaaacctcgagtcgggtgtacctgccagattttctggtagcgggtcccgcactgattttacactgaccatagatccagtggaagaagacgatgtggccgtgtattattgtctgcagactagaaagattcctaggacatttggtgggggtactaagctggagattaagggaagcacgtccggctcagggaagccgggctccggcgagggaagcacgaaggggcaaattcagctggtccagagcggacctgagctgaaaaaacccggcgagactgttaagatcagttgtaaagcatctggctataccttcacccactacagcataaattgggtgaaacgggcccctggaaagggcctcaaatggatgggttggatcaataccgaatctggggagcctacttatgcatatgacttcaaagggagattcgccttttcactcgagacatctgcctctactgcttacctccaaataaacaacctcaagtatgaagatacagccacttacttttgcgccctcgactatgagagtgccatggactactggggacagggaacctccgttaccgtcagttccggcctgagcggcctgactactaccccagctccacgtcctcccacgccagctccaacgatagcaagtcagcccttatctcttcgccctgaggcttgcaggcccgcggcgggcggcgccgttcacacgcgaggactagacttcgcctgcgacatctacatctgggcaccactagccgggacttgcggagtgttgttgttgagcttggtaataacgctctactgcaaagcgagccgcaaaaaagcggcggcggcggcgaaaagcccgtttgcgagcccggcgagcagcgcgcaggaagaagatgcgagcagctgccgcgcgccgagcgaagaagaaggcagctgcgaactgagagtgaagttctctcgctccgcggacgcacccgcttaccagcagggtcagaaccagctatacaacgagttaaacctggggcgccgggaggagtacgacgtgttagacaagcgtagaggtagggacccggagatgggaggcaagcctcggagaaagaacccccaggagggcctgtacaacgaactccagaaggacaagatggctgaggcgtactcggagattggtatgaagggcgagagacgtcgcggaaagggacacgacggcttataccaggggctttccaccgcgaccaaggacacatacgacgcgctgcacatgcaagccttaccacctcga

SEQ ID NO: 147 - DNA, 1422 bp, Artificial Sequence
  Synthesized
caaattcagctggtccagagcggacctgagctgaaaaaacccggcgagactgttaagatcagttgtaaagcatctggctataccttcacccactacagcataaattgggtgaaacgggcccctggaaagggcctcaaatggatgggttggatcaataccgaaactagggagtctacttatgcatatgacttcaaagggagattcgccttttcactcgagacatctgcctctactgcttacctccaaataaacaacctcaagtatgaagatacagccacttacttttgcgccctcgactataagcaggccatggactactggggacagggaacctccgttaccgtcagttccggaagcacgtccggctcagggaagccgggctccggcgagggaagcacgaagggggacattgtgctcactcagtcacctcccagcctggccatgagcctgggaaaaagggccaccatctcctgtagagccagtgagtccgtcacaatccaggggttgcatttaattcactggtatcagcagaagcccgggcagcctccaaagcttcttattcagagagcgtcaaacgttcagtcgggtgtacctgccagattttctggtagcgggtcccgcactgattttacactgaccatagatccagtggaagaagacgatgtggccgtgtattattgtcagcagactagacgtattcctcgtacatttggtgggggtactaagctggagattaagggcctgagcggcctgactactaccccagctccacgtcctcccacgccagctccaacgatagcaagtcagcccttatctcttcgccctgaggcttgcaggcccgcggcgggcggcgccgttcacacgcgaggactagacttcgcctgcgacatctacatctgggcaccactagccgggacttgcggagtgttgttgttgagcttggtaataacgctctactgcaaagcgagccgcaaaaaagcggcggcggcggcgaaaagcccgtttgcgagcccggcgagcagcgcgcaggaagaagatgcgagcagctgccgcgcgccgagcgaagaagaaggcagctgcgaactgagagtgaagttctctcgctccgcggacgcacccgcttaccagcagggtcagaaccagctatacaacgagttaaacctggggcgccgggaggagtacgacgtgttagacaagcgtagaggtagggacccggagatgggaggcaagcctcggagaaagaacccccaggagggcctgtacaacgaactccagaaggacaagatggctgaggcgtactcggagattggtatgaagggcgagagacgtcgcggaaagggacacgacggcttataccaggggctttccaccgcgaccaaggacacatacgacgcgctgcacatgcaagccttaccacctcga

SEQ ID NO: 148 - DNA, 1422 bp, Artificial Sequence
  Synthesized
gacattgtgctcactcagtcacctcccagcctggccatgagcctgggaaaaagggccaccatctcctgtagagccagtgagtccgtcacaatcccggggcagcatttaatccactggtatcagcagaggcccgggcagcctccaaagcttcttattcagcgtgcgtcaaacctcgagtcgggtgtacctgccagattttctggtagcgggtcccgcactgattttacactgaccatagatccagtggaagaagacgatgtggccgtgtattattgtctgcagactagaaagattcctaggacatttggtgggggtactaagctggagattaagggaagcacgtccggctcagggaagccgggctccggcgagggaagcacgaaggggcaaattcagctggtccagagcggacctgagctgaaaaaacccggcgagactgttaagatcagttgtaaagcatctggctataccttcacccactacagcattaattgggtgaaacgggcccctggaaagggcctcaaatggatgggttggatcaataccgaaactcgggagtctacttatgcatatgacttcaaagggagattcgccttttcactcgagacatctgcctctactgcttacctccaaataaacaacctcaagtatgaagatacagccacttacttttgcgccctcgactattggagtgccatggactactggggacagggaacctccgttaccgtcagttccggcctgagcggcctgactactaccccagctccacgtcctcccacgccagctccaacgatagcaagtcagcccttatctcttcgccctgaggcttgcaggcccgcggcgggcggcgccgttcacacgcgaggactagacttcgcctgcgacatctacatctgggcaccactagccgggacttgcggagtgttgttgttgagcttggtaataacgctctactgcaaagcgagccgcaaaaaagcggcggcggcggcgaaaagcccgtttgcgagcccggcgagcagcgcgcaggaagaagatgcgagcagctgccgcgcgccgagcgaagaagaaggcagctgcgaactgagagtgaagttctctcgctccgcggacgcacccgcttaccagcagggtcagaaccagctatacaacgagttaaacctggggcgccgggaggagtacgacgtgttagacaagcgtagaggtagggacccggagatgggaggcaagcctcggagaaagaacccccaggagggcctgtacaacgaactccagaaggacaagatggctgaggcgtactcggagattggtatgaagggcgagagacgtcgcggaaagggacacgacggcttataccaggggctttccaccgcgaccaaggacacatacgacgcgctgcacatgcaagccttaccacctcga

SEQ ID NO: 149 - DNA, 1422 bp, Artificial Sequence
  Synthesized
caaattcagctggtccagagcggacctgagctgaaaaaacccggcgagactgttaagatcagttgtaaagcatctggctataccttcacccactacagcattaattgggtgaaacgggcccctggaaagggcctcaaatggatgggttggatcaataccgaaactcgggagtctacttatgcatatgacttcaaagggagattcgccttttcactcgagacatctgcctctactgcttacctccaaataaacaacctcaagtatgaagatacagccacttacttttgcgccctcgactattggagtgccatggactactggggacagggaacctccgttaccgtcagttccggaagcacgtccggctcagggaagccgggctccggcgagggaagcacgaagggggacattgtgctcactcagtcacctcccagcctggccatgagcctgggaaaaagggccaccatctcctgtagagccagtgagtccgtcacaatccctgggcagcatctcatcaactggtatcagcagaagcccgggcagcctccaaagcttcttattcagcgtgcgtcaaacgtcgagtcgggtgtacctgccagattttctggtagcgggtcccgcactgattttacactgaccatagatccagtggaagaagacgatgtggccgtgtattattgtctgcagactagaggtattcctcggacatttggtgggggtactaagctggagattaagggcctgagcggcctgactactaccccagctccacgtcctcccacgccagctccaacgatagcaagtcagcccttatctcttcgccctgaggcttgcaggcccgcggcgggcggcgccgttcacacgcgaggactagacttcgcctgcgacatctacatctgggcaccactagccgggacttgcggagtgttgttgttgagcttggtaataacgctctactgcaaagcgagccgcaaaaaagcggcggcggcggcgaaaagcccgtttgcgagcccggcgagcagcgcgcaggaagaagatgcgagcagctgccgcgcgccgagcgaagaagaaggcagctgcgaactgagagtgaagttctctcgctccgcggacgcacccgcttaccagcagggtcagaaccagctatacaacgagttaaacctggggcgccgggaggagtacgacgtgttagacaagcgtagaggtagggacccggagatgggaggcaagcctcggagaaagaacccccaggagggcctgtacaacgaactccagaaggacaagatggctgaggcgtactcggagattggtatgaagggcgagagacgtcgcggaaagggacacgacggcttataccaggggctttccaccgcgaccaaggacacatacgacgcgctgcacatgcaagccttaccacctcga

SEQ ID NO: 150 - DNA, 1422 bp, Artificial Sequence
  Synthesized
gacattgtgctcactcagtcacctcccagcctggccatgagcctgggaaaaagggccaccatctcctgtagagccagtgagtccgtcacaatccaggggttgcatttaattcactggtatcagcagaagcccgggcagcctccaaagcttcttattcagagagcgtcaaacgttcagtcgggtgtacctgccagattttctggtagcgggtcccgcactgattttacactgaccatagatccagtggaagaagacgatgtggccgtgtattattgtcagcagactagacgtattcctcgtacatttggtgggggtactaagctggagattaagggaagcacgtccggctcagggaagccgggctccggcgagggaagcacgaaggggcaaattcagctggtccagagcggacctgagctgaaaaaacccggcgagactgttaagatcagttgtaaagcatctggctataccttcacccactacagcataaattgggtgaaacgggcccctggaaagggcctcaaatggatgggttggatcaataccgaatctggggagcctacttatgcatatgacttcaaagggagattcgccttttcactcgagacatctgcctctactgcttacctccaaataaacaacctcaagtatgaagatacagccacttacttttgcgccctcgactatgagagtgccatggactactggggacagggaacctccgttaccgtcagttccggcctgagcggcctgactactaccccagctccacgtcctcccacgccagctccaacgatagcaagtcagcccttatctcttcgccctgaggcttgcaggcccgcggcgggcggcgccgttcacacgcgaggactagacttcgcctgcgacatctacatctgggcaccactagccgggacttgcggagtgttgttgttgagcttggtaataacgctctactgcaaagcgagccgcaaaaaagcggcggcggcggcgaaaagcccgtttgcgagcccggcgagcagcgcgcaggaagaagatgcgagcagctgccgcgcgccgagcgaagaagaaggcagctgcgaactgagagtgaagttctctcgctccgcggacgcacccgcttaccagcagggtcagaaccagctatacaacgagttaaacctggggcgccgggaggagtacgacgtgttagacaagcgtagaggtagggacccggagatgggaggcaagcctcggagaaagaacccccaggagggcctgtacaacgaactccagaaggacaagatggctgaggcgtactcggagattggtatgaagggcgagagacgtcgcggaaagggacacgacggcttataccaggggctttccaccgcgaccaaggacacatacgacgcgctgcacatgcaagccttaccacctcga

SEQ ID NO: 151 - DNA, 1422 bp, Artificial Sequence
  Synthesized
caaattcagctggtccagagcggacctgagctgaaaaaacccggcgagactgttaagatcagttgtaaagcatctggctataccttcacccactacagcattaattgggtgaaacgggcccctggaaagggcctcaaatggatgggttggatcaataccgaaactcgggagtctacttatgcatatgacttcaaagggagattcgccttttcactcgagacatctgcctctactgcttacctccaaataaacaacctcaagtatgaagatacagccacttacttttgcgccctcgactattggagtgccatggactactggggacagggaacctccgttaccgtcagttccggaagcacgtccggctcagggaagccgggctccggcgagggaagcacgaagggggacattgtgctcactcagtcacctcccagcctggccatgagcctgggaaaaagggccaccatctcctgtagagccagtgagtccgtcacaatcccggggcagcatttaatccactggtatcagcagaggcccgggcagcctccaaagcttcttattcagcgtgcgtcaaacctcgagtcgggtgtacctgccagattttctggtagcgggtcccgcactgattttacactgaccatagatccagtggaagaagacgatgtggccgtgtattattgtctgcagactagaaagattcctaggacatttggtgggggtactaagctggagattaagggcctgagcggcctgactactaccccagctccacgtcctcccacgccagctccaacgatagcaagtcagcccttatctcttcgccctgaggcttgcaggcccgcggcgggcggcgccgttcacacgcgaggactagacttcgcctgcgacatctacatctgggcaccactagccgggacttgcggagtgttgttgttgagcttggtaataacgctctactgcaaagcgagccgcaaaaaagcggcggcggcggcgaaaagcccgtttgcgagcccggcgagcagcgcgcaggaagaagatgcgagcagctgccgcgcgccgagcgaagaagaaggcagctgcgaactgagagtgaagttctctcgctccgcggacgcacccgcttaccagcagggtcagaaccagctatacaacgagttaaacctggggcgccgggaggagtacgacgtgttagacaagcgtagaggtagggacccggagatgggaggcaagcctcggagaaagaacccccaggagggcctgtacaacgaactccagaaggacaagatggctgaggcgtactcggagattggtatgaagggcgagagacgtcgcggaaagggacacgacggcttataccaggggctttccaccgcgaccaaggacacatacgacgcgctgcacatgcaagccttaccacctcga

SEQ ID NO: 152 - DNA, 1422 bp, Artificial Sequence
  Synthesized
gacattgtgctcactcagtcacctcccagcctggccatgagcctgggaaaaagggccaccatctcctgtagagccagtgagtccgtcacaatccaggggttgcatttaattcactggtatcagcagaagcccgggcagcctccaaagcttcttattcagagagcgtcaaacgttcagtcgggtgtacctgccagattttctggtagcgggtcccgcactgattttacactgaccatagatccagtggaagaagacgatgtggccgtgtattattgtcagcagactagacgtattcctcgtacatttggtgggggtactaagctggagattaagggaagcacgtccggctcagggaagccgggctccggcgagggaagcacgaaggggcaaattcagctggtccagagcggacctgagctgaaaaaacccggcgagactgttaagatcagttgtaaagcatctggctataccttcacccactacagcataaattgggtgaaacgggcccctggaaagggcctcaaatggatgggttggatcaataccgaaactagggagtctacttatgcatatgacttcaaagggagattcgccttttcactcgagacatctgcctctactgcttacctccaaataaacaacctcaagtatgaagatacagccacttacttttgcgccctcgactataagcaggccatggactactggggacagggaacctccgttaccgtcagttccggcctgagcggcctgactactaccccagctccacgtcctcccacgccagctccaacgatagcaagtcagcccttatctcttcgccctgaggcttgcaggcccgcggcgggcggcgccgttcacacgcgaggactagacttcgcctgcgacatctacatctgggcaccactagccgggacttgcggagtgttgttgttgagcttggtaataacgctctactgcaaagcgagccgcaaaaaagcggcggcggcggcgaaaagcccgtttgcgagcccggcgagcagcgcgcaggaagaagatgcgagcagctgccgcgcgccgagcgaagaagaaggcagctgcgaactgagagtgaagttctctcgctccgcggacgcacccgcttaccagcagggtcagaaccagctatacaacgagttaaacctggggcgccgggaggagtacgacgtgttagacaagcgtagaggtagggacccggagatgggaggcaagcctcggagaaagaacccccaggagggcctgtacaacgaactccagaaggacaagatggctgaggcgtactcggagattggtatgaagggcgagagacgtcgcggaaagggacacgacggcttataccaggggctttccaccgcgaccaaggacacatacgacgcgctgcacatgcaagccttaccacctcga

SEQ ID NO: 153 - protein, 496 aa, Artificial Sequence
  Synthesized
MALPVTALLLPLALLLHAAQPAQIQLVQSGPELKKPGETVKISCKASGYTFTHYSINWVKRAPGKGLKWMGWINTESGEPTYAYDFKGRFAFSLETSASTAYLQINNLKYEDTATYFCALDYESAMDYWGQGTSVTVSSGSTSGSGKPGSGEGSTKGDIVLTQSPPSLAMSLGKRATISCRASESVTIPGQHLINWYQQKPGQPPKLLIQRASNVESGVPARFSGSGSRTDFTLTIDPVEEDDVAVYYCLQTRGIPRTFGGGTKLEIKGLSGLTTTPAPRPPTPAPTIASQPLSLRPEACRPAAGGAVHTRGLDFACDIYIWAPLAGTCGVLLLSLVITLYCKASRKKAAAAAKSPFASPASSAQEEDASSCRAPSEEEGSCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 154 - protein, 496 aa, Artificial Sequence
  Synthesized
MALPVTALLLPLALLLHAAQPADIVLTQSPPSLAMSLGKRATISCRASESVTIPGQHLINWYQQKPGQPPKLLIQRASNVESGVPARFSGSGSRTDFTLTIDPVEEDDVAVYYCLQTRGIPRTFGGGTKLEIKGSTSGSGKPGSGEGSTKGQIQLVQSGPELKKPGETVKISCKASGYTFTHYSINWVKRAPGKGLKWMGWINTESGEPTYAYDFKGRFAFSLETSASTAYLQINNLKYEDTATYFCALDYESAMDYWGQGTSVTVSSGLSGLTTTPAPRPPTPAPTIASQPLSLRPEACRPAAGGAVHTRGLDFACDIYIWAPLAGTCGVLLLSLVITLYCKASRKKAAAAAKSPFASPASSAQEEDASSCRAPSEEEGSCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 155 - protein, 496 aa, Artificial Sequence
  Synthesized
MALPVTALLLPLALLLHAAQPAQIQLVQSGPELKKPGETVKISCKASGYTFTHYSINWVKRAPGKGLKWMGWINTETRESTYAYDFKGRFAFSLETSASTAYLQINNLKYEDTATYFCALDYKQAMDYWGQGTSVTVSSGSTSGSGKPGSGEGSTKGDIVLTQSPPSLAMSLGKRATISCRASESVTIPGQHLIHWYQQRPGQPPKLLIQRASNLESGVPARFSGSGSRTDFTLTIDPVEEDDVAVYYCLQTRKIPRTFGGGTKLEIKGLSGLTTTPAPRPPTPAPTIASQPLSLRPEACRPAAGGAVHTRGLDFACDIYIWAPLAGTCGVLLLSLVITLYCKASRKKAAAAAKSPFASPASSAQEEDASSCRAPSEEEGSCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 156 - protein, 496 aa, Artificial Sequence
  Synthesized
MALPVTALLLPLALLLHAAQPADIVLTQSPPSLAMSLGKRATISCRASESVTIPGQHLIHWYQQRPGQPPKLLIQRASNLESGVPARFSGSGSRTDFTLTIDPVEEDDVAVYYCLQTRKIPRTFGGGTKLEIKGSTSGSGKPGSGEGSTKGQIQLVQSGPELKKPGETVKISCKASGYTFTHYSINWVKRAPGKGLKWMGWINTETRESTYAYDFKGRFAFSLETSASTAYLQINNLKYEDTATYFCALDYKQAMDYWGQGTSVTVSSGLSGLTTTPAPRPPTPAPTIASQPLSLRPEACRPAAGGAVHTRGLDFACDIYIWAPLAGTCGVLLLSLVITLYCKASRKKAAAAAKSPFASPASSAQEEDASSCRAPSEEEGSCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 157 - protein, 496 aa, Artificial Sequence
  Synthesized
MALPVTALLLPLALLLHAAQPAQIQLVQSGPELKKPGETVKISCKASGYTFTHYSINWVKRAPGKGLKWMGWINTETRESTYAYDFKGRFAFSLETSASTAYLQINNLKYEDTATYFCALDYWSAMDYWGQGTSVTVSSGSTSGSGKPGSGEGSTKGDIVLTQSPPSLAMSLGKRATISCRASESVTIQGLHLIHWYQQKPGQPPKLLIQRASNVQSGVPARFSGSGSRTDFTLTIDPVEEDDVAVYYCQQTRRIPRTFGGGTKLEIKGLSGLTTTPAPRPPTPAPTIASQPLSLRPEACRPAAGGAVHTRGLDFACDIYIWAPLAGTCGVLLLSLVITLYCKASRKKAAAAAKSPFASPASSAQEEDASSCRAPSEEEGSCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 158 - protein, 496 aa, Artificial Sequence
  Synthesized
MALPVTALLLPLALLLHAAQPADIVLTQSPPSLAMSLGKRATISCRASESVTIQGLHLIHWYQQKPGQPPKLLIQRASNVQSGVPARFSGSGSRTDFTLTIDPVEEDDVAVYYCQQTRRIPRTFGGGTKLEIKGSTSGSGKPGSGEGSTKGQIQLVQSGPELKKPGETVKISCKASGYTFTHYSINWVKRAPGKGLKWMGWINTETRESTYAYDFKGRFAFSLETSASTAYLQINNLKYEDTATYFCALDYWSAMDYWGQGTSVTVSSGLSGLTTTPAPRPPTPAPTIASQPLSLRPEACRPAAGGAVHTRGLDFACDIYIWAPLAGTCGVLLLSLVITLYCKASRKKAAAAAKSPFASPASSAQEEDASSCRAPSEEEGSCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 159 - protein, 496 aa, Artificial Sequence
  Synthesized
MALPVTALLLPLALLLHAAQPAQIQLVQSGPELKKPGETVKISCKASGYTFTHYSINWVKRAPGKGLKWMGWINTESGEPTYAYDFKGRFAFSLETSASTAYLQINNLKYEDTATYFCALDYESAMDYWGQGTSVTVSSGSTSGSGKPGSGEGSTKGDIVLTQSPPSLAMSLGKRATISCRASESVTIPGQHLIHWYQQRPGQPPKLLIQRASNLESGVPARFSGSGSRTDFTLTIDPVEEDDVAVYYCLQTRKIPRTFGGGTKLEIKGLSGLTTTPAPRPPTPAPTIASQPLSLRPEACRPAAGGAVHTRGLDFACDIYIWAPLAGTCGVLLLSLVITLYCKASRKKAAAAAKSPFASPASSAQEEDASSCRAPSEEEGSCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 160 - protein, 496 aa, Artificial Sequence
  Synthesized
MALPVTALLLPLALLLHAAQPADIVLTQSPPSLAMSLGKRATISCRASESVTIPGQHLINWYQQKPGQPPKLLIQRASNVESGVPARFSGSGSRTDFTLTIDPVEEDDVAVYYCLQTRGIPRTFGGGTKLEIKGSTSGSGKPGSGEGSTKGQIQLVQSGPELKKPGETVKISCKASGYTFTHYSINWVKRAPGKGLKWMGWINTETRESTYAYDFKGRFAFSLETSASTAYLQINNLKYEDTATYFCALDYKQAMDYWGQGTSVTVSSGLSGLTTTPAPRPPTPAPTIASQPLSLRPEACRPAAGGAVHTRGLDFACDIYIWAPLAGTCGVLLLSLVITLYCKASRKKAAAAAKSPFASPASSAQEEDASSCRAPSEEEGSCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 161 - protein, 496 aa, Artificial Sequence
  Synthesized
MALPVTALLLPLALLLHAAQPAQIQLVQSGPELKKPGETVKISCKASGYTFTHYSINWVKRAPGKGLKWMGWINTESGEPTYAYDFKGRFAFSLETSASTAYLQINNLKYEDTATYFCALDYESAMDYWGQGTSVTVSSGSTSGSGKPGSGEGSTKGDIVLTQSPPSLAMSLGKRATISCRASESVTIQGLHLIHWYQQKPGQPPKLLIQRASNVQSGVPARFSGSGSRTDFTLTIDPVEEDDVAVYYCQQTRRIPRTFGGGTKLEIKGLSGLTTTPAPRPPTPAPTIASQPLSLRPEACRPAAGGAVHTRGLDFACDIYIWAPLAGTCGVLLLSLVITLYCKASRKKAAAAAKSPFASPASSAQEEDASSCRAPSEEEGSCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 162 - protein, 496 aa, Artificial Sequence
  Synthesized
MALPVTALLLPLALLLHAAQPADIVLTQSPPSLAMSLGKRATISCRASESVTIPGQHLINWYQQKPGQPPKLLIQRASNVESGVPARFSGSGSRTDFTLTIDPVEEDDVAVYYCLQTRGIPRTFGGGTKLEIKGSTSGSGKPGSGEGSTKGQIQLVQSGPELKKPGETVKISCKASGYTFTHYSINWVKRAPGKGLKWMGWINTETRESTYAYDFKGRFAFSLETSASTAYLQINNLKYEDTATYFCALDYWSAMDYWGQGTSVTVSSGLSGLTTTPAPRPPTPAPTIASQPLSLRPEACRPAAGGAVHTRGLDFACDIYIWAPLAGTCGVLLLSLVITLYCKASRKKAAAAAKSPFASPASSAQEEDASSCRAPSEEEGSCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 163 - protein, 496 aa, Artificial Sequence
  Synthesized
MALPVTALLLPLALLLHAAQPAQIQLVQSGPELKKPGETVKISCKASGYTFTHYSINWVKRAPGKGLKWMGWINTETRESTYAYDFKGRFAFSLETSASTAYLQINNLKYEDTATYFCALDYKQAMDYWGQGTSVTVSSGSTSGSGKPGSGEGSTKGDIVLTQSPPSLAMSLGKRATISCRASESVTIPGQHLINWYQQKPGQPPKLLIQRASNVESGVPARFSGSGSRTDFTLTIDPVEEDDVAVYYCLQTRGIPRTFGGGTKLEIKGLSGLTTTPAPRPPTPAPTIASQPLSLRPEACRPAAGGAVHTRGLDFACDIYIWAPLAGTCGVLLLSLVITLYCKASRKKAAAAAKSPFASPASSAQEEDASSCRAPSEEEGSCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 164 - protein, 496 aa, Artificial Sequence
  Synthesized
MALPVTALLLPLALLLHAAQPADIVLTQSPPSLAMSLGKRATISCRASESVTIPGQHLIHWYQQRPGQPPKLLIQRASNLESGVPARFSGSGSRTDFTLTIDPVEEDDVAVYYCLQTRKIPRTFGGGTKLEIKGSTSGSGKPGSGEGSTKGQIQLVQSGPELKKPGETVKISCKASGYTFTHYSINWVKRAPGKGLKWMGWINTESGEPTYAYDFKGRFAFSLETSASTAYLQINNLKYEDTATYFCALDYESAMDYWGQGTSVTVSSGLSGLTTTPAPRPPTPAPTIASQPLSLRPEACRPAAGGAVHTRGLDFACDIYIWAPLAGTCGVLLLSLVITLYCKASRKKAAAAAKSPFASPASSAQEEDASSCRAPSEEEGSCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 165 - protein, 496 aa, Artificial Sequence
  Synthesized
MALPVTALLLPLALLLHAAQPAQIQLVQSGPELKKPGETVKISCKASGYTFTHYSINWVKRAPGKGLKWMGWINTETRESTYAYDFKGRFAFSLETSASTAYLQINNLKYEDTATYFCALDYKQAMDYWGQGTSVTVSSGSTSGSGKPGSGEGSTKGDIVLTQSPPSLAMSLGKRATISCRASESVTIQGLHLIHWYQQKPGQPPKLLIQRASNVQSGVPARFSGSGSRTDFTLTIDPVEEDDVAVYYCQQTRRIPRTFGGGTKLEIKGLSGLTTTPAPRPPTPAPTIASQPLSLRPEACRPAAGGAVHTRGLDFACDIYIWAPLAGTCGVLLLSLVITLYCKASRKKAAAAAKSPFASPASSAQEEDASSCRAPSEEEGSCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 166 - protein, 496 aa, Artificial Sequence
  Synthesized
MALPVTALLLPLALLLHAAQPADIVLTQSPPSLAMSLGKRATISCRASESVTIPGQHLIHWYQQRPGQPPKLLIQRASNLESGVPARFSGSGSRTDFTLTIDPVEEDDVAVYYCLQTRKIPRTFGGGTKLEIKGSTSGSGKPGSGEGSTKGQIQLVQSGPELKKPGETVKISCKASGYTFTHYSINWVKRAPGKGLKWMGWINTETRESTYAYDFKGRFAFSLETSASTAYLQINNLKYEDTATYFCALDYWSAMDYWGQGTSVTVSSGLSGLTTTPAPRPPTPAPTIASQPLSLRPEACRPAAGGAVHTRGLDFACDIYIWAPLAGTCGVLLLSLVITLYCKASRKKAAAAAKSPFASPASSAQEEDASSCRAPSEEEGSCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 167 - protein, 496 aa, Artificial Sequence
  Synthesized
MALPVTALLLPLALLLHAAQPAQIQLVQSGPELKKPGETVKISCKASGYTFTHYSINWVKRAPGKGLKWMGWINTETRESTYAYDFKGRFAFSLETSASTAYLQINNLKYEDTATYFCALDYWSAMDYWGQGTSVTVSSGSTSGSGKPGSGEGSTKGDIVLTQSPPSLAMSLGKRATISCRASESVTIPGQHLINWYQQKPGQPPKLLIQRASNVESGVPARFSGSGSRTDFTLTIDPVEEDDVAVYYCLQTRGIPRTFGGGTKLEIKGLSGLTTTPAPRPPTPAPTIASQPLSLRPEACRPAAGGAVHTRGLDFACDIYIWAPLAGTCGVLLLSLVITLYCKASRKKAAAAAKSPFASPASSAQEEDASSCRAPSEEEGSCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 168 - protein, 496 aa, Artificial Sequence
  Synthesized
MALPVTALLLPLALLLHAAQPADIVLTQSPPSLAMSLGKRATISCRASESVTIQGLHLIHWYQQKPGQPPKLLIQRASNVQSGVPARFSGSGSRTDFTLTIDPVEEDDVAVYYCQQTRRIPRTFGGGTKLEIKGSTSGSGKPGSGEGSTKGQIQLVQSGPELKKPGETVKISCKASGYTFTHYSINWVKRAPGKGLKWMGWINTESGEPTYAYDFKGRFAFSLETSASTAYLQINNLKYEDTATYFCALDYESAMDYWGQGTSVTVSSGLSGLTTTPAPRPPTPAPTIASQPLSLRPEACRPAAGGAVHTRGLDFACDIYIWAPLAGTCGVLLLSLVITLYCKASRKKAAAAAKSPFASPASSAQEEDASSCRAPSEEEGSCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 169 - protein, 496 aa, Artificial Sequence
  Synthesized
MALPVTALLLPLALLLHAAQPAQIQLVQSGPELKKPGETVKISCKASGYTFTHYSINWVKRAPGKGLKWMGWINTETRESTYAYDFKGRFAFSLETSASTAYLQINNLKYEDTATYFCALDYWSAMDYWGQGTSVTVSSGSTSGSGKPGSGEGSTKGDIVLTQSPPSLAMSLGKRATISCRASESVTIPGQHLIHWYQQRPGQPPKLLIQRASNLESGVPARFSGSGSRTDFTLTIDPVEEDDVAVYYCLQTRKIPRTFGGGTKLEIKGLSGLTTTPAPRPPTPAPTIASQPLSLRPEACRPAAGGAVHTRGLDFACDIYIWAPLAGTCGVLLLSLVITLYCKASRKKAAAAAKSPFASPASSAQEEDASSCRAPSEEEGSCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 170 - protein, 496 aa, Artificial Sequence
  Synthesized
MALPVTALLLPLALLLHAAQPADIVLTQSPPSLAMSLGKRATISCRASESVTIQGLHLIHWYQQKPGQPPKLLIQRASNVQSGVPARFSGSGSRTDFTLTIDPVEEDDVAVYYCQQTRRIPRTFGGGTKLEIKGSTSGSGKPGSGEGSTKGQIQLVQSGPELKKPGETVKISCKASGYTFTHYSINWVKRAPGKGLKWMGWINTETRESTYAYDFKGRFAFSLETSASTAYLQINNLKYEDTATYFCALDYKQAMDYWGQGTSVTVSSGLSGLTTTPAPRPPTPAPTIASQPLSLRPEACRPAAGGAVHTRGLDFACDIYIWAPLAGTCGVLLLSLVITLYCKASRKKAAAAAKSPFASPASSAQEEDASSCRAPSEEEGSCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 171 - DNA, 1488 bp, Artificial Sequence
  Synthesized
atggcgctcccagtgacagccttacttttacctctggcgttattattgcatgcggcccagccggcccaaattcagctggtccagagcggacctgagctgaaaaaacccggcgagactgttaagatcagttgtaaagcatctggctataccttcacccactacagcataaattgggtgaaacgggcccctggaaagggcctcaaatggatgggttggatcaataccgaatctggggagcctacttatgcatatgacttcaaagggagattcgccttttcactcgagacatctgcctctactgcttacctccaaataaacaacctcaagtatgaagatacagccacttacttttgcgccctcgactatgagagtgccatggactactggggacagggaacctccgttaccgtcagttccggaagcacgtccggctcagggaagccgggctccggcgagggaagcacgaagggggacattgtgctcactcagtcacctcccagcctggccatgagcctgggaaaaagggccaccatctcctgtagagccagtgagtccgtcacaatccctgggcagcatctcatcaactggtatcagcagaagcccgggcagcctccaaagcttcttattcagcgtgcgtcaaacgtcgagtcgggtgtacctgccagattttctggtagcgggtcccgcactgattttacactgaccatagatccagtggaagaagacgatgtggccgtgtattattgtctgcagactagaggtattcctcggacatttggtgggggtactaagctggagattaagggcctgagcggcctgactactaccccagctccacgtcctcccacgccagctccaacgatagcaagtcagcccttatctcttcgccctgaggcttgcaggcccgcggcgggcggcgccgttcacacgcgaggactagacttcgcctgcgacatctacatctgggcaccactagccgggacttgcggagtgttgttgttgagcttggtaataacgctctactgcaaagcgagccgcaaaaaagcggcggcggcggcgaaaagcccgtttgcgagcccggcgagcagcgcgcaggaagaagatgcgagcagctgccgcgcgccgagcgaagaagaaggcagctgcgaactgagagtgaagttctctcgctccgcggacgcacccgcttaccagcagggtcagaaccagctatacaacgagttaaacctggggcgccgggaggagtacgacgtgttagacaagcgtagaggtagggacccggagatgggaggcaagcctcggagaaagaacccccaggagggcctgtacaacgaactccagaaggacaagatggctgaggcgtactcggagattggtatgaagggcgagagacgtcgcggaaagggacacgacggcttataccaggggctttccaccgcgaccaaggacacatacgacgcgctgcacatgcaagccttaccacctcga

SEQ ID NO: 172 - DNA, 1488 bp, Artificial Sequence
  Synthesized
atggcgctcccagtgacagccttacttttacctctggcgttattattgcatgcggcccagccggccgacattgtgctcactcagtcacctcccagcctggccatgagcctgggaaaaagggccaccatctcctgtagagccagtgagtccgtcacaatccctgggcagcatctcatcaactggtatcagcagaagcccgggcagcctccaaagcttcttattcagcgtgcgtcaaacgtcgagtcgggtgtacctgccagattttctggtagcgggtcccgcactgattttacactgaccatagatccagtggaagaagacgatgtggccgtgtattattgtctgcagactagaggtattcctcggacatttggtgggggtactaagctggagattaagggaagcacgtccggctcagggaagccgggctccggcgagggaagcacgaaggggcaaattcagctggtccagagcggacctgagctgaaaaaacccggcgagactgttaagatcagttgtaaagcatctggctataccttcacccactacagcataaattgggtgaaacgggcccctggaaagggcctcaaatggatgggttggatcaataccgaatctggggagcctacttatgcatatgacttcaaagggagattcgccttttcactcgagacatctgcctctactgcttacctccaaataaacaacctcaagtatgaagatacagccacttacttttgcgccctcgactatgagagtgccatggactactggggacagggaacctccgttaccgtcagttccggcctgagcggcctgactactaccccagctccacgtcctcccacgccagctccaacgatagcaagtcagcccttatctcttcgccctgaggcttgcaggcccgcggcgggcggcgccgttcacacgcgaggactagacttcgcctgcgacatctacatctgggcaccactagccgggacttgcggagtgttgttgttgagcttggtaataacgctctactgcaaagcgagccgcaaaaaagcggcggcggcggcgaaaagcccgtttgcgagcccggcgagcagcgcgcaggaagaagatgcgagcagctgccgcgcgccgagcgaagaagaaggcagctgcgaactgagagtgaagttctctcgctccgcggacgcacccgcttaccagcagggtcagaaccagctatacaacgagttaaacctggggcgccgggaggagtacgacgtgttagacaagcgtagaggtagggacccggagatgggaggcaagcctcggagaaagaacccccaggagggcctgtacaacgaactccagaaggacaagatggctgaggcgtactcggagattggtatgaagggcgagagacgtcgcggaaagggacacgacggcttataccaggggctttccaccgcgaccaaggacacatacgacgcgctgcacatgcaagccttaccacctcga

SEQ ID NO: 173 - DNA, 1488 bp, Artificial Sequence
  Synthesized
atggcgctcccagtgacagccttacttttacctctggcgttattattgcatgcggcccagccggcccaaattcagctggtccagagcggacctgagctgaaaaaacccggcgagactgttaagatcagttgtaaagcatctggctataccttcacccactacagcataaattgggtgaaacgggcccctggaaagggcctcaaatggatgggttggatcaataccgaaactagggagtctacttatgcatatgacttcaaagggagattcgccttttcactcgagacatctgcctctactgcttacctccaaataaacaacctcaagtatgaagatacagccacttacttttgcgccctcgactataagcaggccatggactactggggacagggaacctccgttaccgtcagttccggaagcacgtccggctcagggaagccgggctccggcgagggaagcacgaagggggacattgtgctcactcagtcacctcccagcctggccatgagcctgggaaaaagggccaccatctcctgtagagccagtgagtccgtcacaatcccggggcagcatttaatccactggtatcagcagaggcccgggcagcctccaaagcttcttattcagcgtgcgtcaaacctcgagtcgggtgtacctgccagattttctggtagcgggtcccgcactgattttacactgaccatagatccagtggaagaagacgatgtggccgtgtattattgtctgcagactagaaagattcctaggacatttggtgggggtactaagctggagattaagggcctgagcggcctgactactaccccagctccacgtcctcccacgccagctccaacgatagcaagtcagcccttatctcttcgccctgaggcttgcaggcccgcggcgggcggcgccgttcacacgcgaggactagacttcgcctgcgacatctacatctgggcaccactagccgggacttgcggagtgttgttgttgagcttggtaataacgctctactgcaaagcgagccgcaaaaaagcggcggcggcggcgaaaagcccgtttgcgagcccggcgagcagcgcgcaggaagaagatgcgagcagctgccgcgcgccgagcgaagaagaaggcagctgcgaactgagagtgaagttctctcgctccgcggacgcacccgcttaccagcagggtcagaaccagctatacaacgagttaaacctggggcgccgggaggagtacgacgtgttagacaagcgtagaggtagggacccggagatgggaggcaagcctcggagaaagaacccccaggagggcctgtacaacgaactccagaaggacaagatggctgaggcgtactcggagattggtatgaagggcgagagacgtcgcggaaagggacacgacggcttataccaggggctttccaccgcgaccaaggacacatacgacgcgctgcacatgcaagccttaccacctcga

SEQ ID NO: 174 - DNA, 1488 bp, Artificial Sequence
  Synthesized
atggcgctcccagtgacagccttacttttacctctggcgttattattgcatgcggcccagccggccgacattgtgctcactcagtcacctcccagcctggccatgagcctgggaaaaagggccaccatctcctgtagagccagtgagtccgtcacaatcccggggcagcatttaatccactggtatcagcagaggcccgggcagcctccaaagcttcttattcagcgtgcgtcaaacctcgagtcgggtgtacctgccagattttctggtagcgggtcccgcactgattttacactgaccatagatccagtggaagaagacgatgtggccgtgtattattgtctgcagactagaaagattcctaggacatttggtgggggtactaagctggagattaagggaagcacgtccggctcagggaagccgggctccggcgagggaagcacgaaggggcaaattcagctggtccagagcggacctgagctgaaaaaacccggcgagactgttaagatcagttgtaaagcatctggctataccttcacccactacagcataaattgggtgaaacgggcccctggaaagggcctcaaatggatgggttggatcaataccgaaactagggagtctacttatgcatatgacttcaaagggagattcgccttttcactcgagacatctgcctctactgcttacctccaaataaacaacctcaagtatgaagatacagccacttacttttgcgccctcgactataagcaggccatggactactggggacagggaacctccgttaccgtcagttccggcctgagcggcctgactactaccccagctccacgtcctcccacgccagctccaacgatagcaagtcagcccttatctcttcgccctgaggcttgcaggcccgcggcgggcggcgccgttcacacgcgaggactagacttcgcctgcgacatctacatctgggcaccactagccgggacttgcggagtgttgttgttgagcttggtaataacgctctactgcaaagcgagccgcaaaaaagcggcggcggcggcgaaaagcccgtttgcgagcccggcgagcagcgcgcaggaagaagatgcgagcagctgccgcgcgccgagcgaagaagaaggcagctgcgaactgagagtgaagttctctcgctccgcggacgcacccgcttaccagcagggtcagaaccagctatacaacgagttaaacctggggcgccgggaggagtacgacgtgttagacaagcgtagaggtagggacccggagatgggaggcaagcctcggagaaagaacccccaggagggcctgtacaacgaactccagaaggacaagatggctgaggcgtactcggagattggtatgaagggcgagagacgtcgcggaaagggacacgacggcttataccaggggctttccaccgcgaccaaggacacatacgacgcgctgcacatgcaagccttaccacctcga

SEQ ID NO: 175 - DNA, 1488 bp, Artificial Sequence
  Synthesized
atggcgctcccagtgacagccttacttttacctctggcgttattattgcatgcggcccagccggcccaaattcagctggtccagagcggacctgagctgaaaaaacccggcgagactgttaagatcagttgtaaagcatctggctataccttcacccactacagcattaattgggtgaaacgggcccctggaaagggcctcaaatggatgggttggatcaataccgaaactcgggagtctacttatgcatatgacttcaaagggagattcgccttttcactcgagacatctgcctctactgcttacctccaaataaacaacctcaagtatgaagatacagccacttacttttgcgccctcgactattggagtgccatggactactggggacagggaacctccgttaccgtcagttccggaagcacgtccggctcagggaagccgggctccggcgagggaagcacgaagggggacattgtgctcactcagtcacctcccagcctggccatgagcctgggaaaaagggccaccatctcctgtagagccagtgagtccgtcacaatccaggggttgcatttaattcactggtatcagcagaagcccgggcagcctccaaagcttcttattcagagagcgtcaaacgttcagtcgggtgtacctgccagattttctggtagcgggtcccgcactgattttacactgaccatagatccagtggaagaagacgatgtggccgtgtattattgtcagcagactagacgtattcctcgtacatttggtgggggtactaagctggagattaagggcctgagcggcctgactactaccccagctccacgtcctcccacgccagctccaacgatagcaagtcagcccttatctcttcgccctgaggcttgcaggcccgcggcgggcggcgccgttcacacgcgaggactagacttcgcctgcgacatctacatctgggcaccactagccgggacttgcggagtgttgttgttgagcttggtaataacgctctactgcaaagcgagccgcaaaaaagcggcggcggcggcgaaaagcccgtttgcgagcccggcgagcagcgcgcaggaagaagatgcgagcagctgccgcgcgccgagcgaagaagaaggcagctgcgaactgagagtgaagttctctcgctccgcggacgcacccgcttaccagcagggtcagaaccagctatacaacgagttaaacctggggcgccgggaggagtacgacgtgttagacaagcgtagaggtagggacccggagatgggaggcaagcctcggagaaagaacccccaggagggcctgtacaacgaactccagaaggacaagatggctgaggcgtactcggagattggtatgaagggcgagagacgtcgcggaaagggacacgacggcttataccaggggctttccaccgcgaccaaggacacatacgacgcgctgcacatgcaagccttaccacctcga

SEQ ID NO: 176 - DNA, 1488 bp, Artificial Sequence
  Synthesized
atggcgctcccagtgacagccttacttttacctctggcgttattattgcatgcggcccagccggccgacattgtgctcactcagtcacctcccagcctggccatgagcctgggaaaaagggccaccatctcctgtagagccagtgagtccgtcacaatccaggggttgcatttaattcactggtatcagcagaagcccgggcagcctccaaagcttcttattcagagagcgtcaaacgttcagtcgggtgtacctgccagattttctggtagcgggtcccgcactgattttacactgaccatagatccagtggaagaagacgatgtggccgtgtattattgtcagcagactagacgtattcctcgtacatttggtgggggtactaagctggagattaagggaagcacgtccggctcagggaagccgggctccggcgagggaagcacgaaggggcaaattcagctggtccagagcggacctgagctgaaaaaacccggcgagactgttaagatcagttgtaaagcatctggctataccttcacccactacagcattaattgggtgaaacgggcccctggaaagggcctcaaatggatgggttggatcaataccgaaactcgggagtctacttatgcatatgacttcaaagggagattcgccttttcactcgagacatctgcctctactgcttacctccaaataaacaacctcaagtatgaagatacagccacttacttttgcgccctcgactattggagtgccatggactactggggacagggaacctccgttaccgtcagttccggcctgagcggcctgactactaccccagctccacgtcctcccacgccagctccaacgatagcaagtcagcccttatctcttcgccctgaggcttgcaggcccgcggcgggcggcgccgttcacacgcgaggactagacttcgcctgcgacatctacatctgggcaccactagccgggacttgcggagtgttgttgttgagcttggtaataacgctctactgcaaagcgagccgcaaaaaagcggcggcggcggcgaaaagcccgtttgcgagcccggcgagcagcgcgcaggaagaagatgcgagcagctgccgcgcgccgagcgaagaagaaggcagctgcgaactgagagtgaagttctctcgctccgcggacgcacccgcttaccagcagggtcagaaccagctatacaacgagttaaacctggggcgccgggaggagtacgacgtgttagacaagcgtagaggtagggacccggagatgggaggcaagcctcggagaaagaacccccaggagggcctgtacaacgaactccagaaggacaagatggctgaggcgtactcggagattggtatgaagggcgagagacgtcgcggaaagggacacgacggcttataccaggggctttccaccgcgaccaaggacacatacgacgcgctgcacatgcaagccttaccacctcga

SEQ ID NO: 177 - DNA, 1488 bp, Artificial Sequence
  Synthesized
atggcgctcccagtgacagccttacttttacctctggcgttattattgcatgcggcccagccggcccaaattcagctggtccagagcggacctgagctgaaaaaacccggcgagactgttaagatcagttgtaaagcatctggctataccttcacccactacagcataaattgggtgaaacgggcccctggaaagggcctcaaatggatgggttggatcaataccgaatctggggagcctacttatgcatatgacttcaaagggagattcgccttttcactcgagacatctgcctctactgcttacctccaaataaacaacctcaagtatgaagatacagccacttacttttgcgccctcgactatgagagtgccatggactactggggacagggaacctccgttaccgtcagttccggaagcacgtccggctcagggaagccgggctccggcgagggaagcacgaagggggacattgtgctcactcagtcacctcccagcctggccatgagcctgggaaaaagggccaccatctcctgtagagccagtgagtccgtcacaatcccggggcagcatttaatccactggtatcagcagaggcccgggcagcctccaaagcttcttattcagcgtgcgtcaaacctcgagtcgggtgtacctgccagattttctggtagcgggtcccgcactgattttacactgaccatagatccagtggaagaagacgatgtggccgtgtattattgtctgcagactagaaagattcctaggacatttggtgggggtactaagctggagattaagggcctgagcggcctgactactaccccagctccacgtcctcccacgccagctccaacgatagcaagtcagcccttatctcttcgccctgaggcttgcaggcccgcggcgggcggcgccgttcacacgcgaggactagacttcgcctgcgacatctacatctgggcaccactagccgggacttgcggagtgttgttgttgagcttggtaataacgctctactgcaaagcgagccgcaaaaaagcggcggcggcggcgaaaagcccgtttgcgagcccggcgagcagcgcgcaggaagaagatgcgagcagctgccgcgcgccgagcgaagaagaaggcagctgcgaactgagagtgaagttctctcgctccgcggacgcacccgcttaccagcagggtcagaaccagctatacaacgagttaaacctggggcgccgggaggagtacgacgtgttagacaagcgtagaggtagggacccggagatgggaggcaagcctcggagaaagaacccccaggagggcctgtacaacgaactccagaaggacaagatggctgaggcgtactcggagattggtatgaagggcgagagacgtcgcggaaagggacacgacggcttataccaggggctttccaccgcgaccaaggacacatacgacgcgctgcacatgcaagccttaccacctcga

SEQ ID NO: 178 - DNA, 1488 bp, Artificial Sequence
  Synthesized
atggcgctcccagtgacagccttacttttacctctggcgttattattgcatgcggcccagccggccgacattgtgctcactcagtcacctcccagcctggccatgagcctgggaaaaagggccaccatctcctgtagagccagtgagtccgtcacaatccctgggcagcatctcatcaactggtatcagcagaagcccgggcagcctccaaagcttcttattcagcgtgcgtcaaacgtcgagtcgggtgtacctgccagattttctggtagcgggtcccgcactgattttacactgaccatagatccagtggaagaagacgatgtggccgtgtattattgtctgcagactagaggtattcctcggacatttggtgggggtactaagctggagattaagggaagcacgtccggctcagggaagccgggctccggcgagggaagcacgaaggggcaaattcagctggtccagagcggacctgagctgaaaaaacccggcgagactgttaagatcagttgtaaagcatctggctataccttcacccactacagcataaattgggtgaaacgggcccctggaaagggcctcaaatggatgggttggatcaataccgaaactagggagtctacttatgcatatgacttcaaagggagattcgccttttcactcgagacatctgcctctactgcttacctccaaataaacaacctcaagtatgaagatacagccacttacttttgcgccctcgactataagcaggccatggactactggggacagggaacctccgttaccgtcagttccggcctgagcggcctgactactaccccagctccacgtcctcccacgccagctccaacgatagcaagtcagcccttatctcttcgccctgaggcttgcaggcccgcggcgggcggcgccgttcacacgcgaggactagacttcgcctgcgacatctacatctgggcaccactagccgggacttgcggagtgttgttgttgagcttggtaataacgctctactgcaaagcgagccgcaaaaaagcggcggcggcggcgaaaagcccgtttgcgagcccggcgagcagcgcgcaggaagaagatgcgagcagctgccgcgcgccgagcgaagaagaaggcagctgcgaactgagagtgaagttctctcgctccgcggacgcacccgcttaccagcagggtcagaaccagctatacaacgagttaaacctggggcgccgggaggagtacgacgtgttagacaagcgtagaggtagggacccggagatgggaggcaagcctcggagaaagaacccccaggagggcctgtacaacgaactccagaaggacaagatggctgaggcgtactcggagattggtatgaagggcgagagacgtcgcggaaagggacacgacggcttataccaggggctttccaccgcgaccaaggacacatacgacgcgctgcacatgcaagccttaccacctcga

SEQ ID NO: 179 - DNA, 1488 bp, Artificial Sequence
  Synthesized
atggcgctcccagtgacagccttacttttacctctggcgttattattgcatgcggcccagccggcccaaattcagctggtccagagcggacctgagctgaaaaaacccggcgagactgttaagatcagttgtaaagcatctggctataccttcacccactacagcataaattgggtgaaacgggcccctggaaagggcctcaaatggatgggttggatcaataccgaatctggggagcctacttatgcatatgacttcaaagggagattcgccttttcactcgagacatctgcctctactgcttacctccaaataaacaacctcaagtatgaagatacagccacttacttttgcgccctcgactatgagagtgccatggactactggggacagggaacctccgttaccgtcagttccggaagcacgtccggctcagggaagccgggctccggcgagggaagcacgaagggggacattgtgctcactcagtcacctcccagcctggccatgagcctgggaaaaagggccaccatctcctgtagagccagtgagtccgtcacaatccaggggttgcatttaattcactggtatcagcagaagcccgggcagcctccaaagcttcttattcagagagcgtcaaacgttcagtcgggtgtacctgccagattttctggtagcgggtcccgcactgattttacactgaccatagatccagtggaagaagacgatgtggccgtgtattattgtcagcagactagacgtattcctcgtacatttggtgggggtactaagctggagattaagggcctgagcggcctgactactaccccagctccacgtcctcccacgccagctccaacgatagcaagtcagcccttatctcttcgccctgaggcttgcaggcccgcggcgggcggcgccgttcacacgcgaggactagacttcgcctgcgacatctacatctgggcaccactagccgggacttgcggagtgttgttgttgagcttggtaataacgctctactgcaaagcgagccgcaaaaaagcggcggcggcggcgaaaagcccgtttgcgagcccggcgagcagcgcgcaggaagaagatgcgagcagctgccgcgcgccgagcgaagaagaaggcagctgcgaactgagagtgaagttctctcgctccgcggacgcacccgcttaccagcagggtcagaaccagctatacaacgagttaaacctggggcgccgggaggagtacgacgtgttagacaagcgtagaggtagggacccggagatgggaggcaagcctcggagaaagaacccccaggagggcctgtacaacgaactccagaaggacaagatggctgaggcgtactcggagattggtatgaagggcgagagacgtcgcggaaagggacacgacggcttataccaggggctttccaccgcgaccaaggacacatacgacgcgctgcacatgcaagccttaccacctcga

SEQ ID NO: 180 - DNA, 1488 bp, Artificial Sequence
  Synthesized
atggcgctcccagtgacagccttacttttacctctggcgttattattgcatgcggcccagccggccgacattgtgctcactcagtcacctcccagcctggccatgagcctgggaaaaagggccaccatctcctgtagagccagtgagtccgtcacaatccctgggcagcatctcatcaactggtatcagcagaagcccgggcagcctccaaagcttcttattcagcgtgcgtcaaacgtcgagtcgggtgtacctgccagattttctggtagcgggtcccgcactgattttacactgaccatagatccagtggaagaagacgatgtggccgtgtattattgtctgcagactagaggtattcctcggacatttggtgggggtactaagctggagattaagggaagcacgtccggctcagggaagccgggctccggcgagggaagcacgaaggggcaaattcagctggtccagagcggacctgagctgaaaaaacccggcgagactgttaagatcagttgtaaagcatctggctataccttcacccactacagcattaattgggtgaaacgggcccctggaaagggcctcaaatggatgggttggatcaataccgaaactcgggagtctacttatgcatatgacttcaaagggagattcgccttttcactcgagacatctgcctctactgcttacctccaaataaacaacctcaagtatgaagatacagccacttacttttgcgccctcgactattggagtgccatggactactggggacagggaacctccgttaccgtcagttccggcctgagcggcctgactactaccccagctccacgtcctcccacgccagctccaacgatagcaagtcagcccttatctcttcgccctgaggcttgcaggcccgcggcgggcggcgccgttcacacgcgaggactagacttcgcctgcgacatctacatctgggcaccactagccgggacttgcggagtgttgttgttgagcttggtaataacgctctactgcaaagcgagccgcaaaaaagcggcggcggcggcgaaaagcccgtttgcgagcccggcgagcagcgcgcaggaagaagatgcgagcagctgccgcgcgccgagcgaagaagaaggcagctgcgaactgagagtgaagttctctcgctccgcggacgcacccgcttaccagcagggtcagaaccagctatacaacgagttaaacctggggcgccgggaggagtacgacgtgttagacaagcgtagaggtagggacccggagatgggaggcaagcctcggagaaagaacccccaggagggcctgtacaacgaactccagaaggacaagatggctgaggcgtactcggagattggtatgaagggcgagagacgtcgcggaaagggacacgacggcttataccaggggctttccaccgcgaccaaggacacatacgacgcgctgcacatgcaagccttaccacctcga

SEQ ID NO: 181 - DNA, 1488 bp, Artificial Sequence
  Synthesized
atggcgctcccagtgacagccttacttttacctctggcgttattattgcatgcggcccagccggcccaaattcagctggtccagagcggacctgagctgaaaaaacccggcgagactgttaagatcagttgtaaagcatctggctataccttcacccactacagcataaattgggtgaaacgggcccctggaaagggcctcaaatggatgggttggatcaataccgaaactagggagtctacttatgcatatgacttcaaagggagattcgccttttcactcgagacatctgcctctactgcttacctccaaataaacaacctcaagtatgaagatacagccacttacttttgcgccctcgactataagcaggccatggactactggggacagggaacctccgttaccgtcagttccggaagcacgtccggctcagggaagccgggctccggcgagggaagcacgaagggggacattgtgctcactcagtcacctcccagcctggccatgagcctgggaaaaagggccaccatctcctgtagagccagtgagtccgtcacaatccctgggcagcatctcatcaactggtatcagcagaagcccgggcagcctccaaagcttcttattcagcgtgcgtcaaacgtcgagtcgggtgtacctgccagattttctggtagcgggtcccgcactgattttacactgaccatagatccagtggaagaagacgatgtggccgtgtattattgtctgcagactagaggtattcctcggacatttggtgggggtactaagctggagattaagggcctgagcggcctgactactaccccagctccacgtcctcccacgccagctccaacgatagcaagtcagcccttatctcttcgccctgaggcttgcaggcccgcggcgggcggcgccgttcacacgcgaggactagacttcgcctgcgacatctacatctgggcaccactagccgggacttgcggagtgttgttgttgagcttggtaataacgctctactgcaaagcgagccgcaaaaaagcggcggcggcggcgaaaagcccgtttgcgagcccggcgagcagcgcgcaggaagaagatgcgagcagctgccgcgcgccgagcgaagaagaaggcagctgcgaactgagagtgaagttctctcgctccgcggacgcacccgcttaccagcagggtcagaaccagctatacaacgagttaaacctggggcgccgggaggagtacgacgtgttagacaagcgtagaggtagggacccggagatgggaggcaagcctcggagaaagaacccccaggagggcctgtacaacgaactccagaaggacaagatggctgaggcgtactcggagattggtatgaagggcgagagacgtcgcggaaagggacacgacggcttataccaggggctttccaccgcgaccaaggacacatacgacgcgctgcacatgcaagccttaccacctcga

SEQ ID NO: 182 - DNA, 1488 bp, Artificial Sequence
  Synthesized
atggcgctcccagtgacagccttacttttacctctggcgttattattgcatgcggcccagccggccgacattgtgctcactcagtcacctcccagcctggccatgagcctgggaaaaagggccaccatctcctgtagagccagtgagtccgtcacaatcccggggcagcatttaatccactggtatcagcagaggcccgggcagcctccaaagcttcttattcagcgtgcgtcaaacctcgagtcgggtgtacctgccagattttctggtagcgggtcccgcactgattttacactgaccatagatccagtggaagaagacgatgtggccgtgtattattgtctgcagactagaaagattcctaggacatttggtgggggtactaagctggagattaagggaagcacgtccggctcagggaagccgggctccggcgagggaagcacgaaggggcaaattcagctggtccagagcggacctgagctgaaaaaacccggcgagactgttaagatcagttgtaaagcatctggctataccttcacccactacagcataaattgggtgaaacgggcccctggaaagggcctcaaatggatgggttggatcaataccgaatctggggagcctacttatgcatatgacttcaaagggagattcgccttttcactcgagacatctgcctctactgcttacctccaaataaacaacctcaagtatgaagatacagccacttacttttgcgccctcgactatgagagtgccatggactactggggacagggaacctccgttaccgtcagttccggcctgagcggcctgactactaccccagctccacgtcctcccacgccagctccaacgatagcaagtcagcccttatctcttcgccctgaggcttgcaggcccgcggcgggcggcgccgttcacacgcgaggactagacttcgcctgcgacatctacatctgggcaccactagccgggacttgcggagtgttgttgttgagcttggtaataacgctctactgcaaagcgagccgcaaaaaagcggcggcggcggcgaaaagcccgtttgcgagcccggcgagcagcgcgcaggaagaagatgcgagcagctgccgcgcgccgagcgaagaagaaggcagctgcgaactgagagtgaagttctctcgctccgcggacgcacccgcttaccagcagggtcagaaccagctatacaacgagttaaacctggggcgccgggaggagtacgacgtgttagacaagcgtagaggtagggacccggagatgggaggcaagcctcggagaaagaacccccaggagggcctgtacaacgaactccagaaggacaagatggctgaggcgtactcggagattggtatgaagggcgagagacgtcgcggaaagggacacgacggcttataccaggggctttccaccgcgaccaaggacacatacgacgcgctgcacatgcaagccttaccacctcga

SEQ ID NO: 183 - DNA, 1488 bp, Artificial Sequence
  Synthesized
atggcgctcccagtgacagccttacttttacctctggcgttattattgcatgcggcccagccggcccaaattcagctggtccagagcggacctgagctgaaaaaacccggcgagactgttaagatcagttgtaaagcatctggctataccttcacccactacagcataaattgggtgaaacgggcccctggaaagggcctcaaatggatgggttggatcaataccgaaactagggagtctacttatgcatatgacttcaaagggagattcgccttttcactcgagacatctgcctctactgcttacctccaaataaacaacctcaagtatgaagatacagccacttacttttgcgccctcgactataagcaggccatggactactggggacagggaacctccgttaccgtcagttccggaagcacgtccggctcagggaagccgggctccggcgagggaagcacgaagggggacattgtgctcactcagtcacctcccagcctggccatgagcctgggaaaaagggccaccatctcctgtagagccagtgagtccgtcacaatccaggggttgcatttaattcactggtatcagcagaagcccgggcagcctccaaagcttcttattcagagagcgtcaaacgttcagtcgggtgtacctgccagattttctggtagcgggtcccgcactgattttacactgaccatagatccagtggaagaagacgatgtggccgtgtattattgtcagcagactagacgtattcctcgtacatttggtgggggtactaagctggagattaagggcctgagcggcctgactactaccccagctccacgtcctcccacgccagctccaacgatagcaagtcagcccttatctcttcgccctgaggcttgcaggcccgcggcgggcggcgccgttcacacgcgaggactagacttcgcctgcgacatctacatctgggcaccactagccgggacttgcggagtgttgttgttgagcttggtaataacgctctactgcaaagcgagccgcaaaaaagcggcggcggcggcgaaaagcccgtttgcgagcccggcgagcagcgcgcaggaagaagatgcgagcagctgccgcgcgccgagcgaagaagaaggcagctgcgaactgagagtgaagttctctcgctccgcggacgcacccgcttaccagcagggtcagaaccagctatacaacgagttaaacctggggcgccgggaggagtacgacgtgttagacaagcgtagaggtagggacccggagatgggaggcaagcctcggagaaagaacccccaggagggcctgtacaacgaactccagaaggacaagatggctgaggcgtactcggagattggtatgaagggcgagagacgtcgcggaaagggacacgacggcttataccaggggctttccaccgcgaccaaggacacatacgacgcgctgcacatgcaagccttaccacctcga

SEQ ID NO: 184 - DNA, 1488 bp, Artificial Sequence
  Synthesized
atggcgctcccagtgacagccttacttttacctctggcgttattattgcatgcggcccagccggccgacattgtgctcactcagtcacctcccagcctggccatgagcctgggaaaaagggccaccatctcctgtagagccagtgagtccgtcacaatcccggggcagcatttaatccactggtatcagcagaggcccgggcagcctccaaagcttcttattcagcgtgcgtcaaacctcgagtcgggtgtacctgccagattttctggtagcgggtcccgcactgattttacactgaccatagatccagtggaagaagacgatgtggccgtgtattattgtctgcagactagaaagattcctaggacatttggtgggggtactaagctggagattaagggaagcacgtccggctcagggaagccgggctccggcgagggaagcacgaaggggcaaattcagctggtccagagcggacctgagctgaaaaaacccggcgagactgttaagatcagttgtaaagcatctggctataccttcacccactacagcattaattgggtgaaacgggcccctggaaagggcctcaaatggatgggttggatcaataccgaaactcgggagtctacttatgcatatgacttcaaagggagattcgccttttcactcgagacatctgcctctactgcttacctccaaataaacaacctcaagtatgaagatacagccacttacttttgcgccctcgactattggagtgccatggactactggggacagggaacctccgttaccgtcagttccggcctgagcggcctgactactaccccagctccacgtcctcccacgccagctccaacgatagcaagtcagcccttatctcttcgccctgaggcttgcaggcccgcggcgggcggcgccgttcacacgcgaggactagacttcgcctgcgacatctacatctgggcaccactagccgggacttgcggagtgttgttgttgagcttggtaataacgctctactgcaaagcgagccgcaaaaaagcggcggcggcggcgaaaagcccgtttgcgagcccggcgagcagcgcgcaggaagaagatgcgagcagctgccgcgcgccgagcgaagaagaaggcagctgcgaactgagagtgaagttctctcgctccgcggacgcacccgcttaccagcagggtcagaaccagctatacaacgagttaaacctggggcgccgggaggagtacgacgtgttagacaagcgtagaggtagggacccggagatgggaggcaagcctcggagaaagaacccccaggagggcctgtacaacgaactccagaaggacaagatggctgaggcgtactcggagattggtatgaagggcgagagacgtcgcggaaagggacacgacggcttataccaggggctttccaccgcgaccaaggacacatacgacgcgctgcacatgcaagccttaccacctcga

SEQ ID NO: 185 - DNA, 1488 bp, Artificial Sequence
  Synthesized
atggcgctcccagtgacagccttacttttacctctggcgttattattgcatgcggcccagccggcccaaattcagctggtccagagcggacctgagctgaaaaaacccggcgagactgttaagatcagttgtaaagcatctggctataccttcacccactacagcattaattgggtgaaacgggcccctggaaagggcctcaaatggatgggttggatcaataccgaaactcgggagtctacttatgcatatgacttcaaagggagattcgccttttcactcgagacatctgcctctactgcttacctccaaataaacaacctcaagtatgaagatacagccacttacttttgcgccctcgactattggagtgccatggactactggggacagggaacctccgttaccgtcagttccggaagcacgtccggctcagggaagccgggctccggcgagggaagcacgaagggggacattgtgctcactcagtcacctcccagcctggccatgagcctgggaaaaagggccaccatctcctgtagagccagtgagtccgtcacaatccctgggcagcatctcatcaactggtatcagcagaagcccgggcagcctccaaagcttcttattcagcgtgcgtcaaacgtcgagtcgggtgtacctgccagattttctggtagcgggtcccgcactgattttacactgaccatagatccagtggaagaagacgatgtggccgtgtattattgtctgcagactagaggtattcctcggacatttggtgggggtactaagctggagattaagggcctgagcggcctgactactaccccagctccacgtcctcccacgccagctccaacgatagcaagtcagcccttatctcttcgccctgaggcttgcaggcccgcggcgggcggcgccgttcacacgcgaggactagacttcgcctgcgacatctacatctgggcaccactagccgggacttgcggagtgttgttgttgagcttggtaataacgctctactgcaaagcgagccgcaaaaaagcggcggcggcggcgaaaagcccgtttgcgagcccggcgagcagcgcgcaggaagaagatgcgagcagctgccgcgcgccgagcgaagaagaaggcagctgcgaactgagagtgaagttctctcgctccgcggacgcacccgcttaccagcagggtcagaaccagctatacaacgagttaaacctggggcgccgggaggagtacgacgtgttagacaagcgtagaggtagggacccggagatgggaggcaagcctcggagaaagaacccccaggagggcctgtacaacgaactccagaaggacaagatggctgaggcgtactcggagattggtatgaagggcgagagacgtcgcggaaagggacacgacggcttataccaggggctttccaccgcgaccaaggacacatacgacgcgctgcacatgcaagccttaccacctcga

SEQ ID NO: 186 - DNA, 1488 bp, Artificial Sequence
  Synthesized
atggcgctcccagtgacagccttacttttacctctggcgttattattgcatgcggcccagccggccgacattgtgctcactcagtcacctcccagcctggccatgagcctgggaaaaagggccaccatctcctgtagagccagtgagtccgtcacaatccaggggttgcatttaattcactggtatcagcagaagcccgggcagcctccaaagcttcttattcagagagcgtcaaacgttcagtcgggtgtacctgccagattttctggtagcgggtcccgcactgattttacactgaccatagatccagtggaagaagacgatgtggccgtgtattattgtcagcagactagacgtattcctcgtacatttggtgggggtactaagctggagattaagggaagcacgtccggctcagggaagccgggctccggcgagggaagcacgaaggggcaaattcagctggtccagagcggacctgagctgaaaaaacccggcgagactgttaagatcagttgtaaagcatctggctataccttcacccactacagcataaattgggtgaaacgggcccctggaaagggcctcaaatggatgggttggatcaataccgaatctggggagcctacttatgcatatgacttcaaagggagattcgccttttcactcgagacatctgcctctactgcttacctccaaataaacaacctcaagtatgaagatacagccacttacttttgcgccctcgactatgagagtgccatggactactggggacagggaacctccgttaccgtcagttccggcctgagcggcctgactactaccccagctccacgtcctcccacgccagctccaacgatagcaagtcagcccttatctcttcgccctgaggcttgcaggcccgcggcgggcggcgccgttcacacgcgaggactagacttcgcctgcgacatctacatctgggcaccactagccgggacttgcggagtgttgttgttgagcttggtaataacgctctactgcaaagcgagccgcaaaaaagcggcggcggcggcgaaaagcccgtttgcgagcccggcgagcagcgcgcaggaagaagatgcgagcagctgccgcgcgccgagcgaagaagaaggcagctgcgaactgagagtgaagttctctcgctccgcggacgcacccgcttaccagcagggtcagaaccagctatacaacgagttaaacctggggcgccgggaggagtacgacgtgttagacaagcgtagaggtagggacccggagatgggaggcaagcctcggagaaagaacccccaggagggcctgtacaacgaactccagaaggacaagatggctgaggcgtactcggagattggtatgaagggcgagagacgtcgcggaaagggacacgacggcttataccaggggctttccaccgcgaccaaggacacatacgacgcgctgcacatgcaagccttaccacctcga

SEQ ID NO: 187 - DNA, 1488 bp, Artificial Sequence
  Synthesized
atggcgctcccagtgacagccttacttttacctctggcgttattattgcatgcggcccagccggcccaaattcagctggtccagagcggacctgagctgaaaaaacccggcgagactgttaagatcagttgtaaagcatctggctataccttcacccactacagcattaattgggtgaaacgggcccctggaaagggcctcaaatggatgggttggatcaataccgaaactcgggagtctacttatgcatatgacttcaaagggagattcgccttttcactcgagacatctgcctctactgcttacctccaaataaacaacctcaagtatgaagatacagccacttacttttgcgccctcgactattggagtgccatggactactggggacagggaacctccgttaccgtcagttccggaagcacgtccggctcagggaagccgggctccggcgagggaagcacgaagggggacattgtgctcactcagtcacctcccagcctggccatgagcctgggaaaaagggccaccatctcctgtagagccagtgagtccgtcacaatcccggggcagcatttaatccactggtatcagcagaggcccgggcagcctccaaagcttcttattcagcgtgcgtcaaacctcgagtcgggtgtacctgccagattttctggtagcgggtcccgcactgattttacactgaccatagatccagtggaagaagacgatgtggccgtgtattattgtctgcagactagaaagattcctaggacatttggtgggggtactaagctggagattaagggcctgagcggcctgactactaccccagctccacgtcctcccacgccagctccaacgatagcaagtcagcccttatctcttcgccctgaggcttgcaggcccgcggcgggcggcgccgttcacacgcgaggactagacttcgcctgcgacatctacatctgggcaccactagccgggacttgcggagtgttgttgttgagcttggtaataacgctctactgcaaagcgagccgcaaaaaagcggcggcggcggcgaaaagcccgtttgcgagcccggcgagcagcgcgcaggaagaagatgcgagcagctgccgcgcgccgagcgaagaagaaggcagctgcgaactgagagtgaagttctctcgctccgcggacgcacccgcttaccagcagggtcagaaccagctatacaacgagttaaacctggggcgccgggaggagtacgacgtgttagacaagcgtagaggtagggacccggagatgggaggcaagcctcggagaaagaacccccaggagggcctgtacaacgaactccagaaggacaagatggctgaggcgtactcggagattggtatgaagggcgagagacgtcgcggaaagggacacgacggcttataccaggggctttccaccgcgaccaaggacacatacgacgcgctgcacatgcaagccttaccacctcga

SEQ ID NO: 188 - DNA, 1488 bp, Artificial Sequence
  Synthesized
atggcgctcccagtgacagccttacttttacctctggcgttattattgcatgcggcccagccggccgacattgtgctcactcagtcacctcccagcctggccatgagcctgggaaaaagggccaccatctcctgtagagccagtgagtccgtcacaatccaggggttgcatttaattcactggtatcagcagaagcccgggcagcctccaaagcttcttattcagagagcgtcaaacgttcagtcgggtgtacctgccagattttctggtagcgggtcccgcactgattttacactgaccatagatccagtggaagaagacgatgtggccgtgtattattgtcagcagactagacgtattcctcgtacatttggtgggggtactaagctggagattaagggaagcacgtccggctcagggaagccgggctccggcgagggaagcacgaaggggcaaattcagctggtccagagcggacctgagctgaaaaaacccggcgagactgttaagatcagttgtaaagcatctggctataccttcacccactacagcataaattgggtgaaacgggcccctggaaagggcctcaaatggatgggttggatcaataccgaaactagggagtctacttatgcatatgacttcaaagggagattcgccttttcactcgagacatctgcctctactgcttacctccaaataaacaacctcaagtatgaagatacagccacttacttttgcgccctcgactataagcaggccatggactactggggacagggaacctccgttaccgtcagttccggcctgagcggcctgactactaccccagctccacgtcctcccacgccagctccaacgatagcaagtcagcccttatctcttcgccctgaggcttgcaggcccgcggcgggcggcgccgttcacacgcgaggactagacttcgcctgcgacatctacatctgggcaccactagccgggacttgcggagtgttgttgttgagcttggtaataacgctctactgcaaagcgagccgcaaaaaagcggcggcggcggcgaaaagcccgtttgcgagcccggcgagcagcgcgcaggaagaagatgcgagcagctgccgcgcgccgagcgaagaagaaggcagctgcgaactgagagtgaagttctctcgctccgcggacgcacccgcttaccagcagggtcagaaccagctatacaacgagttaaacctggggcgccgggaggagtacgacgtgttagacaagcgtagaggtagggacccggagatgggaggcaagcctcggagaaagaacccccaggagggcctgtacaacgaactccagaaggacaagatggctgaggcgtactcggagattggtatgaagggcgagagacgtcgcggaaagggacacgacggcttataccaggggctttccaccgcgaccaaggacacatacgacgcgctgcacatgcaagccttaccacctcga

SEQ ID NO: 189 - protein, 21 aa, Artificial Sequence
  Synthesized
MALPVTALLLPLALLLHAARP

SEQ ID NO: 190 - DNA, 63 bp, Artificial Sequence
  Synthesized
atggcgctcccagtgacagccttacttttacctctggcgttattattgcacgcggctcgtcct

(19) United States
(12) Patent Application Publication
(10) Pub. No.: US 2026/0028412 A1
(43) Pub. Date: Jan. 29, 2026

(54) ANTIBODIES AND FRAGMENTS SPECIFIC FOR B-CELL MATURATION ANTIGEN AND USES THEREOF

(71) Applicant: Precision Biosciences, Inc., Durham, NC (US)

(72) Inventors: Victor Bartsevich, Durham, NC (US); Mark Johnson, Durham, NC (US); Mahmud Hussain, Morrisville, NC (US)

(73) Assignee: Precision Biosciences, Inc., Durham, NC (US)

(21) Appl. No.: 18/041,118

(22) PCT Filed: Aug. 10, 2021
(86) PCT No.: PCT/US2021/045304

§ 371 Date: Feb. 9, 2023

Related U.S. Application Data
(60) Provisional application No. 63/063,923, filed on Aug. 10, 2020.

(51) Int. Cl. C07K 16/28; A61K 40/11; A61K 40/31; A61K 40/42; A61P 35/00; C07K 14/705; C07K 14/725; C12N 5/0783

(52) U.S. Cl. CPC C07K 16/2878; A61K 40/11; A61K 40/31; A61K 40/4202; A61P 35/00; C07K 14/7051; C07K 14/70517; C12N 5/0636; C07K 2317/53; C07K 2317/622; C07K 2317/92; C07K 2319/02; C07K 2319/03; C12N 2510/00

(57) ABSTRACT

The present disclosure provides antibodies, and fragments thereof, having specificity for human B cell maturation antigen, pharmaceutical compositions thereof, and uses thereof. Also provided are chimeric antigen receptors (CARs) comprising such antibodies or antibody fragments, genetically-modified cells comprising such CARs, pharmaceutical compositions comprising such cells, methods for making such cells, and methods of using such cells for the treatment of disorders and diseases, such as cancer.

### FIELD OF THE INVENTION

The present disclosure provides antibodies, or fragments thereof, having specificity for human B cell maturation antigen (BCMA), pharmaceutical compositions thereof, and uses thereof. Also provided are chimeric antigen receptors (CARs) comprising said antibodies or antibody fragments, genetically-modified cells comprising such CARs, pharmaceutical compositions comprising such cells, methods for making such cells, and methods of using such cells for the treatment of disorders and diseases such as cancer.

### REFERENCE TO A SEQUENCE LISTING SUBMITTED AS A TEXT FILE VIA EFS-WEB

The instant application contains a Sequence Listing which has been submitted in ASCII format via EFS-Web and is hereby incorporated by reference in its entirety. Said ASCII copy, created on Aug. 9, 2021, is named P109070051W000-SEQ-EPG, and is 324,172 bytes in size.

### BACKGROUND OF THE INVENTION

Multiple myeloma (MM) is a hematologic malignancy characterized by accumulation of clonal plasma cells in bone marrow often associated with bone lesions. Although hematopoietic stem cell transplantation along with newer drugs such as thalidomide and proteasome inhibitors often induces an initial remission, however, the tumor relapse due to chemoresistance remains a major problem.

B-cell maturation antigen (BCMA) is a tumor necrosis family receptor (TNFR) member expressed cells of the B-cell lineage. BCMA expression is the highest on terminally differentiated B cells. BCMA is involved in mediating the survival of plasma cells for maintaining long-term humoral immunity. The expression of BCMA has been linked to a number of cancers, autoimmune disorders, and infectious diseases. Cancers with increased expression of BCMA include some hematological cancers, such as multiple myeloma, Hodgkin's and non-Hodgkin's lymphoma, various leukemias, and glioblastoma. Given the significant role for BCMA in diseases such as multiple myeloma, antibodies that recognize BCMA, and methods of using such agents, are desired.

### SUMMARY OF THE INVENTION

In one aspect, the invention provides an isolated antibody, or antigen-binding fragment thereof, comprising a variable heavy (VH) region that comprises a complementarity-determining region heavy 1 (CDRH1) domain, a complementarity-determining region heavy 2 (CDRH2) domain, and a complementarity-determining region heavy 3 (CDRH3) domain; and a variable light (VL) region that comprises a complementarity-determining region light 1 (CDRL1) domain, a complementarity-determining region light 2 (CDRL2) domain, and a complementarity-determining region light (CDRL3) domain, wherein the CDRH1 domain, the CDRH2 domain, the CDRH3 domain are from any VH region set forth in any one of SEQ ID NOs: 2, 6, and 10; and wherein the CDRL1 domain, the CDRL2 domain, and the CDRL3 domain are from any VL region set forth in any one of SEQ ID NOs: 4, 8, and 12, wherein the isolated antibody, or antigen-binding fragment thereof, binds (e.g., specifically binds) to human BCMA.

In some embodiments, the CDRH1 domain, the CDRH2 domain, the CDRH3 domain are from a VH region set forth in SEQ ID NO: 2. In some embodiments, the CDRH1 domain, the CDRH2 domain, the CDRH3 domain are from a VH region set forth in SEQ ID NO: 6. In some embodiments, the CDRH1 domain, the CDRH2 domain, the CDRH3 domain are from a VH region set forth in SEQ ID NO: 10. In some embodiments, the CDRL1 domain, the CDRL2 domain, and the CDRL3 domain are from a VL region set forth in SEQ ID NO: 4. In some embodiments, the CDRL1 domain, the CDRL2 domain, and the CDRL3 domain are from a VL region set forth in SEQ ID NO: 8. In some embodiments, the CDRL1 domain, the CDRL2 domain, and the CDRL3 domain are from a VL region set forth in SEQ ID NO: 12.

In some embodiments, the CDRH1 domain, the CDRH2 domain, the CDRH3 domain, the CDRL1 domain, the CDRL2 domain, and the CDRL3 domain are identified by the Kabat numbering scheme. In some embodiments, the CDRH1 domain, the CDRH2 domain, the CDRH3 domain, the CDRL1 domain, the CDRL2 domain, and the CDRL3 domain are identified by the Chothia numbering scheme.

In some embodiments, the CDRH1 domain comprises an amino acid sequence set forth in SEQ ID NO: 14. In some embodiments, the CDRH1 domain comprises an amino acid sequence set forth in SEQ ID NO: 20. In some embodiments, the CDRH1 domain comprises an amino acid sequence set forth in SEQ ID NO: 26.

In some embodiments, the CDRH2 domain comprises an amino acid sequence set forth in SEQ ID NO: 15. In some embodiments, the CDRH2 domain comprises an amino acid sequence set forth in SEQ ID NO: 21. In some embodiments, the CDRH2 domain comprises an amino acid sequence set forth in SEQ ID NO: 27.

In some embodiments, the CDRH3 domain comprises an amino acid sequence set forth in SEQ ID NO: 16. In some embodiments, the CDRH3 domain comprises an amino acid sequence set forth in SEQ ID NO: 22. In some embodiments, the CDRH3 domain comprises an amino acid sequence set forth in SEQ ID NO: 28.

In some embodiments, the CDRL1 domain comprises an amino acid sequence set forth in SEQ ID NO: 17. In some embodiments, the CDRL1 domain comprises an amino acid sequence set forth in SEQ ID NO: 23. In some embodiments, the CDRL1 domain comprises an amino acid sequence set forth in SEQ ID NO: 29.

In some embodiments, the CDRL2 domain comprises an amino acid sequence set forth in SEQ ID NO: 18. In some embodiments, the CDRL2 domain comprises an amino acid sequence set forth in SEQ ID NO: 24. In some embodiments, the CDRL2 domain comprises an amino acid sequence set forth in SEQ ID NO: 30.

In some embodiments, the CDRL3 domain comprises an amino acid sequence set forth in SEQ ID NO: 19. In some embodiments, the CDRL3 domain comprises an amino acid sequence set forth in SEQ ID NO: 25. In some embodiments, the CDRL3 domain comprises an amino acid sequence set forth in SEQ ID NO: 31.

In certain embodiments, the CDRH1 domain comprises an amino acid sequence set forth in SEQ ID NO: 14; the CDRH2 domain comprises an amino acid sequence set forth in SEQ ID NO: 15; and the CDRH3 domain comprises an amino acid sequence set forth in SEQ ID NO: 16. In certain embodiments, the CDRH1 domain comprises an amino acid sequence set forth in SEQ ID NO: 20; the CDRH2 domain comprises an amino acid sequence set forth in SEQ ID NO: 21; and the CDRH3 domain comprises an amino acid sequence set forth in SEQ ID NO: 22. In certain embodiments, the CDRH1 domain comprises an amino acid sequence set forth in SEQ ID NO: 26; the CDRH2 domain comprises an amino acid sequence set forth in SEQ ID NO: 27; and the CDRH3 domain comprises an amino acid sequence set forth in SEQ ID NO: 28.

In certain embodiments, the CDRL1 domain comprises an amino acid sequence set forth in SEQ ID NO: 17; the CDRL2 domain comprises an amino acid sequence set forth in SEQ ID NO: 18; and the CDRL3 domain comprises an amino acid sequence set forth in SEQ ID NO: 19. In certain embodiments, the CDRL1 domain comprises an amino acid sequence set forth in SEQ ID NO: 23; the CDRL2 domain comprises an amino acid sequence set forth in SEQ ID NO: 24; and the CDRL3 domain comprises an amino acid sequence set forth in SEQ ID NO: 25. In certain embodiments, the CDRL1 domain comprises an amino acid sequence set forth in SEQ ID NO: 29; the CDRL2 domain comprises an amino acid sequence set forth in SEQ ID NO: 30; and the CDRL3 domain comprises an amino acid sequence set forth in SEQ ID NO: 31.

In some embodiments, the CDRH1 domain comprises an amino acid sequence set forth in SEQ ID NO: 14; the CDRH2 domain comprises an amino acid sequence set forth in SEQ ID NO: 15; the CDRH3 domain comprises an amino acid sequence set forth in SEQ ID NO: 16; the CDRL1 domain comprises an amino acid sequence set forth in SEQ ID NO: 17; the CDRL2 domain comprises an amino acid sequence set forth in SEQ ID NO: 18; and the CDRL3 domain comprises an amino acid sequence set forth in SEQ ID NO: 19.

In some embodiments, the CDRH1 domain comprises an amino acid sequence set forth in SEQ ID NO: 20; the CDRH2 domain comprises an amino acid sequence set forth in SEQ ID NO: 21; the CDRH3 domain comprises an amino acid sequence set forth in SEQ ID NO: 22; the CDRL1 domain comprises an amino acid sequence set forth in SEQ ID NO: 23; the CDRL2 domain comprises an amino acid sequence set forth in SEQ ID NO: 24; and the CDRL3 domain comprises an amino acid sequence set forth in SEQ ID NO: 25.

In some embodiments, the CDRH1 domain comprises an amino acid sequence set forth in SEQ ID NO: 26; the CDRH2 domain comprises an amino acid sequence set forth in SEQ ID NO: 27; the CDRH3 domain comprises an amino acid sequence set forth in SEQ ID NO: 28; the CDRL1 domain comprises an amino acid sequence set forth in SEQ ID NO: 29; the CDRL2 domain comprises an amino acid sequence set forth in SEQ ID NO: 30; and the CDRL3 domain comprises an amino acid sequence set forth in SEQ ID NO: 31.

In some embodiments, the CDRH1 domain comprises an amino acid sequence set forth in SEQ ID NO: 14; the CDRH2 domain comprises an amino acid sequence set forth in SEQ ID NO: 15; the CDRH3 domain comprises an amino acid sequence set forth in SEQ ID NO: 16; the CDRL1 domain comprises an amino acid sequence set forth in SEQ ID NO: 23; the CDRL2 domain comprises an amino acid sequence set forth in SEQ ID NO: 24; and the CDRL3 domain comprises an amino acid sequence set forth in SEQ ID NO: 25.

In some embodiments, the CDRH1 domain comprises an amino acid sequence set forth in SEQ ID NO: 14; the CDRH2 domain comprises an amino acid sequence set forth in SEQ ID NO: 15; the CDRH3 domain comprises an amino acid sequence set forth in SEQ ID NO: 16; the CDRL1 domain comprises an amino acid sequence set forth in SEQ ID NO: 29; the CDRL2 domain comprises an amino acid sequence set forth in SEQ ID NO: 30; and the CDRL3 domain comprises an amino acid sequence set forth in SEQ ID NO: 31.

In some embodiments, the CDRH1 domain comprises an amino acid sequence set forth in SEQ ID NO: 20; the CDRH2 domain comprises an amino acid sequence set forth in SEQ ID NO: 21; the CDRH3 domain comprises an amino acid sequence set forth in SEQ ID NO: 22; the CDRL1 domain comprises an amino acid sequence set forth in SEQ ID NO: 17; the CDRL2 domain comprises an amino acid sequence set forth in SEQ ID NO: 18; and the CDRL3 domain comprises an amino acid sequence set forth in SEQ ID NO: 19.

In some embodiments, the CDRH1 domain comprises an amino acid sequence set forth in SEQ ID NO: 20; the CDRH2 domain comprises an amino acid sequence set forth in SEQ ID NO: 21; the CDRH3 domain comprises an amino acid sequence set forth in SEQ ID NO: 22; the CDRL1 domain comprises an amino acid sequence set forth in SEQ ID NO: 29; the CDRL2 domain comprises an amino acid sequence set forth in SEQ ID NO: 30; and the CDRL3 domain comprises an amino acid sequence set forth in SEQ ID NO: 31.

In some embodiments, the CDRH1 domain comprises an amino acid sequence set forth in SEQ ID NO: 26; the CDRH2 domain comprises an amino acid sequence set forth in SEQ ID NO: 27; the CDRH3 domain comprises an amino acid sequence set forth in SEQ ID NO: 28; the CDRL1 domain comprises an amino acid sequence set forth in SEQ ID NO: 17; the CDRL2 domain comprises an amino acid sequence set forth in SEQ ID NO: 18; and the CDRL3 domain comprises an amino acid sequence set forth in SEQ ID NO: 19.

In some embodiments, the CDRH1 domain comprises an amino acid sequence set forth in SEQ ID NO: 26; the CDRH2 domain comprises an amino acid sequence set forth in SEQ ID NO: 27; the CDRH3 domain comprises an amino acid sequence set forth in SEQ ID NO: 28; the CDRL1 domain comprises an amino acid sequence set forth in SEQ ID NO: 23; the CDRL2 domain comprises an amino acid sequence set forth in SEQ ID NO: 24; and the CDRL3 domain comprises an amino acid sequence set forth in SEQ ID NO: 25.

In certain embodiments, the VH region comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 2. In certain embodiments, the VH region comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 6. In certain embodiments, the VH region comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 10.

In certain embodiments, the VH region is encoded by a nucleic acid sequence having at least about 80%, 85%, 90%, 91%, 92%, 93%, 94%, 95%, 96%, 97%, 98%, 99%, or more, sequence identity to a sequence set forth in SEQ ID NO: 3. In certain embodiments, the VH region is encoded by a nucleic acid sequence having at least about 80%, 85%, 90%, 91%, 92%, 93%, 94%, 95%, 96%, 97%, 98%, 99%, or more, sequence identity to a sequence set forth in SEQ ID NO: 7. In certain embodiments, the VH region is encoded by a nucleic acid sequence having at least about 80%, 85%, 90%, 91%, 92%, 93%, 94%, 95%, 96%, 97%, 98%, 99%, or more, sequence identity to a sequence set forth in SEQ ID NO: 11.

In certain embodiments, the VL region comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 4. In certain embodiments, the VL region comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 8. In certain embodiments, the VL region comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 12.

In certain embodiments, the VL region is encoded by a nucleic acid sequence having at least about 80%, 85%, 90%, 91%, 92%, 93%, 94%, 95%, 96%, 97%, 98%, 99%, or more, sequence identity to a sequence set forth in SEQ ID NO: 5. In certain embodiments, the VL region is encoded by a nucleic acid sequence having at least about 80%, 85%, 90%, 91%, 92%, 93%, 94%, 95%, 96%, 97%, 98%, 99%, or more, sequence identity to a sequence set forth in SEQ ID NO: 9. In certain embodiments, the VL region is encoded by a nucleic acid sequence having at least about 80%, 85%, 90%, 91%, 92%, 93%, 94%, 95%, 96%, 97%, 98%, 99%, or more, sequence identity to a sequence set forth in SEQ ID NO: 13.

In certain embodiments, the VH region comprises an amino acid sequence having at least about 80%, 85%, 90%, 91%, 92%, 93%, 94%, 95%, 96%, 97%, 98%, 99%, or more, sequence identity to the sequence set forth in SEQ ID NO: 2, and the VL region comprises an amino acid sequence having at least about 80%, 85%, 90%, 91%, 92%, 93%, 94%, 95%, 96%, 97%, 98%, 99%, or more, sequence identity to the sequence set forth in SEQ ID NO: 4.

In certain embodiments, the VH region comprises an amino acid sequence having at least about 80%, 85%, 90%, 91%, 92%, 93%, 94%, 95%, 96%, 97%, 98%, 99%, or more, sequence identity to the sequence set forth in SEQ ID NO: 6, and the VL region comprises an amino acid sequence having at least about 80%, 85%, 90%, 91%, 92%, 93%, 94%, 95%, 96%, 97%, 98%, 99%, or more, sequence identity to the sequence set forth in SEQ ID NO: 8.

In certain embodiments, the VH region comprises an amino acid sequence having at least about 80%, 85%, 90%, 91%, 92%, 93%, 94%, 95%, 96%, 97%, 98%, 99%, or more, sequence identity to the sequence set forth in SEQ ID NO: 10, and the VL region comprises an amino acid sequence having at least about 80%, 85%, 90%, 91%, 92%, 93%, 94%, 95%, 96%, 97%, 98%, 99%, or more, sequence identity to the sequence set forth in SEQ ID NO: 12.

In certain embodiments, the VH region comprises an amino acid sequence having at least about 80%, 85%, 90%, 91%, 92%, 93%, 94%, 95%, 96%, 97%, 98%, 99%, or more, sequence identity to the sequence set forth in SEQ ID NO: 2, and the VL region comprises an amino acid sequence having at least about 80%, 85%, 90%, 91%, 92%, 93%, 94%, 95%, 96%, 97%, 98%, 99%, or more, sequence identity to the sequence set forth in SEQ ID NO: 8.

In certain embodiments, the VH region comprises an amino acid sequence having at least about 80%, 85%, 90%, 91%, 92%, 93%, 94%, 95%, 96%, 97%, 98%, 99%, or more, sequence identity to the sequence set forth in SEQ ID NO: 2, and the VL region comprises an amino acid sequence having at least about 80%, 85%, 90%, 91%, 92%, 93%, 94%, 95%, 96%, 97%, 98%, 99%, or more, sequence identity to the sequence set forth in SEQ ID NO: 12.

In certain embodiments, the VH region comprises an amino acid sequence having at least about 80%, 85%, 90%, 91%, 92%, 93%, 94%, 95%, 96%, 97%, 98%, 99%, or more, sequence identity to the sequence set forth in SEQ ID NO: 6, and the VL region comprises an amino acid sequence having at least about 80%, 85%, 90%, 91%, 92%, 93%, 94%, 95%, 96%, 97%, 98%, 99%, or more, sequence identity to the sequence set forth in SEQ ID NO: 4.

In certain embodiments, the VH region comprises an amino acid sequence having at least about 80%, 85%, 90%, 91%, 92%, 93%, 94%, 95%, 96%, 97%, 98%, 99%, or more, sequence identity to the sequence set forth in SEQ ID NO: 6, and the VL region comprises an amino acid sequence having at least about 80%, 85%, 90%, 91%, 92%, 93%, 94%, 95%, 96%, 97%, 98%, 99%, or more, sequence identity to the sequence set forth in SEQ ID NO: 12.

In certain embodiments, the VH region comprises an amino acid sequence having at least about 80%, 85%, 90%, 91%, 92%, 93%, 94%, 95%, 96%, 97%, 98%, 99%, or more, sequence identity to the sequence set forth in SEQ ID NO: 10, and the VL region comprises an amino acid sequence having at least about 80%, 85%, 90%, 91%, 92%, 93%, 94%, 95%, 96%, 97%, 98%, 99%, or more, sequence identity to the sequence set forth in SEQ ID NO: 4.

In certain embodiments, the VH region comprises an amino acid sequence having at least about 80%, 85%, 90%, 91%, 92%, 93%, 94%, 95%, 96%, 97%, 98%, 99%, or more, sequence identity to the sequence set forth in SEQ ID NO: 10, and the VL region comprises an amino acid sequence having at least about 80%, 85%, 90%, 91%, 92%, 93%, 94%, 95%, 96%, 97%, 98%, 99%, or more, sequence identity to the sequence set forth in SEQ ID NO: 8.

In some embodiments, the VH region is encoded by a nucleic acid sequence having at least about 80%, 85%, 90%, 91%, 92%, 93%, 94%, 95%, 96%, 97%, 98%, 99%, or more, sequence identity to the sequence set forth in SEQ ID NO: 3, and the VL region is encoded by a nucleic acid sequence having at least about 80%, 85%, 90%, 91%, 92%, 93%, 94%, 95%, 96%, 97%, 98%, 99%, or more, sequence identity to the sequence set forth in SEQ ID NO: 5.

In some embodiments, the VH region is encoded by a nucleic acid sequence having at least about 80%, 85%, 90%, 91%, 92%, 93%, 94%, 95%, 96%, 97%, 98%, 99%, or more, sequence identity to the sequence set forth in SEQ ID NO: 7, and the VL region is encoded by a nucleic acid sequence having at least about 80%, 85%, 90%, 91%, 92%, 93%, 94%, 95%, 96%, 97%, 98%, 99%, or more, sequence identity to the sequence set forth in SEQ ID NO: 9.

In some embodiments, the VH region is encoded by a nucleic acid sequence having at least about 80%, 85%, 90%, 91%, 92%, 93%, 94%, 95%, 96%, 97%, 98%, 99%, or more, sequence identity to the sequence set forth in SEQ ID NO: 11, and the VL region is encoded by a nucleic acid sequence having at least about 80%, 85%, 90%, 91%, 92%, 93%, 94%, 95%, 96%, 97%, 98%, 99%, or more, sequence identity to the sequence set forth in SEQ ID NO: 13.

In some embodiments, the VH region is encoded by a nucleic acid sequence having at least about 80%, 85%, 90%, 91%, 92%, 93%, 94%, 95%, 96%, 97%, 98%, 99%, or more, sequence identity to the sequence set forth in SEQ ID NO: 3, and the VL region is encoded by a nucleic acid sequence having at least about 80%, 85%, 90%, 91%, 92%, 93%, 94%, 95%, 96%, 97%, 98%, 99%, or more, sequence identity to the sequence set forth in SEQ ID NO: 9.

In some embodiments, the VH region is encoded by a nucleic acid sequence having at least about 80%, 85%, 90%, 91%, 92%, 93%, 94%, 95%, 96%, 97%, 98%, 99%, or more, sequence identity to the sequence set forth in SEQ ID NO: 3, and the VL region is encoded by a nucleic acid sequence having at least about 80%, 85%, 90%, 91%, 92%, 93%, 94%, 95%, 96%, 97%, 98%, 99%, or more, sequence identity to the sequence set forth in SEQ ID NO: 13.

In some embodiments, the VH region is encoded by a nucleic acid sequence having at least about 80%, 85%, 90%, 91%, 92%, 93%, 94%, 95%, 96%, 97%, 98%, 99%, or more, sequence identity to the sequence set forth in SEQ ID NO: 7, and the VL region is encoded by a nucleic acid sequence having at least about 80%, 85%, 90%, 91%, 92%, 93%, 94%, 95%, 96%, 97%, 98%, 99%, or more, sequence identity to the sequence set forth in SEQ ID NO: 5.

In some embodiments, the VH region is encoded by a nucleic acid sequence having at least about 80%, 85%, 90%, 91%, 92%, 93%, 94%, 95%, 96%, 97%, 98%, 99%, or more, sequence identity to the sequence set forth in SEQ ID NO: 7, and the VL region is encoded by a nucleic acid sequence having at least about 80%, 85%, 90%, 91%, 92%, 93%, 94%, 95%, 96%, 97%, 98%, 99%, or more, sequence identity to the sequence set forth in SEQ ID NO: 13.

In some embodiments, the VH region is encoded by a nucleic acid sequence having at least about 80%, 85%, 90%, 91%, 92%, 93%, 94%, 95%, 96%, 97%, 98%, 99%, or more, sequence identity to the sequence set forth in SEQ ID NO: 11, and the VL region is encoded by a nucleic acid sequence having at least about 80%, 85%, 90%, 91%, 92%, 93%, 94%, 95%, 96%, 97%, 98%, 99%, or more, sequence identity to the sequence set forth in SEQ ID NO: 5.

In some embodiments, the VH region is encoded by a nucleic acid sequence having at least about 80%, 85%, 90%, 91%, 92%, 93%, 94%, 95%, 96%, 97%, 98%, 99%, or more, sequence identity to the sequence set forth in SEQ ID NO: 11, and the VL region is encoded by a nucleic acid sequence having at least about 80%, 85%, 90%, 91%, 92%, 93%, 94%, 95%, 96%, 97%, 98%, 99%, or more, sequence identity to the sequence set forth in SEQ ID NO: 9.

In certain embodiments, the VH region comprises an amino acid sequence set forth in SEQ ID NO: 2. In certain embodiments, the VH region comprises an amino acid sequence set forth in SEQ ID NO: 6. In certain embodiments, the VH region comprises an amino acid sequence set forth in SEQ ID NO: 10.

In certain embodiments, the VH region is encoded by a nucleic acid sequence set forth in SEQ ID NO: 3. In certain embodiments, the VH region is encoded by a nucleic acid sequence set forth in SEQ ID NO: 7. In certain embodiments, the VH region is encoded by a nucleic acid sequence set forth in SEQ ID NO: 11.

In some embodiments, the VL region comprises an amino acid sequence set forth in SEQ ID NO: 4. In some embodiments, the VL region comprises an amino acid sequence set forth in SEQ ID NO: 8. In some embodiments, the VL region comprises an amino acid sequence set forth in SEQ ID NO: 12.

In some embodiments, the VL region is encoded by a nucleic acid sequence set forth in SEQ ID NO: 5. In some embodiments, the VL region is encoded by a nucleic acid sequence set forth in SEQ ID NO: 9. In some embodiments, the VL region is encoded by a nucleic acid sequence set forth in SEQ ID NO: 13.

In some embodiments, (a) the VH region comprises an amino acid sequence set forth in SEQ ID NO: 2, and the VL region comprises an amino acid sequence set forth in SEQ ID NO: 4.

In some embodiments, the VH region comprises an amino acid sequence set forth in SEQ ID NO: 6, and the VL region comprises an amino acid sequence set forth in SEQ ID NO: 8.

In some embodiments, the VH region comprises an amino acid sequence set forth in SEQ ID NO: 10, and the VL region comprises an amino acid sequence set forth in SEQ ID NO: 12.

In some embodiments, the VH region comprises an amino acid sequence set forth in SEQ ID NO: 2, and the VL region comprises an amino acid sequence set forth in SEQ ID NO: 8.

In some embodiments, the VH region comprises an amino acid sequence set forth in SEQ ID NO: 2, and the VL region comprises an amino acid sequence set forth in SEQ ID NO: 12.

In some embodiments, the VH region comprises an amino acid sequence set forth in SEQ ID NO: 6, and the VL region comprises an amino acid sequence set forth in SEQ ID NO: 4.

In some embodiments, the VH region comprises an amino acid sequence set forth in SEQ ID NO: 6, and the VL region comprises an amino acid sequence set forth in SEQ ID NO: 12.

In some embodiments, the VH region comprises an amino acid sequence set forth in SEQ ID NO: 10, and the VL region comprises an amino acid sequence set forth in SEQ ID NO: 4.

In some embodiments, the VH region comprises an amino acid sequence set forth in SEQ ID NO: 10, and the VL region comprises an amino acid sequence set forth in SEQ ID NO: 8.

In certain embodiments, the VH region is encoded by a nucleic acid sequence set forth in SEQ ID NO: 3, and the VL region is encoded by a nucleic acid sequence set forth in SEQ ID NO: 5.

In certain embodiments, the VH region is encoded by a nucleic acid sequence set forth in SEQ ID NO: 7, and the VL region is encoded by a nucleic acid sequence set forth in SEQ ID NO: 9.

In certain embodiments, the VH region is encoded by a nucleic acid sequence set forth in SEQ ID NO: 11, and the VL region is encoded by a nucleic acid sequence set forth in SEQ ID NO: 13.

In certain embodiments, the VH region is encoded by a nucleic acid sequence set forth in SEQ ID NO: 3, and the VL region is encoded by a nucleic acid sequence set forth in SEQ ID NO: 9.

In certain embodiments, the VH region is encoded by a nucleic acid sequence set forth in SEQ ID NO: 3, and the VL region is encoded by a nucleic acid sequence set forth in SEQ ID NO: 13.

In certain embodiments, the VH region is encoded by a nucleic acid sequence set forth in SEQ ID NO: 7, and the VL region is encoded by a nucleic acid sequence set forth in SEQ ID NO: 5.

In certain embodiments, the VH region is encoded by a nucleic acid sequence set forth in SEQ ID NO: 7, and the VL region is encoded by a nucleic acid sequence set forth in SEQ ID NO: 13.

In certain embodiments, the VH region is encoded by a nucleic acid sequence set forth in SEQ ID NO: 11, and the VL region is encoded by a nucleic acid sequence set forth in SEQ ID NO: 5.

In certain embodiments, the VH region is encoded by a nucleic acid sequence set forth in SEQ ID NO: 11, and the VL region is encoded by a nucleic acid sequence set forth in SEQ ID NO: 9, or In some embodiments, the isolated antibody, or antigen binding fragment thereof, comprises a heavy chain constant (CH) region, wherein the HC region comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 77.

In some embodiments, the CH region is encoded by a nucleic acid sequence having at least about 80%, 85%, 90%, 91%, 92%, 93%, 94%, 95%, 96%, 97%, 98%, 99%, or more, sequence identity to a sequence set forth in SEQ ID NO: 78.

In some embodiments, the CH region comprises an amino acid sequence set forth in SEQ ID NO: 77.

In some embodiments, the CH region is encoded by a nucleic acid sequence set forth in SEQ ID NO: 78.

In certain embodiments, the isolated antibody, or antigen binding fragment thereof, comprises a light chain constant (CL) region, wherein the LC region comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 79.

In certain embodiments, the CL region is encoded by a nucleic acid sequence having at least about 80%, 85%, 90%, 91%, 92%, 93%, 94%, 95%, 96%, 97%, 98%, 99%, or more, sequence identity to a sequence set forth in SEQ ID NO: 80.

In certain embodiments, the CL region comprises an amino acid sequence set forth in SEQ ID NO: 79.

In certain embodiments, the CL region is encoded by a nucleic acid sequence set forth in SEQ ID NO: 80.

In some embodiments, the antibody is an intact antibody.

In some embodiments, the antigen-binding fragment of the antibody is an Fab. In some embodiments, the antigen-binding fragment of the antibody is an Fab′. In some embodiments, the antigen-binding fragment of the antibody is an F(ab′)2. In some embodiments, the antigen-binding fragment of the antibody is an Fv.

In particular embodiments, the antigen-binding fragment of the antibody is an scFv.

In some such embodiments, the scFv comprises a linker connecting the VH region and the VL region. In some such embodiments, the VH region, the VL region, and the linker have a 5′ to 3′ orientation of VH region-linker-VL region. In some such embodiments, the VH region, the VL region, and the linker have a 5′ to 3′ orientation of VL region-linker-VH region.

In certain such embodiments, the linker comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 34. In certain such embodiments, the linker comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 35. In certain such embodiments, the linker comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 36. In certain such embodiments, the linker comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 37. In certain such embodiments, the linker comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 38. In certain such embodiments, the linker comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 39. In certain such embodiments, the linker comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 40. In certain such embodiments, the linker comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 41. In certain such embodiments, the linker comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 42. In certain such embodiments, the linker comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 43. In certain such embodiments, the linker comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 44. In certain such embodiments, the linker comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 45. In certain such embodiments, the linker comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 46. In certain such embodiments, the linker comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 47. In certain such embodiments, the linker comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 48. In certain such embodiments, the linker comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 49. In certain such embodiments, the linker comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 50. In certain such embodiments, the linker comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 51.

In certain such embodiments, the linker comprises an amino acid sequence set forth in SEQ ID NO: 34. In certain such embodiments, the linker comprises an amino acid sequence set forth in SEQ ID NO: 35. In certain such embodiments, the linker comprises an amino acid sequence set forth in SEQ ID NO: 36. In certain such embodiments, the linker comprises an amino acid sequence set forth in SEQ ID NO: 37. In certain such embodiments, the linker comprises an amino acid sequence set forth in SEQ ID NO: 38. In certain such embodiments, the linker comprises an amino acid sequence set forth in SEQ ID NO: 39. In certain such embodiments, the linker comprises an amino acid sequence set forth in SEQ ID NO: 40. In certain such embodiments, the linker comprises an amino acid sequence set forth in SEQ ID NO: 41. In certain such embodiments, the linker comprises an amino acid sequence set forth in SEQ ID NO: 42. In certain such embodiments, the linker comprises an amino acid sequence set forth in SEQ ID NO: 43. In certain such embodiments, the linker comprises an amino acid sequence set forth in SEQ ID NO: 44. In certain such embodiments, the linker comprises an amino acid sequence set forth in SEQ ID NO: 45. In certain such embodiments, the linker comprises an amino acid sequence set forth in SEQ ID NO: 46. In certain such embodiments, the linker comprises an amino acid sequence set forth in SEQ ID NO: 47. In certain such embodiments, the linker comprises an amino acid sequence set forth in SEQ ID NO: 48. In certain such embodiments, the linker comprises an amino acid sequence set forth in SEQ ID NO: 49. In certain such embodiments, the linker comprises an amino acid sequence set forth in SEQ ID NO: 50. In certain such embodiments, the linker comprises an amino acid sequence set forth in SEQ ID NO: 51.

In some such embodiments, the scFv comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 81. In some such embodiments, the scFv comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 82. In some such embodiments, the scFv comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 83. In some such embodiments, the scFv comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 84. In some such embodiments, the scFv comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 85. In some such embodiments, the scFv comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 86. In some such embodiments, the scFv comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 87. In some such embodiments, the scFv comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 88. In some such embodiments, the scFv comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 89. In some such embodiments, the scFv comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 90. In some such embodiments, the scFv comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 91. In some such embodiments, the scFv comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 92. In some such embodiments, the scFv comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 93. In some such embodiments, the scFv comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 94. In some such embodiments, the scFv comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 95. In some such embodiments, the scFv comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 96. In some such embodiments, the scFv comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 97. In some such embodiments, the scFv comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 98.

In some such embodiments, the scFv is encoded by a nucleic acid sequence having at least about 80%, 85%, 90%, 91%, 92%, 93%, 94%, 95%, 96%, 97%, 98%, 99%, or more, sequence identity to a sequence set forth in SEQ ID NO: 99. In some such embodiments, the scFv is encoded by a nucleic acid sequence having at least about 80%, 85%, 90%, 91%, 92%, 93%, 94%, 95%, 96%, 97%, 98%, 99%, or more, sequence identity to a sequence set forth in SEQ ID NO: 100. In some such embodiments, the scFv is encoded by a nucleic acid sequence having at least about 80%, 85%, 90%, 91%, 92%, 93%, 94%, 95%, 96%, 97%, 98%, 99%, or more, sequence identity to a sequence set forth in SEQ ID NO: 101. In some such embodiments, the scFv is encoded by a nucleic acid sequence having at least about 80%, 85%, 90%, 91%, 92%, 93%, 94%, 95%, 96%, 97%, 98%, 99%, or more, sequence identity to a sequence set forth in SEQ ID NO: 102. In some such embodiments, the scFv is encoded by a nucleic acid sequence having at least about 80%, 85%, 90%, 91%, 92%, 93%, 94%, 95%, 96%, 97%, 98%, 99%, or more, sequence identity to a sequence set forth in SEQ ID NO: 103. In some such embodiments, the scFv is encoded by a nucleic acid sequence having at least about 80%, 85%, 90%, 91%, 92%, 93%, 94%, 95%, 96%, 97%, 98%, 99%, or more, sequence identity to a sequence set forth in SEQ ID NO: 104. In some such embodiments, the scFv is encoded by a nucleic acid sequence having at least about 80%, 85%, 90%, 91%, 92%, 93%, 94%, 95%, 96%, 97%, 98%, 99%, or more, sequence identity to a sequence set forth in SEQ ID NO: 105. In some such embodiments, the scFv is encoded by a nucleic acid sequence having at least about 80%, 85%, 90%, 91%, 92%, 93%, 94%, 95%, 96%, 97%, 98%, 99%, or more, sequence identity to a sequence set forth in SEQ ID NO: 106. In some such embodiments, the scFv is encoded by a nucleic acid sequence having at least about 80%, 85%, 90%, 91%, 92%, 93%, 94%, 95%, 96%, 97%, 98%, 99%, or more, sequence identity to a sequence set forth in SEQ ID NO: 107. In some such embodiments, the scFv is encoded by a nucleic acid sequence having at least about 80%, 85%, 90%, 91%, 92%, 93%, 94%, 95%, 96%, 97%, 98%, 99%, or more, sequence identity to a sequence set forth in SEQ ID NO: 108. In some such embodiments, the scFv is encoded by a nucleic acid sequence having at least about 80%, 85%, 90%, 91%, 92%, 93%, 94%, 95%, 96%, 97%, 98%, 99%, or more, sequence identity to a sequence set forth in SEQ ID NO: 109. In some such embodiments, the scFv is encoded by a nucleic acid sequence having at least about 80%, 85%, 90%, 91%, 92%, 93%, 94%, 95%, 96%, 97%, 98%, 99%, or more, sequence identity to a sequence set forth in SEQ ID NO: 110. In some such embodiments, the scFv is encoded by a nucleic acid sequence having at least about 80%, 85%, 90%, 91%, 92%, 93%, 94%, 95%, 96%, 97%, 98%, 99%, or more, sequence identity to a sequence set forth in SEQ ID NO: 111. In some such embodiments, the scFv is encoded by a nucleic acid sequence having at least about 80%, 85%, 90%, 91%, 92%, 93%, 94%, 95%, 96%, 97%, 98%, 99%, or more, sequence identity to a sequence set forth in SEQ ID NO: 112. In some such embodiments, the scFv is encoded by a nucleic acid sequence having at least about 80%, 85%, 90%, 91%, 92%, 93%, 94%, 95%, 96%, 97%, 98%, 99%, or more, sequence identity to a sequence set forth in SEQ ID NO: 113. In some such embodiments, the scFv is encoded by a nucleic acid sequence having at least about 80%, 85%, 90%, 91%, 92%, 93%, 94%, 95%, 96%, 97%, 98%, 99%, or more, sequence identity to a sequence set forth in SEQ ID NO: 114. In some such embodiments, the scFv is encoded by a nucleic acid sequence having at least about 80%, 85%, 90%, 91%, 92%, 93%, 94%, 95%, 96%, 97%, 98%, 99%, or more, sequence identity to a sequence set forth in SEQ ID NO: 115. In some such embodiments, the scFv is encoded by a nucleic acid sequence having at least about 80%, 85%, 90%, 91%, 92%, 93%, 94%, 95%, 96%, 97%, 98%, 99%, or more, sequence identity to a sequence set forth in SEQ ID NO: 116.

In some such embodiments, the scFv comprises an amino acid sequence set forth in SEQ ID NO: 81. In some such embodiments, the scFv comprises an amino acid sequence set forth in SEQ ID NO: 82. In some such embodiments, the scFv comprises an amino acid sequence set forth in SEQ ID NO: 83. In some such embodiments, the scFv comprises an amino acid sequence set forth in SEQ ID NO: 84. In some such embodiments, the scFv comprises an amino acid sequence set forth in SEQ ID NO: 85. In some such embodiments, the scFv comprises an amino acid sequence set forth in SEQ ID NO: 86. In some such embodiments, the scFv comprises an amino acid sequence set forth in SEQ ID NO: 87. In some such embodiments, the scFv comprises an amino acid sequence set forth in SEQ ID NO: 88. In some such embodiments, the scFv comprises an amino acid sequence set forth in SEQ ID NO: 89. In some such embodiments, the scFv comprises an amino acid sequence set forth in SEQ ID NO: 90. In some such embodiments, the scFv comprises an amino acid sequence set forth in SEQ ID NO: 91. In some such embodiments, the scFv comprises an amino acid sequence set forth in SEQ ID NO: 92. In some such embodiments, the scFv comprises an amino acid sequence set forth in SEQ ID NO: 93. In some such embodiments, the scFv comprises an amino acid sequence set forth in SEQ ID NO: 94. In some such embodiments, the scFv comprises an amino acid sequence set forth in SEQ ID NO: 95. In some such embodiments, the scFv comprises an amino acid sequence set forth in SEQ ID NO: 96. In some such embodiments, the scFv comprises an amino acid sequence set forth in SEQ ID NO: 97. In some such embodiments, the scFv comprises an amino acid sequence set forth in SEQ ID NO: 98.

In some such embodiments, the scFv is encoded by a nucleic acid sequence set forth in SEQ ID NO: 99. In some such embodiments, the scFv is encoded by a nucleic acid sequence set forth in SEQ ID NO: 100. In some such embodiments, the scFv is encoded by a nucleic acid sequence set forth in SEQ ID NO: 101. In some such embodiments, the scFv is encoded by a nucleic acid sequence set forth in SEQ ID NO: 102. In some such embodiments, the scFv is encoded by a nucleic acid sequence set forth in SEQ ID NO: 103. In some such embodiments, the scFv is encoded by a nucleic acid sequence set forth in SEQ ID NO: 104. In some such embodiments, the scFv is encoded by a nucleic acid sequence set forth in SEQ ID NO: 105. In some such embodiments, the scFv is encoded by a nucleic acid sequence set forth in SEQ ID NO: 106. In some such embodiments, the scFv is encoded by a nucleic acid sequence set forth in SEQ ID NO: 107. In some such embodiments, the scFv is encoded by a nucleic acid sequence set forth in SEQ ID NO: 108. In some such embodiments, the scFv is encoded by a nucleic acid sequence set forth in SEQ ID NO: 109. In some such embodiments, the scFv is encoded by a nucleic acid sequence set forth in SEQ ID NO: 110. In some such embodiments, the scFv is encoded by a nucleic acid sequence set forth in SEQ ID NO: 111. In some such embodiments, the scFv is encoded by a nucleic acid sequence set forth in SEQ ID NO: 112. In some such embodiments, the scFv is encoded by a nucleic acid sequence set forth in SEQ ID NO: 113. In some such embodiments, the scFv is encoded by a nucleic acid sequence set forth in SEQ ID NO: 114. In some such embodiments, the scFv is encoded by a nucleic acid sequence set forth in SEQ ID NO: 115. In some such embodiments, the scFv is encoded by a nucleic acid sequence set forth in SEQ ID NO: 116.

In certain embodiments, the isolated antibody, or antigen-binding fragment thereof, binds (e.g., specifically binds) to a human BCMA comprising the amino acid sequence set forth in SEQ ID NO: 1.

In some embodiments, the isolated antibody, or antigen-binding fragment thereof, binds (e.g., specifically binds) to human BCMA with a binding affinity (KD) of from about 1×10⁻⁹ M to about 1×10⁻⁸ M.

In certain embodiments, the isolated antibody, or antibody fragment thereof, comprises a human variable region framework region. In certain embodiments, the isolated antibody, or antigen-binding fragment thereof, is a fully murine antibody, or antigen-binding fragment thereof. In certain embodiments, the isolated antibody, or antigen-binding fragment thereof, is a chimeric antibody, or antigen-binding fragment thereof. In certain embodiments, the isolated antibody, or antigen-binding fragment thereof, is a humanized antibody, or antigen-binding fragment thereof.

In another aspect, the invention provides an isolated antibody, or antigen-binding fragment thereof, comprising a VH region that comprises a CDRH1 domain, a CDRH2 domain, and a CDRH3 domain of any VH region set forth in any one of SEQ ID NOs: 2, 6, and 10, wherein the isolated antibody, or antigen-binding fragment thereof, specifically binds (e.g., specifically binds) to human BCMA.

In some embodiments, the isolated antibody, or antigen-binding fragment thereof, is a single domain antibody (sdAb).

In some embodiments, the CDRH1 domain, the CDRH2 domain, the CDRH3 domain are from a VH region set forth in SEQ ID NO: 2. In some embodiments, the CDRH1 domain, the CDRH2 domain, the CDRH3 domain are from a VH region set forth in SEQ ID NO: 6. In some embodiments, the CDRH1 domain, the CDRH2 domain, the CDRH3 domain are from a VH region set forth in SEQ ID NO: 10.

In certain embodiments, the CDRH1 domain, the CDRH2 domain, and the CDRH3 domain are identified by the Kabat numbering scheme. In certain embodiments, the CDRH1 domain, the CDRH2 domain, and the CDRH3 domain are identified by the Chothia numbering scheme.

In some embodiments, the CDRH1 domain comprises an amino acid sequence set forth in SEQ ID NO: 14. In some embodiments, the CDRH1 domain comprises an amino acid sequence set forth in SEQ ID NO: 20. In some embodiments, the CDRH1 domain comprises an amino acid sequence set forth in SEQ ID NO: 26.

In some embodiments, the CDRH2 domain comprises an amino acid sequence set forth in SEQ ID NO: 15. In some embodiments, the CDRH2 domain comprises an amino acid sequence set forth in SEQ ID NO: 21. In some embodiments, the CDRH2 domain comprises an amino acid sequence set forth in SEQ ID NO: 27.

In some embodiments, the CDRH3 domain comprises an amino acid sequence set forth in SEQ ID NO: 16. In some embodiments, the CDRH3 domain comprises an amino acid sequence set forth in SEQ ID NO: 22. In some embodiments, the CDRH3 domain comprises an amino acid sequence set forth in SEQ ID NO: 28.

In certain embodiments, the CDRH1 domain comprises an amino acid sequence set forth in SEQ ID NO: 14; the CDRH2 domain comprises an amino acid sequence set forth in SEQ ID NO: 15; and the CDRH3 domain comprises an amino acid sequence set forth in SEQ ID NO: 16. In certain embodiments, the CDRH1 domain comprises an amino acid sequence set forth in SEQ ID NO: 20; the CDRH2 domain comprises an amino acid sequence set forth in SEQ ID NO: 21; and the CDRH3 domain comprises an amino acid sequence set forth in SEQ ID NO: 22. In certain embodiments, the CDRH1 domain comprises an amino acid sequence set forth in SEQ ID NO: 26; the CDRH2 domain comprises an amino acid sequence set forth in SEQ ID NO: 27; and the CDRH3 domain comprises an amino acid sequence set forth in SEQ ID NO: 28.

In certain embodiments, the VH region comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 2. In certain embodiments, the VH region comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 6. In certain embodiments, the VH region comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 10.

In certain embodiments, the VH region is encoded by a nucleic acid sequence having at least about 80%, 85%, 90%, 91%, 92%, 93%, 94%, 95%, 96%, 97%, 98%, 99%, or more, sequence identity to a sequence set forth in SEQ ID NO: 3. In certain embodiments, the VH region is encoded by a nucleic acid sequence having at least about 80%, 85%, 90%, 91%, 92%, 93%, 94%, 95%, 96%, 97%, 98%, 99%, or more, sequence identity to a sequence set forth in SEQ ID NO: 7. In certain embodiments, the VH region is encoded by a nucleic acid sequence having at least about 80%, 85%, 90%, 91%, 92%, 93%, 94%, 95%, 96%, 97%, 98%, 99%, or more, sequence identity to a sequence set forth in SEQ ID NO: 11.

In some embodiments, the VH region comprises an amino acid sequence set forth in SEQ ID NO: 2. In some embodiments, the VH region comprises an amino acid sequence set forth in SEQ ID NO: 6. In some embodiments, the VH region comprises an amino acid sequence set forth in SEQ ID NO: 10.

In some embodiments, the VH region is encoded by a nucleic acid sequence set forth in SEQ ID NO: 3. In some embodiments, the VH region is encoded by a nucleic acid sequence set forth in SEQ ID NO: 7. In some embodiments, the VH region is encoded by a nucleic acid sequence set forth in SEQ ID NO: 11.

In certain embodiments, the isolated antibody, or antigen-binding fragment thereof, binds (e.g., specifically binds) to a human BCMA comprising the amino acid sequence set forth in SEQ ID NO: 1.

In some embodiments, the isolated antibody, or antigen-binding fragment thereof, binds (e.g., specifically binds) to human BCMA with a binding affinity (KD) of from about 1×10⁻⁹ M to about 1×10⁻⁸ M.

In certain embodiments, the isolated antibody, or antibody fragment thereof, comprises a human variable region framework region. In certain embodiments, the isolated antibody, or antigen-binding fragment thereof, is a fully murine antibody, or antigen-binding fragment thereof. In certain embodiments, the isolated antibody, or antigen-binding fragment thereof, is a chimeric antibody, or antigen-binding fragment thereof. In certain embodiments, the isolated antibody, or antigen-binding fragment thereof, is a humanized antibody, or antigen-binding fragment thereof.

In another aspect, the invention provides an isolated antibody, or antigen-binding fragment thereof, which cross-competes for binding to human BCMA with an isolated antibody, or an antigen-binding fragment thereof, described herein.

In another aspect, the invention provides an isolated antibody, or antigen-binding fragment thereof, which binds (e.g., specifically binds) to the same epitope on human BCMA as the isolated antibody, or antigen-binding fragment thereof, described herein.

In another aspect, the invention provides a pharmaceutical composition comprising an isolated antibody, or antigen-binding fragment thereof, described herein and a pharmaceutically acceptable carrier.

In another aspect, the invention provides an immunoconjugate comprising an isolated antibody, or antigen-binding fragment thereof, described herein linked to a therapeutic agent.

In some embodiments, the therapeutic agent is a drug, a cytotoxin, or a radioactive isotope.

In another aspect, the invention provides a pharmaceutical composition comprising an immunoconjugate described herein and a pharmaceutically acceptable carrier.

In another aspect, the invention provides a bispecific molecule comprising an isolated antibody, or antigen-binding fragment thereof, described herein linked to a second functional moiety.

In some embodiments, the second functional moiety has a different binding specificity than the isolated antibody, or antigen binding fragment thereof.

In another aspect, the invention provides a pharmaceutical composition comprising a bispecific molecule described herein and a pharmaceutically acceptable carrier.

In another aspect, the invention provides a polynucleotide comprising a nucleic acid sequence encoding an isolated antibody, or antigen-binding fragment thereof, described herein. In another aspect, the invention provides an expression vector comprising the polynucleotide such a polynucleotide. In another aspect, the invention provides a host cell comprising such an expression vector.

In another aspect, the invention provides a method for detecting BCMA in a whole cell or tissue, comprising: (a) contacting a cell or tissue with an isolated antibody, or antigen-binding fragment thereof, described herein, wherein the isolated antibody, or antigen-binding fragment thereof, comprises a detectable label; and (b) determining the amount of the labeled isolated antibody, or antigen-binding fragment thereof, bound to the cell or tissue by measuring the amount of detectable label associated with the cell or tissue, wherein the amount of bound isolated antibody, or antigen-binding fragment thereof, indicates the amount of BCMA in the cell or tissue.

In another aspect, the invention provides a method of treating a cancer in a subject, comprising administering an effective amount of an isolated antibody, or antigen-binding fragment thereof, described herein, thereby inducing death of a cancer cell in the subject.

In some embodiments, the method reduces the number of the cancer cells. In some embodiments, the method reduces the size of the cancer. In some embodiments, the method eradicates the cancer in the subject.

In certain embodiments, the cancer is selected from the group consisting of multiple myeloma, Non-Hodgkin Lymphoma, Hodgkin Lymphoma, Chronic Lymphocytic Leukemia (CLL), glioblastoma, and Waldenstrom's Macroglobulinemia. In certain embodiments, the cancer is multiple myeloma.

In certain embodiments, the subject is a human.

In certain embodiments, the subject is administered a gamma secretase inhibitor. In some embodiments, an effective amount of the gamma secretase inhibitor is administered. In certain embodiments, the gamma secretase inhibitor is administered prior to administration of the isolated antibody, or antigen-binding fragment thereof. In some embodiments, the gamma secretase inhibitor is administered concurrently with administration of the isolated antibody, or antigen-binding fragment thereof.

In another aspect, the invention provides use of an isolated antibody, or antigen-binding fragment thereof, described herein for the treatment of a cancer.

In some embodiments, the cancer is selected from the group consisting of multiple myeloma, Non-Hodgkin Lymphoma, Hodgkin Lymphoma, Chronic Lymphocytic Leukemia (CLL), glioblastoma, and Waldenstrom's Macroglobulinemia. In some embodiments, the cancer is multiple myeloma.

In certain embodiments, the subject is administered a gamma secretase inhibitor. In some embodiments, an effective amount of the gamma secretase inhibitor is administered. In certain embodiments, the gamma secretase inhibitor is administered prior to administration of the isolated antibody, or antigen-binding fragment thereof. In some embodiments, the gamma secretase inhibitor is administered concurrently with administration of the isolated antibody, or antigen-binding fragment thereof.

In another aspect, the invention provides an isolated antibody, or antigen-binding fragment thereof, described herein for use in treating a cancer in a subject.

In some embodiments, the cancer is selected from the group consisting of multiple myeloma, Non-Hodgkin Lymphoma, Hodgkin Lymphoma, Chronic Lymphocytic Leukemia (CLL), glioblastoma, and Waldenstrom's Macroglobulinemia. In some embodiments, the cancer is multiple myeloma.

In certain embodiments, the subject is administered a gamma secretase inhibitor. In some embodiments, an effective amount of the gamma secretase inhibitor is administered. In certain embodiments, the gamma secretase inhibitor is administered prior to administration of the isolated antibody, or antigen-binding fragment thereof. In some embodiments, the gamma secretase inhibitor is administered concurrently with administration of the isolated antibody, or antigen-binding fragment thereof.

In another aspect, the invention provides a kit for treating a cancer, comprising an isolated antibody, or antigen-binding fragment thereof, described herein.

In some embodiments, the kit further comprises written instructions for using the isolated antibody, or antigen-binding fragment thereof, for treating a subject having the cancer. In some embodiments, the cancer is multiple myeloma.

In another aspect, the invention provides a polynucleotide comprising a nucleic acid sequence encoding a chimeric antigen receptor (CAR), wherein the CAR comprises a human anti-BCMA binding domain, a transmembrane domain, and an intracellular domain, and wherein the anti-BCMA binding domain comprises an isolated antibody, or antigen-binding fragment thereof, described herein.

In some embodiments, the anti-BCMA binding domain comprises an scFv described herein.

In some embodiments, the anti-BCMA binding domain comprises an sdAb described herein.

In certain embodiments, the anti-BCMA binding domain binds (e.g., specifically binds) to a human BCMA comprising an amino acid sequence set forth in SEQ ID NO: 1.

In some embodiments, the transmembrane domain comprises a transmembrane domain of a protein selected from the group consisting of the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137 and CD154. In some embodiments, the transmembrane domain comprises a CD8 transmembrane domain.

In some embodiments, the transmembrane domain comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 56. In some embodiments, the transmembrane domain comprises an amino acid sequence set forth in SEQ ID NO: 56.

In certain embodiments, the CAR comprises a hinge domain connecting the anti-BCMA binding domain and the transmembrane domain.

In certain embodiments, the hinge region comprises an amino acid sequence having at least about 80%, 85%, 90%, 91%, 92%, 93%, 94%, 95%, 96%, 97%, 98%, 99%, or more, sequence identity to the sequence set forth in SEQ ID NO: 54. In certain embodiments, the hinge region comprises an amino acid sequence set forth in SEQ ID NO: 54.

In some embodiments, the intracellular signaling domain comprises a co-stimulatory domain.

In some embodiments, the co-stimulatory domain comprises a Novel 6 (N6) domain, a Novel 1 (N1) domain, a 4-1BB domain, a CD28 domain, or a functional signaling domain obtained from a protein including an MHC class I molecule, TNF receptor proteins, Immunoglobulin-like proteins, cytokine receptors, integrins, signaling lymphocytic activation molecules (SLAM proteins), activating NK cell receptors, BTLA, a Toll ligand receptor, OX40, CD2, CD7, CD27, CD30, CD40, CDS, ICAM-1, LFA-1 (CD11a/CD18), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRT AM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83. In some embodiments, the co-stimulatory domain comprises a Novel 6 (N6) domain. In some embodiments, the co-stimulatory domain comprises a Novel 1 (N1) domain. In some embodiments, the co-stimulatory domain comprises a 4-1BB domain. In some embodiments, the co-stimulatory domain comprises a CD28 domain.

In some embodiments, the co-stimulatory domain comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 58. In some embodiments, the co-stimulatory domain comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 60. In some embodiments, the co-stimulatory domain comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 62. In some embodiments, the co-stimulatory domain comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 64.

In some embodiments, the co-stimulatory domain comprises an amino acid sequence set forth in SEQ ID NO: 58. In some embodiments, the co-stimulatory domain comprises an amino acid sequence set forth in SEQ ID NO: 60. In some embodiments, the co-stimulatory domain comprises an amino acid sequence set forth in SEQ ID NO: 62. In some embodiments, the co-stimulatory domain comprises an amino acid sequence set forth in SEQ ID NO: 64.

In certain embodiments, the intracellular domain comprises a signaling domain. In certain embodiments, the signaling domain is a CD3 zeta signaling domain. In certain embodiments, the signaling domain comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 66. In certain embodiments, the signaling domain comprises an amino acid sequence set forth in SEQ ID NO: 66.

In some embodiments, the sequences encoding the co-stimulatory domain and the signaling domain are expressed in the same frame and as a single polypeptide chain.

In some embodiments, the CAR comprises a spacer connecting the hinge domain to the anti-BCMA binding domain.

In some embodiments, the spacer comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 52. In some embodiments, the spacer comprises an amino acid sequence set forth in SEQ ID NO: 52. In some embodiments, the spacer is encoded by a nucleic acid sequence having at least about 80%, 85%, 90%, 91%, 92%, 93%, 94%, 95%, 96%, 97%, 98%, 99%, or more, sequence identity to a sequence set forth in SEQ ID NO: 53. In some embodiments, the spacer is encoded by a nucleic acid sequence comprising SEQ ID NO: 53.

In certain embodiments, the CAR comprises a signal peptide.

In certain embodiments, the signal peptide comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 68. In certain embodiments, the signal peptide comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 70. In certain embodiments, the signal peptide comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 189.

In certain embodiments, the signal peptide comprises an amino acid sequence set forth in SEQ ID NO: 68. In certain embodiments, the signal peptide comprises an amino acid sequence set forth in SEQ ID NO: 70. In certain embodiments, the signal peptide comprises an amino acid sequence set forth in SEQ ID NO: 189.

In some embodiments, the CAR comprises: (a) an anti-BCMA binding domain described herein comprising a VH region and a VL region; (b) a linker connecting the VH region to the VL region, wherein the anti-BCMA binding domain has a 5′ to 3′ orientation of VH region-linker-VL region or VL region-linker-VH region; (c) a hinge domain connecting the anti-BCMA binding domain to the transmembrane domain; (d) the transmembrane domain; (e) an intracellular co-stimulatory domain; and (f) an intracellular functional signaling domain.

In some embodiments, the CAR comprises: (a) an anti-BCMA binding domain described herein comprising a VH region and a VL region; (b) a linker connecting the VH region to the VL region, wherein the anti-BCMA binding domain has a 5′ to 3′ orientation of VH region-linker-VL region or VL region-linker-VH region and wherein the linker comprises an amino acid sequence having at least about 80%, 85%, 90%, 91%, 92%, 93%, 94%, 95%, 96%, 97%, 98%, 99%, or more, sequence identity to a sequence set forth in any one of SEQ ID NOs: 34-51; (c) a hinge domain connecting the BCMA-binding domain to the transmembrane domain, wherein the hinge domain comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 54; (d) the transmembrane domain, wherein the transmembrane domain comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 56; (e) an intracellular co-stimulatory domain comprising an amino acid sequence having at least about 80%, 85%, 90%, 91%, 92%, 93%, 94%, 95%, 96%, 97%, 98%, 99%, or more, sequence identity to a sequence set forth in any one of SEQ ID NOs: 58, 60, 62, and 64; and (f) an intracellular signaling domain comprising an amino acid sequence having at least about 80%, 85%, 90%, 91%, 92%, 93%, 94%, 95%, 96%, 97%, 98%, 99%, or more, sequence identity to a sequence set forth in SEQ ID NO: 66.

In some embodiments, the CAR comprises: (a) an anti-BCMA binding domain of any described herein comprising a VH region and a VL region; (b) a linker connecting the VH 15 region to the VL region, wherein the anti-BCMA binding domain has a 5′ to 3′ orientation of VH region-linker-VL region or VL region-linker-VH region and wherein the linker comprises an amino acid sequence set forth in any one of SEQ ID NOs: 34-51; (c) a hinge domain connecting the BCMA-binding domain to the transmembrane domain, wherein the hinge domain comprises an amino acid sequence set forth in SEQ ID NO: 54; (d) the transmembrane domain, wherein the transmembrane domain comprises an amino acid sequence set forth in SEQ ID NO: 56; (e) an intracellular co-stimulatory domain comprising an amino acid sequence set forth in any one of SEQ ID NOs: 58, 60, 62, and 64; and (f) an intracellular signaling domain comprising an amino acid sequence set forth in SEQ ID NO: 66.

In some such embodiments, wherein the CAR comprises an anti-BCMA binding domain described herein comprising a VH region and a VL region, the CAR comprises a spacer, wherein the spacer connects the BCMA-binding domain to the hinge domain. In some such embodiments, the spacer comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 52. In some such embodiments, the spacer comprises an amino acid sequence set forth in SEQ ID NO: 52. In some such embodiments, the spacer is encoded by a nucleic acid sequence having at least about 80%, 85%, 90%, 91%, 92%, 93%, 94%, 95%, 96%, 97%, 98%, 99%, or more, sequence identity to a sequence set forth in SEQ ID NO: 53. In some such embodiments, the spacer is encoded by a nucleic acid sequence comprising SEQ ID NO: 53.

In some such embodiments, wherein the CAR comprises an anti-BCMA binding domain described herein comprising a VH region and a VL region, the CAR comprises a signal peptide. In some such embodiments, the signal peptide comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 68, 70, or 189. In some such embodiments, the signal peptide comprises an amino acid sequence set forth in SEQ ID NO: 68, 70, or 189.

In certain embodiments, the CAR comprises: (a) an anti-BCMA binding domain described herein which comprises a VH domain, such as an sdAb; (b) a hinge domain connecting the anti-BCMA binding domain to the transmembrane domain; (c) the transmembrane domain; (d) an intracellular co-stimulatory domain; and (e) an intracellular functional signaling domain.

In certain embodiments, the CAR comprises: (a) an anti-BCMA binding domain described herein which comprises a VH domain, such as an sdAb; (b) a hinge domain connecting the anti-BCMA binding domain to the transmembrane domain, wherein the hinge domain comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 54; (c) the transmembrane domain, wherein the transmembrane domain comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 56; (d) an intracellular co-stimulatory domain comprising an amino acid sequence having at least about 80%, 85%, 90%, 91%, 92%, 93%, 94%, 95%, 96%, 97%, 98%, 99%, or more, sequence identity to a sequence set forth in any one of SEQ ID NOs: 58, 60, 62, and 64; and (e) an intracellular signaling domain comprising an amino acid sequence having at least about 80%, 85%, 90%, 91%, 92%, 93%, 94%, 95%, 96%, 97%, 98%, 99%, or more, sequence identity to a sequence set forth in SEQ ID NO: 66.

In certain embodiments, the CAR comprises: (a) an anti-BCMA binding domain described herein which comprises a VH domain, such as an sdAb; (b) a hinge domain connecting the anti-BCMA binding domain to the transmembrane domain, wherein the hinge domain comprises an amino acid sequence set forth in SEQ ID NO: 54; (c) the transmembrane domain, wherein the transmembrane domain comprises an amino acid sequence set forth in SEQ ID NO: 56; (d) an intracellular co-stimulatory domain comprising an amino acid sequence set forth in any one of SEQ ID NOs: 58, 60, 62, and 64; and (e) an intracellular signaling domain comprising an amino acid sequence set forth in SEQ ID NO: 66.

In some such embodiments, wherein the CAR comprises an anti-BCMA binding domain described herein which comprises a VH domain, such as an sdAb, the CAR comprises a spacer, wherein the spacer connects the anti-BCMA binding domain to the hinge domain. In some such embodiments, the spacer comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 52. In some such embodiments, the spacer comprises an amino acid sequence set forth in SEQ ID NO: 52. In some such embodiments, the spacer is encoded by a nucleic acid sequence having at least about 80%, 85%, 90%, 91%, 92%, 93%, 94%, 95%, 96%, 97%, 98%, 99%, or more, sequence identity to a sequence set forth in SEQ ID NO: 53. In some such embodiments, the spacer is encoded by a nucleic acid sequence comprising SEQ ID NO: 53.

In some such embodiments, wherein the CAR comprises an anti-BCMA binding domain described herein which comprises a VH domain, such as an sdAb, the CAR comprises a signal peptide. In some such embodiments, the signal peptide comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 68, 70, or 189. In some such embodiments, the signal peptide comprises an amino acid sequence set forth in SEQ ID NO: 68, 70, or 189.

In certain embodiments, a CAR described herein comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 117. In certain embodiments, a CAR described herein comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 118. In certain embodiments, a CAR described herein comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 119. In certain embodiments, a CAR described herein comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 120. In certain embodiments, a CAR described herein comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 121. In certain embodiments, a CAR described herein comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 122. In certain embodiments, a CAR described herein comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 123. In certain embodiments, a CAR described herein comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 124. In certain embodiments, a CAR described herein comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 125. In certain embodiments, a CAR described herein comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 126. In certain embodiments, a CAR described herein comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 127. In certain embodiments, a CAR described herein comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 128. In certain embodiments, a CAR described herein comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 129. In certain embodiments, a CAR described herein comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 130. In certain embodiments, a CAR described herein comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 131. In certain embodiments, a CAR described herein comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 132. In certain embodiments, a CAR described herein comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 133. In certain embodiments, a CAR described herein comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 134.

In certain embodiments, a CAR described herein is encoded by a nucleic acid sequence having at least 80%, at least 85%, at least 90%, at least 91%, at least 92%, at least 93%, at least 94%, at least 95%, at least 96%, at least 97%, at least 98%, at least 99%, or more, sequence identity to a sequence set forth in SEQ ID NO: 135. In certain embodiments, a CAR described herein is encoded by a nucleic acid sequence having at least 80%, at least 85%, at least 90%, at least 91%, at least 92%, at least 93%, at least 94%, at least 95%, at least 96%, at least 97%, at least 98%, at least 99%, or more, sequence identity to a sequence set forth in SEQ ID NO: 136. In certain embodiments, a CAR described herein is encoded by a nucleic acid sequence having at least 80%, at least 85%, at least 90%, at least 91%, at least 92%, at least 93%, at least 94%, at least 95%, at least 96%, at least 97%, at least 98%, at least 99%, or more, sequence identity to a sequence set forth in SEQ ID NO: 137. In certain embodiments, a CAR described herein is encoded by a nucleic acid sequence having at least 80%, at least 85%, at least 90%, at least 91%, at least 92%, at least 93%, at least 94%, at least 95%, at least 96%, at least 97%, at least 98%, at least 99%, or more, sequence identity to a sequence set forth in SEQ ID NO: 138. In certain embodiments, a CAR described herein is encoded by a nucleic acid sequence having at least 80%, at least 85%, at least 90%, at least 91%, at least 92%, at least 93%, at least 94%, at least 95%, at least 96%, at least 97%, at least 98%, at least 99%, or more, sequence identity to a sequence set forth in SEQ ID NO: 139. In certain embodiments, a CAR described herein is encoded by a nucleic acid sequence having at least 80%, at least 85%, at least 90%, at least 91%, at least 92%, at least 93%, at least 94%, at least 95%, at least 96%, at least 97%, at least 98%, at least 99%, or more, sequence identity to a sequence set forth in SEQ ID NO: 140. In certain embodiments, a CAR described herein is encoded by a nucleic acid sequence having at least 80%, at least 85%, at least 90%, at least 91%, at least 92%, at least 93%, at least 94%, at least 95%, at least 96%, at least 97%, at least 98%, at least 99%, or more, sequence identity to a sequence set forth in SEQ ID NO: 141. In certain embodiments, a CAR described herein is encoded by a nucleic acid sequence having at least 80%, at least 85%, at least 90%, at least 91%, at least 92%, at least 93%, at least 94%, at least 95%, at least 96%, at least 97%, at least 98%, at least 99%, or more, sequence identity to a sequence set forth in SEQ ID NO: 142. In certain embodiments, a CAR described herein is encoded by a nucleic acid sequence having at least 80%, at least 85%, at least 90%, at least 91%, at least 92%, at least 93%, at least 94%, at least 95%, at least 96%, at least 97%, at least 98%, at least 99%, or more, sequence identity to a sequence set forth in SEQ ID NO: 143. In certain embodiments, a CAR described herein is encoded by a nucleic acid sequence having at least 80%, at least 85%, at least 90%, at least 91%, at least 92%, at least 93%, at least 94%, at least 95%, at least 96%, at least 97%, at least 98%, at least 99%, or more, sequence identity to a sequence set forth in SEQ ID NO: 144. In certain embodiments, a CAR described herein is encoded by a nucleic acid sequence having at least 80%, at least 85%, at least 90%, at least 91%, at least 92%, at least 93%, at least 94%, at least 95%, at least 96%, at least 97%, at least 98%, at least 99%, or more, sequence identity to a sequence set forth in SEQ ID NO: 145. In certain embodiments, a CAR described herein is encoded by a nucleic acid sequence having at least 80%, at least 85%, at least 90%, at least 91%, at least 92%, at least 93%, at least 94%, at least 95%, at least 96%, at least 97%, at least 98%, at least 99%, or more, sequence identity to a sequence set forth in SEQ ID NO: 146. In certain embodiments, a CAR described herein is encoded by a nucleic acid sequence having at least 80%, at least 85%, at least 90%, at least 91%, at least 92%, at least 93%, at least 94%, at least 95%, at least 96%, at least 97%, at least 98%, at least 99%, or more, sequence identity to a sequence set forth in SEQ ID NO: 147. In certain embodiments, a CAR described herein is encoded by a nucleic acid sequence having at least 80%, at least 85%, at least 90%, at least 91%, at least 92%, at least 93%, at least 94%, at least 95%, at least 96%, at least 97%, at least 98%, at least 99%, or more, sequence identity to a sequence set forth in SEQ ID NO: 148. In certain embodiments, a CAR described herein is encoded by a nucleic acid sequence having at least 80%, at least 85%, at least 90%, at least 91%, at least 92%, at least 93%, at least 94%, at least 95%, at least 96%, at least 97%, at least 98%, at least 99%, or more, sequence identity to a sequence set forth in SEQ ID NO: 149. In certain embodiments, a CAR described herein is encoded by a nucleic acid sequence having at least 80%, at least 85%, at least 90%, at least 91%, at least 92%, at least 93%, at least 94%, at least 95%, at least 96%, at least 97%, at least 98%, at least 99%, or more, sequence identity to a sequence set forth in SEQ ID NO: 150. In certain embodiments, a CAR described herein is encoded by a nucleic acid sequence having at least 80%, at least 85%, at least 90%, at least 91%, at least 92%, at least 93%, at least 94%, at least 95%, at least 96%, at least 97%, at least 98%, at least 99%, or more, sequence identity to a sequence set forth in SEQ ID NO: 151. In certain embodiments, a CAR described herein is encoded by a nucleic acid sequence having at least 80%, at least 85%, at least 90%, at least 91%, at least 92%, at least 93%, at least 94%, at least 95%, at least 96%, at least 97%, at least 98%, at least 99%, or more, sequence identity to a sequence set forth in SEQ ID NO: 152.

In certain embodiments, a CAR described herein comprises an amino acid sequence set forth in SEQ ID NO: 117. In certain embodiments, a CAR described herein comprises an amino acid sequence set forth in SEQ ID NO: 118. In certain embodiments, a CAR described herein comprises an amino acid sequence set forth in SEQ ID NO: 119. In certain embodiments, a CAR described herein comprises an amino acid sequence set forth in SEQ ID NO: 120. In certain embodiments, a CAR described herein comprises an amino acid sequence set forth in SEQ ID NO: 121. In certain embodiments, a CAR described herein comprises an amino acid sequence set forth in SEQ ID NO: 122. In certain embodiments, a CAR described herein comprises an amino acid sequence set forth in SEQ ID NO: 123. In certain embodiments, a CAR described herein comprises an amino acid sequence set forth in SEQ ID NO: 124. In certain embodiments, a CAR described herein comprises an amino acid sequence set forth in SEQ ID NO: 125. In certain embodiments, a CAR described herein comprises an amino acid sequence set forth in SEQ ID NO: 126. In certain embodiments, a CAR described herein comprises an amino acid sequence set forth in SEQ ID NO: 127. In certain embodiments, a CAR described herein comprises an amino acid sequence set forth in SEQ ID NO: 128. In certain embodiments, a CAR described herein comprises an amino acid sequence set forth in SEQ ID NO: 129. In certain embodiments, a CAR described herein comprises an amino acid sequence set forth in SEQ ID NO: 130. In certain embodiments, a CAR described herein comprises an amino acid sequence set forth in SEQ ID NO: 131. In certain embodiments, a CAR described herein comprises an amino acid sequence set forth in SEQ ID NO: 132. In certain embodiments, a CAR described herein comprises an amino acid sequence set forth in SEQ ID NO: 133. In certain embodiments, a CAR described herein comprises an amino acid sequence set forth in SEQ ID NO: 134.

In certain embodiments, a CAR described herein is encoded by a nucleic acid sequence set forth in SEQ ID NO: 135. In certain embodiments, a CAR described herein is encoded by a nucleic acid sequence set forth in SEQ ID NO: 136. In certain embodiments, a CAR described herein is encoded by a nucleic acid sequence set forth in SEQ ID NO: 137. In certain embodiments, a CAR described herein is encoded by a nucleic acid sequence set forth in SEQ ID NO: 138. In certain embodiments, a CAR described herein is encoded by a nucleic acid sequence set forth in SEQ ID NO: 139. In certain embodiments, a CAR described herein is encoded by a nucleic acid sequence set forth in SEQ ID NO: 140. In certain embodiments, a CAR described herein is encoded by a nucleic acid sequence set forth in SEQ ID NO: 141. In certain embodiments, a CAR described herein is encoded by a nucleic acid sequence set forth in SEQ ID NO: 142. In certain embodiments, a CAR described herein is encoded by a nucleic acid sequence set forth in SEQ ID NO: 143. In certain embodiments, a CAR described herein is encoded by a nucleic acid sequence set forth in SEQ ID NO: 144. In certain embodiments, a CAR described herein is encoded by a nucleic acid sequence set forth in SEQ ID NO: 145. In certain embodiments, a CAR described herein is encoded by a nucleic acid sequence set forth in SEQ ID NO: 146. In certain embodiments, a CAR described herein is encoded by a nucleic acid sequence set forth in SEQ ID NO: 147. In certain embodiments, a CAR described herein is encoded by a nucleic acid sequence set forth in SEQ ID NO: 148. In certain embodiments, a CAR described herein is encoded by a nucleic acid sequence set forth in SEQ ID NO: 149. In certain embodiments, a CAR described herein is encoded by a nucleic acid sequence set forth in SEQ ID NO: 150. In certain embodiments, a CAR described herein is encoded by a nucleic acid sequence set forth in SEQ ID NO: 151. In certain embodiments, a CAR described herein is encoded by a nucleic acid sequence set forth in SEQ ID NO: 152.

In some embodiments, a CAR described herein comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 153. In some embodiments, a CAR described herein comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 154. In some embodiments, a CAR described herein comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 155. In some embodiments, a CAR described herein comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 156. In some embodiments, a CAR described herein comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 157. In some embodiments, a CAR described herein comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 158. In some embodiments, a CAR described herein comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 159. In some embodiments, a CAR described herein comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 160. In some embodiments, a CAR described herein comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 161. In some embodiments, a CAR described herein comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 162. In some embodiments, a CAR described herein comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 163. In some embodiments, a CAR described herein comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 164. In some embodiments, a CAR described herein comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 165. In some embodiments, a CAR described herein comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 166. In some embodiments, a CAR described herein comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 167. In some embodiments, a CAR described herein comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 168. In some embodiments, a CAR described herein comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 169. In some embodiments, a CAR described herein comprises an amino acid sequence having at least about 80%, 85%, 90%, 91%, 92%, 93%, 94%, 95%, 96%, 97%, 98%, 99%, or more, sequence identity to a sequence set forth in SEQ ID NO: 170.

In some embodiments, a CAR described herein is encoded by a nucleic acid sequence having at least 80%, at least 85%, at least 90%, at least 91%, at least 92%, at least 93%, at least 94%, at least 95%, at least 96%, at least 97%, at least 98%, at least 99%, or more, sequence identity to a sequence set forth in SEQ ID NO: 171. In some embodiments, a CAR described herein is encoded by a nucleic acid sequence having at least 80%, at least 85%, at least 90%, at least 91%, at least 92%, at least 93%, at least 94%, at least 95%, at least 96%, at least 97%, at least 98%, at least 99%, or more, sequence identity to a sequence set forth in SEQ ID NO: 172. In some embodiments, a CAR described herein is encoded by a nucleic acid sequence having at least 80%, at least 85%, at least 90%, at least 91%, at least 92%, at least 93%, at least 94%, at least 95%, at least 96%, at least 97%, at least 98%, at least 99%, or more, sequence identity to a sequence set forth in SEQ ID NO: 173. In some embodiments, a CAR described herein is encoded by a nucleic acid sequence having at least 80%, at least 85%, at least 90%, at least 91%, at least 92%, at least 93%, at least 94%, at least 95%, at least 96%, at least 97%, at least 98%, at least 99%, or more, sequence identity to a sequence set forth in SEQ ID NO: 174. In some embodiments, a CAR described herein is encoded by a nucleic acid sequence having at least 80%, at least 85%, at least 90%, at least 91%, at least 92%, at least 93%, at least 94%, at least 95%, at least 96%, at least 97%, at least 98%, at least 99%, or more, sequence identity to a sequence set forth in SEQ ID NO: 175. In some embodiments, a CAR described herein is encoded by a nucleic acid sequence having at least 80%, at least 85%, at least 90%, at least 91%, at least 92%, at least 93%, at least 94%, at least 95%, at least 96%, at least 97%, at least 98%, at least 99%, or more, sequence identity to a sequence set forth in SEQ ID NO: 176. In some embodiments, a CAR described herein is encoded by a nucleic acid sequence having at least 80%, at least 85%, at least 90%, at least 91%, at least 92%, at least 93%, at least 94%, at least 95%, at least 96%, at least 97%, at least 98%, at least 99%, or more, sequence identity to a sequence set forth in SEQ ID NO: 177. In some embodiments, a CAR described herein is encoded by a nucleic acid sequence having at least 80%, at least 85%, at least 90%, at least 91%, at least 92%, at least 93%, at least 94%, at least 95%, at least 96%, at least 97%, at least 98%, at least 99%, or more, sequence identity to a sequence set forth in SEQ ID NO: 178. In some embodiments, a CAR described herein is encoded by a nucleic acid sequence having at least 80%, at least 85%, at least 90%, at least 91%, at least 92%, at least 93%, at least 94%, at least 95%, at least 96%, at least 97%, at least 98%, at least 99%, or more, sequence identity to a sequence set forth in SEQ ID NO: 179. In some embodiments, a CAR described herein is encoded by a nucleic acid sequence having at least 80%, at least 85%, at least 90%, at least 91%, at least 92%, at least 93%, at least 94%, at least 95%, at least 96%, at least 97%, at least 98%, at least 99%, or more, sequence identity to a sequence set forth in SEQ ID NO: 180. In some embodiments, a CAR described herein is encoded by a nucleic acid sequence having at least 80%, at least 85%, at least 90%, at least 91%, at least 92%, at least 93%, at least 94%, at least 95%, at least 96%, at least 97%, at least 98%, at least 99%, or more, sequence identity to a sequence set forth in SEQ ID NO: 181. In some embodiments, a CAR described herein is encoded by a nucleic acid sequence having at least 80%, at least 85%, at least 90%, at least 91%, at least 92%, at least 93%, at least 94%, at least 95%, at least 96%, at least 97%, at least 98%, at least 99%, or more, sequence identity to a sequence set forth in SEQ ID NO: 182. In some embodiments, a CAR described herein is encoded by a nucleic acid sequence having at least 80%, at least 85%, at least 90%, at least 91%, at least 92%, at least 93%, at least 94%, at least 95%, at least 96%, at least 97%, at least 98%, at least 99%, or more, sequence identity to a sequence set forth in SEQ ID NO: 183. In some embodiments, a CAR described herein is encoded by a nucleic acid sequence having at least 80%, at least 85%, at least 90%, at least 91%, at least 92%, at least 93%, at least 94%, at least 95%, at least 96%, at least 97%, at least 98%, at least 99%, or more, sequence identity to a sequence set forth in SEQ ID NO: 184. In some embodiments, a CAR described herein is encoded by a nucleic acid sequence having at least 80%, at least 85%, at least 90%, at least 91%, at least 92%, at least 93%, at least 94%, at least 95%, at least 96%, at least 97%, at least 98%, at least 99%, or more, sequence identity to a sequence set forth in SEQ ID NO: 185. In some embodiments, a CAR described herein is encoded by a nucleic acid sequence having at least 80%, at least 85%, at least 90%, at least 91%, at least 92%, at least 93%, at least 94%, at least 95%, at least 96%, at least 97%, at least 98%, at least 99%, or more, sequence identity to a sequence set forth in SEQ ID NO: 186. In some embodiments, a CAR described herein is encoded by a nucleic acid sequence having at least 80%, at least 85%, at least 90%, at least 91%, at least 92%, at least 93%, at least 94%, at least 95%, at least 96%, at least 97%, at least 98%, at least 99%, or more, sequence identity to a sequence set forth in SEQ ID NO: 187. In some embodiments, a CAR described herein is encoded by a nucleic acid sequence having at least 80%, at least 85%, at least 90%, at least 91%, at least 92%, at least 93%, at least 94%, at least 95%, at least 96%, at least 97%, at least 98%, at least 99%, or more, sequence identity to a sequence set forth in SEQ ID NO: 188.

In some embodiments, a CAR described herein comprises an amino acid sequence set forth in SEQ ID NO: 153. In some embodiments, a CAR described herein comprises an amino acid sequence set forth in SEQ ID NO: 154. In some embodiments, a CAR described herein comprises an amino acid sequence set forth in SEQ ID NO: 155. In some embodiments, a CAR described herein comprises an amino acid sequence set forth in SEQ ID NO: 156. In some embodiments, a CAR described herein comprises an amino acid sequence set forth in SEQ ID NO: 157. In some embodiments, a CAR described herein comprises an amino acid sequence set forth in SEQ ID NO: 158. In some embodiments, a CAR described herein comprises an amino acid sequence set forth in SEQ ID NO: 159. In some embodiments, a CAR described herein comprises an amino acid sequence set forth in SEQ ID NO: 160. In some embodiments, a CAR described herein comprises an amino acid sequence set forth in SEQ ID NO: 161. In some embodiments, a CAR described herein comprises an amino acid sequence set forth in SEQ ID NO: 162. In some embodiments, a CAR described herein comprises an amino acid sequence set forth in SEQ ID NO: 163. In some embodiments, a CAR described herein comprises an amino acid sequence set forth in SEQ ID NO: 164. In some embodiments, a CAR described herein comprises an amino acid sequence set forth in SEQ ID NO: 165. In some embodiments, a CAR described herein comprises an amino acid sequence set forth in SEQ ID NO: 166. In some embodiments, a CAR described herein comprises an amino acid sequence set forth in SEQ ID NO: 167. In some embodiments, a CAR described herein comprises an amino acid sequence set forth in SEQ ID NO: 168. In some embodiments, a CAR described herein comprises an amino acid sequence set forth in SEQ ID NO: 169. In some embodiments, a CAR described herein comprises an amino acid sequence set forth in SEQ ID NO: 170.

In some embodiments, a CAR described herein is encoded by a nucleic acid sequence set forth in SEQ ID NO: 171. In some embodiments, a CAR described herein is encoded by a nucleic acid sequence set forth in SEQ ID NO: 172. In some embodiments, a CAR described herein is encoded by a nucleic acid sequence set forth in SEQ ID NO: 173. In some embodiments, a CAR described herein is encoded by a nucleic acid sequence set forth in SEQ ID NO: 174. In some embodiments, a CAR described herein is encoded by a nucleic acid sequence set forth in SEQ ID NO: 175. In some embodiments, a CAR described herein is encoded by a nucleic acid sequence set forth in SEQ ID NO: 176. In some embodiments, a CAR described herein is encoded by a nucleic acid sequence set forth in SEQ ID NO: 177. In some embodiments, a CAR described herein is encoded by a nucleic acid sequence set forth in SEQ ID NO: 178. In some embodiments, a CAR described herein is encoded by a nucleic acid sequence set forth in SEQ ID NO: 179. In some embodiments, a CAR described herein is encoded by a nucleic acid sequence set forth in SEQ ID NO: 180. In some embodiments, a CAR described herein is encoded by a nucleic acid sequence set forth in SEQ ID NO: 181. In some embodiments, a CAR described herein is encoded by a nucleic acid sequence set forth in SEQ ID NO: 182. In some embodiments, a CAR described herein is encoded by a nucleic acid sequence set forth in SEQ ID NO: 183. In some embodiments, a CAR described herein is encoded by a nucleic acid sequence set forth in SEQ ID NO: 184. In some embodiments, a CAR described herein is encoded by a nucleic acid sequence set forth in SEQ ID NO: 185. In some embodiments, a CAR described herein is encoded by a nucleic acid sequence set forth in SEQ ID NO: 186. In some embodiments, a CAR described herein is encoded by a nucleic acid sequence set forth in SEQ ID NO: 187. In some embodiments, a CAR described herein is encoded by a nucleic acid sequence set forth in SEQ ID NO: 188.

In certain embodiments, a polynucleotide described herein comprises a promoter that is operably linked to the nucleic acid sequence encoding the CAR.

In some such embodiments, the promoter comprises a nucleic acid sequence having at least about 80%, 85%, 90%, 91%, 92%, 93%, 94%, 95%, 96%, 97%, 98%, 99%, or more, sequence identity to a sequence set forth in SEQ ID NO: 72. In some such embodiments, the promoter comprises a nucleic acid sequence having at least about 80%, 85%, 90%, 91%, 92%, 93%, 94%, 95%, 96%, 97%, 98%, 99%, or more, sequence identity to a sequence set forth in SEQ ID NO: 73.

In some such embodiments, the promoter comprises a nucleic acid sequence set forth in SEQ ID NO: 72. In some such embodiments, the promoter comprises a nucleic acid sequence set forth in SEQ ID NO: 73.

In another aspect, the invention provides a CAR polypeptide encoded by a polynucleotide described herein.

In another aspect, the invention provides a recombinant DNA construct comprising a polynucleotide described herein.

In another aspect, the invention provides a recombinant virus comprising a polynucleotide described herein, wherein the recombinant virus is a recombinant adeno-associated virus (AAV), a recombinant lentivirus, a recombinant adenovirus, or a recombinant retrovirus.

In some embodiments, the recombinant virus is a recombinant AAV.

In some embodiments, the recombinant AAV has a serotype of AAV6.

In another aspect, the invention provides a genetically-modified eukaryotic cell comprising in its genome a polynucleotide described herein comprising a nucleic acid sequence encoding a CAR described herein, wherein the CAR is expressed by the genetically-modified eukaryotic cell.

In certain embodiments, the genetically-modified eukaryotic cell comprises an inactivated T cell receptor (TCR) alpha gene, an inactivated TCR alpha constant region (TRAC) gene, and/or an inactivated TCR beta gene. In certain embodiments, the genetically-modified eukaryotic cell comprises an inactivated TCR alpha gene. In certain embodiments, the genetically-modified eukaryotic cell comprises an inactivated TRAC gene. In certain embodiments, the genetically-modified eukaryotic cell comprises an inactivated TCR beta gene.

In certain embodiments, the polynucleotide is randomly integrated within the genome of the genetically-modified eukaryotic cell.

In some embodiments, the polynucleotide is positioned within the genome of the genetically-modified eukaryotic cell within a target gene, wherein expression of a polypeptide encoded by the target gene is disrupted.

In some embodiments, the target gene is a TCR alpha gene. In some embodiments, the target gene is a TRAC gene. In some embodiments, the target gene is a TCR beta gene.

In some embodiments, the polynucleotide is positioned within a sequence set forth in SEQ ID NO: 74.

In some embodiments, the polynucleotide is positioned between nucleotide positions 13 and 14 of a sequence set forth in SEQ ID NO: 74.

In certain embodiments, the genetically-modified eukaryotic cell is a genetically-modified immune cell.

In certain embodiments, the genetically-modified immune cell is a genetically-modified T cell. In certain embodiments, the genetically-modified immune cell is a genetically-modified NK cell. In certain embodiments, the genetically-modified immune cell is a genetically-modified B cell. In certain embodiments, the genetically-modified immune cell is a genetically-modified macrophage.

In certain embodiments, the genetically-modified eukaryotic cell is a genetically-modified induced pluripotent stem cell (iPSC).

In certain embodiments, the genetically-modified eukaryotic cell is a genetically-modified human cell.

In another aspect, the invention provides a method of producing a genetically-modified eukaryotic cell, the method comprising introducing into a eukaryotic cell a template nucleic acid comprising a polynucleotide described herein comprising a nucleic acid sequence encoding a CAR described herein, wherein the polynucleotide is integrated into the genome of the eukaryotic cell, and wherein the CAR is expressed by the genetically-modified eukaryotic cell.

In some embodiments, the polynucleotide is introduced by a recombinant lentivirus, and the polynucleotide is inserted into the genome of the eukaryotic cell by random integration.

In some embodiments, the target gene is a TCR alpha gene. In some embodiments, the target gene is a TRAC gene. In some embodiments, the target gene is a TCR beta gene.

In certain embodiments, the method comprises introducing into the eukaryotic cell: (a) a nucleic acid encoding an engineered nuclease having specificity for a recognition sequence in the genome of the eukaryotic cell, wherein the engineered nuclease is expressed in the eukaryotic cell; and (b) the template nucleic acid comprising the polynucleotide; wherein the engineered nuclease generates a cleavage site at the recognition sequence, and wherein the polynucleotide is inserted into the genome of the eukaryotic cell at the cleavage site.

In some embodiments, the template nucleic acid is introduced into the eukaryotic cell using a recombinant virus.

In some embodiments, the recombinant virus is a recombinant AAV.

In some embodiments, the recombinant AAV has a serotype of AAV6.

In certain embodiments, the nucleic acid encoding the engineered nuclease is an mRNA.

In some embodiments, the template nucleic acid comprises a 5′ homology arm and a 3′ homology arm which have homology to sequences 5′ upstream and 3′ downstream, respectively, of the cleavage site, wherein the polynucleotide is inserted into the cleavage site by homologous recombination.

In certain embodiments, the engineered nuclease is an engineered meganuclease. In certain embodiments, the engineered nuclease is a zinc finger nuclease. In certain embodiments, the engineered nuclease is a TALEN. In certain embodiments, the engineered nuclease is a compact TALEN. In certain embodiments, the engineered nuclease is a CRISPR system nuclease. In certain embodiments, the engineered nuclease is a megaTAL.

In certain embodiments, the engineered meganuclease comprises an amino acid sequence set forth in SEQ ID NO: 76.

In some embodiments, the recognition sequence is positioned within a target gene, and wherein insertion of the polynucleotide at the cleavage site disrupts expression of a polypeptide encoded by the target gene.

In some embodiments, the target gene is a TCR alpha gene. In some embodiments, the target gene is a TRAC gene. In some embodiments, the target gene is a TCR beta gene.

In some embodiments, the polynucleotide is inserted within a sequence set forth in SEQ ID NO: 74.

In some embodiments, the polynucleotide is inserted between nucleotide positions 13 and 14 of a sequence set forth in SEQ ID NO: 74.

In certain embodiments, the genetically-modified eukaryotic cell is a genetically-modified immune cell.

In certain embodiments, the genetically-modified immune cell is a genetically-modified T cell. In certain embodiments, the genetically-modified immune cell is a genetically-modified NK cell. In certain embodiments, the genetically-modified immune cell is a genetically-modified B cell. In certain embodiments, the genetically-modified immune cell is a genetically-modified macrophage.

In certain embodiments, the genetically-modified eukaryotic cell is a genetically-modified induced pluripotent stem cell (iPSC).

In certain embodiments, the genetically-modified eukaryotic cell is a genetically-modified human cell.

In another aspect, the invention provides a genetically-modified eukaryotic cell produced by a method described herein.

In another aspect, the invention provides a population of eukaryotic cells comprising a plurality of genetically-modified eukaryotic cells described herein.

In some embodiments, at least about 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96, 97%, 98%, 99%, or 100% of the eukaryotic cells in the population are genetically-modified eukaryotic cells described herein. In some embodiments, between about 10% to about 90% of the eukaryotic cells in the population are genetically-modified eukaryotic cells described herein. In some embodiments, between about 20% to about 80% of the eukaryotic cells in the population are genetically-modified eukaryotic cells described herein. In some embodiments, between about 30% to about 70% of the eukaryotic cells in the population are genetically-modified eukaryotic cells described herein. In some embodiments, between about 40% to about 70% of the eukaryotic cells in the population are genetically-modified eukaryotic cells described herein. In some embodiments, between about 40% to about 60% of the eukaryotic cells in the population are genetically-modified eukaryotic cells described herein. In some embodiments, between about 40% to about 50% of the eukaryotic cells in the population are genetically-modified eukaryotic cells described herein. In some embodiments, between about 50% to about 80% of the eukaryotic cells in the population are genetically-modified eukaryotic cells described herein. In some embodiments, between about 50% to about 70% of the eukaryotic cells in the population are genetically-modified eukaryotic cells described herein. In some embodiments, between about 50% to about 60% of the eukaryotic cells in the population are genetically-modified eukaryotic cells described herein. In some embodiments, between about 60% to about 80% of the eukaryotic cells in the population are genetically-modified eukaryotic cells described herein. In some embodiments, between about 70% to about 80% of the eukaryotic cells in the population are genetically-modified eukaryotic cells described herein.

In certain embodiments, the genetically-modified eukaryotic cells in the population express the CAR and comprise an inactivated TCR alpha gene, an inactivated TRAC gene, and/or an inactivated TCR beta gene. In certain embodiments, the genetically-modified eukaryotic cells in the population express the CAR and comprise an inactivated TCR alpha gene. In certain embodiments, the genetically-modified eukaryotic cells in the population express the CAR and comprise an inactivated TRAC gene. In certain embodiments, the genetically-modified eukaryotic cells in the population express the CAR and comprise an inactivated TCR beta gene.

In another aspect, the invention provides a pharmaceutical composition comprising a plurality of genetically-modified eukaryotic cells described herein, or a population of eukaryotic cells described herein, and a pharmaceutically-acceptable carrier.

In another aspect, the invention provides a method of treating a cancer in a subject, the method comprising administering to the subject an effective amount of a pharmaceutical composition described herein to the subject, thereby inducing death of a cancer cell in the subject.

In some embodiments, the method reduces the number of the cancer cells.

In some embodiments, the method reduces the size of the cancer.

In some embodiments, the method eradicates the cancer in the subject.

In certain embodiments, the cancer is selected from the group consisting of multiple myeloma, Non-Hodgkin Lymphoma, Hodgkin Lymphoma, Chronic Lymphocytic Leukemia (CLL), glioblastoma, and Waldenstrom's Macroglobulinemia.

In certain embodiments, the cancer is multiple myeloma.

In some embodiments, the pharmaceutical composition is administered in combination with a cancer therapy selected from the group consisting of chemotherapy, surgery, radiation, and gene therapy.

In certain embodiments, the subject is a human.

In certain embodiments, the subject is administered a gamma secretase inhibitor. In some embodiments, an effective amount of the gamma secretase inhibitor is administered. In certain embodiments, the gamma secretase inhibitor is administered prior to administration of the isolated antibody, or antigen-binding fragment thereof. In some embodiments, the gamma secretase inhibitor is administered concurrently with administration of the isolated antibody, or antigen-binding fragment thereof.

In another aspect, the invention provides the use of a genetically-modified eukaryotic cell described herein for the treatment of a cancer.

In some embodiments, the cancer is selected from the group consisting of multiple myeloma, Non-Hodgkin Lymphoma, Hodgkin Lymphoma, Chronic Lymphocytic Leukemia (CLL), glioblastoma, and Waldenstrom's Macroglobulinemia.

In some embodiments, the cancer is multiple myeloma.

In certain embodiments, the subject is administered a gamma secretase inhibitor. In some embodiments, an effective amount of the gamma secretase inhibitor is administered. In certain embodiments, the gamma secretase inhibitor is administered prior to administration of the isolated antibody, or antigen-binding fragment thereof. In some embodiments, the gamma secretase inhibitor is administered concurrently with administration of the isolated antibody, or antigen-binding fragment thereof.

In another aspect, the invention provides a genetically-modified eukaryotic cell described herein for use in treating a cancer in a subject.

In some embodiments, the cancer is selected from the group consisting of multiple myeloma, Non-Hodgkin Lymphoma, Hodgkin Lymphoma, Chronic Lymphocytic Leukemia (CLL), glioblastoma, and Waldenstrom's Macroglobulinemia.

In some embodiments, the cancer is multiple myeloma.

In certain embodiments, the subject is administered a gamma secretase inhibitor. In some embodiments, an effective amount of the gamma secretase inhibitor is administered. In certain embodiments, the gamma secretase inhibitor is administered prior to administration of the isolated antibody, or antigen-binding fragment thereof. In some embodiments, the gamma secretase inhibitor is administered concurrently with administration of the isolated antibody, or antigen-binding fragment thereof.

In another aspect, the invention provides a kit for treating a cancer, the kit comprising a genetically-modified eukaryotic cell described herein.

In some embodiments, the kit further comprises written instructions for using the genetically-modified eukaryotic cell for treating a subject having the cancer.

In certain embodiments, the cancer is multiple myeloma.

In another aspect, the invention provides a genetically-modified eukaryotic cell described herein use as a medicament.

### BRIEF DESCRIPTION OF THE DRAWINGS

FIGS. 1A and 1B show flow cytometry dot plots of full length anti-BCMA or anti-CD19 antibodies against BCMA expressing K562 cells (K562-BCMA). FIG. 1A) The top Left panel is a negative control that shows the number of cells registering positive after incubation with PBS (0% positive). The top right panel is a secondary antibody negative control and shows the number of cells registering positive after incubation with the secondary antibody only (0.18% positive). Bottom left panel is a non-specific antibody negative control that shows the number of cells registering positive after incubation with an anti-CD19 antibody FMC63 (1.20% positive). The bottom right panel is a positive control showing the number of cells registering positive after incubation with a BCMA specific reference antibody (10.1% positive). FIG. 1B) The left panel shows the number of cells registering positive after incubation with the BCMA-3L/3H full length antibody (1.71% positive). The right panel shows the number of cells registering positive after incubation with the BCMA-3L/20H full length antibody.

FIG. 2. Provides a binding affinity table of full-length antibodies against a His-tagged BCMA protein. Shown is the binding kinetics including the K_{D}, Kₒₙ, and K_{off} metrics for the BCMA-3L/3H, the BCMA-3L/20H, a positive control BCMA reference antibody, and a CD19-specific (FMC63) negative control antibody.

FIGS. 3A-3G show flow cytometry dot plots of CD3, CD8, CD4, CD62L, CD27, and CD45RO cell surface protein expression in transfected human T cells. FIG. 3A) Flow cytometry dot plots from cells that were transfected with the TRC 1-2L.1592 meganuclease only. FIGS. 3B-G are flow cytometry dot plots from cells that were transfected with the TRC 1-2L.1592 meganuclease and further transduced with the following CAR constructs: FIG. 3B) a CAR construct having a reference BCMA specific scFv positive control and 4-1BB co-stimulatory domain; FIG. 3C) a CAR construct having a reference BCMA specific scFv positive control and an N6 co-stimulatory domain; FIG. 3D) a CAR construct having a BCMA-3L/3H scFv and an N6 co-stimulatory domain; FIG. 3E) a CAR construct having a BCMA-3L/51cH scFv and an N6 co-stimulatory domain; FIG. 3F) a CAR construct having a BCMA-20L/51cH scFv; and an N6 co-stimulatory domain FIG. 3G) a CAR construct having a BCMA-3L/20H scFv and an N6 co-stimulatory domain. Within each of FIGS. 3A-3G are flow cytometry dot plots showing a four quadrant gate with cells stained with antibodies specific for CD3, CD8, CD4, CD62L, CD27, and CD45RO cell surface protein as follows: 1) All cells with CAR expression on the Y axis and CD3 expression on the X axis; 2) Cells gated on CD3 knock out (KO) and CAR+ cells (for FIGS. B-G only) and from this population is shown CD8 expression on the Y axis and CD4 expression on the X axis; 3) Cells gated on CD3 KO, CAR+ (for FIGS. B-G only), CD4 positive cells and from this population is shown CD62L expression on the Y axis and CD45RO expression on the X axis (CD62L^{HI}CD45RO^{HI} are transitional memory cells); 4) Cells gated CD3 KO, CAR+ (for FIGS. B-G only), CD4 positive cells and from this population is shown CD62L expression on the Y axis and CD27 expression on the X axis; 5) Cells gated on CD3 KO, CAR+ (for FIGS. B-G only), CD8 positive cells and from this population is shown CD62L expression on the Y axis and CD45RO expression on the X axis (CD62L^{HI}CD45RO^{HI} are transitional memory cells); 6) Cells gated on CD3 KO, CAR+ (for FIGS. B-G only), CD8 positive cells and from this population is shown CD62L expression on the Y axis and CD27 expression on the X axis. Control cells transfected with the TRC 1-2L.1592 meganuclease of FIG. 3A were first gated only on CD3 KO cells (and not CAR expression) since no CAR construct was transduced in these cells.

FIGS. 4A-4E provide graphs showing real time in vitro killing of 293T cells expressing BCMA or 293T cells expressing CD19 by control human T cells that have been transduced with the with the TRC 1-2L.1592 meganuclease only or by human T cells transduced with one of the following CAR construct: a positive control BCMA scFv with an N6 or 4-1BB co-stimulatory domain, a BCMA-3L/3H scFv with an N6 co-stimulatory domain, a BCMA-3L/20H scFv with an N6 co-stimulatory domain, a BCMA-3L/51cH scFv with an N6 co-stimulatory domain, or a BCMA-20L/51cH scFv with an N6 co-stimulatory domain. The magnitude of cell killing is expressed as a reduction in cell index (y-axis) over time (x-axis). FIGS. 4A-C show the cell killing of BCMA expressing 293T cells after incubation with CAR T cells at a 1:2, 1:4, and 1:8 ratio, respectively, of CAR T cells to BCMA expressing 293T cells. FIG. 4D shows the percent cytolysis of BCMA expressing 293T cells after incubation with CAR T cells at a 1:8 ratio of CAR T cells to BCMA expressing 293T cells. FIG. 4E shows the cell killing of negative control CD19 expressing 293T cells after incubation with CAR T cells at a 1:2 ratio, respectively, of CAR T cells to BCMA expressing 293T cells.

FIGS. 5A-5F provide graphs showing the total luciferase flux and survival curves of NSG mice administered MM.1S luciferase expressing tumor cells. The mice were either untreated or treated with control TCR KO CAR T cells, CAR T cells having a positive control BCMA reference scFv with a 4-1BB or N6 co-stimulatory domain, CAR T cells having a BCMA 3L/3H scFv with an N6 or N1 co-stimulatory domain, or CAR T cells having a 3L/20H scFv with an N6 or N1 co-stimulatory domain (FIGS. 5D, 5E, and 5F only). FIG. 5A represents the total dorsal luciferase flux and FIG. 5B the total ventral flux in animals treated with CAR T cells having the indicated constructs for up to 100 days. FIG. 5C provides a survival curve of treated animals with CAR T cells having the indicated CAR constructs. FIG. 5D provides the average total dorsal luciferase flux and FIG. 5E the total ventral flux in animals treated with either 1e6 or 5e6 CAR T cells with the indicated CAR constructs. FIG. 5F provides a survival curve of animals treated with either 1e6 or 5e6 CAR T cells having the indicated CAR constructs.

FIGS. 6A-6F provide the stress test results of repeated exposure of BCMA CAR T cells to BCMA expressing target cells. FIGS. 6A, 6B, and 6C provide the percent killing of MM.1S BCMA expressing tumor cells, K562 BCMA expressing cells or negative control K562 CD19 expressing cells by the indicated CAR T cells at a 1:1, 1:2, 1:4, or 1:8 ratio of CAR T cells to the indicated target cells after 6 days of co-culture. FIGS. 6D, 6E, and 6F provide the percent killing of MM.1S BCMA expressing tumor cells, K562 BCMA expressing cells or negative control K562 CD19 expressing cells by the indicated CAR T cells at a 1:1, 1:2, 1:4, or 1:8 ratio of CAR T cells to the indicated target cells after 9 days of co-culture. The co-cultured CAR T cells expressed a reference positive control BCMA scFv with either a 4-1BB or N6 co-stimulatory domain, a BCMA-3L/3H scFv with an N6 co-stimulatory domain, a BCMA-3L/51cH scFv with an N6 co-stimulatory domain, a BCMA-3L/20H scFv with an N6 co-stimulatory domain, or a BCMA-20L/51cH scFv with an N6 co-stimulatory domain.

FIGS. 7A and 7B show immunoblots incubated with BCMA-3L/20H or the BCMA positive control primary antibody, followed by addition of the secondary AlexaFluor 647 anti-human IgG fluorescent antibody, are presented on the left-hand side of the figure. Positive blots, as determined by detectable AlexaFluor 647 fluorescent signal, indicate binding of BCMA-3L/20H or the BCMA positive antibody to the individual library protein expressed in transfected cells. Immunoblots shown on the right-hand side of the FIGS. represent detectable signal by ZsGreen fluorescence, demonstrating transfection efficiency of individual cDNA constructs into the overlaid HEK293 cells. FIG. 7A represents staining for the TNFRSF17 (BCMA) protein and FIG. 7B represents staining for a representative potential off target ADGRG7 cellular protein.

FIG. 8 shows flow cytometry plots showing the percentage of cells that are TCR-CAR⁺ in three BCMA-3L/20H CAR T cell Demo batches (DEMO 27, DEMO 32, and DEMO 46) postdepletion of residual unedited TCR⁺ cells. Anti-TCRα/β and anti-idiotype antibodies were used to detect gene-edited TCR-T cells that are CAR⁺ cells. The CAR expression is shown on the vertical axis and the TCR expression is shown on the horizontal axis. TCR⁻ cell frequencies and BCMA-3L/20H CAR T cell frequencies are displayed in the right-hand panels.

FIGS. 9A-9C show flow cytometry plots showing the percentage of TCR-CAR⁺CD4⁺ and TCR⁻CAR⁺CD8⁺ cells that are naïve (Tn; upper right quadrant), central memory (Tcm; lower right quadrant), and effector memory (Tem; lower left quadrant) phenotype in three BCMA-3L/20H CAR T cell Demo batches (Demo 27, Demo 32, and Demo 46), using anti-CD45RA and anti-CCR7 antibodies. In the left-hand column, anti-CD4 and anti-CD8 antibodies were used to detect the CD4⁺ and CD8⁺ composition of TCR-CAR⁺ T cells

FIG. 10 shows flow cytometry plots showing the percentage of K562 cells (no endogenous BCMA expression), BK562 cells (K562 cells transfected with BCMA), and 10 MM.1S cells that express BCMA using an anti-BCMA antibody.

FIGS. 11A-11C provides graphs showing BCMA-3L/20H CAR T cell proliferative responses following co-culture with BCMA+ and BCMA− tumor cell lines. BCMA-3L/20H CAR T cells from three Demo batches (Demo 27, Demo 32, and Demo 46) were cocultured with (FIG. 11A) BCMA⁺MM.1S cells, (FIG. 11B) BK562 cells (K562 cells expressing BCMA), or (FIG. 11C) BCMA⁻ K562 negative control cells. BCMA-3L/20H CAR T cell proliferative responses against the target cells at E:T ratios ranging from 1:0.5 to 1:5 were measured after 5 days of coculture. The dotted horizontal line represents the input number of PBCAR269A cells (1×10⁵ cells). Significance was calculated by two-way analysis of variance with Dunnett's multiple comparisons test (*=p<0.05, **=p<0.01, ***=p<0.001).

FIGS. 12A-12C provides graphs showing BCMA-3L/20H CAR T cell cytotoxic response against BCMA+ and BCMA− tumor cell lines. BCMA-3L/20H CAR T cells from three Demo batches (Demo 27, Demo 32, and Demo 46) were cocultured at the indicated E:T ratios with (FIG. 12A) BCMA⁺MM.1S cells, (FIG. 12B) BCMA⁺K562-BCMA cells, and (FIG. 12C) BCMA− K562 cells, and the cytotoxic response of BCMA-3L/20H CAR T cells was assessed after 5 days of coculture. Significance was calculated by two-way analysis of variance with Dunnett's multiple comparisons test (*=p<0.05, **=p<0.01, ***=p<0.001).

FIGS. 13A-13D provides graphs showing BCMA-3L/20H CAR T cell mediated cytokine production in response to BCMA+ and BCMA− tumor cell lines. BCMA-3L/20H CAR T cells were cocultured at an E:T ratio of 1:2 with BCMA⁺MM.1S cells and BCMA− K562 cells for 48 hours in medium in the absence of exogenous cytokines. The secretion of cytokines (FIG. 13A) IFNγ, (FIG. 13B) IL-2, (FIG. 13C) TNFα, and (FIG. 13D) IL-6 were measured by ProteinSimple multiplex assay. Significance was calculated by two-way analysis of variance with Dunnett's multiple comparisons test (***=p<0.001).

FIG. 14 provides a Kaplan-Meier survival plot of NSG mice that were implanted with 2.5×10⁶ MM.1S-ffLuc cells IV into a tail vein and treated with control or BCMA− 3L/20H CAR T cells. On Day 1 (9 days post-implantation), animals were administered vehicle control, TCR⁻ control T cells, or BCMA-3L/20H CAR T cell via IV injection. Cryopreserved TCR⁻ control T cells or BCMA-3L/20H CAR T cells were thawed, washed, and resuspended in sterile diluent and injected at a dose of 1.0×10⁶, 5.0×10⁶, or 1.5×10⁷ BCMA-3L/20H CAR T cells or 1.5×10⁷ TCR⁻ control T cells in a total volume of 0.2 mL per animal. Percent survival was plotted for each treatment group. Abbreviations: F=female; ffLuc=firefly luciferase; IV=intravenous; M=male.

FIG. 15 provides a graph showing individual times to endpoint of NSG mice were implanted with 2.5×10⁶ MM.1S-ffLuc cells IV into a tail vein and treated with control or BCMA-3L/20H CAR T cells. On Day 1 (9 days post-implantation), animals were administered vehicle control, TCR⁻ control T cells, or BCMA-3L/20H CAR T cells via IV injection. Cryopreserved TCR⁻ control T cells or BCMA-3L/20H CAR T cells were thawed, washed, and resuspended in sterile diluent and injected at a dose of 1.0×10⁶, 5.0×10⁶, or 1.5×10⁷ PBCAR269A cells or 1.5×10⁷ TCR⁻ control T cells in a total volume of 0.2 mL per animal. Time to endpoints were plotted for each animal in each group. Abbreviations: F=female; ffLuc=firefly luciferase; IV=intravenous; M=male.

FIG. 16 provides a graph showing luciferase flux distribution on day 36 in NSG mice that were implanted with 2.5×10⁶ MM.1S-ffLuc cells IV into a tail vein and treated with control or BCMA-3L/20H CAR T cells. NSG mice were implanted with 2.5×10⁶ MM.1S-ffLuc cells IV into a tail vein. On Day 1 (9 days post-implantation), animals were administered vehicle control, TCR⁻ control T cells, or BCMA-3L/20H CAR T cells via IV injection Cryopreserved TCR⁻ control T cells or BCMA-3L/20H CAR T cells were thawed, washed, and resuspended in sterile diluent and injected at a dose of 1.0×10⁶, 5.0×10⁶, or 1.5×10⁷ BCMA-3L/20H CAR T cells or 1.5×10⁷ TCR⁻ control T cells in a total volume of 0.2 mL per animal. Median flux data were plotted for each treatment group. Statistical significance was calculated by the Mann-Whitney U test (**=0.001<p≤0.01). Abbreviations: F=female; ffLuc=firefly luciferase; IV=intravenous; M=male.

FIG. 17 provides a graph showing the median luciferase flux distribution in NSG mice that were implanted with 2.5×10⁶ MM.1S-ffLuc cells IV into a tail vein and treated with control or BCMA-3L/20H CAR T cells. NSG mice were implanted with 2.5×10⁶ MM.1S-ffLuc cells IV into a tail vein. On Day 1 (9 days post-implantation), animals were administered vehicle control, TCR⁻ control T cells, or BCMA-3L/20H CAR T cells via IV injection Cryopreserved TCR⁻ control T cells or BCMA-3L/20H CAR T cells were thawed, washed, and resuspended in sterile diluent and injected at a dose of 1.0×10⁶, 5.0×10⁶, or 1.5×10⁷ BCMA-3L/20H CAR T cells or 1.5×10⁷ TCR⁻ control T cells in a total volume of 0.2 mL per animal. Median flux data were plotted for each treatment group.

FIG. 18 provides a graph showing the mean luciferase flux distribution in NSG mice that were implanted with 2.5×10⁶ MM.1S-ffLuc cells IV into a tail vein and treated with control or BCMA-3L/20H CAR T cells. NSG mice were implanted with 2.5×10⁶ MM.1S-ffLuc cells IV into a tail vein. On Day 1 (9 days post-implantation), animals were administered vehicle control, TCR⁻ control T cells, or BCMA-3L/20H CAR T cells via IV injection. Cryopreserved TCR⁻ control T cells or BCMA-3L/20H CAR T cells were thawed, washed, and resuspended in sterile diluent and injected at a dose of 1.0×10⁶, 5.0×10⁶, or 1.5×10⁷ BCMA-3L/20H CAR T cells or 1.5×10⁷ TCR⁻ control T cells in a total volume of 0.2 mL per animal. Mean flux data (±SEM) were plotted for each treatment group.

FIG. 19 provides a Kaplan-Meier survival plot of NSG mice that were implanted with 2.5×10⁶ MM.1S-ffLuc cells IV into a tail vein and treated with control or BCMA− 3L/20H CAR T cells. NSG mice were implanted with 2.5×10⁶ MM.1S-ffLuc cells IV into a tail vein. On Day 1 (8 days post-implantation), animals were administered TCR⁻ control T cells, or BCMA-3L/20H CAR T cells via IV injection. Cryopreserved TCR⁻ control T cells (1.5×10⁷) or BCMA-3L/20H CAR T cells (5.0×10⁶ or 1.5×10⁷) were thawed, washed, and resuspended in sterile diluent and injected in a total volume of 0.2 mL per animal. Percent survival was plotted for each treatment group.

FIG. 20 provides a graph showing individual times to endpoint of NSG mice were implanted with 2.5×10⁶ MM.1S-ffLuc cells IV into a tail vein and treated with control or BCMA-3L/20H CAR T cells. NSG mice were implanted with 2.5×10⁶ MM.1S-ffLuc cells IV into a tail vein. On Day 1 (8 days post-implantation), animals were administered TCR⁻ control T cells, or BCMA-3L/20H CAR T cells via IV injection. Cryopreserved TCR⁻ control T cells (1.5×10⁷) or BCMA-3L/20H CAR T cells (5.0×10⁶ or 1.5×10⁷) were thawed, washed, and resuspended in sterile diluent and injected in a total volume of 0.2 mL per animal. Time to endpoints were plotted for each animal in each group.

FIG. 21 provides a graph showing luciferase flux distribution on day 36 in NSG mice that were implanted with 2.5×10⁶ MM.1S-ffLuc cells IV into a tail vein and treated with control or BCMA-3L/20H CAR T cells. NSG mice were implanted with 2.5×10⁶ MM.1S-ffLuc cells IV into a tail vein. On Day 1 (8 days post-implantation), animals were administered TCR⁻ control T cells, or BCMA-3L/20H CAR T cells via IV injection. Cryopreserved TCR⁻ control T cells (1.5×10⁷) or BCMA-3L/20H CAR T cells (5.0×10⁶ or 1.5×10⁷) were thawed, washed, and resuspended in sterile diluent and injected in a total volume of 0.2 mL per animal. Median flux data at Day 43 was plotted for each treatment group. Statistical significance was calculated by the Mann-Whitney U test (***=p<0.001).

FIG. 22 provides a graph showing the median luciferase flux distribution in NSG mice that were implanted with 2.5×10⁶ MM.1S-ffLuc cells IV into a tail vein and treated with control or BCMA-3L/20H CAR T cells. NSG mice were implanted with 2.5×10⁶ MM.1S-ffLuc cells IV into a tail vein. On Day 1 (8 days post-implantation), animals were administered TCR⁻ control T cells, or BCMA-3L/20H CAR T cells via IV injection. Cryopreserved TCR⁻ control T cells (1.5×10⁷) or BCMA-3L/20H CAR T cells (5.0×10⁶ or 1.5×10⁷) were thawed, washed, and resuspended in sterile diluent and injected in a total volume of 0.2 mL per animal. Median flux data over time were plotted for each treatment group.

FIG. 23 provides a graph showing MM.1S tumor cell frequencies in blood of NSG mice that were implanted with 2.5×10⁶ MM.1S-ffLuc cells IV into a tail vein and treated with control or BCMA-3L/20H CAR T cells. NSG mice were implanted with 2.5×10⁶ MM.1S-ffLuc cells IV into a tail vein. On Day 1 (8 days post-implantation), animals were administered TCR⁻ control T cells, or BCMA-3L/20H CAR T cells via IV injection. Cryopreserved TCR⁻ control T cells (1.5×10⁷) or BCMA-3L/20H CAR T cells (5.0×10⁶ or 1.5×10⁷) were thawed, washed, and resuspended in sterile diluent and injected in a total volume of 0.2 mL per animal. Blood samples were collected on Days 3, 10, and 17 for all groups and Days 43, 52, and 60 (study endpoints) and analyzed by flow cytometry to determine the percentage of MM.1S cells using an anti-BCMA antibody.

FIG. 24 provides a graph showing hCD8⁺ and hCD4⁺ T cell frequencies in the blood of NSG mice that were implanted with 2.5×10⁶ MM.1S-ffLuc cells IV into a tail vein and treated with control or BCMA-3L/20H CAR T cells. NSG mice were implanted with 2.5×10⁶ MM.1S-ffLuc cells IV into a tail vein. On Day 1 (8 days post-implantation), animals were administered TCR⁻ control T cells, or BCMA-3L/20H CAR T cells via IV injection. Cryopreserved TCR⁻ control T cells (1.5×10⁷) or BCMA-3L/20H CAR T cells (5.0×10⁶ or 1.5×10⁷) were thawed, washed, and resuspended in sterile diluent and injected in a total volume of 0.2 mL per animal. Blood samples were collected on Days 3, 10, and 17 for all groups and Days 43, 52, and 60 (study endpoints) and analyzed by flow cytometry to determine the percentage of hCD8⁺ and hCD4⁺ cells using anti-hCD8 and anti-hCD4 antibodies.

FIG. 25 provides a graph showing hCD4⁺ T cell frequencies in the bone marrow of NSG mice that were implanted with 2.5×10⁶ MM.1S-ffLuc cells IV into a tail vein and treated with control or BCMA-3L/20H CAR T cells. NSG mice were implanted with 2.5×10⁶ MM.1S-ffLuc cells IV into a tail vein. On Day 1 (8 days post-implantation), animals were administered TCR⁻ control T cells, or BCMA-3L/20H CAR T cells via IV injection. Cryopreserved TCR⁻ control T cells (1.5×10⁷) or BCMA-3L/20H CAR T cells (5.0×10⁶ or 1.5×10⁷) were thawed, washed, and resuspended in sterile diluent and injected in a total volume of 0.2 mL per animal. Bone marrow samples were collected on Days 43, 52, and 60 and analyzed by flow cytometry to determine the percentage of hCD4⁺ cells using an anti-hCD4 antibody.

FIG. 26 provides a graph showing hCD8⁺ T cell frequencies in the bone marrow of NSG mice that were implanted with 2.5×10⁶ MM.1S-ffLuc cells IV into a tail vein and treated with control or BCMA-3L/20H CAR T cells. NSG mice were implanted with 2.5×10⁶ MM.1S-ffLuc cells IV into a tail vein. On Day 1 (8 days post-implantation), animals were administered TCR⁻ control T cells, or BCMA-3L/20H CAR T cells via IV injection. Cryopreserved TCR⁻ control T cells (1.5×10⁷) or BCMA-3L/20H CAR T cells (5.0×10⁶ or 1.5×10⁷) were thawed, washed, and resuspended in sterile diluent and injected in a total volume of 0.2 mL per animal. Bone marrow samples were collected on Days 43, 52, and 60 and analyzed by flow cytometry to determine the percentage of hCD8⁺ cells using an anti-hCD8 antibody.

FIG. 27 provides a graph showing MM.1S tumor cell frequencies in bone marrow of NSG mice that were implanted with 2.5×10⁶ MM.1S-ffLuc cells IV into a tail vein and treated with control or BCMA-3L/20H CAR T cells. NSG mice were implanted with 2.5×10⁶ MM.1S-ffLuc cells IV into a tail vein. On Day 1 (8 days post-implantation), animals were administered TCR⁻ control T cells, or BCMA-3L/20H CAR T cells via IV injection. Cryopreserved TCR⁻ control T cells (1.5×10⁷) or BCMA-3L/20H CAR T cells (5.0×10⁶ or 1.5×10⁷) were thawed, washed, and resuspended in sterile diluent and injected in a total volume of 0.2 mL per animal. Bone marrow samples were collected on Days 43, 52, and 60 and analyzed by flow cytometry to determine the percentage of MM.1S cells using an anti-BCMA antibody.

FIG. 28 provides a graph showing individual times to endpoint of NSG mice that were implanted with 2.5×10⁶ MM.1S-ffLuc cells IV into a tail vein and treated with control or BCMA-3L/20H CAR T cells. NSG mice (n=8 or 10 per group) were sub lethally irradiated on Day 1 and injected IV on Day 2 with freshly thawed diluent vehicle, freshly thawed 3.0×10⁷ unedited TCR⁺ control T cells, or freshly thawed 3.0×10⁷ BCMA-3L/20H CAR T cells. Time to endpoint was recorded for each animal that died of its disease or was euthanized due to disease progression.

FIG. 29 provides a graph showing Median GvHD scores of animals administered vehicle control, unedited TCR⁺ control T cell, or BCMA-3L/20H CAR T cell infusion. NSG mice (n=8 or 10 per group) were sublethally irradiated on Day 1 and injected IV on Day 2 with freshly thawed diluent vehicle, freshly thawed 3.0×10⁷ unedited TCR⁺ control T cells, or freshly thawed 3.0×10⁷ BCMA-3L/20H CAR T cells. Clinical observations were scored based on the degree of loss of weight, activity, posture, fur texture, and skin integrity. At each time point, animals were scored in all categories, with a maximum possible score of 10 per animal. Animal 3 (Group 3) and Animals 2 and 3 (Group 4) are excluded from graph and statistical analysis on Day 12 due to animal deaths.

FIG. 30 provides a graph showing body weight change over time after vehicle control, unedited TCR⁺ control T cell, or BCMA-3L/20H CAR T cell infusion. NSG mice (n=8 or 10 per group) were sublethally irradiated on Day 1 and injected IV on Day 2 with freshly thawed diluent vehicle, freshly thawed 3.0×10⁷ unedited TCR⁺ control T cells, or freshly thawed 3.0×10⁷ gene-edited BCMA-3L/20H CAR T cells. Animals were weighed daily for 30 days, then triweekly until the completion of the study on Day 47. Animal 3 (Group 3) and Animals 2 and 3 (Group 4) are excluded from graph and statistical analysis on Day 12 due to animal deaths. An asterisk (*=p<0.001) represents a statistically significant difference compared with concurrent vehicle control groups

FIG. 31 provides a Kaplan-Meier survival curve after BCMA-3L/20H CAR T cell or unedited TCR⁺ control T cell infusion. NSG mice (n=8 or 10 per group) were sublethally irradiated on Day 1 and injected IV on Day 2 with freshly thawed diluent vehicle, freshly thawed 3.0×10⁷ unedited TCR⁺ control T cells, or freshly thawed 3.0×10⁷ BCMA-3L/20H CAR T cells.

FIGS. 32A and 32B provides graphs showing organ weights of NSG mice. FIG. 32A and FIG. 32B indicate the weights of the indicated organs for female and male mice, respectively. NSG mice (n=8 or 10 per group) were sublethally irradiated on Day 1 and injected IV on Day 2 with freshly thawed diluent vehicle, freshly thawed 3.0×10⁷ unedited TCR⁺ control T cells, or freshly thawed 3.0×10⁷ BCMA-3L/20H CAR T cells. Significance was calculated by two-tailed Student's t-tests (*=p<0.001).

### BRIEF DESCRIPTION OF THE SEQUENCES

SEQ ID NO: 1 sets forth the amino acid sequence of human BCMA.

SEQ ID NO: 2 sets forth the amino acid sequence of the BCMA-3 antibody VH region.

SEQ ID NO: 3 sets forth the nucleic acid sequence of the BCMA-3 antibody VH region.

SEQ ID NO: 4 sets forth the amino acid sequence of the BCMA-3 antibody VL region.

SEQ ID NO: 5 sets forth the nucleic acid sequence of the BCMA-3 antibody VL region.

SEQ ID NO: 6 sets forth the amino acid sequence of the BCMA-20 antibody VH region.

SEQ ID NO: 7 sets forth the nucleic acid sequence of the BCMA-20 antibody VH region.

SEQ ID NO: 8 sets forth the amino acid sequence of the BCMA-20 antibody VL region.

SEQ ID NO: 9 sets forth the nucleic acid sequence of the BCMA-20 antibody VL region.

SEQ ID NO: 10 sets forth the amino acid sequence of the BCMA-51c antibody VH region.

SEQ ID NO: 11 sets forth the nucleic acid sequence of the BCMA-51c antibody VH region.

SEQ ID NO: 12 sets forth the amino acid sequence of the BCMA-51c antibody VL region.

SEQ ID NO: 13 sets forth the nucleic acid sequence of the BCMA-51c antibody VL region.

SEQ ID NO: 14 sets forth the amino acid sequence of the BCMA-3 antibody CDRH1 domain.

SEQ ID NO: 15 sets forth the amino acid sequence of the BCMA-3 antibody CDRH2 domain.

SEQ ID NO: 16 sets forth the amino acid sequence of the BCMA-3 antibody CDRH3 domain.

SEQ ID NO: 17 sets forth the amino acid sequence of the BCMA-3 antibody CDRL1 domain.

SEQ ID NO: 18 sets forth the amino acid sequence of the BCMA-3 antibody CDRL2 domain.

SEQ ID NO: 19 sets forth the amino acid sequence of the BCMA-3 antibody CDRL3 domain.

SEQ ID NO: 20 sets forth the amino acid sequence of the BCMA-20 antibody CDRH1 domain.

SEQ ID NO: 21 sets forth the amino acid sequence of the BCMA-20 antibody CDRH2 domain.

SEQ ID NO: 22 sets forth the amino acid sequence of the BCMA-20 antibody CDRH3 domain.

SEQ ID NO: 23 sets forth the amino acid sequence of the BCMA-20 antibody CDRL1 domain.

SEQ ID NO: 24 sets forth the amino acid sequence of the BCMA-20 antibody CDRL2 domain.

SEQ ID NO: 25 sets forth the amino acid sequence of the BCMA-20 antibody CDRL3 domain.

SEQ ID NO: 26 sets forth the amino acid sequence of the BCMA-51c antibody CDRH1 domain.

SEQ ID NO: 27 sets forth the amino acid sequence of the BCMA-51c antibody CDRH2 domain.

SEQ ID NO: 28 sets forth the amino acid sequence of the BCMA-51c antibody CDRH3 domain.

SEQ ID NO: 29 sets forth the amino acid sequence of the BCMA-51c antibody CDRL1 domain.

SEQ ID NO: 30 sets forth the amino acid sequence of the BCMA-51c antibody CDRL2 domain.

SEQ ID NO: 31 sets forth the amino acid sequence of the BCMA-51c antibody CDRL3 domain.

SEQ ID NO: 32 sets forth the nucleic acid sequence of a polypeptide linker.

SEQ ID NO: 33 sets forth the nucleic acid sequence of a polypeptide linker.

SEQ ID NO: 34 sets forth the amino acid sequence of a polypeptide linker.

SEQ ID NO: 35 sets forth the amino acid sequence of a polypeptide linker.

SEQ ID NO: 36 sets forth the amino acid sequence of a polypeptide linker.

SEQ ID NO: 37 sets forth the amino acid sequence of a polypeptide linker.

SEQ ID NO: 38 sets forth the amino acid sequence of a polypeptide linker.

SEQ ID NO: 39 sets forth the amino acid sequence of a polypeptide linker.

SEQ ID NO: 40 sets forth the amino acid sequence of a polypeptide linker.

SEQ ID NO: 41 sets forth the amino acid sequence of a polypeptide linker.

SEQ ID NO: 42 sets forth the amino acid sequence of a polypeptide linker.

SEQ ID NO: 43 sets forth the amino acid sequence of a polypeptide linker.

SEQ ID NO: 44 sets forth the amino acid sequence of a polypeptide linker.

SEQ ID NO: 45 sets forth the amino acid sequence of a polypeptide linker.

SEQ ID NO: 46 sets forth the amino acid sequence of a polypeptide linker.

SEQ ID NO: 47 sets forth the amino acid sequence of a polypeptide linker.

SEQ ID NO: 48 sets forth the amino acid sequence of a polypeptide linker.

SEQ ID NO: 49 sets forth the amino acid sequence of a polypeptide linker.

SEQ ID NO: 50 sets forth the amino acid sequence of a polypeptide linker.

SEQ ID NO: 51 sets forth the amino acid sequence of a polypeptide linker.

SEQ ID NO: 52 sets forth the amino acid sequence of a spacer sequence.

SEQ ID NO: 53 sets forth the nucleic acid sequence of a spacer sequence.

SEQ ID NO: 54 sets forth the amino acid sequence of a CD8 hinge domain.

SEQ ID NO: 55 sets forth the nucleic acid sequence of a CD8 hinge domain.

SEQ ID NO: 56 sets forth the amino acid sequence of CD8 transmembrane.

SEQ ID NO: 57 sets forth the nucleic acid sequence of CD8 transmembrane.

SEQ ID NO: 58 sets forth the amino acid sequence of an N1 co-stimulatory domain.

SEQ ID NO: 59 sets forth the nucleic acid sequence of an N1 co-stimulatory domain.

SEQ ID NO: 60 sets forth the amino acid sequence of an N6 co-stimulatory domain.

SEQ ID NO: 61 sets forth the nucleic acid sequence of an N6 co-stimulatory domain.

SEQ ID NO: 62 sets forth the amino acid sequence of a 4-1BB co-stimulatory domain.

SEQ ID NO: 63 sets forth the nucleic acid sequence of a 4-1BB co-stimulatory domain.

SEQ ID NO: 64 sets forth the amino acid sequence of a CD28 co-stimulatory domain.

SEQ ID NO: 65 sets forth the nucleic acid sequence of a CD28 co-stimulatory domain.

SEQ ID NO: 66 sets forth the amino acid sequence of a CD3 zeta signaling domain.

SEQ ID NO: 67 sets forth the nucleic acid sequence of a CD3 zeta signaling domain.

SEQ ID NO: 68 sets forth the amino acid sequence of a CD8 signal peptide.

SEQ ID NO: 69 sets forth the nucleic acid sequence of a CD8 signal peptide.

SEQ ID NO: 70 sets forth the amino acid sequence of a CD8 signal peptide.

SEQ ID NO: 71 sets forth the nucleic acid sequence of a CD8 signal peptide.

SEQ ID NO: 72 sets forth the nucleic acid sequence of a JeT promoter.

SEQ ID NO: 73 sets forth the nucleic acid sequence of an EF1 alpha promoter.

SEQ ID NO: 74 sets forth the nucleic acid sequence of the TRC 1-2 recognition sequence (sense).

SEQ ID NO: 75 sets forth the nucleic acid sequence of the TRC 1-2 recognition sequence (antisense).

SEQ ID NO: 76 sets forth the amino acid sequence of a TRC 1-2L.1592 meganuclease.

SEQ ID NO: 77 sets forth the amino acid sequence of a heavy chain constant region.

SEQ ID NO: 78 sets forth the nucleic acid sequence of a heavy chain constant region.

SEQ ID NO: 79 sets forth the amino acid sequence of a light chain constant region.

SEQ ID NO: 80 sets forth the nucleic acid sequence of a light chain constant region.

SEQ ID NO: 81 sets forth the amino acid sequence of a BCMA-3H/3L scFv.

SEQ ID NO: 82 sets forth the amino acid sequence of a BCMA-3L/3H scFv.

SEQ ID NO: 83 sets forth the amino acid sequence of a BCMA-20H/20L scFv.

SEQ ID NO: 84 sets forth the amino acid sequence of a BCMA-20L/20H scFv.

SEQ ID NO: 85 sets forth the amino acid sequence of a BCMA-51cH/51cL scFv.

SEQ ID NO: 86 sets forth the amino acid sequence of a BCMA-51cL/51cH scFv.

SEQ ID NO: 87 sets forth the amino acid sequence of a BCMA-3H/20L scFv.

SEQ ID NO: 88 sets forth the amino acid sequence of a BCMA-3L/20H scFv.

SEQ ID NO: 89 sets forth the amino acid sequence of a BCMA-3H/51cL scFv.

SEQ ID NO: 90 sets forth the amino acid sequence of a BCMA-3L/51cH scFv.

SEQ ID NO: 91 sets forth the amino acid sequence of a BCMA-20H/3L scFv.

SEQ ID NO: 92 sets forth the amino acid sequence of a BCMA-20L/3H scFv.

SEQ ID NO: 93 sets forth the amino acid sequence of a BCMA-20H/51cL scFv.

SEQ ID NO: 94 sets forth the amino acid sequence of a BCMA-20L/51cH scFv.

SEQ ID NO: 95 sets forth the amino acid sequence of a BCMA-51cH/3L scFv.

SEQ ID NO: 96 sets forth the amino acid sequence of a BCMA-51cL/3H scFv.

SEQ ID NO: 97 sets forth the amino acid sequence of a BCMA-51cH/20L scFv.

SEQ ID NO: 98 sets forth the amino acid sequence of a BCMA-51cL/20H scFv.

SEQ ID NO: 99 sets forth the nucleic acid sequence of a BCMA-3H/3L scFv.

SEQ ID NO: 100 sets forth the nucleic acid sequence of a BCMA-3L/3H scFv.

SEQ ID NO: 101 sets forth the nucleic acid sequence of a BCMA-20H/20L scFv.

SEQ ID NO: 102 sets forth the nucleic acid sequence of a BCMA-20L/20H scFv.

SEQ ID NO: 103 sets forth the nucleic acid sequence of a BCMA-51cH/51cL scFv.

SEQ ID NO: 104 sets forth the nucleic acid sequence of a BCMA-51cL/51cH scFv.

SEQ ID NO: 105 sets forth the nucleic acid sequence of a BCMA-3H/20L scFv.

SEQ ID NO: 106 sets forth the nucleic acid sequence of a BCMA-3L/20H scFv.

SEQ ID NO: 107 sets forth the nucleic acid sequence of a BCMA-3H/51cL scFv.

SEQ ID NO: 108 sets forth the nucleic acid sequence of a BCMA-3L/51cH scFv.

SEQ ID NO: 109 sets forth the nucleic acid sequence of a BCMA-20H/3L scFv.

SEQ ID NO: 110 sets forth the nucleic acid sequence of a BCMA-20L/3H scFv.

SEQ ID NO: 111 sets forth the nucleic acid sequence of a BCMA-20H/51cL scFv.

SEQ ID NO: 112 sets forth the nucleic acid sequence of a BCMA-20L/51cH scFv.

SEQ ID NO: 113 sets forth the nucleic acid sequence of a BCMA-51cH/3L scFv.

SEQ ID NO: 114 sets forth the nucleic acid sequence of a BCMA-51cL/3H scFv.

SEQ ID NO: 115 sets forth the nucleic acid sequence of a BCMA-51cH/20L scFv.

SEQ ID NO: 116 sets forth the nucleic acid sequence of a BCMA-51cL/20H scFv.

SEQ ID NO: 117 sets forth the amino acid sequence of a BCMA-3H/3L-Spacer-CD8-CD8-N6-CD3z CAR.

SEQ ID NO: 118 sets forth the amino acid sequence of a BCMA-3L/3H-Spacer-CD8-CD8-N6-CD3z CAR.

SEQ ID NO: 119 sets forth the amino acid sequence of a BCMA-20H/20L-Spacer-CD8-CD8-N6-CD3z CAR.

SEQ ID NO: 120 sets forth the amino acid sequence of a BCMA-20L/20H-Spacer-CD8-CD8-N6-CD3z CAR.

SEQ ID NO: 121 sets forth the amino acid sequence of a BCMA-51cH/51cL-Spacer-CD8-CD8-N6-CD3z CAR.

SEQ ID NO: 122 sets forth the amino acid sequence of a BCMA-51cL/51cH-Spacer-CD8-CD8-N6-CD3z CAR.

SEQ ID NO: 123 sets forth the amino acid sequence of a BCMA-3H/20L-Spacer-CD8-CD8-N6-CD3z CAR.

SEQ ID NO: 124 sets forth the amino acid sequence of a BCMA-3L/20H-Spacer-CD8-CD8-N6-CD3z CAR.

SEQ ID NO: 125 sets forth the amino acid sequence of a BCMA-3H/51cL-Spacer-CD8-CD8-N6-CD3z CAR.

SEQ ID NO: 126 sets forth the amino acid sequence of a BCMA-3L/51cH-Spacer-CD8-CD8-N6-CD3z CAR.

SEQ ID NO: 127 sets forth the amino acid sequence of a BCMA-20H/3L-Spacer-CD8-CD8-N6-CD3z CAR.

SEQ ID NO: 128 sets forth the amino acid sequence of a BCMA-20L/3H-Spacer-CD8-CD8-N6-CD3z CAR.

SEQ ID NO: 129 sets forth the amino acid sequence of a BCMA-20H/51cL-Spacer-CD8-CD8-N6-CD3z CAR.

SEQ ID NO: 130 sets forth the amino acid sequence of a BCMA-20L/51cH-Spacer-CD8-CD8-N6-CD3z CAR.

SEQ ID NO: 131 sets forth the amino acid sequence of a BCMA-51cH/3L-Spacer-CD8-CD8-N6-CD3z CAR.

SEQ ID NO: 132 sets forth the amino acid sequence of a BCMA-51cL/3H-Spacer-CD8-CD8-N6-CD3z CAR.

SEQ ID NO: 133 sets forth the amino acid sequence of a BCMA-51cH/20L-Spacer-CD8-CD8-N6-CD3z CAR.

SEQ ID NO: 134 sets forth the amino acid sequence of a BCMA-51cL/20H-Spacer-CD8-CD8-N6-CD3z CAR.

SEQ ID NO: 135 sets forth the nucleic acid sequence of a BCMA-3H/3L-Spacer-CD8-CD8-N6-CD3z CAR.

SEQ ID NO: 136 sets forth the nucleic acid sequence of a BCMA-3L/3H-Spacer-CD8-CD8-N6-CD3z CAR.

SEQ ID NO: 137 sets forth the nucleic acid sequence of a BCMA-20H/20L-Spacer-CD8-CD8-N6-CD3z CAR.

SEQ ID NO: 138 sets forth the nucleic acid sequence of a BCMA-20L/20H-Spacer-CD8-CD8-N6-CD3z CAR.

SEQ ID NO: 139 sets forth the nucleic acid sequence of a BCMA-51cH/51cL-Spacer-CD8-CD8-N6-CD3z CAR.

SEQ ID NO: 140 sets forth the nucleic acid sequence of a BCMA-51cL/51cH-Spacer-CD8-CD8-N6-CD3z CAR.

SEQ ID NO: 141 sets forth the nucleic acid sequence of a BCMA-3H/20L-Spacer-CD8-CD8-N6-CD3z CAR.

SEQ ID NO: 142 sets forth the nucleic acid sequence of a BCMA-3L/20H-Spacer-CD8-CD8-N6-CD3z CAR.

SEQ ID NO: 143 sets forth the nucleic acid sequence of a BCMA-3H/51cL-Spacer-CD8-CD8-N6-CD3z CAR.

SEQ ID NO: 144 sets forth the nucleic acid sequence of a BCMA-3L/51cH-Spacer-CD8-CD8-N6-CD3z CAR.

SEQ ID NO: 145 sets forth the nucleic acid sequence of a BCMA-20H/3L-Spacer-CD8-CD8-N6-CD3z CAR.

SEQ ID NO: 146 sets forth the nucleic acid sequence of a BCMA-20L/3H-Spacer-CD8-CD8-N6-CD3z CAR.

SEQ ID NO: 147 sets forth the nucleic acid sequence of a BCMA-20H/51cL-Spacer-CD8-CD8-N6-CD3z CAR.

SEQ ID NO: 148 sets forth the nucleic acid sequence of a BCMA-20L/51cH-Spacer-CD8-CD8-N6-CD3z CAR.

SEQ ID NO: 149 sets forth the nucleic acid sequence of a BCMA-51cH/3L-Spacer-CD8-CD8-N6-CD3z CAR.

SEQ ID NO: 150 sets forth the nucleic acid sequence of a BCMA-51cL/3H-Spacer-CD8-CD8-N6-CD3z CAR.

SEQ ID NO: 151 sets forth the nucleic acid sequence of a BCMA-51cH/20L-Spacer-CD8-CD8-N6-CD3z CAR.

SEQ ID NO: 152 sets forth the nucleic acid sequence of a BCMA-51cL/20H-Spacer-CD8-CD8-N6-CD3z CAR.

SEQ ID NO: 153 sets forth the amino acid sequence of a CD8(+A)SP-BCMA-3H/3L-Spacer-CD8-CD8-N6-CD3z CAR.

SEQ ID NO: 154 sets forth the amino acid sequence of a CD8(+A)SP-BCMA-3L/3H-Spacer-CD8-CD8-N6-CD3z CAR.

SEQ ID NO: 155 sets forth the amino acid sequence of a CD8(+A)SP-BCMA-20H/20L-Spacer-CD8-CD8-N6-CD3z CAR.

SEQ ID NO: 156 sets forth the amino acid sequence of a CD8(+A)SP-BCMA-20L/20H-Spacer-CD8-CD8-N6-CD3z CAR.

SEQ ID NO: 157 sets forth the amino acid sequence of a CD8(+A)SP-BCMA-51cH/51cL-Spacer-CD8-CD8-N6-CD3z CAR.

SEQ ID NO: 158 sets forth the amino acid sequence of a CD8(+A)SP-BCMA-51cL/51cH-Spacer-CD8-CD8-N6-CD3z CAR.

SEQ ID NO: 159 sets forth the amino acid sequence of a CD8(+A)SP-BCMA-3H/20L-Spacer-CD8-CD8-N6-CD3z CAR.

SEQ ID NO: 160 sets forth the amino acid sequence of a CD8(+A)SP-BCMA-3L/20H-Spacer-CD8-CD8-N6-CD3z CAR.

SEQ ID NO: 161 sets forth the amino acid sequence of a CD8(+A)SP-BCMA-3H/51cL-Spacer-CD8-CD8-N6-CD3z CAR.

SEQ ID NO: 162 sets forth the amino acid sequence of a CD8(+A)SP-BCMA-3L/51cH-Spacer-CD8-CD8-N6-CD3z CAR.

SEQ ID NO: 163 sets forth the amino acid sequence of a CD8(+A)SP-BCMA-20H/3L-Spacer-CD8-CD8-N6-CD3z CAR.

SEQ ID NO: 164 sets forth the amino acid sequence of a CD8(+A)SP-BCMA-20L/3H-Spacer-CD8-CD8-N6-CD3z CAR.

SEQ ID NO: 165 sets forth the amino acid sequence of a CD8(+A)SP-BCMA-20H/51cL-Spacer-CD8-CD8-N6-CD3z CAR.

SEQ ID NO: 166 sets forth the amino acid sequence of a CD8(+A)SP-BCMA-20L/51cH-Spacer-CD8-CD8-N6-CD3z CAR.

SEQ ID NO: 167 sets forth the amino acid sequence of a CD8(+A)SP-BCMA-51cH/3L-Spacer-CD8-CD8-N6-CD3z CAR.

SEQ ID NO: 168 sets forth the amino acid sequence of a CD8(+A)SP-BCMA-51cL/3H-Spacer-CD8-CD8-N6-CD3z CAR.

SEQ ID NO: 169 sets forth the amino acid sequence of a CD8(+A)SP-BCMA-51cH/20L-Spacer-CD8-CD8-N6-CD3z CAR.

SEQ ID NO: 170 sets forth the amino acid sequence of a CD8(+A)SP-BCMA-51cL/20H-Spacer-CD8-CD8-N6-CD3z CAR.

SEQ ID NO: 171 sets forth the nucleic acid sequence of a CD8(+A)SP-BCMA-3H/3L-Spacer-CD8-CD8-N6-CD3z CAR.

SEQ ID NO: 172 sets forth the nucleic acid sequence of a CD8(+A)SP-BCMA-3L/3H-Spacer-CD8-CD8-N6-CD3z CAR.

SEQ ID NO: 173 sets forth the nucleic acid sequence of a CD8(+A)SP-BCMA-20H/20L-Spacer-CD8-CD8-N6-CD3z CAR.

SEQ ID NO: 174 sets forth the nucleic acid sequence of a CD8(+A)SP-BCMA-20L/20H-Spacer-CD8-CD8-N6-CD3z CAR.

SEQ ID NO: 175 sets forth the nucleic acid sequence of a CD8(+A)SP-BCMA-51cH/51cL-Spacer-CD8-CD8-N6-CD3z CAR.

SEQ ID NO: 176 sets forth the nucleic acid sequence of a CD8(+A)SP-BCMA-51cL/51cH-Spacer-CD8-CD8-N6-CD3z CAR.

SEQ ID NO: 177 sets forth the nucleic acid sequence of a CD8(+A)SP-BCMA-3H/20L-Spacer-CD8-CD8-N6-CD3z CAR.

SEQ ID NO: 178 sets forth the nucleic acid sequence of a CD8(+A)SP-BCMA-3L/20H-Spacer-CD8-CD8-N6-CD3z CAR.

SEQ ID NO: 179 sets forth the nucleic acid sequence of a CD8(+A)SP-BCMA-3H/51cL-Spacer-CD8-CD8-N6-CD3z CAR.

SEQ ID NO: 180 sets forth the nucleic acid sequence of a CD8(+A)SP-BCMA-3L/51cH-Spacer-CD8-CD8-N6-CD3z CAR.

SEQ ID NO: 181 sets forth the nucleic acid sequence of a CD8(+A)SP-BCMA-20H/3L-Spacer-CD8-CD8-N6-CD3z CAR.

SEQ ID NO: 182 sets forth the nucleic acid sequence of a CD8(+A)SP-BCMA-20L/3H-Spacer-CD8-CD8-N6-CD3z CAR.

SEQ ID NO: 183 sets forth the nucleic acid sequence of a CD8(+A)SP-BCMA-20H/51cL-Spacer-CD8-CD8-N6-CD3z CAR.

SEQ ID NO: 184 sets forth the nucleic acid sequence of a CD8(+A)SP-BCMA-20L/51cH-Spacer-CD8-CD8-N6-CD3z CAR.

SEQ ID NO: 185 sets forth the nucleic acid sequence of a CD8(+A)SP-BCMA-51cH/3L-Spacer-CD8-CD8-N6-CD3z CAR.

SEQ ID NO: 186 sets forth the nucleic acid sequence of a CD8(+A)SP-BCMA-51cL/3H-Spacer-CD8-CD8-N6-CD3z CAR.

SEQ ID NO: 187 sets forth the nucleic acid sequence of a CD8(+A)SP-BCMA-51cH/20L-Spacer-CD8-CD8-N6-CD3z CAR.

SEQ ID NO: 188 sets forth the nucleic acid sequence of a CD8(+A)SP-BCMA-51cL/20H-Spacer-CD8-CD8-N6-CD3z CAR.

SEQ ID NO: 189 sets forth the amino acid sequence of a CD8 signal peptide.

SEQ ID NO: 190 sets forth a nucleic acid sequence encoding a CD8 signal peptide.

### DETAILED DESCRIPTION

All publications, patents and other references cited herein are incorporated by reference in their entirety into the present disclosure.

In practicing the presently disclosed subject matter, many conventional techniques in molecular biology, microbiology, cell biology, biochemistry, and immunology are used, which are within the skill of the art. These techniques are described in greater detail in, for example, Molecular Cloning: a Laboratory Manual 3rd edition, J. F. Sambrook and D. W. Russell, ed. Cold Spring Harbor Laboratory Press 2001; Recombinant Antibodies for Immunotherapy, Melvyn Little, ed. Cambridge University Press 2009; Oligonucleotide Synthesis” (M. J. Gait, ed., 1984); “Animal Cell Culture” (R. I. Freshney, ed., 1987); “Methods in Enzymology” (Academic Press, Inc.); “Current Protocols in Molecular Biology” (F. M. Ausubel et al., eds., 1987, and periodic updates); “PCR: The Polymerase Chain Reaction”, (Mullis et al., ed., 1994); “A Practical Guide to Molecular Cloning” (Perbal Bernard V., 1988); “Phage Display: A Laboratory Manual” (Barbas et al., 2001). The contents of these references and other references containing standard protocols, widely known to and relied upon by those of skill in the art, including manufacturers' instructions are hereby incorporated by reference as part of the present disclosure.

### 1. Definitions

In the description that follows, certain conventions will be followed as regards the usage of terminology. Generally, terms used herein are intended to be interpreted consistently with the meaning of those terms as they are known to those of skill in the art.

An “antigen-binding protein” is a protein or polypeptide that comprises an antigen-binding region or antigen-binding portion, that is, has a strong affinity to another molecule to which it binds. Antigen-binding proteins encompass, for example, antibodies, chimeric antigen receptors (CARs) and fusion proteins.

The term “antibody” as referred to herein refers to a glycoprotein comprising at least two heavy (H) chains and two light (L) chains inter-connected by disulfide bonds. Each heavy chain is comprised of a heavy chain variable region (abbreviated herein as VH) and a heavy chain constant (CH) region. The heavy chain constant region is comprised of three domains, CH1, CH2 and CH3. Each light chain is comprised of a light chain variable region (abbreviated herein as VL) and a light chain constant (CL) region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1) of the classical complement system.

The term “antigen-binding portion” or “antigen-binding region” of an antibody, as used herein, refers to that region or portion of the antibody that binds to the antigen and which confers antigen specificity to the antibody; fragments of antigen-binding proteins, for example, antibodies includes one or more fragments of an antibody that retain the ability to specifically bind to an antigen (e.g., a BCMA polypeptide). It has been shown that the antigen-binding function of an antibody can be performed by fragments of a full-length antibody. Examples of antigen-binding fragments encompassed within the term “antibody fragments” of an antibody include an antigen binding protein comprising a portion, i.e., an antigen binding region, of an intact antibody, such that the protein retains the antigen binding specificity of the antibody. Examples of antibody fragments include Fab, Fab′, F(ab′)2, and Fv fragments; diabodies; tandem diabodies (taDb), linear antibodies (e.g., U.S. Pat. No. 5,641,870, Example 2; Zapata et al., Protein Eng. 8(10):1057-1062 (1995)); one-armed antibodies, single variable domain antibodies, minibodies, single-chain antibody molecules; multispecific antibodies formed from antibody fragments (e.g., including but not limited to, Db-Fc, taDb-Fc, taDb-CH3, (scFV)4-Fc, di-scFv, bi-scFv, or tandem (di,tri)-scFv); and Bi-specific T-cell engagers (BiTEs). An “isolated antibody” or “isolated antigen-binding protein” is one which has been separated and/or recovered from a component of its natural environment.

Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules. These are known as single chain Fv (scFv); see e.g., Bird et al., 1988 Science 242:423-426; and Huston et al., 1988 Proc. Natl. Acad. Sci. 85:5879-5883. These antibody fragments are obtained using conventional techniques known to those of skill in the art, and the fragments are screened for utility in the same manner as are intact antibodies.

As used herein, the term “single-chain variable fragment” or “scFv” is a fusion protein of the variable regions of the heavy (VH) and light chains (VL) of an immunoglobulin (e.g., mouse or human) covalently linked to form a VH-VL or VL-VH heterodimer. The heavy (VH) and light chains (VL) are either joined directly or joined by a peptide-encoding linker, which connects the N-terminus of the VH with the C-terminus of the VL, or the C-terminus of the VH with the N-terminus of the VL. The linker is usually rich in glycine for flexibility, as well as serine or threonine for solubility. The linker can link the heavy chain variable region and the light chain variable region of the extracellular antigen-binding domain. Non-limiting examples of linkers are disclosed in Shen et al., Anal. Chem. 80(6):1910-1917 (2008) and WO 2014/087010, the contents of which are hereby incorporated by reference in their entireties. In certain embodiments, the linker comprises amino acids having the sequence set forth in any one of SEQ ID NOs: 34-51, and variants thereof.

Despite removal of the constant regions and the introduction of a linker, scFv proteins retain the specificity of the original immunoglobulin. Single chain Fv polypeptide antibodies can be expressed from a nucleic acid comprising VH- and VL-encoding sequences as described by Huston, et al. (Proc. Nat. Acad. Sci. USA, 85:5879-5883, 1988). See, also, U.S. Pat. Nos. 5,091,513, 5,132,405 and 4,956,778; and U.S. Patent Publication Nos. 20050196754 and 20050196754. Antagonistic scFvs having inhibitory activity have been described (see, e.g., Zhao et al., Hyrbidoma (Larchmt) 2008 27(6):455-51; Peter et al., J Cachexia Sarcopenia Muscle 2012 Aug. 12; Shieh et al., J Imunol 2009 183(4):2277-85; Giomarelli et al., Thromb Haemost 2007 97(6):955-63; Fife eta., J Clin Inst 2006 116(8):2252-61; Brocks et al., Immunotechnology 1997 3(3):173-84; Moosmayer et al., Ther Immunol 1995 2(10:31-40). Agonistic scFvs having stimulatory activity have been described (see, e.g., Peter et al., J Bioi Chern 2003 25278(38):36740-7; Xie et al., Nat Biotech 1997 15(8):768-71; Ledbetter et al., Crit Rev Immunol 1997 17(5-6):427-55; Ho et al., BioChim Biophys Acta 2003 1638(3):257-66).

As used herein, “F(ab)” refers to a fragment of an antibody structure that binds to an antigen but is monovalent and does not have a Fc portion, for example, an antibody digested by the enzyme papain yields two F(ab) fragments and an Fc fragment (e.g., a heavy (H) chain constant region; Fc region that does not bind to an antigen).

As used herein, “F(ab′)2” refers to an antibody fragment generated by pepsin digestion of whole IgG antibodies, wherein this fragment has two antigen binding (ab′) (bivalent) regions, wherein each (ab′) region comprises two separate amino acid chains, a part of a H chain and a light (L) chain linked by an S—S bond for binding an antigen and where the remaining H chain portions are linked together. A “F(ab′)2” fragment can be split into two individual Fab′ fragments.

As used herein, “CDRs” are defined as the complementarity determining regions of an antibody which are the hypervariable regions of immunoglobulin heavy and light chains. See, e.g., Kabat et al., Sequences of Proteins of Immunological Interest, 4th U.S. Department of Health and Human Services, National Institutes of Health (1987). The term “hypervariable region” or “HVR” as used herein refers to each of the regions of an antibody variable domain which are hypervariable in sequence (“complementarity determining regions” or “CDRs”) and/or form structurally defined loops (“hypervariable loops”) and/or contain the antigen-contacting residues (“antigen contacts”). Generally, antibodies comprise three heavy chain and three light chain CDRs or CDR regions in the variable region. CDRs provide the majority of contact residues for the binding of the antibody to the antigen or epitope.

The term “monoclonal antibody” as used herein refers to an antibody obtained from a population of substantially homogeneous antibodies, i.e., the individual antibodies comprising the population are identical and bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different epitopes. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the presently disclosed subject matter may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

The term “recombinant antibody””, as used herein, refers to antibodies that are prepared, expressed, created or isolated by recombinant means not existing in nature. In certain embodiments, a recombinant antibody is a recombinant murine antibody. Such recombinant murine antibodies have variable regions in which the framework and CDR regions are derived from murine germline immunoglobulin sequences. In certain embodiments, however, such recombinant murine antibodies can be subjected to in vitro mutagenesis (or, when an animal transgenic for murine Ig sequences is used, in vivo somatic mutagenesis) and thus the amino acid sequences of the VH and VL regions of the recombinant antibodies are sequences that, while derived from and related to murine germline VH and VL sequences, may not naturally exist within the murine antibody germline repertoire in vivo.

As used herein, the terms “recombinant” or “engineered,” with respect to a protein, means having an altered amino acid sequence as a result of the application of genetic engineering techniques to nucleic acids that encode the protein and cells or organisms that express the protein. With respect to a nucleic acid, the term “recombinant” or “engineered” means having an altered nucleic acid sequence as a result of the application of genetic engineering techniques. Genetic engineering techniques include, but are not limited to, PCR and DNA cloning technologies; transfection, transformation, and other gene transfer technologies; homologous recombination; site-directed mutagenesis; and gene fusion. In accordance with this definition, a protein having an amino acid sequence identical to a naturally-occurring protein, but produced by cloning and expression in a heterologous host, is not considered recombinant or engineered.

The term “humanized antibody” is intended to refer to antibodies in which CDRs from a mammalian species (other than a human), such as a mouse, are grafted onto human framework regions. Additional framework region modifications may be made within the human framework sequences.

The term “chimeric antibody” is intended to refer to antibodies in which the variable region sequences are derived from one species and the constant region sequences are derived from another species, such as an antibody in which the variable region sequences are derived from a mouse antibody and the constant region sequences are derived from a human antibody.

As used herein, an antibody that “specifically binds to human BCMA” is intended to refer to an antibody that binds to human BCMA with a K_{D} of about 5×10⁻⁷ M or less, about 1×10⁻⁷ M or less, about 5×10⁻⁸ M or less, about 1×10⁻⁸ M or less, about 5×10⁻⁹ M or less, about 1×10⁻⁹ M or less, about 5×10⁻¹⁰ M or less, about 1×10⁻¹⁰ M or less, about 5×10⁻¹¹ M or less, or about 1×10⁻¹¹ M or less. Methods for determining whether two molecules specifically bind are well known in the art and include, for example, equilibrium dialysis, surface plasmon resonance, and the like. Conversely, as used herein, the term “does not detectably bind” refers to an antibody that does not bind a cell (e.g., a genetically-modified cell) at a level significantly greater than background, e.g., binds to the cell at a level less than 10%, 8%, 6%, 5%, or 1% above background. In some embodiments, the antibody binds to the cell at a level less than 10%, 8%, 6%, 5%, or 1% more than an isotype control antibody. In one example, the binding is detected by Western blotting, flow cytometry, ELISA, antibody panning, and/or Biacore analysis.

An “antibody that competes for binding” or “antibody that cross-competes for binding” with a reference antibody for binding to an antigen (e.g., BCMA) refers to an antibody that blocks binding of the reference antibody to the antigen (e.g., BCMA) in a competition assay by 50% or more, and conversely, the reference antibody blocks binding of the antibody to the antigen (e.g., BCMA) in a competition assay by 50% or more. An exemplary competition assay is described in “Antibodies”, Harlow and Lane (Cold Spring Harbor Press, Cold Spring Harbor, NY).

As used herein, “isotype” refers to the antibody class (e.g., IgM or IgG1) that is encoded by the heavy chain constant region genes.

The phrases “an antibody recognizing an antigen” and “an antibody specific for an antigen” are used interchangeably herein with the term “an antibody which binds specifically to an antigen (e.g., a BCMA polypeptide).”

The terms “BCMA” and “B-cell maturation antigen” are used interchangeably, and include variants, isoforms, species homologs of human BCMA, and analogs having at least one common epitope with BCMA (e.g., human BCMA). An exemplary human BCMA sequence can be found under Entrez Gene Accession No.: NP_001183.

An “immunoconjugate” is an antibody conjugated to one or more heterologous molecule(s), including, but not limited to, a cytotoxic agent.

An “effective amount” of an antigen binding protein, e.g., an anti-BCMA antibody, or an antigen-binding fragment thereof, a pharmaceutical composition comprising thereof, refers to an amount effective, at dosages and for periods of time necessary, to achieve the desired therapeutic or prophylactic result, e.g., treating a tumor (e.g., multiple myeloma).

A “gamma secretase inhibitor” refers to a compound, such as a small molecule, that inhibits the activity of gamma secretase. Gamma secretase is a protease complex that cleaves BCMA.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In certain embodiments, antibodies of the presently disclosed subject matter are used to delay development of a disease or to slow the progression of a disease, e.g., a tumor (multiple myeloma).

As used herein, a “chimeric antigen receptor” or “CAR” refers to an engineered receptor that grafts specificity for an antigen (e.g., BCMA) or other ligand or molecule onto an immune effector cell (e.g., a T cell or NK cell). A CAR comprises at least an extracellular ligand-binding domain or moiety, a transmembrane domain, and an intracellular domain, wherein the intracellular domain comprises one or more signaling domains and/or co-stimulatory domains.

An extracellular ligand-binding domain or moiety of a CAR can be, for example, an antibody, or antibody fragment. In this context, the term “antibody fragment” can refer to at least one portion of an antibody, that retains the ability to specifically interact with (e.g., by binding, steric hindrance, stabilizing/destabilizing, spatial distribution) an epitope of an antigen. Examples of antibody fragments include, but are not limited to, any antibody fragments described elsewhere herein and including Fab, Fab′, F(ab′)2, Fv fragments, scFv antibody fragments, disulfide-linked Fvs (sdFv), a Fd fragment consisting of the VH and CH1 domains, linear antibodies, single domain antibodies such as sdAb (either VL or VH), camelid VHH domains, multi-specific antibodies formed from antibody fragments such as a bivalent fragment comprising two Fab fragments linked by a disulfide bridge at the hinge region, and an isolated CDR or other epitope binding fragments of an antibody. An antigen binding fragment can also be incorporated into single domain antibodies, maxibodies, minibodies, nanobodies, intrabodies, diabodies, triabodies, tetrabodies, v-NAR and bis-scFv (see, e.g., Hollinger and Hudson, Nature Biotechnology 23:1126-1136, 2005). Antigen binding fragments can also be grafted into scaffolds based on polypeptides such as a fibronectin type III (Fn3) (see U.S. Pat. No. 6,703,199, which describes fibronectin polypeptide minibodies).

In particular examples, the extracellular ligand-binding domain or moiety is in the form of a single-chain variable fragment (scFv) derived from a monoclonal antibody, which provides specificity for a particular epitope or antigen (e.g., an epitope or antigen preferentially present on the surface of a cell, such as a cancer cell or other disease-causing cell or particle). In some embodiments, the scFv is attached via a linker sequence. In some embodiments, the scFv is murine or humanized.

In some embodiments, the extracellular domain of a CAR comprises an autoantigen (see, Payne et al. (2016) Science, Vol. 353 (6295): 179-184), which is recognized by autoantigen-specific B cell receptors on B lymphocytes, thus directing T cells to specifically target and kill autoreactive B lymphocytes in antibody-mediated autoimmune diseases. Such CARs can be referred to as chimeric autoantibody receptors (CAARs), and are encompassed by the present disclosure.

The intracellular domain of a CAR can include one or more cytoplasmic signaling domains that transmit an activation signal to the T cell following antigen binding. Such cytoplasmic signaling domains can include, without limitation, a CD3 zeta signaling domain, such as that disclosed in SEQ ID NO: 66, and variants thereof.

The intracellular domain of a CAR can also include one or more intracellular co-stimulatory domains that transmit a proliferative and/or cell-survival signal after ligand binding. In some cases, the co-stimulatory domain can comprise one or more TRAF-binding domains. Intracellular co-stimulatory domains can be any of those known in the art and can include, without limitation, those co-stimulatory domains disclosed in WO 2018/067697 including, for example, Novel 1 (“N1”; SEQ ID NO: 58) and Novel 6 (“N6”; SEQ ID NO: 60). Further examples of co-stimulatory domains include 4-1BB (SEQ ID NO: 62), CD28 (SEQ ID NO: 64), or a functional signaling domain obtained from a protein including an MHC class I molecule, TNF receptor proteins, Immunoglobulin-like proteins, cytokine receptors, integrins, signaling lymphocytic activation molecules (SLAM proteins), activating NK cell receptors, BTLA, a Toll ligand receptor, OX40, CD2, CD7, CD27, CD30, CD40, CDS, ICAM-1, LFA-1 (CD11a/CD18), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRT AM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83.

A CAR further includes additional structural elements, including a transmembrane domain that is attached to the extracellular ligand-binding domain via a hinge or spacer sequence. The transmembrane domain can be derived from any membrane-bound or transmembrane protein. For example, the transmembrane polypeptide can be a subunit of the T-cell receptor (e.g., an α, β, γ or ζ, polypeptide constituting CD3 complex), IL2 receptor p55 (a chain), p75 (β chain) or 7 chain, subunit chain of Fc receptors (e.g., Fcy receptor III) or CD proteins such as the CD8 alpha chain. In certain examples, the transmembrane domain is a CD8 alpha domain set forth in SEQ ID NO: 56, and variants thereof. Alternatively, the transmembrane domain can be synthetic and can comprise predominantly hydrophobic residues such as leucine and valine.

The hinge region refers to any oligo- or polypeptide that functions to link the transmembrane domain to the extracellular ligand-binding domain. For example, a hinge region may comprise up to 300 amino acids, 10 to 100 amino acids or 25 to 50 amino acids. Hinge regions may be derived from all or part of naturally occurring molecules, such as from all or part of the extracellular region of CD8, CD4 or CD28, or from all or part of an antibody constant region. Alternatively, the hinge region may be a synthetic sequence that corresponds to a naturally occurring hinge sequence or may be an entirely synthetic hinge sequence. In particular examples, a hinge domain can comprise a part of a human CD8 alpha chain, FcγRllla receptor or IgG1. In certain examples, the hinge region can be a CD8 alpha domain set forth in SEQ ID NO: 54, and variants thereof.

As used herein, the term with respect to recombinant proteins, the term “modification” means any insertion, deletion, or substitution of an amino acid residue in the recombinant sequence relative to a reference sequence (e.g., a wild-type or a native sequence).

As used herein, the terms “cleave” or “cleavage” refer to the hydrolysis of phosphodiester bonds within the backbone of a recognition sequence within a target sequence that results in a double-stranded break within the target sequence, referred to herein as a “cleavage site”.

As used herein, the terms “nuclease” and “endonuclease” refers to enzymes which cleave a phosphodiester bond within a polynucleotide chain.

As used herein, the term “meganuclease” refers to an endonuclease that binds double-stranded DNA at a recognition sequence that is greater than 12 base pairs. In some embodiments, the recognition sequence for a meganuclease of the present disclosure is 22 base pairs. A meganuclease can be an endonuclease that is derived from I-CreI, and can refer to an engineered variant of I-CreI that has been modified relative to natural I-CreI with respect to, for example, DNA-binding specificity, DNA cleavage activity, DNA-binding affinity, or dimerization properties. Methods for producing such modified variants of I-CreI are known in the art (e.g., WO 2007/047859, incorporated by reference in its entirety). A meganuclease as used herein binds to double-stranded DNA as a heterodimer. A meganuclease may also be a “single-chain meganuclease” in which a pair of DNA-binding domains is joined into a single polypeptide using a peptide linker. The term “homing endonuclease” is synonymous with the term “meganuclease.” Meganucleases of the present disclosure are substantially non-toxic when expressed in the targeted cells described herein such that cells can be transfected and maintained at 37° C. without observing deleterious effects on cell viability or significant reductions in meganuclease cleavage activity when measured using the methods described herein.

As used herein, the term “single-chain meganuclease” refers to a polypeptide comprising a pair of nuclease subunits joined by a linker. A single-chain meganuclease has the organization: N-terminal subunit-Linker-C-terminal subunit. The two meganuclease subunits will generally be non-identical in amino acid sequence and will bind non-identical DNA sequences. Thus, single-chain meganucleases typically cleave pseudo-palindromic or non-palindromic recognition sequences. A single-chain meganuclease may be referred to as a “single-chain heterodimer” or “single-chain heterodimeric meganuclease” although it is not, in fact, dimeric. For clarity, unless otherwise specified, the term “meganuclease” can refer to a dimeric or single-chain meganuclease.

As used herein, the term “megaTAL” refers to a single-chain endonuclease comprising a transcription activator-like effector (TALE) DNA binding domain with an engineered, sequence-specific homing endonuclease.

As used herein, the term “compact TALEN” refers to an endonuclease comprising a DNA-binding domain with one or more TAL domain repeats fused in any orientation to any portion of the I-TevI homing endonuclease or any of the endonucleases listed in Table 2 in U.S. Application No. 20130117869 (which is incorporated by reference in its entirety), including but not limited to MmeI, EndA, Endl, I-BasI, I-TevII, I-TevIII, I-TwoI, MspI, MvaI, NucA, and NucM. Compact TALENs do not require dimerization for DNA processing activity, alleviating the need for dual target sites with intervening DNA spacers. In some embodiments, the compact TALEN comprises 16-22 TAL domain repeats.

As used herein, the terms “CRISPR” or “CRISPR nuclease” or “CRISPR system nuclease” refers to a CRISPR (clustered regularly interspaced short palindromic repeats)-associated (Cas) endonuclease or a variant thereof, such as Cas9, that associates with a guide RNA that directs nucleic acid cleavage by the associated endonuclease by hybridizing to a recognition site in a polynucleotide. In certain embodiments, the CRISPR nuclease is a class 2 CRISPR enzyme. In some of these embodiments, the CRISPR nuclease is a class 2, type II enzyme, such as Cas9. In other embodiments, the CRISPR nuclease is a class 2, type V enzyme, such as Cpf1. The guide RNA comprises a direct repeat and a guide sequence (often referred to as a spacer in the context of an endogenous CRISPR system), which is complementary to the target recognition site. In certain embodiments, the CRISPR system further comprises a tracrRNA (trans-activating CRISPR RNA) that is complementary (fully or partially) to the direct repeat sequence (sometimes referred to as a tracr-mate sequence) present on the guide RNA. In particular embodiments, the CRISPR nuclease can be mutated with respect to a corresponding wild-type enzyme such that the enzyme lacks the ability to cleave one strand of a target polynucleotide, functioning as a nickase, cleaving only a single strand of the target DNA. Non-limiting examples of CRISPR enzymes that function as a nickase include Cas9 enzymes with a D10A mutation within the RuvC I catalytic domain, or with a H840A, N854A, or N863A mutation. Given a predetermined DNA locus, recognition sequences can be identified using a number of programs known in the art (Kornel Labun; Tessa G. Montague; James A. Gagnon; Summer B. Thyme; Eivind Valen. (2016). CHOPCHOP v2: a web tool for the next generation of CRISPR genome engineering. Nucleic Acids Research; doi:10.1093/nar/gkw398; Tessa G. Montague; Jose M. Cruz; James A. Gagnon; George M. Church; Eivind Valen. (2014). CHOPCHOP: a CRISPR/Cas9 and TALEN web tool for genome editing. Nucleic Acids Res. 42. W401-W407).

As used herein, the term “TALEN” refers to an endonuclease comprising a DNA-binding domain comprising a plurality of TAL domain repeats fused to a nuclease domain or an active portion thereof from an endonuclease or exonuclease, including but not limited to a restriction endonuclease, homing endonuclease, S1 nuclease, mung bean nuclease, pancreatic DNAse I, micrococcal nuclease, and yeast HO endonuclease. See, for example, Christian et al. (2010) Genetics 186:757-761, which is incorporated by reference in its entirety. Nuclease domains useful for the design of TALENs include those from a Type IIs restriction endonuclease, including but not limited to FokI, FoM, StsI, HhaI, HindIII, Nod, BbvCI, EcoRI, BglI, and AlwI. Additional Type IIs restriction endonucleases are described in International Publication No. WO 2007/014275, which is incorporated by reference in its entirety. In some embodiments, the nuclease domain of the TALEN is a FokI nuclease domain or an active portion thereof. TAL domain repeats can be derived from the TALE (transcription activator-like effector) family of proteins used in the infection process by plant pathogens of the Xanthomonas genus. TAL domain repeats are 33-34 amino acid sequences with divergent 12th and 13th amino acids. These two positions, referred to as the repeat variable dipeptide (RVD), are highly variable and show a strong correlation with specific nucleotide recognition. Each base pair in the DNA target sequence is contacted by a single TAL repeat with the specificity resulting from the RVD. In some embodiments, the TALEN comprises 16-22 TAL domain repeats. DNA cleavage by a TALEN requires two DNA recognition regions (i.e., “half-sites”) flanking a nonspecific central region (i.e., the “spacer”). The term “spacer” in reference to a TALEN refers to the nucleic acid sequence that separates the two nucleic acid sequences recognized and bound by each monomer constituting a TALEN. The TAL domain repeats can be native sequences from a naturally-occurring TALE protein or can be redesigned through rational or experimental means to produce a protein that binds to a pre-determined DNA sequence (see, for example, Boch et al. (2009) Science 326(5959):1509-1512 and Moscou and Bogdanove (2009) Science 326(5959):1501, each of which is incorporated by reference in its entirety). See also, U.S. Publication No. 20110145940 and International Publication No. WO 2010/079430 for methods for engineering a TALEN to recognize and bind a specific sequence and examples of RVDs and their corresponding target nucleotides. In some embodiments, each nuclease (e.g., FokI) monomer can be fused to a TAL effector sequence that recognizes and binds a different DNA sequence, and only when the two recognition sites are in close proximity do the inactive monomers come together to create a functional enzyme. It is understood that the term “TALEN” can refer to a single TALEN protein or, alternatively, a pair of TALEN proteins (i.e., a left TALEN protein and a right TALEN protein) which bind to the upstream and downstream half-sites adjacent to the TALEN spacer sequence and work in concert to generate a cleavage site within the spacer sequence. Given a predetermined DNA locus or spacer sequence, upstream and downstream half-sites can be identified using a number of programs known in the art (Kornel Labun; Tessa G. Montague; James A. Gagnon; Summer B. Thyme; Eivind Valen. (2016). CHOPCHOP v2: a web tool for the next generation of CRISPR genome engineering. Nucleic Acids Research; doi:10.1093/nar/gkw398; Tessa G. Montague; Jose M. Cruz; James A. Gagnon; George M. Church; Eivind Valen. (2014). CHOPCHOP: a CRISPR/Cas9 and TALEN web tool for genome editing. Nucleic Acids Res. 42. W401-W407). It is also understood that a TALEN recognition sequence can be defined as the DNA binding sequence (i.e., half-site) of a single TALEN protein or, alternatively, a DNA sequence comprising the upstream half-site, the spacer sequence, and the downstream half-site.

As used herein, the terms “zinc finger nuclease” or “ZFN” refers to a chimeric protein comprising a zinc finger DNA-binding domain fused to a nuclease domain from an endonuclease or exonuclease, including but not limited to a restriction endonuclease, homing endonuclease, S1 nuclease, mung bean nuclease, pancreatic DNAse I, micrococcal nuclease, and yeast HO endonuclease. Nuclease domains useful for the design of zinc finger nucleases include those from a Type IIs restriction endonuclease, including but not limited to FokI, FoM, and StsI restriction enzyme. Additional Type IIs restriction endonucleases are described in International Publication No. WO 2007/014275, which is incorporated by reference in its entirety. The structure of a zinc finger domain is stabilized through coordination of a zinc ion. DNA binding proteins comprising one or more zinc finger domains bind DNA in a sequence-specific manner. The zinc finger domain can be a native sequence or can be redesigned through rational or experimental means to produce a protein which binds to a pre-determined DNA sequence ˜18 basepairs in length, comprising a pair of nine basepair half-sites separated by 2-10 basepairs. See, for example, U.S. Pat. Nos. 5,789,538, 5,925,523, 6,007,988, 6,013,453, 6,200,759, and International Publication Nos. WO 95/19431, WO 96/06166, WO 98/53057, WO 98/54311, WO 00/27878, WO 01/60970, WO 01/88197, and WO 02/099084, each of which is incorporated by reference in its entirety. By fusing this engineered protein domain to a nuclease domain, such as FokI nuclease, it is possible to target DNA breaks with genome-level specificity. The selection of target sites, zinc finger proteins and methods for design and construction of zinc finger nucleases are known to those of skill in the art and are described in detail in U.S. Publications Nos. 20030232410, 20050208489, 2005064474, 20050026157, 20060188987 and International Publication No. WO 07/014275, each of which is incorporated by reference in its entirety. In the case of a zinc finger, the DNA binding domains typically recognize an 18-bp recognition sequence comprising a pair of nine basepair “half-sites” separated by a 2-10 basepair “spacer sequence”, and cleavage by the nuclease creates a blunt end or a 5′ overhang of variable length (frequently four basepairs). It is understood that the term “zinc finger nuclease” can refer to a single zinc finger protein or, alternatively, a pair of zinc finger proteins (i.e., a left ZFN protein and a right ZFN protein) that bind to the upstream and downstream half-sites adjacent to the zinc finger nuclease spacer sequence and work in concert to generate a cleavage site within the spacer sequence. Given a predetermined DNA locus or spacer sequence, upstream and downstream half-sites can be identified using a number of programs known in the art (Mandell J G, Barbas C F 3rd. Zinc Finger Tools: custom DNA-binding domains for transcription factors and nucleases. Nucleic Acids Res. 2006 Jul. 1; 34 (Web Server issue):W516-23). It is also understood that a zinc finger nuclease recognition sequence can be defined as the DNA binding sequence (i.e., half-site) of a single zinc finger nuclease protein or, alternatively, a DNA sequence comprising the upstream half-site, the spacer sequence, and the downstream half-site.

As used herein, the terms “target site” or “target sequence” refers to a region of the chromosomal DNA of a cell comprising a recognition sequence for a nuclease. As used herein wherein referring a nuclease, the term “specificity” means the ability of a nuclease to recognize and cleave double-stranded DNA molecules only at a particular sequence of base pairs referred to as the recognition sequence, or only at a particular set of recognition sequences. The set of recognition sequences will share certain conserved positions or sequence motifs, but may be degenerate at one or more positions. A highly-specific nuclease is capable of cleaving only one or a very few recognition sequences. Specificity can be determined by any method known in the art.

As used herein, the terms “recognition sequence” or “recognition site” refers to a DNA sequence that is bound and cleaved by a nuclease. In the case of a meganuclease, a recognition sequence comprises a pair of inverted, 9 basepair “half sites” which are separated by four basepairs. In the case of a single-chain meganuclease, the N-terminal domain of the protein contacts a first half-site and the C-terminal domain of the protein contacts a second half-site. Cleavage by a meganuclease produces four basepair 3′ overhangs. “Overhangs,” or “sticky ends” are short, single-stranded DNA segments that can be produced by endonuclease cleavage of a double-stranded DNA sequence. In the case of meganucleases and single-chain meganucleases derived from I-CreI, the overhang comprises bases 10-13 of the 22 basepair recognition sequence. In the case of a compact TALEN, the recognition sequence comprises a first CNNNGN sequence that is recognized by the I-TevI domain, followed by a non-specific spacer 4-16 basepairs in length, followed by a second sequence 16-22 bp in length that is recognized by the TAL-effector domain (this sequence typically has a 5′ T base). Cleavage by a compact TALEN produces two basepair 3′ overhangs. In the case of a CRISPR nuclease, the recognition sequence is the sequence, typically 16-24 basepairs, to which the guide RNA binds to direct cleavage. Full complementarity between the guide sequence and the recognition sequence is not necessarily required to effect cleavage. Cleavage by a CRISPR nuclease can produce blunt ends (such as by a class 2, type II CRISPR nuclease) or overhanging ends (such as by a class 2, type V CRISPR nuclease), depending on the CRISPR nuclease. In those embodiments wherein a CpfI CRISPR nuclease is utilized, cleavage by the CRISPR complex comprising the same will result in 5′ overhangs and in certain embodiments, 5 nucleotide 5′ overhangs. Each CRISPR nuclease enzyme also requires the recognition of a PAM (protospacer adjacent motif) sequence that is near the recognition sequence complementary to the guide RNA. The precise sequence, length requirements for the PAM, and distance from the target sequence differ depending on the CRISPR nuclease enzyme, but PAMs are typically 2-5 base pair sequences adjacent to the target/recognition sequence. PAM sequences for particular CRISPR nuclease enzymes are known in the art (see, for example, U.S. Pat. No. 8,697,359 and U.S. Publication No. 20160208243, each of which is incorporated by reference in its entirety) and PAM sequences for novel or engineered CRISPR nuclease enzymes can be identified using methods known in the art, such as a PAM depletion assay (see, for example, Karvelis et al. (2017) Methods 121-122:3-8, which is incorporated herein in its entirety). In the case of a zinc finger, the DNA binding domains typically recognize an 18-bp recognition sequence comprising a pair of nine basepair “half-sites” separated by 2-10 basepairs and cleavage by the nuclease creates a blunt end or a 5′ overhang of variable length (frequently four basepairs).

As used herein, the term “recognition half-site,” “recognition sequence half-site,” or simply “half-site” means a nucleic acid sequence in a double-stranded DNA molecule that is recognized and bound by a monomer of a homodimeric or heterodimeric meganuclease or by one subunit of a single-chain meganuclease or by one subunit of a single-chain meganuclease, or by a monomer of a TALEN or zinc finger nuclease.

As used herein, the term “a control” or “a control cell” refers to a cell that provides a reference point for measuring changes in genotype or phenotype of a genetically-modified cell. A control cell may comprise, for example: (a) a wild-type cell, i.e., of the same genotype as the starting material for the genetic alteration which resulted in the genetically-modified cell; (b) a cell of the same genotype as the genetically-modified cell but which has been transformed with a null construct (i.e., with a construct which has no known effect on the trait of interest); or, (c) a cell genetically identical to the genetically-modified cell but which is not exposed to conditions or stimuli or further genetic modifications that would induce expression of altered genotype or phenotype.

As used herein, a “co-stimulatory domain” refers to a polypeptide domain which transmits an intracellular proliferative and/or cell-survival signal upon activation. Activation of a co-stimulatory domain may occur following homodimerization of two co-stimulatory domain polypeptides. Activation may also occur, for example, following activation of a construct comprising the co-stimulatory domain (e.g., a CAR). Generally, a co-stimulatory domain can be derived from a transmembrane co-stimulatory receptor, particularly from an intracellular portion of a co-stimulatory receptor. Non-limiting examples of co-stimulatory domains include, but are not limited to, those co-stimulatory domains described elsewhere herein. As used herein, a “co-stimulatory signal” refers to an intracellular signal induced by a co-stimulatory domain that promotes cell proliferation, expansion of a cell population in vitro and/or in vivo, promotes cell survival, modulates (e.g., upregulates or downregulates) the secretion of cytokines, and/or modulates the production and/or secretion of other immunomodulatory molecules.

As used herein, “detectable cell-surface expression of an endogenous TCR” refers to the ability to detect one or more components of the TCR complex (e.g., an alpha/beta TCR complex) on the cell surface of a T cell (e.g., a CAR T cell), or a population of T cells (e.g., CAR T cells) described herein, using standard experimental methods. Such methods can include, for example, immunostaining and/or flow cytometry specific for components of the TCR itself, such as a TCR alpha or TCR beta chain, or for components of the assembled cell surface TCR complex, such as CD3. Methods for detecting cell surface expression of an endogenous TCR (e.g., an alpha/beta TCR) on an immune cell include those described in MacLeod et al. (2017) Molecular Therapy 25(4): 949-961.

Similarly, the term “no detectable CD3 on the cell surface” refers to lack of detection of CD3 on the surface of a T cell (e.g., a CAR T cell) described herein, or population of T cells (e.g., CAR T cells) described herein, as detected using standard experimental methods in the art. Methods for detecting cell surface expression of CD3 on an immune cell include those described in MacLeod et al. (2017).

As used herein, the terms “DNA-binding affinity” or “binding affinity” means the tendency of a nuclease to non-covalently associate with a reference DNA molecule (e.g., a recognition sequence or an arbitrary sequence). Binding affinity is measured by a dissociation constant, Kd. As used herein, a nuclease has “altered” binding affinity if the Kd of the nuclease for a reference recognition sequence is increased or decreased by a statistically significant percent change relative to a reference nuclease.

The term “effector function” refers to a specialized function of a cell. Effector function of a T cell, for example, may be cytolytic activity or helper activity including the secretion of cytokines. An intracellular signaling domain, such as CD3 zeta, can provide an activation signal to the cell in response to binding of the extracellular domain. As discussed, the activation signal can induce an effector function of the cell such as, for example, cytolytic activity or cytokine secretion.

The term “effective amount” or “therapeutically effective amount”, as it relates to CARs of the invention and genetically-modified cells comprising such CARs, refers to an amount sufficient to effect beneficial or desirable biological and/or clinical results. The amount will vary depending on the therapeutic (e.g., a genetically-modified cell such as a CAR T cell, CAR NK cell) formulation or composition, the disease and its severity, and the age, weight, physical condition and responsiveness of the subject to be treated. In specific embodiments, an effective amount of a cell comprising a CAR described herein, or pharmaceutical compositions described herein, reduces at least one symptom or the progression of a disease (e.g., cancer). For example, an effective amount of the pharmaceutical compositions or genetically-modified cells described herein reduces the level of proliferation or metastasis of cancer, causes a partial or full response or remission of cancer, or reduces at least one symptom of cancer in a subject.

The term “emulsion” refers to, without limitation, any oil-in-water, water-in-oil, water-in-oil-in-water, or oil-in-water-in-oil dispersions or droplets, including lipid structures that can form as a result of hydrophobic forces that drive apolar residues (e.g., long hydrocarbon chains) away from water and polar head groups toward water, when a water immiscible phase is mixed with an aqueous phase.

As used herein, the term “genetically-modified” refers to a cell or organism in which, or in an ancestor of which, a genomic DNA sequence has been deliberately modified by recombinant technology. As used herein, the term “genetically-modified” encompasses the term “transgenic.” For example, in some embodiments, a genetically-modified cell is an immune cell, such as, for example, a genetically-modified human T cell, NK cell, B cell, and others.

As used herein, the term “homologous recombination” or “HR” refers to the natural, cellular process in which a double-stranded DNA-break is repaired using a homologous DNA sequence as the repair template (see, e.g. Cahill et al. (2006), Front. Biosci. 11:1958-1976). The homologous DNA sequence may be an endogenous chromosomal sequence or an exogenous nucleic acid that was delivered to the cell.

As used herein, the term “non-homologous end-joining” or “NHEJ” refers to the natural, cellular process in which a double-stranded DNA-break is repaired by the direct joining of two non-homologous DNA segments (see, e.g. Cahill et al. (2006), Front. Biosci. 11:1958-1976). DNA repair by non-homologous end-joining is error-prone and frequently results in the untemplated addition or deletion of DNA sequences at the site of repair. In some instances, cleavage at a target recognition sequence results in NHEJ at a target recognition site. Nuclease-induced cleavage of a target site in the coding sequence of a gene followed by DNA repair by NHEJ can introduce mutations into the coding sequence, such as frameshift mutations, that disrupt gene function. Thus, engineered nucleases can be used to effectively knock-out a gene in a population of cells.

As used herein, a “human T cell” or “T cell” refers to a T cell isolated from a human donor. In some cases, the human donor is not the subject treated according to the method (i.e., the T cells are allogeneic), but instead a healthy human donor. In some cases, the human donor is the subject treated according to the method. T cells, and cells derived therefrom, can include, for example, isolated T cells that have not been passaged in culture, or T cells that have been passaged and maintained under cell culture conditions without immortalization.

As used herein, the terms “human natural killer cell” or “human NK cell” or “natural killer cell” or “NK cell” refers to a type of cytotoxic lymphocyte critical to the innate immune system. The role NK cells play is analogous to that of cytotoxic T-cells in the vertebrate adaptive immune response. NK cells provide rapid responses to virally infected cells and respond to tumor formation, acting at around 3 days after infection. Human NK cells, and cells derived therefrom, include isolated NK cells that have not been passaged in culture, NK cells that have been passaged and maintained under cell culture conditions without immortalization, and NK cells that have been immortalized and can be maintained under cell culture conditions indefinitely.

As used herein, the term “linker” refers to a peptide or a short oligopeptide sequence used to join two subunits into a single polypeptide. A linker may have a sequence that is found in natural proteins or may be an artificial sequence that is not found in any natural protein. A linker may be flexible and lacking in secondary structure or may have a propensity to form a specific three-dimensional structure under physiological conditions. In one particular embodiment, a linker may have a length of about 2 to 10 amino acids. In another embodiment, a linker may have a length of about 10 to 80 amino acids. In yet another embodiment, a linker may have a length of more than 80 amino acids. In a particular embodiment, a linker may be arranged between antibody VH and VL regions. In some examples, such linkers may have an amino acid sequence as set forth in any one of SEQ ID NOs: 34-51, and variants thereof. In particular examples, a linker may have an amino acid sequence as set forth in SEQ ID NO: 34, and variants thereof. In another embodiment, a linker may be arranged between the transmembrane domain and the intracellular domain of a CAR. In other embodiments, a linker, also referred to herein as a “spacer” may be positioned between an anti-BCMA binding domain and the transmembrane domain of a CAR. Such spacers can include, for example, a spacer set forth in SEQ ID NO: 52, and variants thereof. In particular examples, the spacer set forth in SEQ ID NO: 52 is encoded by a nucleic acid sequence comprising SEQ ID NO: 53.

In some embodiments, a linker joins two single chain subunits of an engineered meganuclease described herein. In some such embodiments, a meganuclease linker may include a sequence that substantially comprises glycine and serine. In other such embodiments, a meganuclease linker may include, without limitation, any of those encompassed by U.S. Pat. Nos. 8,445,251, 9,340,777, 9,434,931, and 10,041,053, each of which is incorporated by reference in its entirety. In further such embodiments, a meganuclease linker may comprise residues 154-195 of SEQ ID NO: 76.

As used herein, the term “operably linked” is intended to mean a functional linkage between two or more elements. For example, an operable linkage between a nucleic acid sequence encoding a nuclease described herein and a regulatory sequence (e.g., a promoter) is a functional link that allows for expression of the nucleic acid sequence encoding the nuclease. Operably linked elements may be contiguous or non-contiguous. When used to refer to the joining of two protein coding regions, by operably linked is intended that the coding regions are in the same reading frame.

As used herein, the term “recombinant DNA construct,” “recombinant construct,” “expression cassette,” “expression construct,” “chimeric construct,” “construct,” and “recombinant DNA fragment” are used interchangeably herein and are single or double-stranded polynucleotides. A recombinant construct comprises an artificial combination of nucleic acid fragments, including, without limitation, regulatory and coding sequences that are not found together in nature. For example, a recombinant DNA construct may comprise regulatory sequences and coding sequences that are derived from different sources, or regulatory sequences and coding sequences derived from the same source and arranged in a manner different than that found in nature. Such a construct may be used by itself or may be used in conjunction with a vector.

As used herein, the terms “recombinant” or “engineered,” with respect to a protein, means having an altered amino acid sequence as a result of the application of genetic engineering techniques to nucleic acids that encode the protein and cells or organisms that express the protein. With respect to a nucleic acid, the term “recombinant” or “engineered” means having an altered nucleic acid sequence as a result of the application of genetic engineering techniques. Genetic engineering techniques include, but are not limited to, PCR and DNA cloning technologies; transfection, transformation, and other gene transfer technologies; homologous recombination; site-directed mutagenesis; and gene fusion. In accordance with this definition, a protein having an amino acid sequence identical to a naturally-occurring protein, but produced by cloning and expression in a heterologous host, is not considered recombinant or engineered.

Although the recombinant construct as a whole does not occur in nature, portions of the construct may be found in nature. For example, a recombinant DNA construct may comprise regulatory sequences and coding sequences that are derived from different sources, or regulatory sequences and coding sequences derived from the same source and arranged in a manner different than that found in nature. Such a construct may be used by itself or may be used in conjunction with a vector.

As used herein, the term “reduces” or “reduced” or “reduced expression” refers to any reduction in the symptoms or severity of a disease or any reduction in the proliferation or number of cancerous cells. In either case, such a reduction may be up to 5%, 10%, 20%, 30%, 40%, 50%, 60%, 70%, 80%, 90%, 95%, or up to 100%. Accordingly, the term “reduced” encompasses both a partial reduction and a complete reduction of a disease state. The term reduced can also refer to a reduction in the percentage of cells in a population of cells that express an endogenous polypeptide (i.e., an endogenous alpha/beta T cell receptor or CD3) at the cell surface when compared to a population of control cells.

As used herein, the term “T cell receptor alpha gene” or “TCR alpha gene” refer to the locus in a T cell which encodes the T cell receptor alpha subunit. The T cell receptor alpha gene can refer to NCBI Gene ID number 6955, before or after rearrangement. Following rearrangement, the T cell receptor alpha gene comprises an endogenous promoter, rearranged V and J segments, the endogenous splice donor site, an intron, the endogenous splice acceptor site, and the T cell receptor alpha constant region locus, which comprises the subunit coding exons.

As used herein, the term “T cell receptor alpha constant region” or “TCR alpha constant region” or “TRAC” refers to a coding sequence of the T cell receptor alpha gene. The TCR alpha constant region includes the wild-type sequence, and functional variants thereof, identified by NCBI Gene ID NO. 28755.

As used herein, the term “vector” or “recombinant DNA vector” may be a construct that includes a replication system and sequences that are capable of transcription and translation of a polypeptide-encoding sequence in a given host cell. If a vector is used, then the choice of vector is dependent upon the method that will be used to transform host cells as is well known to those skilled in the art. Vectors can include, without limitation, plasmid vectors and recombinant AAV vectors, or any other vector known in the art suitable for delivering a gene to a target cell. The skilled artisan is well aware of the genetic elements that must be present on the vector in order to successfully transform, select and propagate host cells comprising any of the isolated nucleotides or nucleic acid sequences of the invention. In some embodiments, a “vector” also refers to a viral vector. Viral vectors can include, without limitation, retroviral vectors, lentiviral vectors, adenoviral vectors, and adeno-associated viral vectors (AAV).

As used herein, the term “wild-type” refers to the most common naturally occurring allele (i.e., polynucleotide sequence) in the allele population of the same type of gene, wherein a polypeptide encoded by the wild-type allele has its original functions. The term “wild-type” also refers to a polypeptide encoded by a wild-type allele. Wild-type alleles (i.e., polynucleotides) and polypeptides are distinguishable from mutant or variant alleles and polypeptides, which comprise one or more mutations and/or substitutions relative to the wild-type sequence(s). Whereas a wild-type allele or polypeptide can confer a normal phenotype in an organism, a mutant or variant allele or polypeptide can, in some instances, confer an altered phenotype. Wild-type nucleases are distinguishable from recombinant or non-naturally-occurring nucleases. The term “wild-type” can also refer to a cell, an organism, and/or a subject which possesses a wild-type allele of a particular gene, or a cell, an organism, and/or a subject used for comparative purposes.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, up to 10%, up to 5%, and still up to 1% of a given value. Alternatively, particularly with respect to biological systems or processes, the term can mean within an order of magnitude, within 5-fold, or within 2-fold, of a value.

As described herein, any concentration range, percentage range, ratio range or integer range is to be understood to include the value of any integer within the recited range and, when appropriate, fractions thereof (such as one tenth and one hundredth of an integer), unless otherwise indicated.

### 2. Anti-BCMA Antibodies and Fragments Thereof

The antibodies of the presently disclosed subject matter are characterized by particular functional features or properties of the antibodies. For example, the antibodies bind specifically to BCMA (e.g., bind to human BCMA). Particularly, the antibodies bind specifically to a human BCMA having an amino acid sequence of SEQ ID NO: 1:

(SEQ ID NO: 1)
MLQMAGQCSQNEYFDSLLHACIPCQLRCSSNTPPLTCQRYCNASVTNSV
KGTNAILWTCLGLSLIISLAVFVLMFLLRKINSEPLKDEFKNTGSGLLG
MANIDLEKSRTGDEIILPRGLEYTVEECTCEDCIKSKPKVDSDHCFPLP
AMEEGATILVTTKTNDYCKSLPAALSATEIEKSISAR

In certain embodiments, an antibody of the presently disclosed subject matter binds (e.g., specifically binds) to BCMA with high affinity, for example with a K_{D} of 1×10⁻⁶ M or less, e.g., about 1×10⁻⁷ M or less, about 1×10⁻⁸ M or less, about 1×10⁻⁹ M or less, about 1×10⁻¹⁰ M or less, or about 1×10-” M or less. In certain embodiments, a presently disclosed anti-BCMA antibody binds (e.g., specifically binds) to BCMA (e.g., human BCMA) with a K_{D} of from about 1×10⁻¹ M to about 1×10⁻⁶ M, e.g., from about 1×10⁻¹ M to about 1×10⁻⁹ M, from about 1×10⁻¹⁰ M to about 1×10⁻⁹ M, from 1×10⁻⁹ M to about 1×10⁻⁸ M, from about 1×10⁻⁸ M to about 1×10⁻⁷ M, or from about 1×10⁻⁷ M to about 1×10⁻⁶ M. In certain embodiments, a presently disclosed anti-BCMA antibody binds (e.g., specifically binds) to BCMA (e.g., human BCMA) with a K_{D} of about 1×10⁻⁸ M or less. In certain embodiments, a presently disclosed anti-BCMA antibody binds (e.g., specifically binds) to BCMA (e.g., human BCMA) with a K_{D} of from about 1×10⁻⁹ M to about 1×10⁻¹⁰ M. In certain embodiments, a presently disclosed anti-BCMA antibody binds (e.g., specifically binds) to BCMA (e.g., human BCMA) with a K_{D} of from about 1×10⁻⁹ M to about 2.5×10⁻⁹ M. In certain embodiments, a presently disclosed anti-BCMA antibody binds (e.g., specifically binds) to BCMA (e.g., human BCMA) with a K_{D} of from about 1.38×10⁻⁹ M to about 2.14×10⁻⁹ M. In certain embodiments, a presently disclosed anti-BCMA antibody binds (e.g., specifically binds) to BCMA (e.g., human BCMA) with a K_{D} of about 1.38×10⁻⁹ M. In certain embodiments, a presently disclosed anti-BCMA antibody binds (e.g., specifically binds) to BCMA (e.g., human BCMA) with a K_{D} of about 2.14×10⁻⁹ M.

The heavy and light chains of an antibody of the presently disclosed subject matter can be full-length (e.g., an antibody can include at least one (e.g., one or two) complete heavy chains, and at least one (e.g., one or two) complete light chains), or can be an antigen-binding portion or fragment (e.g., a Fab, F(ab′)2, Fv, or a single chain Fv fragment (“scFv”)). In certain embodiments, the antibody heavy chain constant region is chosen from, e.g., IgG1, IgG2, IgG3, IgG4, IgM, IgAQ1, IgA2, IgD, and IgE, particularly chosen from, e.g., IgG1, IgG2, IgG3, and IgG4, more particularly, IgG1 (e.g., human IgG1). Thus, while in some examples an antibody described herein is an antigen-binding fragment, the antibody can also be in the form of an intact antibody comprising two VH regions, two VL regions, and appropriate heavy and light chain constant regions. In some embodiments, the antibody light chain constant region is chosen from, e.g., kappa or lambda, particularly kappa. In some embodiments, the antibody heavy chain constant region comprises an amino acid sequence set forth in SEQ ID NO: 77, or variants thereof described herein. In certain embodiments, the antibody light chain constant region comprises an amino acid sequence set forth in SEQ ID NO: 79, or variants thereof described herein.

In some embodiments, the presently disclosed subject matter includes antibodies that have an scFv sequence fused to one or more constant domains to form an antibody with an Fc region of a human immunoglobulin to yield a bivalent protein, increasing the overall avidity and stability of the antibody. In addition, the Fc portion allows the direct conjugation of other molecules, including but not limited to fluorescent dyes, cytotoxins, radioisotopes etc. to the antibody for example, for use in antigen quantitation studies, to immobilize the antibody for affinity measurements, for targeted delivery of a therapeutic agent, to test for Fc-mediated cytotoxicity using immune effector cells and many other applications.

In constructing a recombinant immunoglobulin, appropriate amino acid sequences for constant regions of various immunoglobulin isotypes and methods for the production of a wide array of antibodies are known to those of skill in the art.

The presently disclosed subject matter provides antibodies (e.g., monoclonal antibodies) that specifically bind to BCMA (e.g., human BCMA). The VH region amino acid sequences of anti-BCMA antibodies BCMA-3, BCMA-20, and BCMA-51c are set forth in SEQ ID NOs: 2, 6, and 10, respectively. The VL region amino acid sequences of BCMA-3, BCMA-20, and BCMA-51c are set forth in SEQ ID NOs: 4, 8, and 12, respectively. The amino acid sequences of the VH and VL regions of each antibody are summarized below:

BCMA-3
VH region:
(SEQ ID NO: 2)
QIQLVQSGPELKKPGETVKISCKASGYTFTHYSINWVKRAPGKGLKWMG
WINTESGEPTYAYDFKGRFAFSLETSASTAYLQINNLKYEDTATYFCAL
DYESAMDYWGQGTSVTVSS
VL region:
(SEQ ID NO: 4)
DIVLTQSPPSLAMSLGKRATISCRASESVTIPGQHLINWYQQKPGQPPK
LLIQRASNVESGVPARFSGSGSRTDFTLTIDPVEEDDVAVYYCLQTRGI
PRTFGGGTKLEIK
BCMA-20
VH region:
(SEQ ID NO: 6)
QIQLVQSGPELKKPGETVKISCKASGYTFTHYSINWVKRAPGKGLKWMG
WINTETRESTYAYDFKGRFAFSLETSASTAYLQINNLKYEDTATYFCAL
DYKQAMDYWGQGTSVTVSS
VL region:
(SEQ ID NO: 8)
DIVLTQSPPSLAMSLGKRATISCRASESVTIPGQHLIHWYQQRPGQPPK
LLIQRASNLESGVPARFSGSGSRTDFTLTIDPVEEDDVAVYYCLQTRKI
PRTFGGGTKLEIK
BCMA-51c
VH region:
(SEQ ID NO: 10)
QIQLVQSGPELKKPGETVKISCKASGYTFTHYSINWVKRAPGKGLKWMG
WINTETRESTYAYDFKGRFAFSLETSASTAYLQINNLKYEDTATYFCAL
DYWSAMDYWGQGTSVTVSS
VL region:
(SEQ ID NO: 12)
DIVLTQSPPSLAMSLGKRATISCRASESVTIQGLHLIHWYQQKPGQPPK
LLIQRASNVQSGVPARFSGSGSRTDFTLTIDPVEEDDVAVYYCQQTRRI
PRTFGGGTKLEIK

Given that each of the BCMA-3, BCMA-20, and BCMA51c antibodies can bind to BCMA, the VH and VL sequences can be “mixed and matched” to create other anti-BCMA binding molecules. BCMA binding of such “mixed and matched” antibodies can be tested using the binding assays known in the art, including for example, ELISAs, Western blots, RIAs, Biacore analysis. When VH and VL chains are mixed and matched, a VH sequence from a particular VH/VL pairing is replaced with a structurally similar VH sequence. Likewise, a VL sequence from a particular VH/VL pairing is replaced with a structurally similar VL sequence.

In certain embodiments, the presently disclosed subject matter provides antibodies that comprise the heavy chain CDRs (CDRH1, CDRH2, and CDRH3) and light chain CDRs (CDRL1, CDRL2, and CDRL3) of the BCMA-3, BCMA-20, and BCMA-51c antibodies, or antibodies comprising VH and VL combinations thereof. The identification of CDR sequences within a VH or VL region has been described by Kabat et al., J. Biol. Chem. 252, 6609-6616 (1977) and Kabat et al., Sequences of protein of immunological interest. (1991), and by Chothia et al., J. Mol. Biol. 196:901-917 (1987) and by MacCallum et al., J. Mol. Biol. 262:732-745 (1996). In particular examples, the CDR sequences of the VH and VL regions are identified by the Kabat numbering scheme. In particular examples, the CDR sequences of the VH and VL regions are identified by the Chothia numbering scheme.

The amino acid sequences of the CDRH1 domains of BCMA-3, BCMA-20, and BCMA-51c, as determined by the Kabat numbering scheme, are set forth in SEQ ID NOs: 14, 20, and 26, respectively. The amino acid sequences of the CDRH2 domains of BCMA-3, BCMA-20, and BCMA-51c, as determined by the Kabat numbering scheme, are shown in SEQ ID NOs: 15, 21, and 27, respectively. The amino acid sequences of the CDRH3 domains of BCMA-3, BCMA-20, and BCMA-51c, as determined by the Kabat numbering scheme, are set forth in SEQ ID NOs: 16, 22, and 28, respectively. The amino acid sequences of the CDRL1 domains of BCMA-3, BCMA-20, and BCMA-51c, as determined by the Kabat numbering scheme, are set forth in SEQ ID NOs: 17, 23, and 29, respectively. The amino acid sequences of the CDRL2 domains of BCMA-3, BCMA-20, and BCMA-51c, as determined by the Kabat numbering scheme, are set forth in SEQ ID NOs: 18, 24, and 30, respectively. The amino acid sequences of the CDRL3 domains of BCMA-3, BCMA-20, and BCMA-51c, as determined by the Kabat numbering scheme, are shown in SEQ ID NOs: 19, 25, and 31, respectively. The amino acid sequences of the CDR domains of each antibody are summarized as follows:

BCMA-3
CDRH1:
(SEQ ID NO: 14)
HYSIN
CDRH2:
(SEQ ID NO: 15)
WINTESGEPTYAYDFKG
CDRH3:
(SEQ ID NO: 16)
DYESAMDY
CDRL1:
(SEQ ID NO: 17)
RASESVTIPGQHLIN
CDRL2:
(SEQ ID NO: 18)
RASNVES
CDRL3:
(SEQ ID NO: 19)
LQTRGIPRT
BCMA-20
CDRH1:
(SEQ ID NO: 20)
HYSIN
CDRH2:
(SEQ ID NO: 21)
WINTETRESTYAYDFKG
CDRH3:
(SEQ ID NO: 22)
DYKQAMDY
CDRL1:
(SEQ ID NO: 23)
RASESVTIPGQHLIH
CDRL2:
(SEQ ID NO: 24)
RASNLES
CDRL3:
(SEQ ID NO: 25)
LQTRKIPRT
BCMA-51c
CDRH1:
(SEQ ID NO: 26)
HYSIN
CDRH2:
(SEQ ID NO: 27)
WINTETRESTYAYDFKG
CDRH3:
(SEQ ID NO: 28)
DYWSAMDY
CDRL1:
(SEQ ID NO: 29)
RASESVTIQGLHLIH
CDRL2:
(SEQ ID NO: 30)
RASNVQS
CDRL3:
(SEQ ID NO: 31)
QQTRRIPRT

Given that each of these antibodies can bind to BCMA and that antigen-binding specificity is provided primarily by the six CDR domains, the CDRH1, CDRH2, CDRH3, CDRL1, CDRL2, and CDRL3 sequences can be “mixed and matched” (i.e., CDRs from different antibodies can be mixed and match, although each antibody typically contains a CDRH1, CDRH2, CDRH3, CDRL1, CDRL2, and CDRL3 domain) to create other anti-BCMA binding molecules. BCMA binding of such “mixed and matched” antibodies can be tested using the binding assays described above. When VH CDR sequences are mixed and matched, the CDRH1, CDRH2 and/or CDRH3 sequence from a particular VH sequence is replaced with a structurally similar CDR sequence(s). Likewise, when VL CDR sequences are mixed and matched, the CDRL1, CDRL2 and/or CDRL3 sequence from a particular VL sequence may be replaced with a structurally similar CDR sequence(s). It will be readily apparent to the ordinarily skilled artisan that novel VH and VL sequences can be created by substituting one or more VH and/or VL CDR region sequences with structurally similar sequences from the CDR sequences of the antibodies disclosed herein.

The constant region/framework region of the anti-BCMA antibodies disclosed herein can be altered, for example, by amino acid substitution, to modify the properties of the antibody (e.g., to increase or decrease one or more of: antigen binding affinity, Fc receptor binding, antibody carbohydrate, for example, glycosylation, fucosylation etc, the number of cysteine residues, effector cell function, effector cell function, complement function or introduction of a conjugation site). In some embodiments, the heavy chain constant (CH) region of an antibody disclosed herein comprises SEQ ID NO: 77. In some embodiments, the light chain (CL) constant region of an antibody disclosed herein comprises SEQ ID NO: 79.

(SEQ ID NO: 77)
ASTKGPSVFPLAPCSRSTSESTAALGCLVKDYFPEPVTVSWNSGALTSG
VHTFPAVLQSSGLYSLSSVVTVPSSSLGTKTYTCNVDHKPSNTKVDKRV
ESKYGPPCPPCPAPEFLGGPSVFLFPPKPKDTLMISRTPEVTCVVVDVS
QEDPEVQFNWYVDGVEVHNAKTKPREEQFNSTYRVVSVLTVLHQDWLNG
KEYKCKVSNKGLPSSIEKTISKAKGQPREPQVYTLPPSQEEMTKNQVSL
TCLVKGFYPSDIAVEWESNGQPENNYKTTPPVLDSDGSFFLYSRLTVDK
SRWQEGNVFSCSVMHEALHNHYTQKSLSLSPGK
(SEQ ID NO: 79)
RTVAAPSVFIFPPSDEQLKSGTASVVCLLNNFYPREAKVQWKVDNALQS
GNSQESVTEQDSKDSTYSLSSTLTLSKADYEKHKVYACEVTHQGLSSPV
TKSFNRGEC

2.1. Anti-BCMA Antibodies that Cross-compete for Binding to BCMA with Anti-BCMA Antibodies of the Invention

The presently disclosed subject matter provides antibodies that cross-compete with any of the disclosed anti-BCMA antibodies for binding to BCMA (e.g., human BCMA). For example, and not by way of limitation, the cross-competing antibodies can bind to the same epitope region, e.g., same epitope, adjacent epitope, or overlapping as any of the anti-BCMA antibodies of the presently disclosed subject matter. In certain embodiments, the reference antibody for cross-competition studies can be any one of the anti-BCMA antibodies disclosed herein, e.g., BCMA-3, BCMA-20, BCMA-51c, or antibodies comprising VH and VL combinations thereof.

Such cross-competing antibodies can be identified based on their ability to cross-compete with any one of the presently disclosed anti-BCMA antibodies in standard BCMA binding assays. For example, Biacore analysis, ELISA assays or flow cytometry can be used to demonstrate cross-competition with the antibodies of the presently disclosed subject matter. The ability of a test antibody to inhibit the binding of, for example, any one of the presently disclosed anti-BCMA antibodies to human BCMA demonstrates that the test antibody can compete with any one of the presently disclosed anti-BCMA antibodies for binding to human BCMA and thus binds to the same epitope region on human BCMA as any one of the presently disclosed anti-BCMA antibodies. In certain embodiments, the cross-competing antibody binds to the same epitope on human BCMA as any one of the presently disclosed anti-BCMA antibodies.

### 2.2. Characterization of Antibody Binding to BCMA

Antibodies of the presently disclosed subject matter can be tested for binding to BCMA by, for example, standard ELISA. To determine if the selected anti-BCMA antibodies bind to unique epitopes, each antibody can be biotinylated using commercially available reagents (Pierce, Rockford, Ill.). Competition studies using unlabeled monoclonal antibodies and biotinylated monoclonal antibodies can be performed using BCMA coated-ELISA plates as described above. Biotinylated mAb binding can be detected with a strep-avidin-alkaline phosphatase probe.

To determine the isotype of purified antibodies, isotype ELISAs can be performed using reagents specific for antibodies of a particular isotype. Anti-BCMA human IgGs can be further tested for reactivity with BCMA antigen by Western blotting.

In certain embodiments, K_{D} is measured by a radiolabeled antigen binding assay (RIA). In certain embodiments, an RIA is performed with the Fab version of an antibody of interest and its antigen. For example, solution binding affinity of Fabs for antigen is measured by equilibrating Fab with a minimal concentration of (125I)-labeled antigen in the presence of a titration series of unlabeled antigen, then capturing bound antigen with an anti-Fab antibody-coated plate (see, e.g., Chen et al., J. Mol. Biol. 293:865-881(1999)).

In certain embodiments, K_{D} is measured using a BIACORE® surface plasmon resonance assay. For example, an assay using a BIACORE®-2000 or a BIACORE®-3000 (BIAcore, Inc., Piscataway, N.J.).

The antibodies of the present invention may be prepared and purified using known methods in the art. For example, cDNA sequences encoding a heavy chain and a light chain may be cloned and engineered into an expression vector. The engineered immunoglobulin expression vector may then be stably transfected into a mammalian host cell, such as a Chinese Hamster Ovary (CHO) cells (e.g., GS-CHO) or NS0 cells. Stable clones may be verified for expression of an antibody specifically binding to human BCMA. Positive clones may be expanded into serum-free culture medium for antibody production in bioreactors. Media, into which an antibody has been secreted, may be purified by conventional techniques. For example, the medium may be conveniently applied to a Protein A column that has been equilibrated with a compatible buffer, such as phosphate buffered saline. The column is washed to remove nonspecific binding components. The bound antibody is eluted, for example, by pH gradient and antibody fractions are detected, such as by SDS-PAGE, and then pooled. The antibody may be further purified, concentrated and/or sterile filtered using common techniques. Soluble aggregate and multimers may be effectively removed by common techniques, including size exclusion, hydrophobic interaction, ion exchange, or hydroxyapatite chromatography. The product may subsequently be processed for use, for example, in a pharmaceutical formulation.

### 3. Single-Domain Fragments and Single-Chain Variable Fragments (scFvs)

In some examples, the anti-BCMA antibodies described herein can be in the form of an anti-BCMA single-domain antibody (sdAb) fragment comprising the CDRH1, CDRH2, and CDRH3 domains, or comprising a VH region, or variants thereof, of any antibody described herein (e.g., BCMA-3, BCMA-20, and BCMA-51c).

An anti-BCMA antibody described herein can also be in the form of an anti-BCMA single-chain variable fragment (scFv). An scFv is a fusion protein of the variable regions of the VH region and VL region of any antibody described herein or variants thereof, that are covalently linked to form a VH-VL or VL-VH heterodimer. The VH region and VL region are either joined directly or joined by a peptide-encoding linker, which connects the N-terminus of the VH with the C-terminus of the VL, or the C-terminus of the VH with the N-terminus of the VL. The linker is usually rich in glycine for flexibility, as well as serine or threonine for solubility. Non-limiting examples of linkers useful for connecting a VH region and VL region in an scFv include those set forth in any one of SEQ ID NOs: 34-51, and variants thereof. In particular examples of the invention, the linker comprises an amino acid sequence set forth in SEQ ID NO: 34, or variants thereof.

(SEQ ID NO: 34)
GSTSGSGKPGSGEGSTKG

The invention encompasses scFvs, either with or without a linker, generated from the VH and VL regions, and variants thereof, of any antibody described herein (e.g., BCMA-3, BCMA-20, and BCMA-51c), or of any antibody comprising the CDRH1, CDRH2, CDRH3, CDRL1, CDRL2, and/or CDRL3 domains described herein. The invention further 30 encompasses scFvs, either with or without a linker, that are prepared by mixing and matching the VH region and VL regions, and variants thereof, of any antibody disclosed herein (e.g., BCMA-3, BCMA-20, and BCMA-51c), or of any antibody comprising the CDRH1, CDRH2, CDRH3, CDRL1, CDRL2, and/or CDRL3 domains described herein.

The scFvs encompassed by the invention can have a 5′ to 3′ orientation of, for example, VH-VL, VL-VH, VH-linker-VL, or VL-linker-VH. In particular examples, an scFv encompassed by the invention is a BCMA-3H/3L scFv (SEQ ID NO: 81), a BCMA-3L/3H scFv (SEQ ID NO: 82), a BCMA-20H/20L scFv (SEQ ID NO: 83), a BCMA-20L/20H scFv (SEQ ID NO: 84), a BCMA-51cH/51cL scFv (SEQ ID NO: 85), a BCMA-51cL/51cH scFv (SEQ ID NO: 86), a BCMA-3H/20L scFv (SEQ ID NO: 87), a BCMA-3L/20H scFv (SEQ ID NO: 88), a BCMA-3H/51cL scFv (SEQ ID NO: 89), a BCMA-3L/51cH scFv (SEQ ID NO: 90), a BCMA-20H/3L scFv (SEQ ID NO: 91), a BCMA-20L/3H scFv (SEQ ID NO: 92), a BCMA-20H/51cL scFv (SEQ ID NO: 93), a BCMA-20L/51cH scFv (SEQ ID NO: 94), a BCMA-51cH/3L scFv (SEQ ID NO: 95), a BCMA-51cL/3H scFv (SEQ ID NO: 96), a BCMA-51cH/20L scFv (SEQ ID NO: 97), or a BCMA-51cL/20H scFv (SEQ ID NO: 98), and variants thereof. Thus, in some embodiments, the scFv is a BCMA-3H/3L scFv (SEQ ID NO: 81). In some embodiments, the scFv is a BCMA-3H/3L scFv (SEQ ID NO: 81). In some embodiments, the scFv is a BCMA-3L/3H scFv (SEQ ID NO: 82). In some embodiments, the scFv is a BCMA-20H/20L scFv (SEQ ID NO: 83). In some embodiments, the scFv is a BCMA-20L/20H scFv (SEQ ID NO: 84). In some embodiments, the scFv is a BCMA-51cH/51cL scFv (SEQ ID NO: 85). In some embodiments, the scFv is a BCMA− 51cL/51cH scFv (SEQ ID NO: 86). In some embodiments, the scFv is a BCMA-3H/20L scFv (SEQ ID NO: 87). In some embodiments, the scFv is a BCMA-3L/20H scFv (SEQ ID NO: 88). In some embodiments, the scFv is a BCMA-3H/51cL scFv (SEQ ID NO: 89). In some embodiments, the scFv is a BCMA-3L/51cH scFv (SEQ ID NO: 90). In some embodiments, the scFv is a BCMA-20H/3L scFv (SEQ ID NO: 91). In some embodiments, the scFv is a BCMA-20L/3H scFv (SEQ ID NO: 92). In some embodiments, the scFv is a BCMA-20H/51cL scFv (SEQ ID NO: 93). In some embodiments, the scFv is a BCMA− 20L/51cH scFv (SEQ ID NO: 94). In some embodiments, the scFv is a BCMA-51cH/3L scFv (SEQ ID NO: 95). In some embodiments, the scFv is a BCMA-51cL/3H scFv (SEQ ID NO: 96). In some embodiments, the scFv is a BCMA-51cH/20L scFv (SEQ ID NO: 97). In some embodiments, the scFv is a BCMA-51cL/20H scFv (SEQ ID NO: 98).

### 4. Homologous Antibodies

In certain embodiments, an antibody of the presently disclosed subject matter comprises heavy and light chain variable regions comprising amino acid sequences that are homologous to the amino acid sequences of the antibodies described herein (e.g., BCMA-3, BCMA-20, and BCMA-51c), and wherein the antibodies retain the desired functional properties of the anti-BCMA antibodies of the presently disclosed subject matter.

For example, the presently disclosed subject matter provides an isolated antibody, or antigen-binding portion thereof, comprising a heavy chain variable region and a light chain variable region, wherein: (a) the heavy chain variable region comprises an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous to an amino acid sequence set forth in any one of SEQ ID NOs: 2, 6, and 10; and/or (b) the light chain variable region comprises an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous to an amino acid sequence set forth in any one of SEQ ID NOs: 4, 8, and 12; wherein the antibody, or antigen-binding fragment thereof, binds (e.g., specifically binds) to human BCMA.

In other examples, the presently disclosed subject matter provides an isolated antibody, or antigen-binding portion thereof, comprising a heavy chain variable region and a light chain variable region, wherein: (a) the heavy chain variable region is encoded by a nucleic acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous to a sequence set forth in any one of SEQ ID NOs: 3, 7, and 11; and/or (b) the light chain variable region is encoded by a nucleic acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous to a sequence set forth in any one of SEQ ID NOs: 5, 9, and 13; wherein the antibody, or antigen-binding fragment thereof, binds (e.g., specifically binds) to human BCMA.

In certain embodiments, the VH and/or VL amino acid sequences can be at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous to the sequences set forth above. An antibody having VH and VL regions having high (i.e., 80% or greater) homology to the VH and VL regions of the sequences set forth above, can be obtained by mutagenesis (e.g., site-directed or PCR-mediated mutagenesis), followed by testing of the encoded altered antibody for retained function (i.e., the binding affinity) using the binding assays described herein.

As used herein, the percent homology between two amino acid sequences is equivalent to the percent identity between the two sequences. The percent identity or homology between the two sequences is a function of the number of identical positions shared by the sequences (i.e., % homology=# of identical positions/total # of positions ×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent homology between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent homology between two amino acid sequences can be determined using the Needleman and Wunsch (J. Mol. Biol. 48:444-453 (1970)) algorithm which has been incorporated into the GAP program in the GCG software package (available at gcg.com), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the protein sequences of the presently disclosed subject matter can further be used as a “query sequence” to perform a search against public databases to, for example, identify related sequences. Such searches can be performed using the XBLAST program (version 2.0) of Altschul, et al. (1990) J. Mol. Biol. 215:403-10. BLAST protein searches can be performed with the XBLAST program, score=50, wordlength=3 to obtain amino acid sequences homologous to the antibody molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. (See ncbi.nlm.nih.gov).

### 5. Immunoconjugates

The presently disclosed subject matter provides an anti-BCMA antibody, or a fragment thereof, conjugated to a therapeutic moiety, such as a cytotoxin, a drug (e.g., an immunosuppressant) or a radiotoxin. Such conjugates are referred to herein as “immunoconjugates”. Immunoconjugates that include one or more cytotoxins are referred to as “immunotoxins.” A cytotoxin or cytotoxic agent includes any agent that is detrimental to (e.g., kills) cells. Examples include taxol (such as ricin, diphtheria, gelonin), cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents also include, for example, calecheamicin, aureastatin, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

Other examples of therapeutic cytotoxins that can be conjugated to an anti-BCMA antibody disclosed herein include duocarmycins, calicheamicins, maytansines and auristatins, and derivatives thereof. An example of a calicheamicin antibody conjugate is commercially available (Mylotarg™; Wyeth-Ayerst).

Cytotoxins can be conjugated to anti-BCMA antibody disclosed herein using linker technology available in the art. Examples of linker types that have been used to conjugate a cytotoxin to an antibody include, but are not limited to, hydrazones, thioethers, esters, disulfides and peptide-containing linkers. A linker can be chosen that is, for example, susceptible to cleavage by low pH within the lysosomal compartment or susceptible to cleavage by proteases, such as proteases preferentially expressed in tumor tissue such as cathepsins (e.g., cathepsins B, C, D). For further discussion of types of cytotoxins, linkers and methods for conjugating therapeutic agents to antibodies, see also Saito, G. et al. (2003) Adv. Drug Deliv. Rev. 55:199-215; Trail, P. A. et al. (2003) Cancer Immunol. Immunother. 52:328-337; Payne, G. (2003) Cancer Cell 3:207-212; Allen, T. M. (2002) Nat. Rev. Cancer 2:750-763; Pastan, I. and Kreitman, R. J. (2002) Curr. Opin. Investig. Drugs 3:1089-1091; Senter, P. D. and Springer, C. J. (2001) Adv. Drug Deliv. Rev. 53:247-264.

Anti-BCMA antibodies of the presently disclosed subject matter also can be conjugated to a radioactive isotope to generate cytotoxic radiopharmaceuticals, also referred to as radioimmunoconjugates. Examples of radioactive isotopes that can be conjugated to antibodies for use diagnostically or therapeutically include, but are not limited to, 90Y, 131I, 225Ac, 213Bi, 223Ra and 227Th. Methods for preparing radioimmunconjugates are established in the art. Examples of radioimmunoconjugates are commercially available, including Zevalin™ (IDEC Pharmaceuticals) and Bexxar™ (Corixa Pharmaceuticals), and similar methods can be used to prepare radioimmunoconjugates using the antibodies of the invention.

The antibody conjugates of the presently disclosed subject matter can be used to modify a given biological response, and the drug moiety is not to be construed as limited to classical chemical therapeutic agents. For example, the drug moiety may be a protein or polypeptide possessing a desired biological activity. Such proteins may include, for example, an enzymatically active toxin, or active fragment thereof, such as abrin, ricin A, pseudomonas exotoxin, or diphtheria toxin; a protein such as tumor necrosis factor (TNF) or interferon-γ; or, biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other growth factors.

Techniques for conjugating such therapeutic moiety to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982).

### 6. Multispecific Molecules

The presently disclosed subject matter provides multispecific, e.g., bispecific, molecules comprising an anti-BCMA antibody, or a fragment thereof, disclosed herein. An antibody of the presently disclosed subject matter, or antigen-binding portions thereof, can be derivatized or linked to another functional molecule, e.g., another peptide or protein (e.g., another antibody or ligand for a receptor) to generate a bispecific molecule that binds to at least two different binding sites or target molecules. The antibody of the presently disclosed subject matter can in fact be derivatized or linked to more than one other functional molecule to generate multispecific molecules that bind to more than two different binding sites and/or target molecules; such multispecific molecules are also intended to be encompassed by the term “bispecific molecule” as used herein. To create a bispecific molecule, a presently disclosed anti-BCMA antibody can be functionally linked (e.g., by chemical coupling, genetic fusion, noncovalent association or otherwise) to one or more other binding molecules, such as another antibody, antibody fragment, peptide or binding mimetic, such that a bispecific molecule results.

The presently disclosed subject matter provides bispecific molecules comprising at least a first binding specificity for BCMA and a second binding specificity for a second target epitope. The second target epitope can be a BCMA epitope, or a non-BCMA epitope, e.g., a different antigen. In certain embodiments, the bispecific molecule is multispecific, the molecule can further include a third binding specificity. Where a first portion of a bispecific antibody binds to an antigen on a tumor cell for example and a second portion of a bispecific antibody recognizes an antigen on the surface of a human immune effector cell, the antibody is capable of recruiting the activity of that effector cell by specifically binding to the effector antigen on the human immune effector cell. In certain embodiments, bispecific antibodies, therefore, are able to form a link between effector cells, for example, T cells and tumor cells, thereby enhancing effector function. In certain embodiments, a bispecific antibody of the present disclosure comprises at least a first binding to BCMA and at least a second binding to an immune cell.

The bispecific molecules of the presently disclosed subject matter can be prepared by conjugating the constituent binding specificities using methods known in the art. For example, each binding specificity of the bispecific molecule can be generated separately and then conjugated to one another. When the binding specificities are proteins or peptides, a variety of coupling or cross-linking agents can be used for covalent conjugation. Examples of cross-linking agents include protein A, carbodiimide, N-succinimidyl-S-acetyl-thioacetate (SATA), 5,5′-dithiobis(2-nitrobenzoic acid) (DTNB), o-phenylenedimaleimide (oPDM), N-succinimidyl-3-(2-pyridyldithio)propionate (SPDP), and sulfosuccinimidyl 4-(N-maleimidomethyl) cyclohaxane-1-carboxylate (sulfo-SMCC) (see e.g., Karpovsky et al. (1984) J. Exp. Med. 160:1686; Liu, M A et al. (1985) Proc. Natl. Acad. Sci. USA 82:8648). Other methods include those described in Paulus (1985) Behring Ins. Mitt. No. 78, 118-132; Brennan et al. (1985) Science 229:81-83), and Glennie et al. (1987) J. Immunol. 139: 2367-2375). Particular conjugating agents are SATA and sulfo-SMCC, both available from Pierce Chemical Co. (Rockford, Ill.).

When the binding specificities are antibodies, they can be conjugated via sulfhydryl bonding of the C-terminus hinge regions of the two heavy chains. In certain embodiments, the hinge region is modified to contain an odd number of sulfhydryl residues, in some embodiments one, prior to conjugation.

Alternatively, both binding specificities can be encoded in the same vector and expressed and assembled in the same host cell. This method is particularly useful where the bispecific molecule is a mAbxmAb, mAbxFab, FabxF(ab′)2 or ligandxFab fusion protein.

Binding of the bispecific molecules to their specific targets can be confirmed by, for example, enzyme-linked immunosorbent assay (ELISA), radioimmunoassay (RIA), FACS analysis, bioassay (e.g., growth inhibition), or Western Blot assay. Each of these assays generally detects the presence of protein-antibody complexes of particular interest by employing a labeled reagent (e.g., an antibody) specific for the complex of interest. Alternatively, the complexes can be detected using any of a variety of other immunoassays. For example, the antibody can be radioactively labeled and used in a radioimmunoassay (MA) (see, for example, Weintraub, B., Principles of Radioimmunoassays, Seventh Training Course on Radioligand Assay Techniques, The Endocrine Society, March, 1986, which is incorporated by reference herein). The radioactive isotope can be detected by such means as the use of a 7 counter or a scintillation counter or by autoradiography.

### 7. Pharmaceutical Compositions and Methods of Treatment

Anti-BCMA antibodies of the presently disclosed subject matter can be administered for therapeutic treatments to a patient suffering from a tumor (e.g., multiple myeloma) in an amount sufficient to prevent, inhibit, or reduce the progression of the tumor. Progression includes, e.g., the growth, invasiveness, metastases and/or recurrence of the tumor. Amounts effective for this use will depend upon the severity of the disease and the general state of the patient's own immune system. Dosing schedules will also vary with the disease state and status of the patient, and will typically range from a single bolus dosage or continuous infusion to multiple administrations per day (e.g., every 4-6 hours), or as indicated by the treating physician and the patient's condition.

The identification of medical conditions treatable by anti-BCMA antibodies of the presently disclosed subject matter is well within the ability and knowledge of one skilled in the art. For example, human individuals who are either suffering from multiple myeloma or who are at risk of developing multiple myeloma are suitable for administration of the presently disclosed anti-BCMA antibodies. A clinician skilled in the art can readily determine, for example, by the use of clinical tests, physical examination and medical/family history, if an individual is a candidate for such treatment.

In certain embodiments, the presently disclosed subject matter provides a method of treating a tumor by administering a presently disclosed anti-BCMA antibody in combination with one or more other agents. For example, the presently disclosed subject matter provides a method of treating a tumor by administering a presently disclosed anti-BCMA antibody with an antineoplastic agent. The anti-BCMA antibody can be chemically or biosynthetically linked to one or more of the antineoplastic agents.

Non-limiting examples of suitable tumors include multiple myeloma, Non-Hodgkin Lymphoma, Hodgkin Lymphoma, Chronic Lymphocytic Leukemia (CLL), glioblastoma, and Waldenstrom's Macroglobulinemia. In certain embodiments, the tumor is multiple myeloma.

Any suitable method or route can be used to administer a presently disclosed anti-BCMA antibody, and optionally, to co-administer antineoplastic agents. Routes of administration include, for example, oral, intravenous, intraperitoneal, subcutaneous, or intramuscular administration. It should be emphasized, however, that the presently disclosed subject matter is not limited to any particular method or route of administration.

It is noted that the presently disclosed anti-BCMA antibody can be administered as a conjugate, which binds specifically to the receptor and delivers a toxic, lethal payload following ligand-toxin internalization.

It is understood that anti-BCMA antibodies of the presently disclosed subject matter can be administered in the form of a composition additionally comprising a pharmaceutically acceptable carrier. Suitable pharmaceutically acceptable carriers include, for example, one or more of water, saline, phosphate buffered saline, dextrose, glycerol, ethanol and the like, as well as combinations thereof. Pharmaceutically acceptable carriers may further comprise minor amounts of auxiliary substances such as wetting or emulsifying agents, preservatives or buffers, which enhance the shelf life or effectiveness of the binding proteins. The compositions of the injection can, as is well known in the art, be formulated so as to provide quick, sustained or delayed release of the active ingredient after administration to the mammal.

The presently disclosed subject matter also provides use of antibodies and nucleic acids that encode them for treatment of a tumor (e.g., multiple myeloma), for diagnostic and prognostic applications as well as use as research tools for the detection of BCMA in cells and tissues. Pharmaceutical compositions comprising the disclosed antibodies and nucleic acids are encompassed by the presently disclosed subject matter. Vectors comprising the nucleic acids of the presently disclosed subject matter for antibody-based treatment by vectored immunotherapy are also contemplated by the presently disclosed subject matter. Vectors include expression vectors which enable the expression and secretion of antibodies, as well as vectors which are directed to cell surface expression of the antigen binding proteins, such as chimeric antigen receptors.

Cells comprising the nucleic acids, for example cells that have been transfected with the vectors of the invention are also encompassed by the presently disclosed subject matter. Examples of such cells are further described elsewhere herein.

In some embodiments of the methods, the antibodies, or antigen-binding fragments thereof, or the genetically-modified cells or pharmaceutical compositions described herein, are administered in combination with a gamma secretase inhibitor. Gamma secretase is a protease complex known to cleave BCMA. The use of gamma secretase inhibitors has been proposed to prevent the cleavage of BCMA and the subsequent generation of soluble BCMA protein in the serum, which may bind the antibodies or cells of the invention and potentially reduce their efficacy. A number of gamma secretase inhibitors are known in the art, and methods of using gamma secretase inhibitors in combination with BCMA antibodies, antibody fragments, or genetically-modified cells expressing BCMA-specific receptors (e.g., BCMA CAR T cells) have been reported (e.g., WO2017/019496, WO2018/151836, WO2018/201056, WO2019/090003, WO2019/090364). Examples of gamma secretase inhibitors useful with the invention include, without limitation, nirogacestat, crenigacastat (LY3039478), LY411575, avagacestat (BMS-708163), AL101 (BMS-906024), AL102 (BMS-986115), R0492087 (RG-4733), MK-0752, and CPX-POM. In various embodiments of the invention, an effective dose of a gamma secretase inhibitor can be administered to a subject in combination with a BCMA antibody, or antigen-binding fragment thereof, or genetically-modified cell or pharmaceutical composition described herein. In some cases, the gamma secretase inhibitor can be administered prior to administration of the BCMA antibody, or antigen-binding fragment thereof, or genetically-modified cell or pharmaceutical composition described herein. In some cases, the gamma secretase inhibitor can be administered concurrently with the BCMA antibody, or antigen-binding fragment thereof, or genetically-modified cell or pharmaceutical composition described herein.

### 8. Kits

The presently disclosed subject matter provides kits for the treatment or prevention of a tumor (e.g., multiple myeloma). In certain embodiments, the kit comprises a therapeutic composition containing an effective amount of an anti-BCMA antibody in unit dosage form. In some embodiments, the kit comprises a sterile container which contains a therapeutic or prophylactic vaccine; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

If desired, the anti-BCMA antibody is provided together with instructions for administering the cell to a subject having or at risk of developing a tumor (e.g., multiple myeloma). The instructions will generally include information about the use of the composition for the treatment or prevention of a tumor (e.g., multiple myeloma). In other embodiments, the instructions include at least one of the following: description of the therapeutic agent; dosage schedule and administration for treatment or prevention of a neoplasia (e.g., multiple myeloma) or symptoms thereof; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

### 9. Chimeric Antigen Receptors (CARs)

Provided herein are host cells and genetically-modified cells expressing a CAR having specificity for human BCMA. Generally, a CAR comprises at least an extracellular domain, a transmembrane domain, and an intracellular domain. The intracellular domain, or cytoplasmic domain, can comprise, for example, at least one co-stimulatory domain and one or more signaling domains. The extracellular domain of a CAR can comprise, for example, a target-specific binding element (e.g., an antibody or antibody fragment that specifically binds to BCMA) otherwise referred to herein as an extracellular ligand-binding domain or anti-BCMA binding domain.

The CAR of the present disclosure is engineered to specifically bind to human BCMA, an antigen that is expressed on the surface of certain human cancers. The amino acid sequence of human BCMA is provided in SEQ ID NO: 1.

The extracellular ligand-binding domain or moiety of a CAR (i.e., the anti-BCMA binding domain) can be, for example, an antibody or antibody fragment, particularly any anti-BCMA antibody, or antigen-binding fragment thereof, described herein. An antibody fragment can, for example, be at least one portion of an antibody, that retains the ability to specifically interact with (e.g., by binding, steric hindrance, stabilizing/destabilizing, spatial distribution) an epitope of an antigen. Examples of antibody fragments include, but are not limited to, Fab, Fab′, F(ab′)2, Fv fragments, scFv antibody fragments, disulfide-linked Fvs (sdFv), a Fd fragment consisting of the VH and CH1 domains, linear antibodies, single-domain antibodies (sdAbs), camelid VHH domains, multi-specific antibodies formed from antibody fragments such as a bivalent fragment comprising two Fab fragments linked by a disulfide bridge at the hinge region, and an isolated CDR or other epitope binding fragments of an antibody. An antigen binding fragment can also be incorporated into single domain antibodies, maxibodies, minibodies, nanobodies, intrabodies, diabodies, triabodies, tetrabodies, v-NAR and bis-scFv (see, e.g., Hollinger and Hudson, Nature Biotechnology 23:1126-1136, 2005). Antigen binding fragments can also be grafted into scaffolds based on polypeptides such as a fibronectin type III (Fn3) (see U.S. Pat. No. 6,703,199, which describes fibronectin polypeptide minibodies).

In certain instances, the extracellular ligand-binding domain or moiety of a CAR is in the form of a single-chain variable fragment (scFv) derived from an anti-BCMA antibody, or antigen-binding fragment thereof, described herein, which provides specificity for human BCMA. As described elsewhere herein, the VH and VL regions of an scFv can be arranged such that the VH region is the 5′ domain and the VL region is the 3′ domain, or they can be arranged such that the VL region is the 5′ domain and the VH region is the 3′ domain. In certain embodiments, the VH region and VL region are connected by a polypeptide by a linker such as, for examples, those linkers described elsewhere herein. In some embodiments, the scFv is murine or humanized. In various examples, the anti-BCMA binding domain of the CAR can comprise any scFv described herein such as, for example, scFvs comprising an amino acid sequence set forth in any one of SEQ ID NOs: 81-98, and variants thereof.

The extracellular ligand-binding domain of a CAR can also comprise an autoantigen (see, Payne et al. (2016), Science 353 (6295): 179-184), that can be recognized by autoantigen-specific B cell receptors on B lymphocytes, thus directing T cells to specifically target and kill autoreactive B lymphocytes in antibody-mediated autoimmune diseases. Such CARs can be referred to as chimeric autoantibody receptors (CAARs), and their use is encompassed by the invention. The extracellular ligand-binding domain of a CAR can also comprise a naturally-occurring ligand for an antigen of interest, or a fragment of a naturally-occurring ligand which retains the ability to bind the antigen of interest.

A CAR comprises a transmembrane domain which links the extracellular ligand-binding domain with the intracellular signaling and co-stimulatory domains via a hinge region or spacer sequence. The transmembrane domain can be derived from any membrane-bound or transmembrane protein. For example, the transmembrane polypeptide can be a subunit of the T-cell receptor (e.g., an α, β, γ or ζ, polypeptide constituting CD3 complex), IL2 receptor p55 (a chain), p75 (β chain) or γ chain, subunit chain of Fc receptors (e.g., Fcy receptor III) or CD proteins such as the CD8 alpha chain. For example, transmembrane domains of particular use in this invention may be derived from TCRα, TCRβ, TCRζ, CD3ζ, CD3P, CD37, CD36, CD4, CD5, CD8, CD9, CD16, CD22, CD28, CD32, CD33, CD34, CD37, CD45, CD64, CD80, CD86, CD134, CD137, and CD154. However, any transmembrane domain is contemplated for use herein as long as the domain is capable of anchoring a CAR comprising the extracellular domain to a cell membrane. Transmembrane domains can also be identified using any method known in the art or described herein. In particular embodiments, the transmembrane domain of the CAR is a CD8 transmembrane domain comprising an amino acid sequence set forth in SEQ ID NO: 56, and variants thereof.

(SEQ ID NO: 56)
IYIWAPLAGTCGVLLLSLVITLYC

In some embodiments, a CAR disclosed herein further comprises a hinge region. The hinge region refers to any oligo- or polypeptide that functions to link the transmembrane domain to the extracellular ligand-binding domain. For example, a hinge region may comprise up to 300 amino acids, 10 to 100 amino acids or 25 to 50 amino acids. Hinge regions may be derived from all or part of naturally occurring molecules, such as from all or part of the extracellular region of CD8, CD4 or CD28, or from all or part of an antibody constant region. Alternatively, the hinge region may be a synthetic sequence that corresponds to a naturally occurring hinge sequence or may be an entirely synthetic hinge sequence. In particular examples, a hinge domain can comprise a part of a human CD8 alpha chain, FcγRllla receptor or IgG1. In certain embodiments, the hinge region of the CAR is a CD8 hinge region comprising an amino acid sequence set forth in SEQ ID NO: 54, and variants thereof.

(SEQ ID NO: 54)
TTTPAPRPPTPAPTIASQPLSLRPEACRPAAGGAVHTRGLDFACD

Intracellular signaling domains of a CAR are responsible for activation of at least one of the normal effector functions of the cell in which the CAR has been placed and/or activation of proliferative and cell survival pathways. The term “effector function” refers to a specialized function of a cell. Effector function of a T cell, for example, may be cytolytic activity or helper activity including the secretion of cytokines. The intracellular signaling domain can include one or more cytoplasmic signaling domains that transmit an activation signal to the T cell following antigen binding. Such cytoplasmic signaling domains can include, without limitation, a CD3 zeta signaling domain comprising an amino acid sequence set forth in SEQ ID NO: 66, and variants thereof.

(SEQ ID NO: 66)
RVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATK
DTYDALHMQALPPR

The intracellular domain of a CAR can also include one or more intracellular co-stimulatory domains that transmit a proliferative and/or cell-survival signal after ligand binding. In some cases, the co-stimulatory domain can comprise one or more TRAF-binding domains. Intracellular co-stimulatory domains can be any of those known in the art and can include, without limitation, those co-stimulatory domains disclosed in WO 2018/067697 including, for example, Novel 1 (“N1”; SEQ ID NO: 58), Novel 6 (“N6”; SEQ ID NO: 60), 4-1BB (SEQ ID NO: 62), CD28 (SEQ ID NO: 64), or variants thereof.

(SEQ ID NO: 58)
KHSRKKFVHLLKRPFIKTTGAAQMEDASSCRCPQEEEGECDL
(SEQ ID NO: 60)
KASRKKAAAAAKSPFASPASSAQEEDASSCRAPSEEEGSCEL
(SEQ ID NO: 62)
KRGRKKLLYIFKQPFMRPVQTTQEEDGCSCRFPEEEEGGCEL
(SEQ ID NO: 64)
RSKRSRLLHSDYMNMTPRRPGPTRKHYQPYAPPRDFAAYRS

Further examples of co-stimulatory domains can include a functional signaling domain obtained from a protein including an MHC class I molecule, TNF receptor proteins, Immunoglobulin-like proteins, cytokine receptors, integrins, signaling lymphocytic activation molecules (SLAM proteins), activating NK cell receptors, BTLA, a Toll ligand receptor, OX40, CD2, CD7, CD27, CD30, CD40, CDS, ICAM-1, LFA-1 (CD11a/CD18), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRT AM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83.

The intracellular domains of a CAR described herein may be linked to each other in a specified or random order. In particular embodiments, the co-stimulatory domain is proximal to the transmembrane domain relative to the intracellular signaling domain. In certain embodiments, the intracellular domain of a CAR described herein may contain short polypeptide linker or spacer regions, between 2 to 30 amino acids in length. In other embodiments, the intracellular domain of a CAR described herein may contain short polypeptide linker or spacer regions, between 2 to 10 amino acids in length. In some embodiments, the linker or spacer regions may include an amino acid sequence that substantially comprises glycine and serine.

CARs of the invention can, in some examples, further comprise a spacer sequence that is positioned between the extracellular hinge domain and the anti-BCMA binding domain. In certain examples, the spacer can comprise an amino acid sequence set forth in SEQ ID NO: 52, or variants thereof. In particular examples, the spacer of SEQ ID NO: 52 is encoded by a nucleic acid sequence comprising SEQ ID NO: 53.

(SEQ ID NO: 52)
GLSGL
(SEQ ID NO: 53)
GGCCTGAGCGGCCTG

CARs of the invention can also comprise a signal peptide. Such signal peptides can be positioned at the 5′ end of the polypeptide, typically connected to the anti-BCMA binding domain. In some examples, the CAR comprises a signal peptide comprising an amino acid sequence set forth in SEQ ID NO: 68, or variants thereof. In some examples, the signal peptide can comprise an amino acid sequence set forth in SEQ ID NO: 70, and variants thereof. In some examples, the signal peptide can comprise an amino acid sequence set forth in SEQ ID NO: 189, and variants thereof.

(SEQ ID NO: 68)
MALPVTALLLPLALLLHAAQP
(SEQ ID NO: 70)
MALPVTALLLPLALLLHAAQPA
(SEQ ID NO: 189)
MALPVTALLLPLALLLHAARP

The invention encompasses any CAR described herein. In particular examples, CARs of the invention comprise an amino acid sequence set forth in any one of SEQ ID NOs: 117-134, and variants thereof. Such CARs comprise: (a) scFvs described herein, which include the VH and VL region of the BCMA-3, BCMA-20, or BCMA-51c antibodies described herein, or combinations thereof, which are connected by a linker set forth in SEQ ID NO: 34; (b) a spacer set forth in SEQ ID NO: 52 (e.g., encoded by SEQ ID NO: 53); (c) a CD8 hinge domain set forth in SEQ ID NO: 54; (d) a CD8 transmembrane domain set forth in SEQ ID NO: 56; (e) an N6 co-stimulatory domain set forth in SEQ ID NO: 60; and (f) a CD3 zeta signaling domain set forth in SEQ ID NO: 66. In further examples, CARs of the invention comprise an amino acid sequence set forth in any one of SEQ ID NOs: 153-170, and variants thereof. These CARs comprise the same elements as those of SEQ ID NOs: 117-134, and further comprise a 5′ signal peptide set forth in SEQ ID NO: 70.

Further, it is to be understood that any of the polynucleotides described herein that encode a CAR can be prepared by a routine method, such as recombinant technology. Methods for preparing a CAR described herein may involve, in some embodiments, the generation of a polynucleotide that encodes a polypeptide comprising each of the domains of the CAR (e.g., at least an extracellular domain, a transmembrane domain, and an intracellular domain).

### 10. Methods for Producing Recombinant Viruses (i.e., Viral Vectors)

In some embodiments, the present disclosure provides recombinant viruses, such as recombinant AAVs for use in the compositions and methods described herein. Recombinant AAV are typically produced in mammalian cell lines such as HEK-293. Because the viral cap and rep genes are removed from the vector to prevent its self-replication and to make room for the therapeutic gene(s) to be delivered (e.g. the endonuclease gene), it is necessary to provide these in trans in the packaging cell line. In addition, it is necessary to provide the “helper” (e.g. adenoviral) components necessary to support replication (Cots D, Bosch A, Chillon M (2013) Curr. Gene Ther. 13(5): 370-81). Frequently, recombinant AAVs are produced using a triple-transfection in which a cell line is transfected with a first plasmid encoding the “helper” components, a second plasmid comprising the cap and rep genes, and a third plasmid comprising the viral ITRs containing the intervening DNA sequence to be packaged into the virus. Viral particles comprising a genome (ITRs and intervening gene(s) of interest) encased in a capsid are then isolated from cells by freeze-thaw cycles, sonication, detergent, or other means known in the art. Particles are then purified using cesium-chloride density gradient centrifugation or affinity chromatography and subsequently delivered to the gene(s) of interest to cells, tissues, or an organism such as a human patient. Accordingly, methods are provided herein for producing recombinant AAVs comprising at least one nucleic acid (e.g., a polynucleotide encoding a CAR) described herein.

In some embodiments, genetic transfer is accomplished via lentivirus (e.g., a lentiviral vector). Lentiviruses, in contrast to other retroviruses, in some contexts may be used for transducing certain non-dividing cells. Non-limiting examples of recombinant lentiviruses include those derived from a lentivirus, such as Human Immunodeficiency Virus 1 (HIV-1), HIV-2, an Simian Immunodeficiency Virus (SrV), Human T-lymphotropic virus 1 (HTLV-1), HTLV-2 or equine infection anemia virus (E1AV). For example, recombinant lentiviruses have been generated by multiply attenuating the HIV virulence genes, for example, the genes env, vif, vpr, vpu and nef are deleted, making the vector safer for therapeutic purposes. Recombinant lentiviruses are known in the art, see Naldini et ah, (1996 and 1998); Zufferey et ah, (1997); Dull et ah, 1998, U.S. Pat. Nos. 6,013,516; and 5,994,136). In some embodiments, these recombinant viruses are plasmid-based or virus-based, and are configured to carry the essential sequences for incorporating foreign nucleic acid, for selection, and for transfer of the nucleic acid into a host cell. Known lentiviruses can be readily obtained from depositories or collections such as the American Type Culture Collection (“ATCC”; 10801 University Blvd., Manassas, Va. 20110-2209), or isolated from known sources using commonly available techniques.

In specific embodiments, recombinant lentiviruses are prepared using a plasmid encoding the gag, pol, tat, and rev genes cloned from human immunodeficiency virus (HIV) and a second plasmid encoding the envelope protein from vesicular stomatitis virus (VSV-G) used to pseudotype viral particles. A transfer vector, such as the pCDH-EF1-MCS vector, can be used with a suitable promoter such as the JeT promoter or the EF1 promoter. A CAR described herein can then be inserted downstream of the promoter, followed by an IRES and GFP. All three plasmids can then be transfected into lentivirus cells, such as the Lenti-X-293T cells, and lentivirus can then be harvested, concentrated and screened after a suitable incubation time. Accordingly, methods are provided herein for producing recombinant lentiviruses comprising at least one nucleic acid (e.g., a polynucleotide encoding a CAR) described herein. Likewise, methods are provided herein for producing recombinant lentiviruses encoding a CAR described herein.

### 11. Genetically-Modified Cells and Populations Thereof

Provided herein are cells that are genetically-modified to express a CAR described herein. In specific embodiments, a genetically-modified cell of the invention comprises a polynucleotide encoding a CAR described herein. In certain embodiments of the present disclosure, a polynucleotide or expression cassette which encodes a CAR described herein is present (i.e., integrated) within the genome of the genetically-modified cell or, alternatively, is not integrated into the genome of the cell. In some embodiments, where the polynucleotide or expression cassette is not integrated into the genome, the polynucleotide or expression cassette is present in the genetically-modified cell in a recombinant DNA construct, in an mRNA, in a viral genome, or in another polynucleotide which is not integrated into the genome of the cell.

Thus, in some examples, genetically-modified cells of the invention can contain a polynucleotide encoding a CAR described herein, positioned within the genome of the cell. In certain embodiments, genetically-modified cells contain a polynucleotide encoding a CAR described herein, positioned within the endogenous T cell receptor alpha gene, the endogenous T cell receptor alpha gene, or the T cell receptor beta gene of the cell. In certain other embodiments, a polynucleotide encoding a CAR described herein is positioned within the endogenous T cell receptor alpha constant region gene, such as within exon 1 of the T cell receptor alpha constant region gene. In particular examples, a polynucleotide encoding a CAR described herein is positioned specifically within SEQ ID NO: 74 (i.e., the TRC 1-2 recognition sequence) within the T cell receptor alpha constant region (i.e., TRAC) gene. In further examples, a polynucleotide encoding a CAR described herein is positioned between positions 13 and 14 of SEQ ID NO: 74 (i.e., the TRC 1-2 recognition sequence) within the TRAC gene.

(SEQ ID NO: 74)
TGGCCTGGAGCAACAAATCTGA

The genetically-modified cells comprising a CAR described herein can be, for example, eukaryotic cells. In some such examples, the genetically-modified cells are human cells. In further examples, the genetically-modified cells are immune cells, such as T cells, NK cells, macrophages, monocytes, neutrophils, eosinophils, cytotoxic T lymphocytes, or regulatory T cells. A population of immune cells can be obtained from any source, such as peripheral blood mononuclear cells (PBMCs), cord blood, tissue from site of an infection, ascites, pleural effusion, bone marrow, tissues such as spleen, lymph node, thymus, or tumor tissue. A source suitable for obtaining the type of cell desired would be evident to one of skill in the art. In some embodiments, the population of immune cells is derived from PBMCs. Immune cells useful for the invention may also be derived from pluripotent stem cells (e.g., induced pluripotent stem cells) that have been differentiated into an immune cell.

In some particular embodiments, the genetically-modified cells of the invention are T cells or NK cells, particularly human T cells or human NK cells, or cells derived therefrom. Such cells can be, for example, primary T cells or primary NK cells. In certain embodiments, any number of T cell and NK cell lines available in the art may be used. In some embodiments, T cells and NK cells are obtained from a unit of blood collected from a subject using any number of techniques known to the skilled artisan, such as those described herein above. In one embodiment, cells from the circulating blood of an individual are obtained by apheresis.

Methods of preparing cells capable of expressing a CAR described herein may comprise expanding isolated cells ex vivo. Expanding cells may involve any method that results in an increase in the number of cells capable of expressing a CAR described herein, for example, by allowing the cells to proliferate or stimulating the cells to proliferate. Methods for stimulating expansion of cells will depend on the type of cell used for expression of a CAR and will be evident to one of skill in the art. In some embodiments, the cells expressing a CAR described herein are expanded ex vivo prior to administration to a subject.

Genetically-modified cells comprising a CAR described herein can exhibit increased proliferation when compared to appropriate control cells that do not comprise a CAR. In some embodiments, cells comprising a CAR described herein further exhibit increased activation and proliferation in vitro or in vivo following stimulation with an appropriate antigen. For example, cells, such as CAR T cells and CAR NK cells, can exhibit increased activation, proliferation, and/or increased cytokine secretion compared to a control cell lacking the CARs described herein. Increased cytokine secretion can include the increased secretion of IFN-γ, IL-2, TNF-a, among others. Methods for measuring cell activation and cytokine production are well known in the art, and some suitable methods are provided in the examples herein.

Genetically-modified cells of the invention can be further modified to express one or more inducible suicide genes, the induction of which provokes cell death and allows for selective destruction of the cells in vitro or in vivo. In some examples, a suicide gene can encode a cytotoxic polypeptide, a polypeptide that has the ability to convert a non-toxic pro-drug into a cytotoxic drug, and/or a polypeptide that activates a cytotoxic gene pathway within the cell. That is, a suicide gene is a nucleic acid that encodes a product that causes cell death by itself or in the presence of other compounds. A representative example of such a suicide gene is one that encodes thymidine kinase of herpes simplex virus. Additional examples are genes that encode thymidine kinase of varicella zoster virus and the bacterial gene cytosine deaminase that can convert 5-fluorocytosine to the highly toxic compound 5-fluorouracil. Suicide genes also include as non-limiting examples genes that encode caspase-9, caspase-8, or cytosine deaminase. In some examples, caspase-9 can be activated using a specific chemical inducer of dimerization (CID). A suicide gene can also encode a polypeptide that is expressed at the surface of the cell that makes the cells sensitive to therapeutic and/or cytotoxic monoclonal antibodies. In further examples, a suicide gene can encode recombinant antigenic polypeptide comprising an antigenic motif recognized by the 20 anti-CD20 mAb Rituximab and an epitope that allows for selection of cells expressing the suicide gene. See, for example, the RQR8 polypeptide described in WO2013153391, which comprises two Rituximab-binding epitopes and a QBEnd10-binding epitope. For such a gene, Rituximab can be administered to a subject to induce cell depletion when needed. In further examples, a suicide gene may include a QBEnd10-binding epitope expressed in combination with a truncated EGFR polypeptide.

The present disclosure further provides a population of genetically-modified cells comprising a plurality of genetically-modified cells described herein, which comprise in their genome a polynucleotide encoding a CAR described herein. Thus, in various embodiments of the invention, a population of genetically-modified cells is provided wherein at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or up to 100%, of cells in the population are genetically-modified cells that comprise a polynucleotide encoding a CAR described herein.

Cells modified by the methods and compositions described herein can express a CAR described herein and further lack expression of an endogenous T cell receptor (e.g., an alpha/beta T cell receptor) due to inactivation of the TCR alpha gene, the TRAC gene, and/or the TCR beta region gene. The T cell alpha chain and TCR beta chain are required for assembly of the endogenous alpha/beta T cell receptor; therefore, disrupted expression of one or both of these chains also disrupts assembly of the endogenous alpha/beta T cell receptor on the cell surface. This further results in a lack of detectable expression of CD3 on the cell surface, because CD3 is also a component of the endogenous alpha/beta T cell receptor.

Thus, further provided is a population of cells comprising a plurality of genetically-modified cells described herein which comprise a polynucleotide encoding a CAR described herein, and which express the CAR (i.e., are CAR+). In some such embodiments, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or up to 100%, of cells in the population are a genetically-modified cell described herein that is CAR+. Also provided is a population of cells comprising a plurality of such genetically-modified cells comprising a polynucleotide encoding a CAR described here (i.e., are CAR+), that also comprise an inactivated TCR alpha gene, an inactivated TRAC gene, and/or an inactivated TCR beta gene (i.e., are TCR−). Such cells do not have detectable cell surface expression of an endogenous T cell receptor (i.e., an alpha/beta T cell receptor). In some such embodiments, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or up to 100%, of cells in the population are such genetically-modified cells that are TCR−/CAR+.

### 12. Methods for Producing Genetically-Modified Cells

The present disclosure provides methods for producing genetically-modified cells (e.g., T cells or NK cells) comprising a CAR described herein. In specific embodiments, methods are provided for modifying a cell to comprise a polynucleotide encoding a CAR described herein. In other aspects of the present disclosure, a polynucleotide or an expression cassette encoding a CAR described herein is integrated into the genome of the cell or, in alternative embodiments, is not integrated into the genome of the cell.

In certain embodiments, the polynucleotide encoding a CAR described herein can be introduced into the genome of a cell by random integration using a lentivirus. Such cells can be further modified to comprise an inactivated TCR alpha gene, an inactivated TRAC gene, and/or an inactivated TCR beta gene, such that the resulting cell expresses the CAR but does not express an endogenous alpha/beta T cell receptor on the cell surface.

In other embodiments, the methods of the invention for producing a genetically-modified cell comprise introducing into the cell a first nucleic acid comprising a polynucleotide encoding an engineered nuclease having specificity for a recognition sequence in the genome of the cell, wherein the engineered nuclease is expressed in the cell. The method further comprises introducing into the cell a template nucleic acid comprising a polynucleotide encoding a CAR described herein. According to the method, the engineered nuclease generates a cleavage site at the recognition sequence, and the polynucleotide is inserted into the genome at said cleavage site. As discussed elsewhere, genetically-modified cells produced by the method can be, for example, genetically-modified immune cells, such as genetically-modified T cells or genetically-modified NK cells, and cells derived therefrom.

The template nucleic acid can be introduced into the cell by any number of means, such as using a virus (i.e., a viral vector). In particular examples of the method, a virus used to introduce the template nucleic acid is a recombinant AAV (i.e., a recombinant AAV vector). Such recombinant AAVs can comprise the template nucleic acid within a viral capsid. This and other methods for introducing the template nucleic acid are further detailed below.

The first nucleic acid, which encodes the engineered nuclease, can also be introduced by any number of means, such as introduction as an mRNA that is expressed by the cell. This and other methods of introducing the first nucleic acid encoding the engineered nuclease, are further detailed below.

In some examples of this method, the nuclease recognition sequence is within a target gene, and expression of the polypeptide encoded by the target gene is disrupted following insertion of the polynucleotide at the cleavage site. The target gene can be, for example, a gene encoding a component of the alpha/beta T cell receptor, such as the TCR alpha gene, the TRAC gene, or the TCR beta gene. In particular examples, the target gene is a TRAC gene. In such cases, the polynucleotide can be inserted anywhere within the TCR alpha gene, the TRAC gene, or the TCR beta gene, so long as it is inserted in a manner that allows for expression of the CAR. Further, in certain embodiments of the method, the recognition sequence comprises SEQ ID NO: 74, also referred to as the TRC 1-2 recognition sequence, which is present within the T cell receptor alpha constant region gene. Cleavage of SEQ ID NO: 74 by an engineered meganuclease would be expected to produce a cleavage site between positions 13 and 14 of the recognition sequence. As such, in some examples of the method, the polynucleotide encoding a CAR described herein is inserted into the genome between positions 13 and 14 of SEQ ID NO: 74.

The use of nucleases for disrupting expression of an endogenous TCR gene has been disclosed, including the use of zinc finger nucleases (ZFNs), transcription activator-like effector nucleases (TALENs), megaTALs, and CRISPR systems (e.g., Osborn et al. (2016), Molecular Therapy 24(3): 570-581; Eyquem et al. (2017), Nature 543: 113-117; U.S. Pat. No. 8,956,828; U.S. Publication No. US2014/0301990; U.S. Publication No. US2012/0321667). The specific use of engineered meganucleases for cleaving DNA targets in the human TRAC gene has also been previously disclosed. For example, International Publication No. WO 2014/191527, which disclosed variants of the I-OnuI meganuclease that were engineered to target a recognition sequence within exon 1 of the TCR alpha constant region gene. Moreover, in International Publication Nos. WO 2017/062439 and WO 2017/062451, Applicants disclosed engineered meganucleases which have specificity for recognition sequences in exon 1 of the TCR alpha constant region gene. These included “TRC 1-2 meganucleases” which have specificity for the TRC 1-2 recognition sequence (SEQ ID NO: 74) in exon 1 of the TRAC gene. The '439 and '451 publications also disclosed methods for targeted insertion of a CAR coding sequence or an exogenous TCR coding sequence into a cleavage site in the TCR alpha constant region gene.

Thus, any engineered nuclease can be used for targeted insertion of the polynucleotide encoding a CAR described herein including, for example, an engineered meganuclease, a zinc finger nuclease, a TALEN, a compact TALEN, a CRISPR system nuclease, or a megaTAL.

Zinc-finger nucleases (ZFNs) can be engineered to recognize and cut pre-determined sites in a genome. ZFNs are chimeric proteins comprising a zinc finger DNA-binding domain fused to a nuclease domain from an endonuclease or exonuclease (e.g., Type IIs restriction endonuclease, such as the FokI restriction enzyme). The zinc finger domain can be a native sequence or can be redesigned through rational or experimental means to produce a protein which binds to a pre-determined DNA sequence ˜18 basepairs in length. By fusing this engineered protein domain to the nuclease domain, it is possible to target DNA breaks with genome-level specificity. ZFNs have been used extensively to target gene addition, removal, and substitution in a wide range of eukaryotic organisms (reviewed in S. Durai et al., Nucleic Acids Res 33, 5978 (2005)).

Likewise, TAL-effector nucleases (TALENs) can be generated to cleave specific sites in genomic DNA. Like a ZFN, a TALEN comprises an engineered, site-specific DNA-binding domain fused to an endonuclease or exonuclease (e.g., Type IIs restriction endonuclease, such as the FokI restriction enzyme) (reviewed in Mak, et al. (2013) Curr Opin Struct Biol. 23:93-9). In this case, however, the DNA binding domain comprises a tandem array of TAL-effector domains, each of which specifically recognizes a single DNA basepair.

Compact TALENs are an alternative endonuclease architecture that avoids the need for dimerization (Beurdeley, et al. (2013) Nat Commun. 4:1762). A Compact TALEN comprises an engineered, site-specific TAL-effector DNA-binding domain fused to the nuclease domain from the I-TevI homing endonuclease or any of the endonucleases listed in Table 2 in U.S. Application No. 20130117869. Compact TALENs do not require dimerization for DNA processing activity, so a Compact TALEN is functional as a monomer.

Engineered endonucleases based on the CRISPR/Cas system are also known in the art (Ran, et al. (2013) Nat Protoc. 8:2281-2308; Mali et al. (2013) Nat Methods. 10:957-63). A CRISPR system comprises two components: (1) a CRISPR nuclease; and (2) a short “guide RNA” comprising a ˜20 nucleotide targeting sequence that directs the nuclease to a location of interest in the genome. The CRISPR system may also comprise a tracrRNA. By expressing multiple guide RNAs in the same cell, each having a different targeting sequence, it is possible to target DNA breaks simultaneously to multiple sites in the genome.

Engineered meganucleases that bind double-stranded DNA at a recognition sequence that is greater than 12 base pairs can be used for the presently disclosed methods. A meganuclease can be an endonuclease that is derived from I-CreI and can refer to an engineered variant of I-CreI that has been modified relative to natural I-CreI with respect to, for example, DNA-binding specificity, DNA cleavage activity, DNA-binding affinity, or dimerization properties. Methods for producing such modified variants of I-CreI are known in the art (e.g. WO 2007/047859, incorporated by reference in its entirety). A meganuclease as used herein binds to double-stranded DNA as a heterodimer. A meganuclease may also be a “single-chain meganuclease” in which a pair of DNA-binding domains is joined into a single polypeptide using a peptide linker.

Nucleases referred to as megaTALs are single-chain endonucleases comprising a transcription activator-like effector (TALE) DNA binding domain with an engineered, sequence-specific homing endonuclease.

In particular embodiments, the nucleases used to practice the invention are single-chain meganucleases. A single-chain meganuclease comprises an N-terminal subunit and a C-terminal subunit joined by a linker peptide. Each of the two domains recognizes half of the recognition sequence (i.e., a recognition half-site) and the site of DNA cleavage is at the middle of the recognition sequence near the interface of the two subunits. DNA strand breaks are offset by four base pairs such that DNA cleavage by a meganuclease generates a pair of four base pair, 3′ single-strand overhangs. For example, nuclease-mediated insertion using engineered single-chain meganucleases has been disclosed in International Publication Nos. WO 2017/062439 and WO 2017/062451. Nuclease-mediated insertion of the polynucleotide can also be accomplished, for example, using an engineered single-chain meganuclease comprising an amino acid sequence of SEQ ID NO: 76:

(SEQ ID NO: 76)
MNTKYNKEFLLYLAGFVDGDGSIYAVIYPHQRAKFKHFLKLLFTVSQST
KRRWFLDKLVDEIGVGYVYDLPRTSEYRLSEIKPLHNFLTQLQPFLKLK
QKQANLVLKIIEQLPSAKESPDKFLEVCTWVDQIAALNDSRTRKTTSET
VRAVLDSLPGSVGGLSPSQASSAASSASSSPGSGISEALRAGAGSGTGY
NKEFLLYLAGFVDGDGSIYACIRPRQGSKFKHRLTLGFAVGQKTQRRWF
LDKLVDEIGVGYVYDRGSVSEYVLSEIKPLHNFLTQLQPFLKLKQKQAN
LVLKIIEQLPSAKESPDKFLEVCTWVDQIAALNDSKTRKTTSETVRAVL
DSLSEKKKSSP

In some embodiments, mRNA encoding the engineered nuclease is delivered to the cell because this reduces the likelihood that the gene encoding the engineered nuclease will integrate into the genome of the cell.

The mRNA encoding an engineered nuclease can be produced using methods known in the art such as in vitro transcription. In some embodiments, the mRNA comprises a modified 5′ cap. Such modified 5′ caps are known in the art and can include, without limitation, an anti-reverse cap analogs (ARCA) (U.S. Pat. No. 7,074,596), 7-methyl-guanosine, CleanCap® analogs, such as Cap 1 analogs (Trilink; San Diego, CA), or enzymatically capped using, for example, a vaccinia capping enzyme or the like. In some embodiments, the mRNA may be polyadenylated. The mRNA may contain various 5′ and 3′ untranslated sequence elements to enhance expression of the encoded engineered nuclease and/or stability of the mRNA itself. Such elements can include, for example, posttranslational regulatory elements such as a woodchuck hepatitis virus posttranslational regulatory element. The mRNA may contain modifications of naturally-occurring nucleosides to nucleoside analogs. Any nucleoside analogs known in the art are envisioned for use in the present methods. Such nucleoside analogs can include, for example, those described in U.S. Pat. No. 8,278,036. In particular embodiments, nucleoside modifications can include a modification of uridine to pseudouridine, and/or a modification of uridine to N1-methyl pseudouridine.

Purified nuclease proteins can be delivered into cells to cleave genomic DNA, which allows for homologous recombination or non-homologous end-joining at the cleavage site with an exogenous nucleic acid molecule encoding a polypeptide of interest as described herein, by a variety of different mechanisms known in the art, including those further detailed herein.

In another particular embodiment, a nucleic acid encoding an engineered nuclease can be introduced into the cell using a single-stranded DNA template. The single-stranded DNA can further comprise a 5′ and/or a 3′ AAV inverted terminal repeat (ITR) upstream and/or downstream of the sequence encoding the engineered nuclease. In other embodiments, the single-stranded DNA can further comprise a 5′ and/or a 3′ homology arm upstream and/or downstream of the sequence encoding the engineered nuclease.

In other embodiments, genes encoding a nuclease of the invention are introduced into a cell using a linearized DNA template. Such linearized DNA templates can be produced by methods known in the art. For example, a plasmid DNA encoding a nuclease can be digested by one or more restriction enzymes such that the circular plasmid DNA is linearized prior to being introduced into a cell.

Purified engineered nuclease proteins, or nucleic acids encoding engineered nucleases, can be delivered into cells to cleave genomic DNA by a variety of different mechanisms known in the art, including those further detailed herein below.

In some embodiments, the nuclease proteins, or DNA/mRNA encoding the nuclease, are coupled to a cell penetrating peptide or targeting ligand to facilitate cellular uptake. Examples of cell penetrating peptides known in the art include poly-arginine (Jearawiriyapaisarn, et al. (2008) Mol Ther. 16:1624-9), TAT peptide from the HIV virus (Hudecz et al. (2005), Med. Res. Rev. 25: 679-736), MPG (Simeoni, et al. (2003) Nucleic Acids Res. 31:2717-2724), Pep-1 (Deshayes et al. (2004) Biochemistry 43: 7698-7706, and HSV-1 VP-22 (Deshayes et al. (2005) Cell Mol Life Sci. 62:1839-49. In an alternative embodiment, engineered nucleases, or DNA/mRNA encoding nucleases, are coupled covalently or non-covalently to an antibody that recognizes a specific cell-surface receptor expressed on target cells such that the nuclease protein/DNA/mRNA binds to and is internalized by the target cells. Alternatively, engineered nuclease protein/DNA/mRNA can be coupled covalently or non-covalently to the natural ligand (or a portion of the natural ligand) for such a cell-surface receptor. (McCall, et al. (2014) Tissue Barriers. 2(4):e944449; Dinda, et al. (2013) Curr Pharm Biotechnol. 14:1264-74; Kang, et al. (2014) Curr Pharm Biotechnol. 15(3):220-30; Qian et al. (2014) Expert Opin Drug Metab Toxicol. 10(11):1491-508).

In some embodiments, nuclease proteins, or DNA/mRNA encoding nucleases, are encapsulated within biodegradable hydrogels for injection or implantation within the desired region of the liver (e.g., in proximity to hepatic sinusoidal endothelial cells or hematopoietic endothelial cells, or progenitor cells which differentiate into the same). Hydrogels can provide sustained and tunable release of the therapeutic payload to the desired region of the target tissue without the need for frequent injections, and stimuli-responsive materials (e.g., temperature- and pH-responsive hydrogels) can be designed to release the payload in response to environmental or externally applied cues (Kang Derwent et al. (2008) Trans Am Ophthalmol Soc. 106:206-214).

In some embodiments, nuclease proteins, or DNA/mRNA encoding nucleases, are coupled covalently or non-covalently to a nanoparticle or encapsulated within such a nanoparticle using methods known in the art (Sharma, et al. (2014) Biomed Res Int. 2014). A nanoparticle is a nanoscale delivery system whose length scale is <1 μm or <100 nm. Such nanoparticles may be designed using a core composed of metal, lipid, polymer, or biological macromolecule, and multiple copies of the nuclease proteins, mRNA, or DNA can be attached to or encapsulated with the nanoparticle core. This increases the copy number of the protein/mRNA/DNA that is delivered to each cell and, so, increases the intracellular expression of each nuclease to maximize the likelihood that the target recognition sequences will be cut. The surface of such nanoparticles may be further modified with polymers or lipids (e.g., chitosan, cationic polymers, or cationic lipids) to form a core-shell nanoparticle whose surface confers additional functionalities to enhance cellular delivery and uptake of the payload (Jian et al. (2012) Biomaterials. 33(30): 7621-30). Nanoparticles may additionally be advantageously coupled to targeting molecules to direct the nanoparticle to the appropriate cell type and/or increase the likelihood of cellular uptake. Examples of such targeting molecules include antibodies specific for cell-surface receptors and the natural ligands (or portions of the natural ligands) for cell surface receptors.

In some embodiments, the nuclease proteins or DNA/mRNA encoding the nucleases are encapsulated within liposomes or complexed using cationic lipids (see, e.g., LIPOFECTAMINE™, Life Technologies Corp., Carlsbad, CA; Zuris et al. (2015) Nat Biotechnol. 33: 73-80; Mishra et al. (2011) J Drug Deliv. 2011:863734). The liposome and lipoplex formulations can protect the payload from degradation, enhance accumulation and retention at the target site, and facilitate cellular uptake and delivery efficiency through fusion with and/or disruption of the cellular membranes of the target cells.

In some embodiments, nuclease proteins, or DNA/mRNA encoding nucleases, are encapsulated within polymeric scaffolds (e.g., PLGA) or complexed using cationic polymers (e.g., PEI, PLL) (Tamboli et al. (2011) Ther Deliv. 2(4): 523-536). Polymeric carriers can be designed to provide tunable drug release rates through control of polymer erosion and drug diffusion, and high drug encapsulation efficiencies can offer protection of the therapeutic payload until intracellular delivery to the desired target cell population.

In some embodiments, nuclease proteins, or DNA/mRNA encoding nucleases, are combined with amphiphilic molecules that self-assemble into micelles (Tong et al. (2007) J Gene Med. 9(11): 956-66). Polymeric micelles may include a micellar shell formed with a hydrophilic polymer (e.g., polyethyleneglycol) that can prevent aggregation, mask charge interactions, and reduce nonspecific interactions.

In some embodiments, nuclease proteins, or DNA/mRNA encoding nucleases, are formulated into an emulsion or a nanoemulsion (i.e., having an average particle diameter of <1 nm) for administration and/or delivery to the target cell. The term “emulsion” refers to, without limitation, any oil-in-water, water-in-oil, water-in-oil-in-water, or oil-in-water-in-oil dispersions or droplets, including lipid structures that can form as a result of hydrophobic forces that drive apolar residues (e.g., long hydrocarbon chains) away from water and polar head groups toward water, when a water immiscible phase is mixed with an aqueous phase. These other lipid structures include, but are not limited to, unilamellar, paucilamellar, and multilamellar lipid vesicles, micelles, and lamellar phases. Emulsions are composed of an aqueous phase and a lipophilic phase (typically containing an oil and an organic solvent). Emulsions also frequently contain one or more surfactants. Nanoemulsion formulations are well known, e.g., as described in U.S. Pat. Nos. 6,015,832, 6,506,803, 6,635,676, 6,559,189, and 7,767,216, each of which is incorporated herein by reference in its entirety.

In some embodiments, nuclease proteins, or DNA/mRNA encoding nucleases, are covalently attached to, or non-covalently associated with, multifunctional polymer conjugates, DNA dendrimers, and polymeric dendrimers (Mastorakos et al. (2015) Nanoscale. 7(9): 3845-56; Cheng et al. (2008) J Pharm Sci. 97(1): 123-43). The dendrimer generation can control the payload capacity and size, and can provide a high payload capacity. Moreover, display of multiple surface groups can be leveraged to improve stability, reduce nonspecific interactions, and enhance cell-specific targeting and drug release.

In some embodiments, genes encoding a nuclease are delivered using a recombinant virus (i.e., a viral vector). Such recombinant viruses are known in the art and include recombinant retroviruses (i.e., retroviral vectors), recombinant lentiviruses (i.e., lentiviral vectors), recombinant adenoviruses (i.e., adenoviral vectors), and recombinant adeno-associated viruses (AAVs) (i.e., AAV vectors) (reviewed in Vannucci, et al. (2013 New Microbiol. 36:1-22). Recombinant AAVs useful in the invention can have any serotype that allows for transduction of the virus into a target cell type and expression of the nuclease gene in the target cell. In particular embodiments, recombinant AAVs have a serotype of AAV2 or AAV6. Recombinant AAVs can be single-stranded AAVs. AAVs can also be self-complementary such that they do not require second-strand DNA synthesis in the host cell (McCarty, et al. (2001) Gene Ther. 8:1248-54).

If the nuclease genes are delivered in DNA form (e.g. plasmid) and/or via a virus (e.g. AAV) they must be operably linked to a promoter. In some embodiments, this can be a viral promoter such as endogenous promoters from the viral vector (e.g. the LTR of a lentiviral vector) or the well-known cytomegalovirus- or SV40 virus-early promoters. In a particular embodiment, nuclease genes are operably linked to a promoter that drives gene expression preferentially in the target cell (e.g., a T cell or NK cell).

In particular embodiments, an mRNA encoding an engineered nuclease of the invention can be a polycistronic mRNA encoding two or more nucleases that are simultaneously expressed in the cell. A polycistronic mRNA can encode two or more nucleases that target different recognition sequences in the same target gene. Alternatively, a polycistronic mRNA can encode at least one nuclease described herein and at least one additional nuclease targeting a separate recognition sequence positioned in the same gene, or targeting a second recognition sequence positioned in a second gene such that cleavage sites are produced in both genes. A polycistronic mRNA can comprise any element known in the art to allow for the translation of two or more genes (i.e., cistrons) from the same mRNA molecule including, but not limited to, an IRES element, a T2A element, a P2A element, an E2A element, and an F2A element.

The invention further provides for the introduction of a template nucleic acid comprising a polynucleotide described herein (i.e., encoding a CAR described herein), wherein the polynucleotide is inserted into a cleavage site in the targeted gene. In some embodiments, the template nucleic acid comprises a 5′ homology arm and a 3′ homology arm flanking the polynucleotide and elements of the insert. Such homology arms have sequence homology to corresponding sequences 5′ upstream and 3′ downstream of the nuclease recognition sequence where a cleavage site is produced. In general, homology arms can have a length of at least 50 base pairs, at least 100 base pairs, and up to 2000 base pairs or more, and can have at least 90%, at least 95%, or more, sequence homology to their corresponding sequences in the genome.

The polynucleotide encoding the CAR can further comprise additional control sequences. For example, the sequence can include homologous recombination enhancer sequences, Kozak sequences, polyadenylation sequences, transcriptional termination sequences, selectable marker sequences (e.g., antibiotic resistance genes), origins of replication, and the like. Sequences encoding engineered nucleases can also include at least one nuclear localization signal. Examples of nuclear localization signals are known in the art (see, e.g., Lange et al., J. Biol. Chem., 2007, 282:5101-5105). The polynucleotide encoding the CAR can further comprise a promoter that is operably linked to the CAR coding sequence. Promoters useful for expression of CARs in eukaryotic cells, and particularly in immune cells such as T cells and NK cells, are known in the art. In certain examples, the polynucleotide includes a promoter comprising an amino acid sequence set forth in SEQ ID NO: 72 (i.e., a JeT promoter). In some examples, the polynucleotide includes a promoter comprising an amino acid sequence set forth in SEQ ID NO: 73 (i.e., an EF1 alpha promoter).

(SEQ ID NO: 72)
GGGCGGAGTTAGGGCGGAGCCAATCAGCGTGCGCCGTTCCGAAAGTTGC
CTTTTATGGCTGGGCGGAGAATGGGCGGTGAACGCCGATGATTATATAA
GGACGCGCCGGGTGTGGCACAGCTAGTTCCGTCGCAGCCGGGATTTGGG
TCGCGGTTCTTGTTTGT
(SEQ ID NO: 73)
GGGCAGAGCGCACATCGCCCACAGTCCCCGAGAAGTTGGGGGGAGGGGT
CGGCAATTGAACGGGTGCCTAGAGAAGGTGGCGCGGGGTAAACTGGGAA
AGTGATGTCGTGTACTGGCTCCGCCTTTTTCCCGAGGGTGGGGGAGAAC
CGTATATAAGTGCAGTAGTCGCCGTGAACGTTCTTTTTCGCAACGGGTT
TGCCGCCAGAACACAG

A template nucleic acid, comprising a polynucleotide described herein (i.e., a polynucleotide encoding a CAR described here), can be introduced into the cell by any of the means previously discussed. In a particular embodiment, the template nucleic acid is introduced by way of a virus, such as a recombinant AAV. Recombinant AAVs useful for introducing a template nucleic acid can have any serotype that allows for transduction of the virus into the cell and insertion of the polynucleotide into the cell genome. In particular embodiments, the recombinant AAV has a serotype of AAV2 or AAV6. Recombinant AAVs can be single-stranded AAV vectors. Recombinant AAVs can also be self-complementary such that they do not require second-strand DNA synthesis in the host cell (McCarty, et al. (2001) Gene Ther. 8: 1248-54).

In another alternative, the template nucleic acid can be introduced into the cell using a single-stranded DNA template. The single-stranded DNA can comprise the polynucleotide and, in particular embodiments, can comprise 5′ and 3′ homology arms to promote insertion of the polynucleotide into the cleavage site by homologous recombination. The single-stranded DNA can further comprise a 5′ AAV inverted terminal repeat (ITR) sequence 5′ upstream of the 5′ homology arm, and a 3′ AAV ITR sequence 3′ downstream of the 3′ homology arm.

In another particular embodiment, the template nucleic acid can be introduced into the cell by transfection with a linearized DNA template. In some examples, a plasmid DNA can be digested by one or more restriction enzymes such that the circular plasmid DNA is linearized prior to transfection into the cell.

In particular embodiments, the period of cell proliferation and/or expansion of the cell population, and/or delay cell exhaustion, is prolonged following introduction of a polynucleotide described herein (i.e., a polynucleotide encoding a CAR described herein) when compared to control cells. Methods of measuring cell expansion and exhaustion (such as T cell or NK cell expansion and exhaustion) are known in the art and disclosed elsewhere herein.

T cells modified by the present invention may require activation prior to introduction of a nuclease and/or an exogenous sequence of interest. For example, T cells can be contacted with anti-CD3 and anti-CD28 antibodies that are soluble or conjugated to a support (i.e., beads) for a period of time sufficient to activate the cells.

### 13. Pharmaceutical Compositions Comprising Genetically-Modified Cells

In one aspect of the invention, the present disclosure provides a pharmaceutical composition comprising a genetically-modified cell described herein, a population of genetically-modified cells described herein, or a population of cells described herein, and a pharmaceutically-acceptable carrier. Such pharmaceutical compositions can be prepared in accordance with known techniques. See, e.g., Remington, The Science and Practice of Pharmacy (21st ed. 2005). In the manufacture of a pharmaceutical formulation, according to the present disclosure, cells are typically admixed with a pharmaceutically acceptable carrier and the resulting composition is administered to a subject (e.g., a human). The pharmaceutically acceptable carrier must, of course, be acceptable in the sense of being compatible with any other ingredients in the formulation and must not be deleterious to the subject. In some embodiments, the pharmaceutical compositions of the present disclosure further comprise one or more additional agents useful in the treatment of a disease (e.g., cancer) in a subject. In additional embodiments, where the genetically-modified cell is, for example, a genetically-modified human T cell or NK cell (or a cell derived therefrom), pharmaceutical compositions of the present disclosure can further include biological molecules, such as cytokines (e.g., IL-2, IL-7, IL-15, and/or IL-21), which promote in vivo cell proliferation and engraftment. Pharmaceutical compositions comprising genetically-modified cells of the present disclosure can be administered in the same composition as an additional agent or biological molecule or, alternatively, can be co-administered in separate compositions.

The present disclosure also provides genetically-modified cells, or populations thereof, described herein for use as a medicament. The present disclosure further provides the use of genetically-modified cells, or populations thereof, described herein in the manufacture of a medicament for treating a disease in a subject in need thereof. In one such aspect, the medicament is useful for cancer immunotherapy in subjects in need thereof.

In some embodiments, the pharmaceutical compositions and medicaments of the present disclosure are useful for treating any disease state that can be targeted by adoptive immunotherapy. In a particular embodiment, the pharmaceutical compositions and medicaments of the present disclosure are useful as immunotherapy in the treatment of cancer. In some embodiments, the pharmaceutical composition is useful for treating a BCMA-related disease by killing a BCMA expressing (i.e., BCMA-positive) target cell. In particular examples, the pharmaceutical composition is useful for treating multiple myeloma, Non-Hodgkin Lymphoma, Hodgkin Lymphoma, Chronic Lymphocytic Leukemia (CLL), glioblastoma, and Waldenstrom's Macroglobulinemia.

### 14. Methods of Administering Genetically-Modified Cells

In another aspect of the invention, a genetically-modified cell described herein, a population of cells described herein, or a pharmaceutical composition described herein, is administered to a subject in need thereof.

For example, an effective amount of such genetically-modified cells, populations, or pharmaceutical compositions can be administered to a subject having a disease or disorder. The genetically-modified cells administered to the subject, which express a CAR described herein, facilitate the reduction of the proliferation, reduce the number, or kill target cells in the recipient. Unlike antibody therapies, genetically-modified cells of the present disclosure are able to replicate and expand in vivo, resulting in long-term persistence that can lead to sustained control of a disease.

Examples of possible routes of administration include parenteral, (e.g., intravenous (IV), intramuscular (IM), intradermal, subcutaneous (SC), or infusion) administration. Moreover, the administration may be by continuous infusion or by single or multiple boluses. In specific embodiments, the agent is infused over a period of less than about 12 hours, less than about 10 hours, less than about 8 hours, less than about 6 hours, less than about 4 hours, less than about 3 hours, less than about 2 hours, or less than about 1 hour. In still other embodiments, the infusion occurs slowly at first and then is increased over time.

In some of these embodiments wherein cancer is treated with the presently disclosed genetically-modified cells, the subject administered the genetically-modified cells is further administered an additional therapeutic agent or treatment, including, but not limited to gene therapy, radiation, surgery, or a chemotherapeutic agent(s) (i.e., chemotherapy).

When an “effective amount” or “therapeutic amount” is indicated, the precise amount of the compositions of the present disclosure to be administered can be determined by a physician with consideration of individual differences in age, weight, tumor size (if present), extent of infection or metastasis, and condition of the patient (subject). In some embodiments, a pharmaceutical composition comprising the genetically-modified cells described herein is administered at a dosage of 10⁴ to 10⁹ cells/kg body weight, including all integer values within those ranges. In further embodiments, the dosage is 10⁵ to 10⁷ cells/kg body weight, including all integer values within those ranges. In some embodiments, cell compositions are administered multiple times at these dosages. The genetically-modified cells can be administered by using infusion techniques that are commonly known in immunotherapy (see, e.g., Rosenberg et al., New Eng. J. of Med. 319: 1676, 1988). The optimal dosage and treatment regime for a particular patient can readily be determined by one skilled in the art of medicine by monitoring the patient for signs of disease and adjusting the treatment accordingly.

In some embodiments, the administration of genetically-modified cells of the present disclosure reduces at least one symptom of a target disease or condition. For example, administration of genetically-modified cells of the present disclosure can reduce at least one symptom of a cancer, such as multiple myeloma or other BCMA-related cancers. Symptoms of cancers, such as BCMA-related cancers, are well known in the art and can be determined by known techniques.

### 15. Variants

The present invention encompasses variants of the polypeptide and polynucleotide sequences described herein. As used herein, “variants” is intended to mean substantially similar sequences. A “variant” polypeptide is intended to mean a polypeptide derived from the “native” polypeptide by deletion or addition of one or more amino acids at one or more internal sites in the native protein and/or substitution of one or more amino acids at one or more sites in the native polypeptide. As used herein, a “native” polynucleotide or polypeptide comprises a parental sequence from which variants are derived. Variant polypeptides encompassed by the embodiments are biologically active. That is, they continue to possess the desired biological activity of the native protein. Such variants may result, for example, from human manipulation. Biologically active variants of polypeptides described herein will have at least about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, or about 99%, sequence identity to the amino acid sequence of the native polypeptide, as determined by sequence alignment programs and parameters described elsewhere herein. A biologically active variant of a polypeptide may differ from that polypeptide or subunit by as few as about 1-40 amino acid residues, as few as about 1-20, as few as about 1-10, as few as about 5, as few as 4, 3, 2, or even 1 amino acid residue.

The polypeptides may be altered in various ways including amino acid substitutions, deletions, truncations, and insertions. Methods for such manipulations are generally known in the art. For example, amino acid sequence variants can be prepared by mutations in the DNA. Methods for mutagenesis and polynucleotide alterations are well known in the art. See, for example, Kunkel (1985) Proc. Natl. Acad. Sci. USA 82:488-492; Kunkel et al. (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein. Guidance as to appropriate amino acid substitutions that do not affect biological activity of the protein of interest may be found in the model of Dayhoff et al. (1978) Atlas of Protein Sequence and Structure (Natl. Biomed. Res. Found., Washington, D.C.), herein incorporated by reference. Conservative substitutions, such as exchanging one amino acid with another having similar properties, may be optimal.

For polynucleotides, a “variant” comprises a deletion and/or addition of one or more nucleotides at one or more sites within the native polynucleotide. One of skill in the art will recognize that variants of the nucleic acids of the embodiments will be constructed such that the open reading frame is maintained. For polynucleotides, conservative variants include those sequences that, because of the degeneracy of the genetic code, encode the amino acid sequence of one of the polypeptides of the embodiments. Variant polynucleotides include synthetically derived polynucleotides, such as those generated, for example, by using site-directed mutagenesis but which still encode a polypeptide or RNA. Generally, variants of a particular polynucleotide of the embodiments will have at least about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or more sequence identity to that particular polynucleotide as determined by sequence alignment programs and parameters described elsewhere herein. Variants of a particular polynucleotide (e.g., the reference polynucleotide) can also be evaluated by comparison of the percent sequence identity between the polypeptide encoded by a variant polynucleotide and the polypeptide encoded by the reference polynucleotide.

The deletions, insertions, and substitutions of the protein sequences encompassed herein are not expected to produce radical changes in the characteristics of the polypeptide. However, when it is difficult to predict the exact effect of the substitution, deletion, or insertion in advance of doing so, one skilled in the art will appreciate that the effect will be evaluated by screening the polypeptide for its biological activity.

### EXAMPLES

This invention is further illustrated by the following examples, which should not be construed as limiting. Those skilled in the art will recognize, or be able to ascertain, using no more than routine experimentation, numerous equivalents to the specific substances and procedures described herein. Such equivalents are intended to be encompassed in the scope of the claims that follow the examples below.

### Example 1

### Binding of BCMA Antibodies to Cells Expressing BCMA Cell Surface Protei.

### 1. Methods

### Antibodies:

Ani-human BCMA antibodies were selected from phage display libraries. Then 96 clones were screened by ELISA against recombinant BCMA, and candidates with positive ELISA scores were used for CAR T generation and in vitro functional evaluation. In cell killing assays the 3 antibodies were identified as the best candidates based on their activity and specificity. These antibodies are referred to herein as BCMA-3, BCMA-20, and BCMA− 51c. The variable heavy chain (VH) regions of BCMA-3, BCMA-20, and BCMA-51c are set forth in SEQ ID NOs: 2, 6, and 10, respectively. The variable light chain (VL) regions of BCMA-3, BCMA-20, and BCMA-51c are set forth in SEQ ID NOs: 4, 8, and 12, respectively. As discussed throughout the Examples, the VH and VL regions of these antibodies were mixed and matched to generate a number of single-chain variable fragments.

In the present studies, two full-length IgG4 antibodies were prepared comprising (a) the BCMA-3L/3H regions, or (b) the BCMA-3L/20H variable regions, in addition to a heavy chain constant region (SEQ ID NO: 77) and a light chain constant region (SEQ ID NO: 79). These antibodies, along with a positive control IgG4 anti-BCMA antibody (comprising the VH and VL regions of the C11D5.3 antibody described in WO 2010/104949), were each evaluated for their ability to bind cell surface human BCMA. Each antibody was manufactured according to standard antibody manufacturing procedures.

### Cell Lines:

Parental K562 cell lines (ATCC) were transfected with plasmid DNA encoding human CD19 or human BCMA proteins (Invivogen). Bulk transfected cell lines were grown in X-vivo with 5% FBS supplemented with 10 μg/ml Blasticidin selection reagent. Single cell clones were selected for by limiting dilution assay and subsequent detectable expression of cell surface CD19 or BCMA proteins by flow cytometric analysis using commercial antibodies. The K562-BCMA and K562-CD19 cell lines were maintained in Xvivo+5% FBS with 10 ug/ml Blasticidin at a starting concentration of 5e5 cells/ml in T-25 flasks. Cell lines were split after 2-3 days of growth and re-seeded in fresh X-vivo medium+5% FBS with 10 ug/ml Blasticidin at 5e5 cells/ml.

### Analysis of Antibody Specificity by Flow Cytometry

For analysis of full-length antibody specificity, cell lines were pipetted out of T-25 flasks and added to separate 15 ml tubes. Cells were spun down at 1350 RPM for 5 minutes, washed with 10 ml of Xvivo+5% FBS, and spun again. To acquire target cell concentrations and viabilities, 10 ul of each cell line was added to a 96-well plate. 10 ul of trypan blue was added to each sample, mixed, and cell concentrations and viabilities were acquired on the Countess. 2e5 viable cells were added to separate wells on a 96-well round bottom plate. The plate was spun, cells were washed twice in 100 ul PBS, and spun at 1350 RPM for 5 minutes between washes. After washing, 0.75 ug/well of each full-length antibody diluted in 100 ul PBS total volume was added to an aliquot of both K562-BCMA and K562-CD19 target cell lines. Samples were mixed, covered, and incubated for 15 minutes at room temperature.

At the end of the incubation, cells were spun and washed in PBS as before. 100 ul of PBS containing goat anti-human IgG (diluted 1 to 400) was then added to each sample and mixed. For negative controls, goat anti-human IgG secondary antibody was added to one well of each target population in the absence of any FMC63 or BCMA IgG4 antibody. As positive controls, one well of each target population was stained with a commercially available PE conjugated antibody. Samples were mixed and incubated for 15 minutes while covered in the hood.

After 15 minutes, cells were spun and washed in PBS as before. Samples were run and data was collected on the Cytoflex S.

### 2. Results

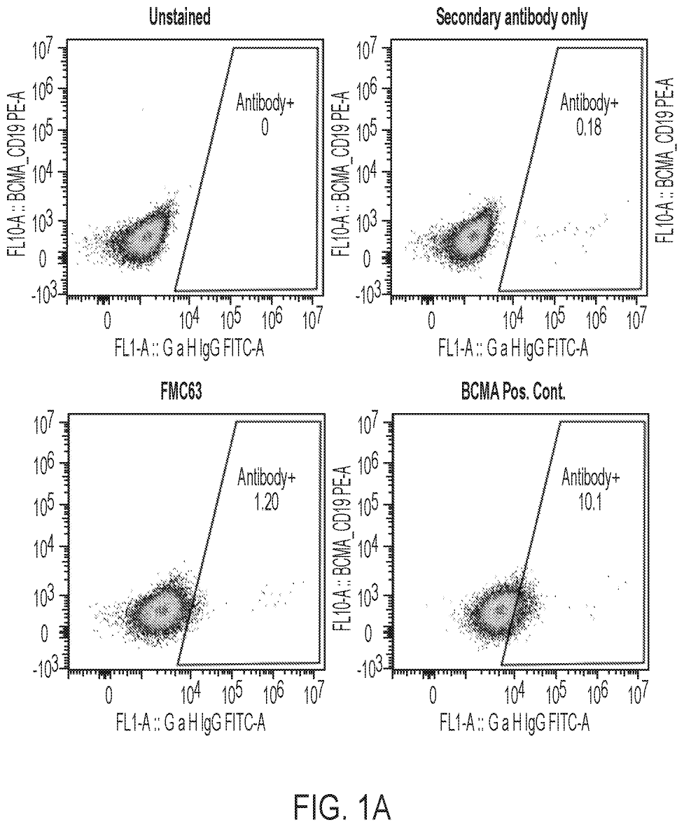

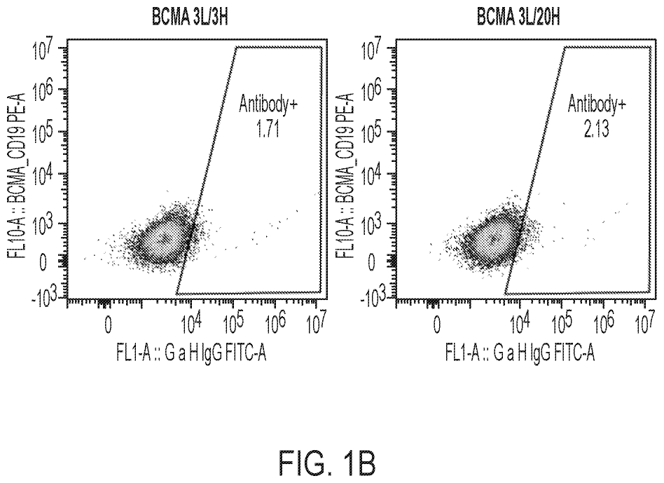

To determine the binding specificity of candidate anti-BCMA full length antibodies, human IgG4 antibodies expressing control or novel protein sequences were manufactured and added to target cell lines expressing either BCMA (K562-BCMA) or CD19 (K562-CD19). Target cell lines that were incubated with only PBS medium (FIG. 1A, top left) or the secondary human anti-goat IgG antibody (FIG. 1A, top right) failed to show detectable shifts in mean fluorescence intensity (MFI) (y-axis). Importantly, the full-length antibody expressing the anti-CD19 targeting moiety FMC63 also failed to cause a shift in detectable MFI on K562-BCMA cell lines (FIG. 1A, bottom left), indicating a lack of non-specific antibody binding to the cell surface. As previously noted, a positive control anti-BCMA full length antibody was added to K562-BCMA target cells (FIG. 1A, bottom right). Using 0.75 ug of antibody per 2e5 cells, a shift in MFI was detectable (10% of population above background), indicating specific binding of the positive control BCMA reference antibody to the K562-BCMA cell line. By comparison, the candidate anti-BCMA antibodies, designated BCMA-3L/3H and BCMA-3L/20H respectively, showed only a minor shift in detectable MFI above background compared to the reference positive control BCMA antibody (FIG. 1B). Of note, the reference positive control antibody, BCMA-3L/3H, and BCMA-3L/20H anti-BCMA antibodies failed to show any binding on control K562-CD19 cell lines (data not shown).

### 3. Conclusions

The anti-BCMA antibodies including the reference positive control antibody, BCMA− 3L/3H, and BCMA-3L/20H all showed binding on K562-BCMA target cell lines above background. Notably, the greatest increase in detectable MFI above background was seen using the reference positive control antibody, indicating distinct differences in binding strength of the positive control antibody compared to the BCMA-3L/3H, and BCMA-3L/20H antibodies. None of the anti-BCMA antibodies showed shifts in staining on K562-CD19 cells, implying a lack of non-specific binding. Conversely, the anti-CD19 targeting antibody FMC63 only showed positive staining on K562-CD19 cells. Collectively, antibodies are specific for the intended target.

### Example 2

### Affinity of BCMA Antibodies to BCMA Protein

### 1. Methods

### Antibodies

Full length human IgG4 antibodies expressing control or candidate anti-BCMA targeting moieties were produced as described in Example 1.

### Antibody Affinity Scouting

Antibody binding affinity was analyzed using a technique similar to surface plasmon resonance. Briefly, the Octet96RED technology is an analytical technique that compares the interference pattern of white light reflected on two surfaces. The first surface is an internal reference layer, while the second surface is a layer of a protein of interest, like BCMA, on a biosensor tip. Molecules that interact with the protein of interest on the second layer, through binding and dissociation, can shift the interference pattern of white light that is detectable by the Octet system. The magnitude of the interaction between molecules in solution and the immobilized protein of interest is directly proportional to the extent of interference. These measurements can be utilized to monitor binding specificity, rates of association, rates of dissociation, and/or concentration of candidate antibodies. Affinity scouting was completed with control and candidate anti-BCMA antibodies by adding BCMA protein to the biosensor tip. The supplied antibodies were utilized as analytes to measure the interference caused by antibody binding to the protein.

### 2. Results

Four full length antibodies were analyzed using the OctetRED96 technology for assessment of binding affinity by measurement of the equilibrium dissociation constant (K_{D}). Within the group of four were the two candidate anti-BCMA antibodies (BCMA-3L/3H and BCMA-3L/20H), the positive anti-BCMA control described in Example 1, and the anti-CD19 negative control described in Example 1. In the experiment, His-tagged BCMA protein was loaded onto anti-His antibody coated biosensor tips and supplied antibodies were utilized as analytes to measure K_{D}. The lowest K_{D} was reported for the positive control BCMA reference antibody, was an experimental K_{D} of <1.0e⁻¹² molar (M). By comparison, antibodies BCMA− 3L/3H and BCMA-3L/20H were reported to a have K_{D} of 1.38e⁻⁹ and 2.14e⁻⁹ M, respectively. For confirmation of antibody specificity, the negative control antibody expressing a CD19-targeting moiety failed to report a measurable K_{D}.

### 3. Conclusions

The equilibrium dissociation constant (K_{D}) is inversely proportional to the binding affinity of a given antibody. In the experiment, the lowest K_{D} and therefore highest binding affinity, was reported for the positive control BCMA antibody. By comparison, the candidate anti-BCMA antibodies each reported K_{D} at least several logs higher. Such a reported difference in binding affinity between the positive control BCMA, and the BCMA-3L/3H and BCMA-3L/20H antibodies described herein, clearly indicates that these BCMA-binding antibodies are functionally distinct.

### Example 3

### Design of BCMA CARs and Construction of AAVs

To build anti-BCMA CARs, single-chain variable fragments (scFvs) were designed using the VH and VL region sequences of the four murine anti-BCMA antibodies previously described herein: BCMA-3, BCMA-20, and BCMA-51c. The VH and VL domains of these antibodies were mixed and matched creating various scFvs that were subsequently tested as CARs. Among those scFvs tested were a BCMA-3L/3H scFv (SEQ ID NO: 82), a BCMA-3L/51cH scFv (SEQ ID NO: 90), a BCMA-20L/51cH scFv (SEQ ID NO: 94), a BCMA-3L/20H scFv (SEQ ID NO: 88).

The variable regions from the heavy and light chains for each antibody were cloned and joined by a linker set forth in SEQ ID NO: 34 to form the scFv. To construct a CAR, the scFv was joined to a spacer sequence (SEQ ID NO: 52 encoded by SEQ ID NO: 53), a CD8 hinge domain (SEQ ID NO: 54), a CD8 transmembrane domain (SEQ ID NO: 56), and an 15 intracellular domain comprising an N6 co-stimulatory domain (SEQ ID NO: 60) and a CD3 (intracellular signaling domain (SEQ ID NO: 66). In some experiments, the N6 co-stimulatory domain was replaced with an N1 (SEQ ID NO: 58) or 4-1BB (SEQ ID NO: 62). When these CAR molecules interact with BCMA+ target cells, the receptors cluster together in the cytoplasmic membrane and transduce signals through the N6-CD3 ζ tails. A coding sequence for a signal peptide set forth in SEQ ID NO: 70 was included at the 5′ end of the CAR.

The CAR constructs described above were placed under the control of a JeT promoter (a synthetic promoter containing four SP1 sites). The following studies utilize a nuclease-mediated targeted insertion approach to produce BCMA-specific CAR T cells. The target insertion site is an engineered meganuclease recognition sequence in the T cell receptor alpha constant region (TRAC) gene, referred to as TRC 1-2 (SEQ ID NO: 74). For preparation of an AAV for delivery, regions of homology to the sequences flanking the TRC 1-2 recognition sequence were added to each end of the CAR construct to enable homology-driven insertion into edited TRAC alleles. This construct was then cloned into an AAV6 packaging plasmid and used to transfect packaging cells along with RepCap and a helper plasmid for AAV6 particle production. The design of the CAR constructs tested herein are provided in Table 1 and the amino acid sequences of each CAR are further provided below.

TABLE 1
CAR Construct Design

| | Signal | | | | | Intracellular |
|---|---|---|---|---|---|---|
| CAR SEQ ID | Peptide | scFv | Hinge | Transmembrane | Co-stimulatory | signaling |
| SEQ ID NO: 154 | CD8 + A | 3L/3H | CD8 | CD8 | N6 | CD3 ζ |
| SEQ ID NO: 162 | CD8 + A | 3L/51cH | CD8 | CD8 | N6 | CD3 ζ |
| SEQ ID NO: 166 | CD8 + A | 20L/51cH | CD8 | CD8 | N6 | CD3 ζ |
| SEQ ID NO: 160 | CD8 + A | 3L/20H | CD8 | CD8 | N6 | CD3 ζ |

(SEQ ID NO: 154)
MALPVTALLLPLALLLHAAQPADIVLTQSPPSLAMSLGKRATISCRASESVTIPGQHLI
NWYQQKPGQPPKLLIQRASNVESGVPARFSGSGSRTDFTLTIDPVEEDDVAVYYCLQ
TRGIPRTFGGGTKLEIKGSTSGSGKPGSGEGSTKGQIQLVQSGPELKKPGETVKISCKA
SGYTFTHYSINWVKRAPGKGLKWMGWINTESGEPTYAYDFKGRFAFSLETSASTAY
LQINNLKYEDTATYFCALDYESAMDYWGQGTSVTVSSGLSGLTTTPAPRPPTPAPTIA
SQPLSLRPEACRPAAGGAVHTRGLDFACDIYIWAPLAGTCGVLLLSLVITLYCKASRK
KAAAAAKSPFASPASSAQEEDASSCRAPSEEEGSCELRVKFSRSADAPAYQQGQNQL
YNELNLGRREEYDVLDKRRGRDPEMGGKPRRKNPQEGLYNELQKDKMAEAYSEIG
MKGERRRGKGHDGLYQGLSTATKDTYDALHMQALPPR
(SEQ ID NO: 162)
MALPVTALLLPLALLLHAAQPADIVLTQSPPSLAMSLGKRATISCRASESVTIPGQHLI
NWYQQKPGQPPKLLIQRASNVESGVPARFSGSGSRTDFTLTIDPVEEDDVAVYYCLQ
TRGIPRTFGGGTKLEIKGSTSGSGKPGSGEGSTKGQIQLVQSGPELKKPGETVKISCKA
SGYTFTHYSINWVKRAPGKGLKWMGWINTETRESTYAYDFKGRFAFSLETSASTAY
LQINNLKYEDTATYFCALDYWSAMDYWGQGTSVTVSSGLSGLTTTPAPRPPTPAPTI
ASQPLSLRPEACRPAAGGAVHTRGLDFACDIYIWAPLAGTCGVLLLSLVITLYCKASR
KKAAAAAKSPFASPASSAQEEDASSCRAPSEEEGSCELRVKFSRSADAPAYQQGQNQ
LYNELNLGRREEYDVLDKRRGRDPEMGGKPRRKNPQEGLYNELQKDKMAEAYSEI
GMKGERRRGKGHDGLYQGLSTATKDTYDALHMQALPPR
(SEQ ID NO: 166)
MALPVTALLLPLALLLHAAQPADIVLTQSPPSLAMSLGKRATISCRASESVTIPGQHLI
HWYQQRPGQPPKLLIQRASNLESGVPARFSGSGSRTDFTLTIDPVEEDDVAVYYCLQ
TRKIPRTFGGGTKLEIKGSTSGSGKPGSGEGSTKGQIQLVQSGPELKKPGETVKISCKA
SGYTFTHYSINWVKRAPGKGLKWMGWINTETRESTYAYDFKGRFAFSLETSASTAY
LQINNLKYEDTATYFCALDYWSAMDYWGQGTSVTVSSGLSGLTTTPAPRPPTPAPTI
ASQPLSLRPEACRPAAGGAVHTRGLDFACDIYIWAPLAGTCGVLLLSLVITLYCKASR
KKAAAAAKSPFASPASSAQEEDASSCRAPSEEEGSCELRVKFSRSADAPAYQQGQNQ
LYNELNLGRREEYDVLDKRRGRDPEMGGKPRRKNPQEGLYNELQKDKMAEAYSEI
GMKGERRRGKGHDGLYQGLSTATKDTYDALHMQALPPR
(SEQ ID NO: 160)
MALPVTALLLPLALLLHAAQPADIVLTQSPPSLAMSLGKRATISCRASESVTIPGQHLI
NWYQQKPGQPPKLLIQRASNVESGVPARFSGSGSRTDFTLTIDPVEEDDVAVYYCLQ
TRGIPRTFGGGTKLEIKGSTSGSGKPGSGEGSTKGQIQLVQSGPELKKPGETVKISCKA
SGYTFTHYSINWVKRAPGKGLKWMGWINTETRESTYAYDFKGRFAFSLETSASTAY
LQINNLKYEDTATYFCALDYKQAMDYWGQGTSVTVSSGLSGLTTTPAPRPPTPAPTI
ASQPLSLRPEACRPAAGGAVHTRGLDFACDIYIWAPLAGTCGVLLLSLVITLYCKASR
KKAAAAAKSPFASPASSAQEEDASSCRAPSEEEGSCELRVKFSRSADAPAYQQGQNQ
LYNELNLGRREEYDVLDKRRGRDPEMGGKPRRKNPQEGLYNELQKDKMAEAYSEI
GMKGERRRGKGHDGLYQGLSTATKDTYDALHMQALPPR

### Example 4

Production of BCMA Specific CAR T cells and In Vitro Cell Killing Assay

### 1. Methods

### CAR T Cell Manufacturing and Flow Cytometric Analysis

In this study, frozen PBMCs from a qualified, healthy donor were thawed and rested overnight in Xuri medium (GE Healthcare)+5% FBS+10 ng/ml IL-2 (Cellgenix). The next day, cells were pooled, washed, and enumerated using trypan blue dye and a hemocytometer. For T cell enrichment, anti-human CD4 and anti-human CD8 microbeads (Miltenyi) were used in accordance with the manufacturer's instructions. Post-isolation, an aliquot of enriched T cells was stained for flow cytometric analysis to determine purity using commercially available antibodies for anti-CD3 (clone UCHT1), anti-CD4 (clone OKT4), and anti-CD8 (clone RPA-T8) and recovered cell number was determined by hemocytometer and trypan blue staining as before. T cells were activated using Transact (Miltenyi) in Xuri medium (GE Healthcare) supplemented with 5% FBS and 10 ng/ml IL-2 (Cellgenix) at a concentration of 1e6 viable cells/ml. After 3 days of stimulation, cells were collected, enumerated, and split evenly into 6 separate aliquots. Each aliquot of activated T cells was re-suspended in Maxcyte electroporation buffer and 1 μg of RNA encoding the TRC 1-2L.1592 meganuclease (SEQ ID NO: 76), which recognizes and cleaves the TRC 1-2 recognition sequence (SEQ ID NO: 74) in the T cell receptor alpha constant (TRAC) locus, was added per 1e6 viable cells. Cells were electroporated on the Maxcyte in separate OC-400 cuvettes. Post-electroporation, cells were pipetted to a 6-well plate containing equal volume pre-warmed serum-free Xuri medium with 30 ng/ml IL-2 (Cellgenix). After 20 minutes, cells were transferred to a 15 ml tube and serum free Xuri medium with 30 ng/ml IL-2 was added to a final volume of 14 ml.

Cells were counted in duplicate using the NC-200. Viable cell counts were enumerated and electroporated T cells were split into 7 equal aliquots. AAVs encoding the CARs described in Table 1, or a positive control anti-BCMA CAR, were added to appropriate cell aliquots and incubated for 6 hours at 37 degrees at 2.5e4 vector genomes/cell. After 6 hours, serum free medium was carefully removed and fresh Xuri medium+5% FBS+30 ng/ml IL-2 was added to final concentration of 1e6 cells/ml. Cultures were carried out for 5 days in complete Xuri medium supplemented with 5% FBS and 30 ng/ml IL-2 prior to conducting a flow cytometric analysis of CD3 and CAR expression to determine the frequency of TRAC knock-out and CAR knock-in cells. To detect CAR expression, BCMA biotinylated protein followed by secondary staining with a fluorescently conjugated streptavidin antibody was used. In addition, the frequencies of CD4 and CD8 cells were determined using anti-CD4 and anti-CD8 antibodies. A panel of surface markers were also measured to assess the degree to which the CAR T cells have differentiated in culture. Specifically, CD62L (clone SK11), CD45RO (clone UCHL1), and CD27 (Clone M-T271) levels were measured.

Unedited, remaining CD3+ cells from each condition were depleted from each sample using the manufacturer's protocol. Briefly, the residual frequency and number of CD3+ cells in each condition were calculated from the cell counts and the flow cytometric analysis above. Cells from each condition were spun down and washed in CliniMACS buffer (Miltenyi). Cells were spun once again and resuspended in CliniMACS buffer at a concentration of 1e8 cells/ml. To each was added 1 ul of CD3 microbeads (Miltenyi) per 1e6 CD3+ cells remaining in the sample. Samples were then placed on the nutator for 30 minutes to allow microbead binding to target cells. After the 30-minute incubation, CliniMACS buffer was added to each sample to a final concentration of 10 ml. Cells were spun down and supernatant was decanted. Cells were resuspended in 500 ul of CliniMACS buffer per sample. Each sample was added to a separate equilibrated LD magnetic column (Miltenyi) and flow through, representing the edited CD3 negative fraction, was collected. LD columns were washed twice with 1 ml of CliniMACS buffer per wash, after all added volume had run through the column.

After all samples had been depleted of remaining CD3+ cells, samples were spun down and supernatants were decanted. Cells were resuspended in 10 ml of Xuri media+5% FBS and cell counts were acquired on the NC-200 at a 1 to 5 dilution. Samples were diluted to a final concentration of 1e6 viable cells/ml in Xuri medium+5% FBS supplemented with 10 ng/ml IL-15 (Cellgenix) and IL-21 (Cellgenix).

After 5 additional days of incubation, T cells from each condition were added to separate 50 ml tubes and spun down at 1350 RPM for 5 minutes. Supernatants were decanted and cells were resuspended in 20 ml Xuri media+5% FBS. Cell counts were acquired on the NC-200 at a 1:10 dilution. To assess CAR T cell frequency and cellular phenotype, the same flow cytometric antibody panel described above was utilized. Total viable cell numbers and concentrations were calculated based on NC-200 counts and flow cytometric analysis five days following CD3 depletion and 10 days following the completion of the electroporation and transduction of the cells.

### ACEA Killing Assay

Briefly, 50 ul of Xuri cell culture media were added to ACEA plates. Plates were placed into the xCelligence instrument at 37 C and allowed to equilibrate for 30-60 minutes, after which a background measurement for starting cell index was taken. Plates were removed from the instrument and 1.0e⁴ 293-BCMA or control 293-CD19 expressing target cells were seeded in appropriate wells in 100 ul of Xuri media. Target cells lines were left undisturbed and cells bound to the plate surface at room temperature for 30-60 minutes in the hood. Plates were then covered and placed in the xCelligence instrument. Cell index measurements were initiated and acquired every 15 minutes. Target cell lines grew overnight to an appropriate cell index prior to addition of candidate CAR T cells.

After overnight incubation, cell index measurements were paused, and experimental plates were removed from the instrument. CAR T cell samples were counted and aliquots in triplicate replicate wells at effector to target ratios (E:T) of 1:2, 1:4, and 1:8 for both 293-BCMA and control 293-CD19 targets in 50 ul Xuri media. Effector only and target only wells were also created on each plate. Final volumes of all wells were 200 ul. After CAR T cell addition, plates were allowed to sit covered in the hood for 60 minutes to equilibrate wells. Plates were then placed back into the xCelligence instrument and cell index measurements were re-started, with acquisition occurring every 15 minutes over the length of the experiment.

### 2. Results

These experiments evaluated the BCMA CARs described in Table 1 (i.e., the experimental BCMA CARs) and a BCMA positive control CAR. The BCMA positive control CAR comprised the same VH and VL regions of the full length BCMA positive control antibody described in Example 1, and further comprised the same signal peptide, linker, spacer, hinge domain, transmembrane domain, co-stimulatory domain, and intracellular signaling domain, as the experimental BCMA CARs. In certain examples, the BCMA positive control CAR included a 4-1BB co-stimulatory domain in place of the N6 domain.

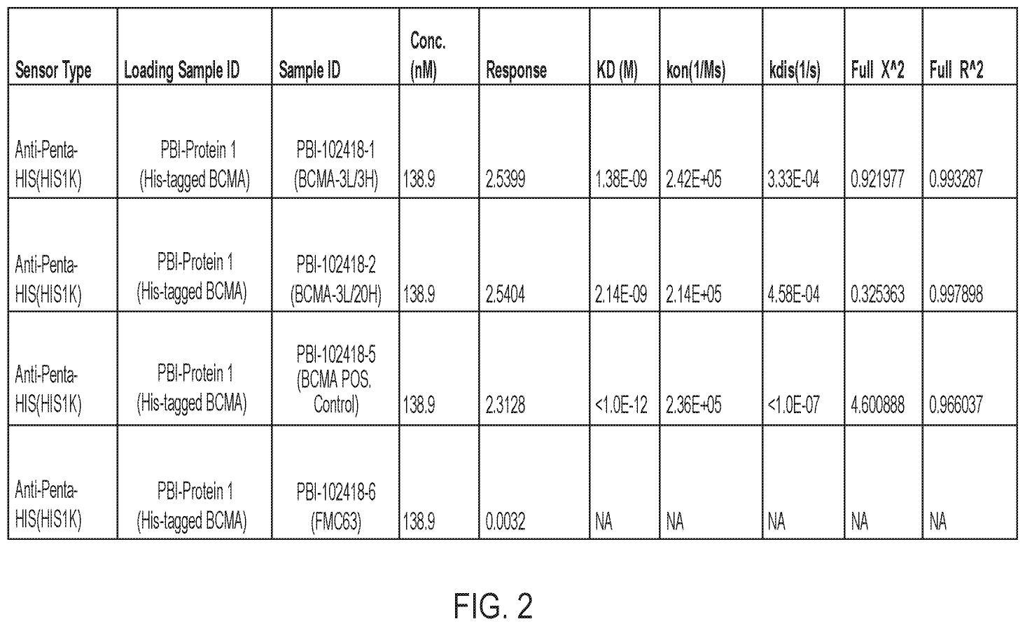

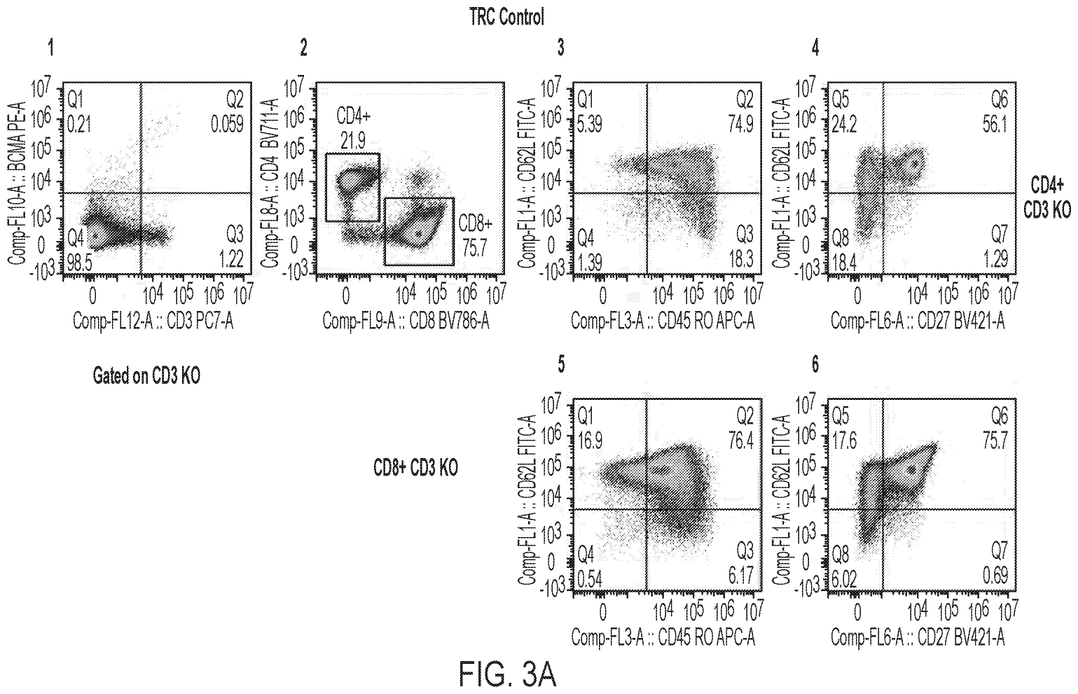

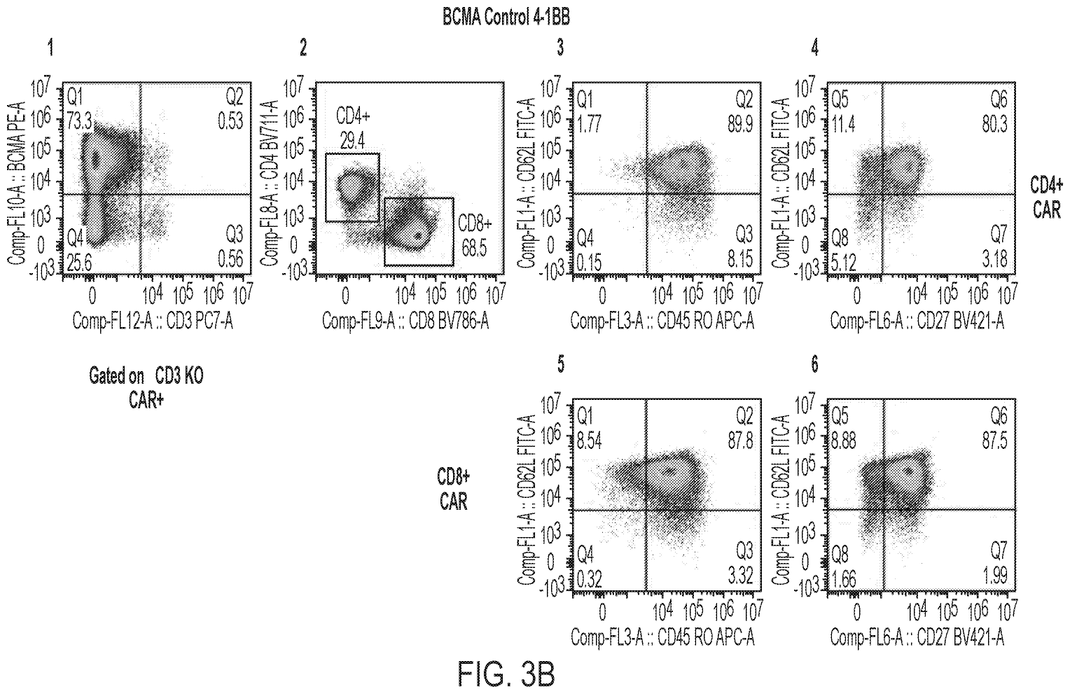

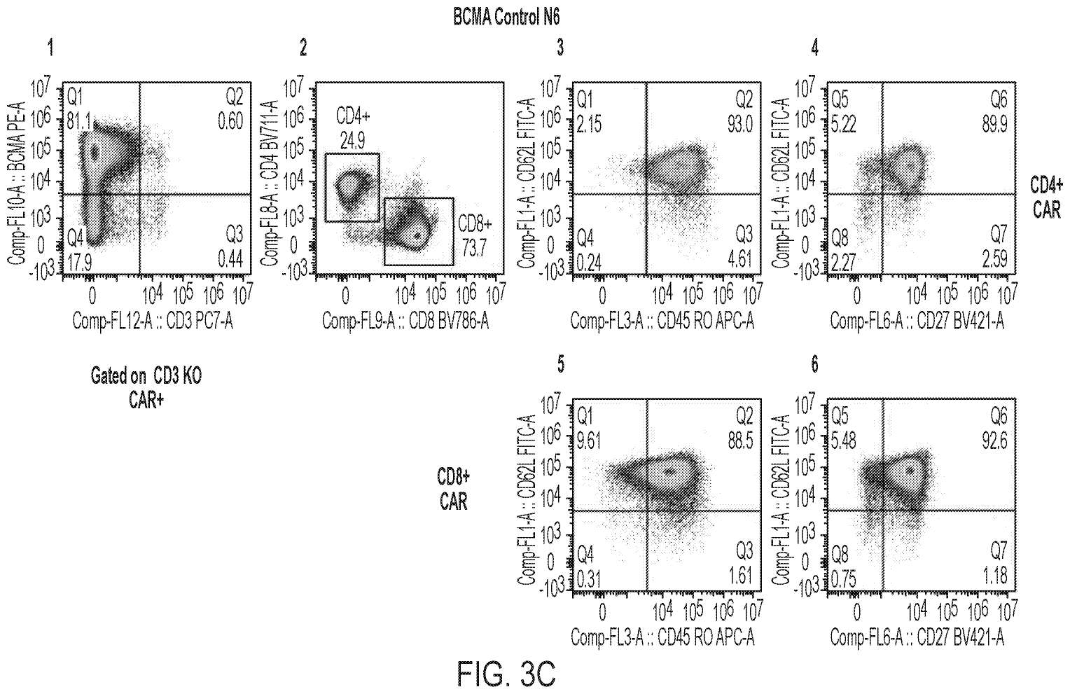

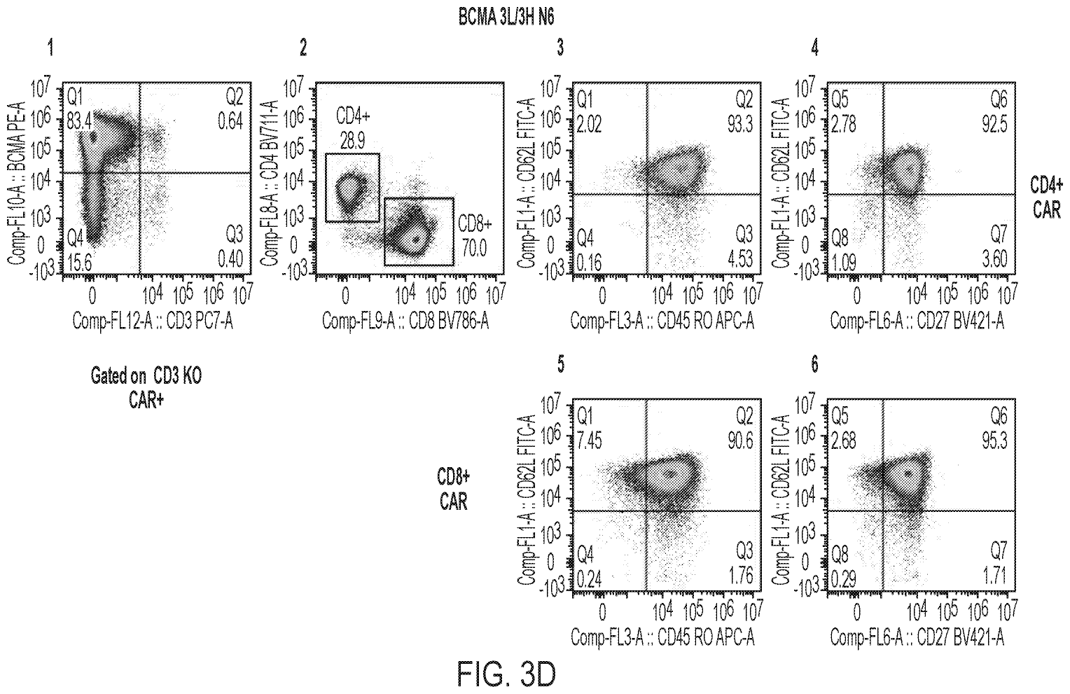

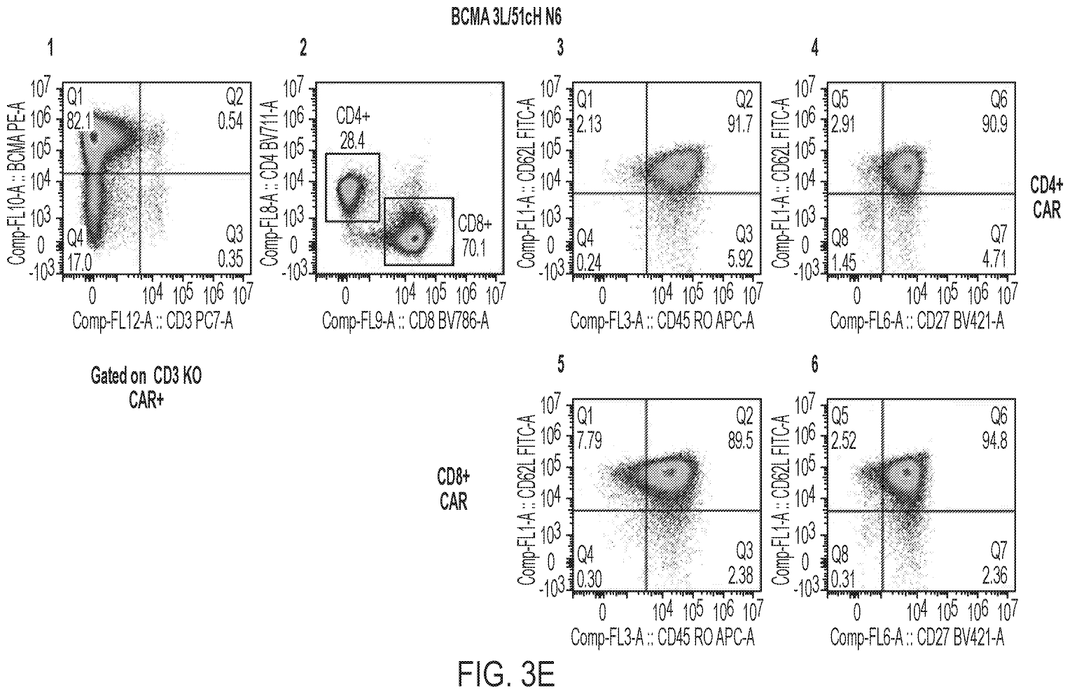

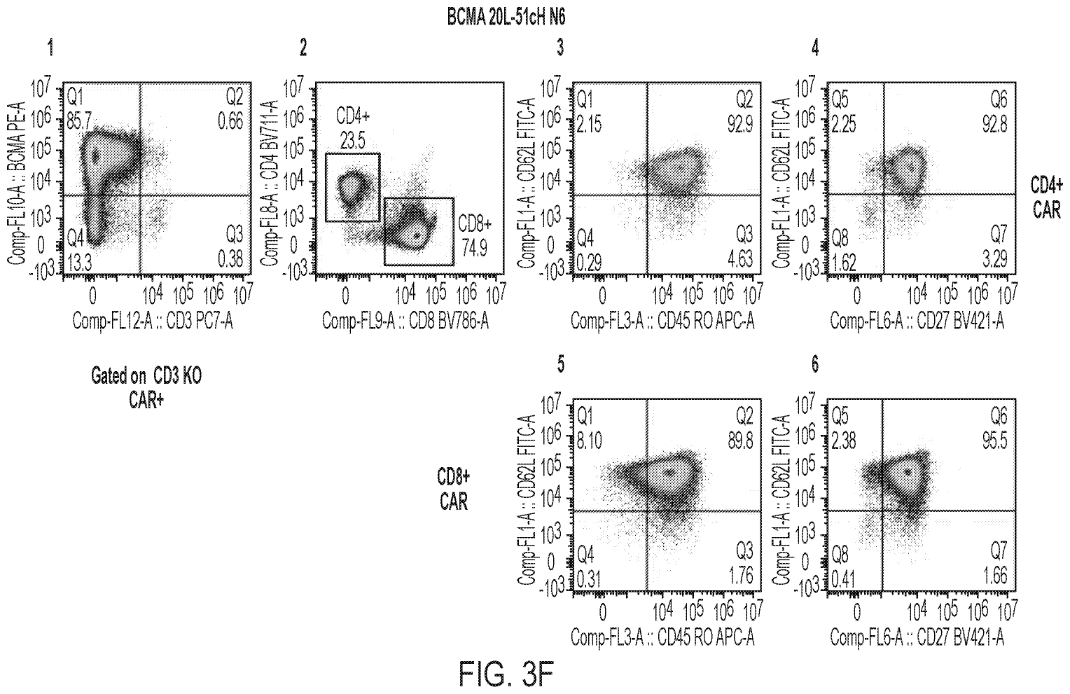

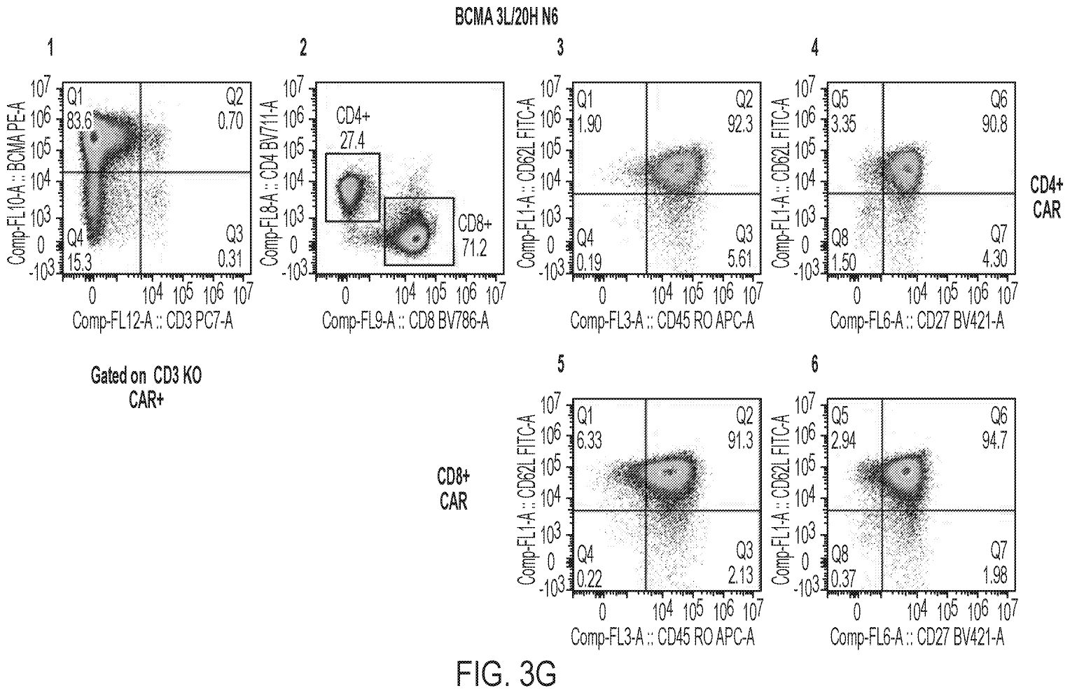

To access cellular expansion and differentiation prior to in vitro analysis of CAR function, phenotypic analysis of CAR and control T cells was done by flow cytometric analysis (FIG. 3). Control T cells electroporated with the TRAC nuclease, but not transduced with AAV encoding a BCMA CAR, failed to express detectable BCMA-binding CAR frequencies above background (FIG. 3A, 1^{st} panel). By comparison, all experimental conditions transduced to express either the BCMA positive control CAR or the experimental BCMA CARs described in Table 1, showed detectable CAR frequencies of >70% in total viable cells, with skewing towards CD8+ T cells within CAR+ populations (FIGS. 3B-3G, 2^{nd} panel). Furthermore, positive control BCMA scFv CAR T cells tethered to either an intracellular 4-1BB (FIG. 3B) or Novel 6 (N6) (FIG. 3C) co-stimulatory domain showed comparable frequencies of differentiated cellular subsets in both the CD4+CAR+ and CD8+CAR+populations, with the vast majority classified as translational memory T cells (CD62L^{HI}CD45RO^{HI}, middle right panels). CAR T cells expressing the experimental BCMA CARs described in Table 1 also exhibited a high proportion of translation memory T cells at the end of the manufacturing run (FIGS. 3D-G, panels 3 and 5). Lastly, staining for surface expression of CD27, another phenotypic marker, showed high frequencies (>80%) in all anti-BCMA CAR T cell populations tested (FIGS. 3B-3G, panels 4 and 6).

To monitor the magnitude and specificity of target cell killing, CAR or control T cells were co-cultured at various E:T ratios with 293-BCMA or 293-CD19 target cells. For this assay, the magnitude of cell killing is expressed as a reduction in cell index (y-axis) over time (x-axis). To measure this, an electrical current is run through the xCelligence plate every 15 minutes. If no target cells are present, the current runs unimpeded and the cell index remains at background levels. As target cells are added, bind to the plate, and proliferate, the electrical signal is impeded in a correlative manner. If target cells are killed, as by CAR T cells, they would subsequently fail to bind to the plate surface and cell index would decrease.

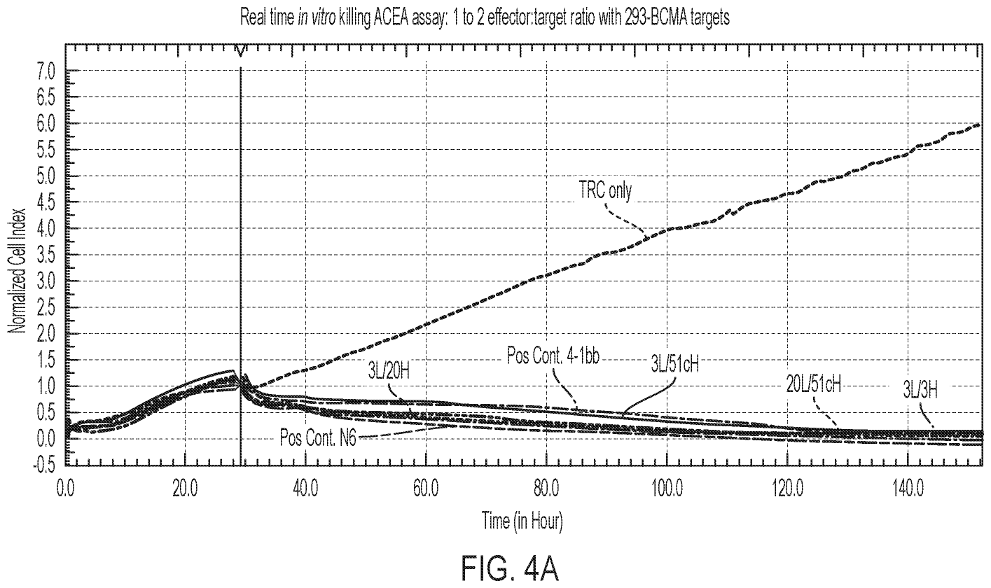

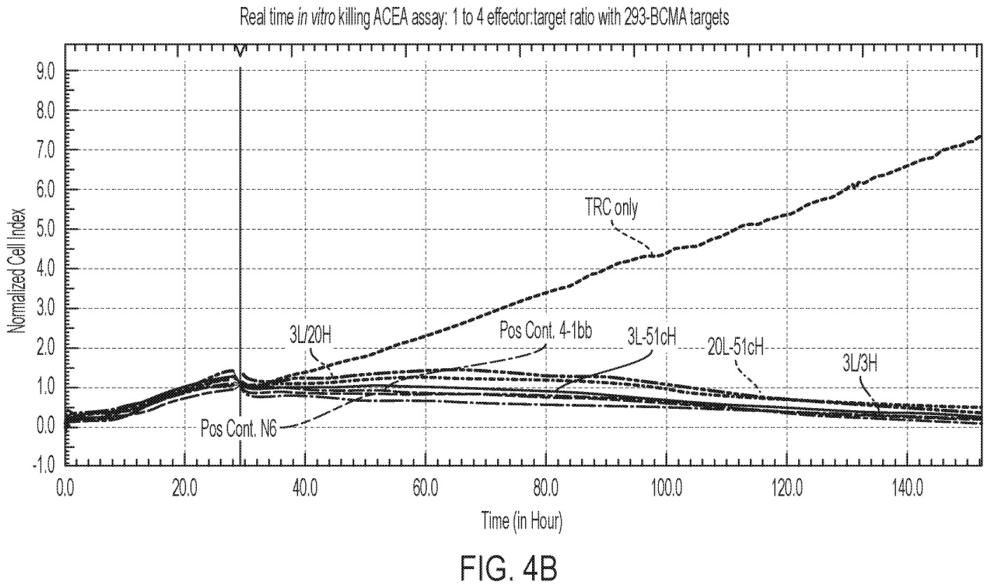

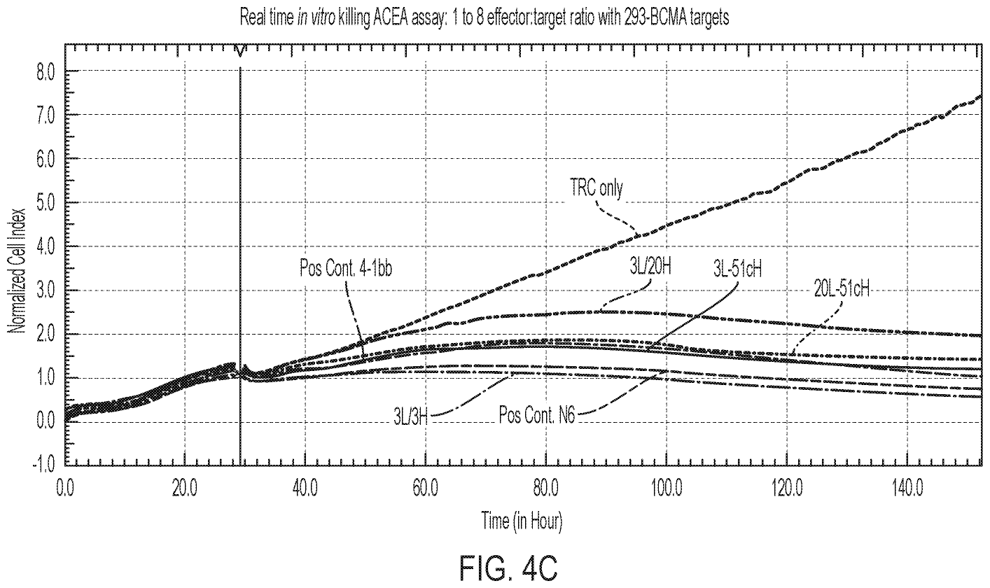

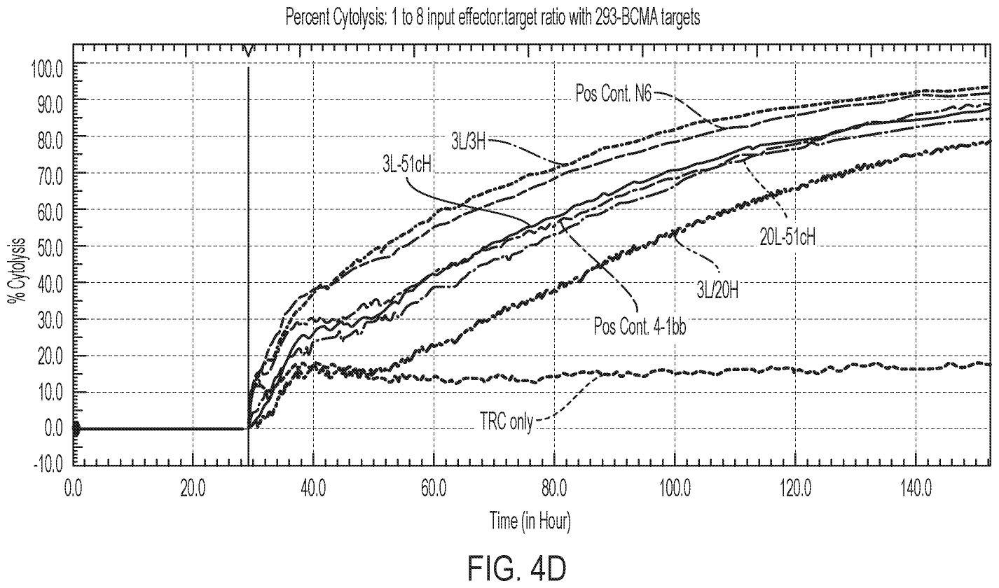

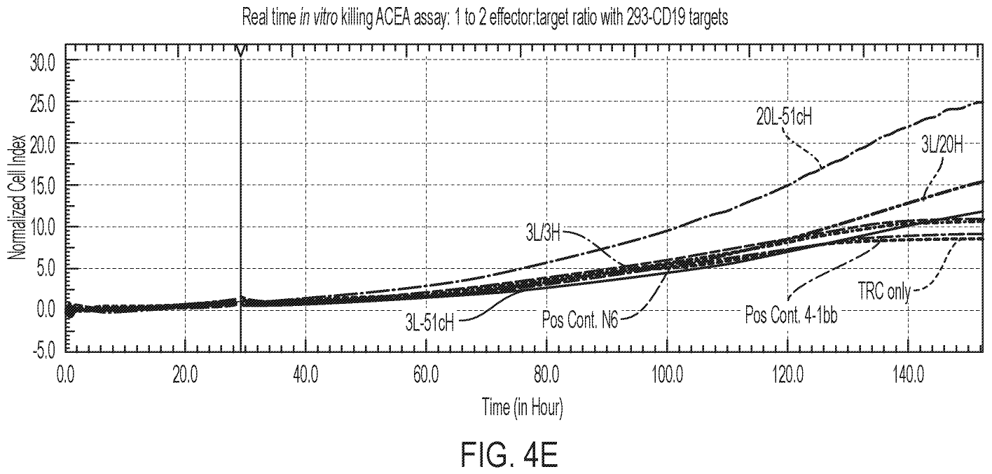

At an input ratio of 1:2 (FIG. 4A) or 1:4 (FIG. 4B), the two positive control BCMA CARs (comprising a 4-1BB or a N6 co-stimulatory domain) each eliminated 293-BCMA targets based on a reduction in acquired cell index to background levels. By comparison, the CAR T cells expressing the experimental CARs described in Table 1 showed a similar magnitude of 293-BCMA target killing at identical input E:T ratios (FIG. 4A-B), with only minor fluctuations in cell killing between constructs detected. Importantly, edited control T cells failed to kill target cell lines, with detectable cell index (indicating target cell expansion) increasing over the length of the experiment.

To determine if differences in BCMA+ target cell killing occurs when co-cultured at more stringent E:T ratios, target cell killing was analyzed at a 1:8 input ratio with 293-BCMA cells. Interestingly, differences in overall cell index (FIG. 4C) and percent cytolysis of targets (FIG. 4D) were seen between CAR populations. Specifically, the positive control BCMA CAR comprising an N6 co-stimulatory domain, and the BCMA-3L/3H CAR having an N6 co-stimulatory domain, showed the greatest reduction in target cells as determined by both cell index (FIG. 4C) and cytolysis (FIG. 4D). The positive control BCMA CAR comprising a 4-1BB co-stimulatory domain, the BCMA-3L/51cH CAR, and the BCMA-20L/51cH CAR were similar, and slightly less effective than the positive control BCMA CAR comprising the 4-1BB co-stimulatory domain and the BCMA-3L/3H CAR, with the BCMA-3L/20H CAR showing the least efficient 293-BCMA target cell clearance. Conversely, when anti-BCMA CAR T cells were co-cultured with 293-CD19 expressing target lines, no detectable target cell cytolysis was noted, with cell index of target cells increasing over time to the point of saturation (FIG. 4E).

### 3. Conclusions

Differences in CAR T cell function utilizing constructs targeting the same antigen (e.g., BCMA) can be attributed to a number of factors. Cellular phenotype, such as ratio of CD4:CD8 CAR T cells, the frequency of CAR+ cells in the total population, and the phenotype of CAR+ cells as determined by expression of various cell surface proteins, have each been shown to correlate with CAR cytotoxic potential. Importantly, over the 10-day manufacturing run, the phenotype of CAR T cells was comparable between cells expressing the control BCMA CAR and cells expressing the experimental BCMA CARs described in Table 1, with all populations showing a predominance of CD8-to-CD4 CAR T cells with a translational memory phenotype. This consistency in manufacturing supports the idea that any differences in CAR T cell function can be attributed to the scFv itself, as opposed to CAR T cell production. To assess this, CAR T cells expressing the positive control BCMA CAR, tethered to either the 4-1BB or N6 co-stimulatory domains, or the experimental BCMA CARs described in Table 1, were co-cultured with BCMA+ and BCMA− target cell lines in an in vitro killing assay. Although the magnitude of target cell clearance was comparable at 1:2 and 1:4 E:T ratios, notable differences in CAR T cell function were seen under more stringent conditions. Importantly, CAR T cells could be stratified based on target cell killing, with the positive control BCMA CAR with an N6 co-stimulatory domain, and the BCMA− 3L/3H CAR showing the highest degree of BCMA+ target clearance. Collectively, these data suggest that differences related to in vitro killing activity between anti-BCMA CAR T cells are attributable to the scFvs, and not to differences in cellular manufacturing or cellular phenotype.

### Example 5

Production of BCMA Specific CAR T cells and In Vivo Efficacy in a Murine MM.1S Tumor Model Cell Killing Assay

### 1. Methods

### MM.1S Luciferase Cell Line

Parental MM.1S cells were purchased from ATCC. To make a luciferase reporter line for in vivo imaging studies, MM.1S were transduced with a lentiviral vector expressing firefly luciferase at various MOI (Imanis Life Sciences). Transduced cells were grown in selection medium containing Puromycin and single cell clones were picked by limiting dilutions assays and in vitro determination of luciferase expression. To confirm critical characteristics of the single cell clone, in vivo growth kinetics, detectable luciferase expression by BLI, tumor dosing, and trafficking of tumor in vivo with candidate clones was assayed in a pilot study in NSG recipient mice (data not shown). For the in vivo tumor CAR T cell efficacy study, MM.1S luciferase expressing, single cell clone cells were grown in RPMI 1640 with 15% FBS under Puromycin selection. Luciferase expression was evaluated by an in vitro BrightGlo (ProMega) reporter assay prior to in vivo dosing.

CAR T cells

CAR T cell manufacturing was similar to the process described in Example 4. Briefly, at the end of the manufacturing run, CAR T cells were enumerated on the NC-200. Cells were then added to separate 15 ml tubes and spun down at 1350 RPM for 5 minutes. Supernatants were decanted, cells were resuspended in fresh PBS, and spun down as before. Samples were diluted to a final concentration of 1e6 CAR T cells or 5e6 CAR T cells/200 ul PBS, based on cell counts and flow cytometric analysis for phenotype, and transferred to labeled 1.7 ml tubes. Samples were placed on ice and transferred to vivarium staff. Injections of 200 ul IV were administered to groups of NSG mice pre-treated with MM.1S luciferase cells (n=5 per condition). A separate cohort of mice received PBS alone acted as a vehicle control.

### NSG In Vivo Tumor Study

Female NSG mice were purchased commercially from Jackson laboratories. On day 0, recipient animals were treated with 2.5e6 MM.1S luciferase cells in 200 ul PBS IV (12.5e6 MM.1S cells per ml). To ensure MM.1S were detectable prior to in vivo CAR T cell administration, BLI measurements were acquired on day 1, 3, and 7 post-MM.1S luciferase treatment. CAR T, control cells, or PBS vehicle were dosed on day 8 post-MM.1S luciferase treatment, with animal divided into groups of 5. BLI assessment and weights were acquired twice a week over the course of the experiment and animals were euthanized in accordance with pre-determined humane endpoints.

### 2. Results

To evaluate the efficacy of anti-BCMA CAR T cells in an in vivo treatment model, candidate and control cells were administered to NSG mice 8 days after dosing with the multiple myeloma cell line, MM.1S, expressing a luciferase reporter gene. Mice were administered CAR T cells expressing the BCMA-3L/3H or BCMA-3L/20H CARs described in Table 1, or the positive control BCMA CARs (comprising 4-1BB or N6) previously described.

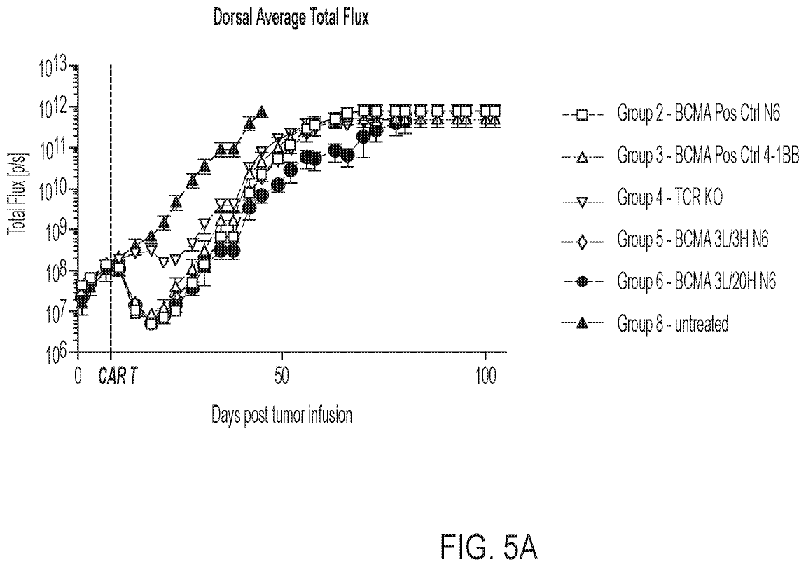

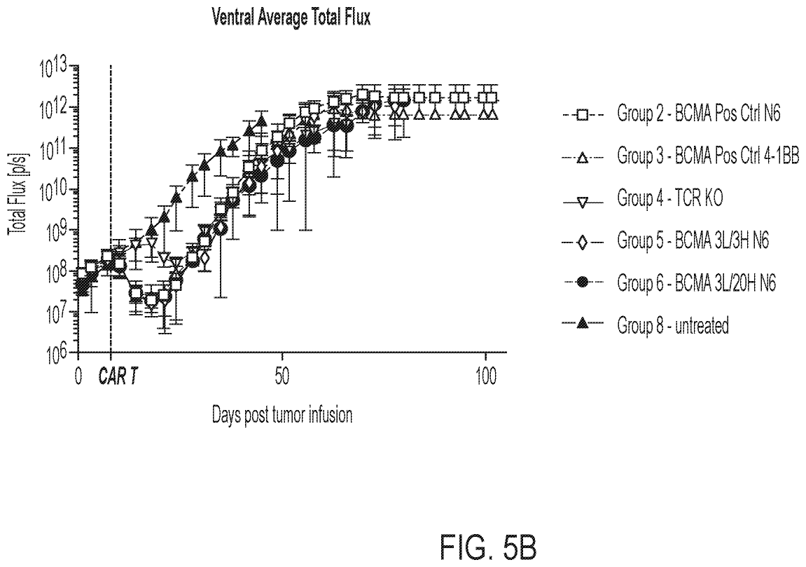

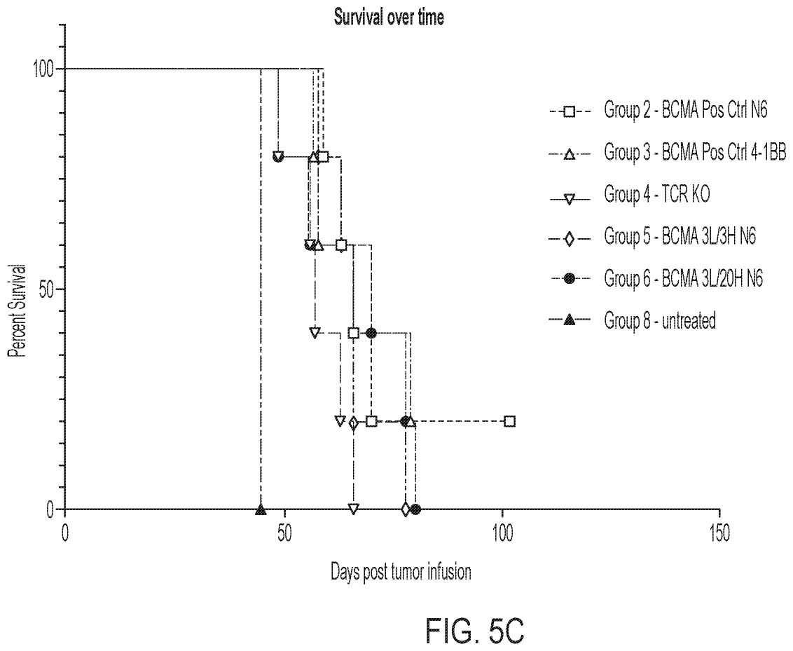

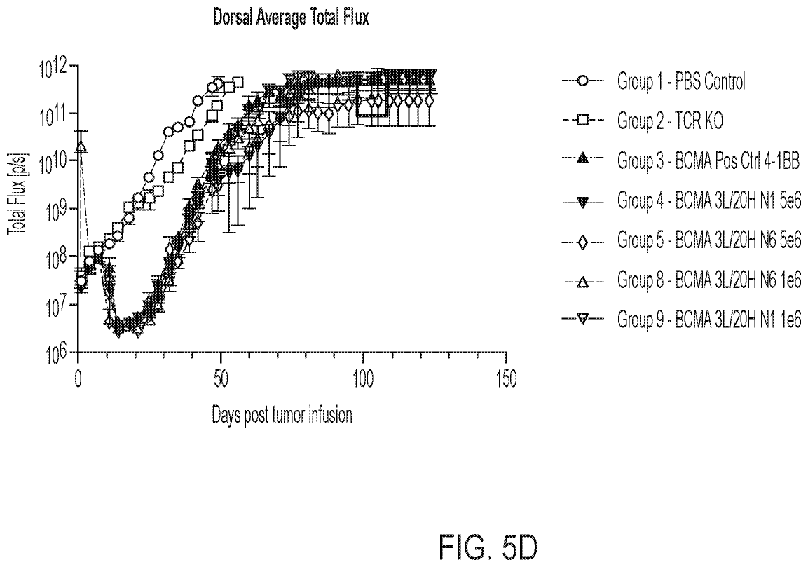

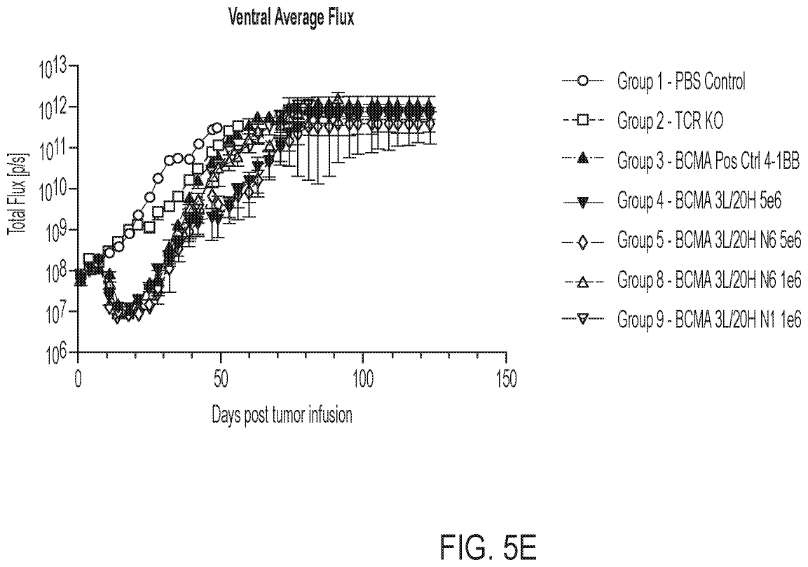

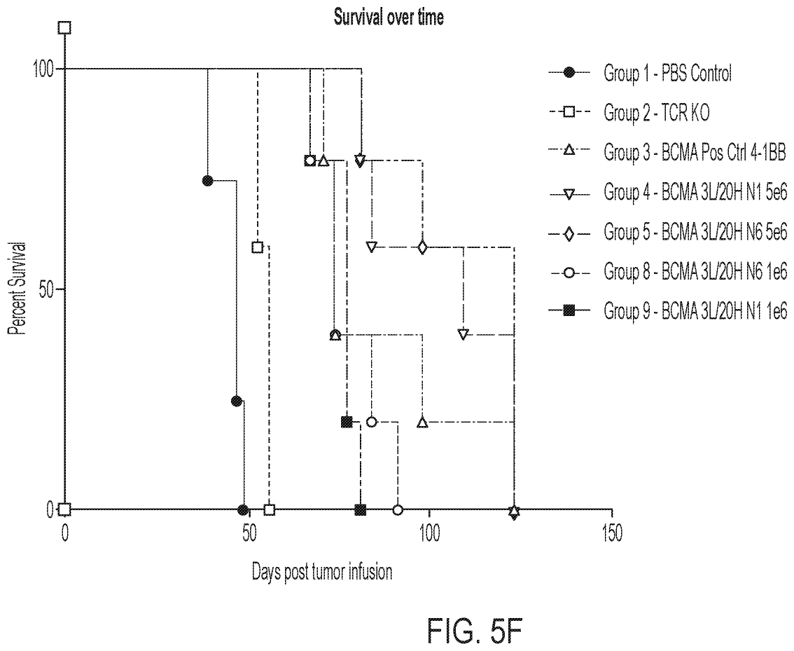

BLI measurements, indicative of tumor growth and in vivo burden, were acquired twice a week on both dorsal and ventral planes. At experimental time points immediately after T cell or vehicle administration, detectable BLI measurements dropped by a log or more in animals receiving anti-BCMA test or candidate CAR T cells (FIGS. 5A and 5B). By comparison, recipients of PBS alone (dark blue) or TRAC edited CAR”” T cells (purple) failed to see a commensurate drop in detectable luciferase expression at these time points. Due to tumor growth, animals receiving PBS vehicle or TRAC edited only cells survived out to day 45 and day 66 post-MM.1S luciferase injection, respectively (FIGS. 5A and 5B). Despite drops in detectable luciferase flux after CAR T cell administration, BLI recovered and increased between 2-3 weeks post-CAR T cell dosing in all remaining groups. NSG mice receiving BCMA-targeting CAR T cell therapy survived to day 78 or longer post-MM.1S luciferase injection.

To determine what effect CAR T cell dosing may have on in vivo efficacy, NSG recipient mice were administered either 1e6 or 5e6 anti-BCMA CAR T cells IV on day 8 post-MM.1S injection (2.5e6 cells IV, day 0). Animals receiving cells expressing a positive control BCMA CAR comprising a 4-1BB co-stimulatory domain, or receiving cells expressing a BCMA-3L/20H CAR, showed a substantial drop in detectable luciferase activity at time points immediately after CAR T cell dosing (FIGS. 5D and 5E). Tumor growth in PBS vehicle and TRAC edited control cell recipients steadily increased over time, with recipient animals dropping out of study on day 49 and 56 respectively (FIG. 5F). Although a notable difference in detectable BLI was not seen between animals receiving different doses of the same CAR T cells immediately after treatment (FIGS. 5D and 5E), overall survival correlated with an increase in initial CAR T cell dose. For example, while NSG mice receiving low dose BCMA-3L/20H CAR T cells dropped off study between day 71 and 90 post-MM.1S treatment, 60% of high dose recipients survived out to study end on day 123 (FIG. 5F).

### 3. Conclusions

The MM.1S multiple myeloma cell line represents a stringent model for the evaluation of anti-BCMA targeted therapies for the discovery of novel therapeutics. To this end, positive control BCMA CARs and experimental CAR T cells were compared head-to-head in an in vivo treatment model in NSG recipient mice. Tumor growth kinetics, as determined by BLI intensity and overall survival, were consistent across studies, and notable reductions in tumor growth were dependent on the expression of a BCMA targeting moiety. It is noteworthy in both studies that reductions in tumor growth were seen at time points immediately after CAR T cell dosing, suggesting that cytolytic activity of administered cellular therapies is rapid and considerable. Differences between in vivo efficacy were seen between anti-BCMA CAR products. All CAR therapies resulted in delayed tumor growth and increased overall survival. Furthermore, overall survival directly correlated with CAR T cell dose as recipients of high dose CAR T cells showed prolonged inhibition of tumor growth and survival. The results from the second study suggest that dosing of higher CAR T cell numbers may result in more long-term progression free survival or complete ablation of tumor burden.

### Example 6

### BCMA Specific CAR T Cell Stress Test

### 1. Methods

### Cell Production

CAR T cells were prepared that expressed either the control BCMA CARs previously described (comprising either the 4-1BB or N6 co-stimulatory domains) or expressed the experimental BCMA CARs described in Table 1. Anti-BCMA CAR and control T cells were manufactured as described in Example 4. The target K562-BCMA and K562-CD19 cell lines were expanded in Xuri medium+5% FBS supplemented with 10 ug/ml Blasticidin. MM.1S multiple myeloma cell lines were expanded in DMEM+15% FBS. Prior to assay initiation, target cell lines were stained with commercial anti-BCMA and anti-CD19 antibodies to evaluate antigen expression levels.

### Repeated Antigen Encounter Assay

TRAC edited only, and anti-BCMA CAR T cells were enumerated, spun down in 50 ml tubes, and re-suspended in fresh Xuri medium+5% FBS. For the repeated antigen encounter assay (stress test), CAR T cells were resuspended at 1e6 CAR T cells/ml. 100 ul (1e5 CAR T cells/well) of TRAC edited control and each anti-BCMA CAR T cell population were added to 6 individuals wells on 96-well round bottom plates. Target cell lines, comprised of MM.1S, K562-BCMA, or control K562-CD19 cells, were re-suspended at a concentration of 1e6 viable cells/ml. To duplicate wells of each T cell population, 100 ul of each target line was added to allow for replicate sampling of T cells and target lines over the course of the assay.

Stock T cell populations (1e5 CAR T cells/100 ul) were serially diluted two-fold in 15 ml tubes to final concentrations of 5.0e4, 2.5e4, and 1.3e4 CAR T cells/100 ul. As above, 100 ul of each diluted T cell population were added to 6 separate wells on 96-well round bottom plates. To duplicate wells, 100 ul of each of the three target cell lines was added for replicate sampling. In sum, duplicate wells were created for each T cell:target cell line at an effector:target ratio of 1:1, 1:2, 1:4, and 1:8. In addition, control wells containing T cells only or target cell lines only were plated (data not shown). Plates were covered and placed in 37 C degree incubators+5% CO2 for analysis at experimental time points.

On Day 6 and 9 of the experiment, 96-well round bottom plates were removed from the incubator and placed in biosafety cabinets. For flow cytometric analysis, samples were mixed and 50 ul of cells were transferred to fresh 96-well round bottom tubes. Plates were spun, supernatant was decanted, and samples were washed with 100 ul PBS/well and spun. Ghost dye BV510, to eliminate dead and dying cells during analysis, was diluted 1:400 in PBS. 100 ul was added to each experimental well. Samples were mixed, covered, and incubated at room temperature for 15 minutes. After incubation, plates were spun, supernatant was decanted, and samples were washed with 100 ul PBS/well and spun. For cellular analysis, a cocktail of antibodies was added to PBS comprised of the following: anti-CD3 (clone OKT3), BCMA biotinylated protein, anti-CD4 (clone OKT4), anti-CD8 (clone RPA-T8), anti-Lag3 (clone 11C3C65), anti-PD-1 (clone EH12.2H7), and anti-Tim3 (clone F38-2E2). 50 ul of antibody cocktail were added to each well and samples were mixed. Plates were incubated at room temperature for 15 minutes, after which plates were spun at 1350 RPM for 5 minutes. Cell pellets were washed twice with 100 ul PBS per wash. Lastly, streptavidin BCMA was diluted in PBS to make a stock concentration. 50 ul was added to each well and samples were mixed. Plates were incubated at room temperature for 15 minutes, after which plates were spun at 1350 RPM for 5 minutes. Cell pellets were washed with 100 ul PBS twice. Samples were re-suspended in 100 ul of PBS.

Sample acquisition was completed on a Becton Coulter Cytoflex S. A fixed volume was acquired for each sample to ensure accuracy in target re-seeding. After analysis was completed, target cell lines in fresh medium were added to experimental wells to re-establish the initial E:T ratio for subsequent experimental time points.

### 2. Results

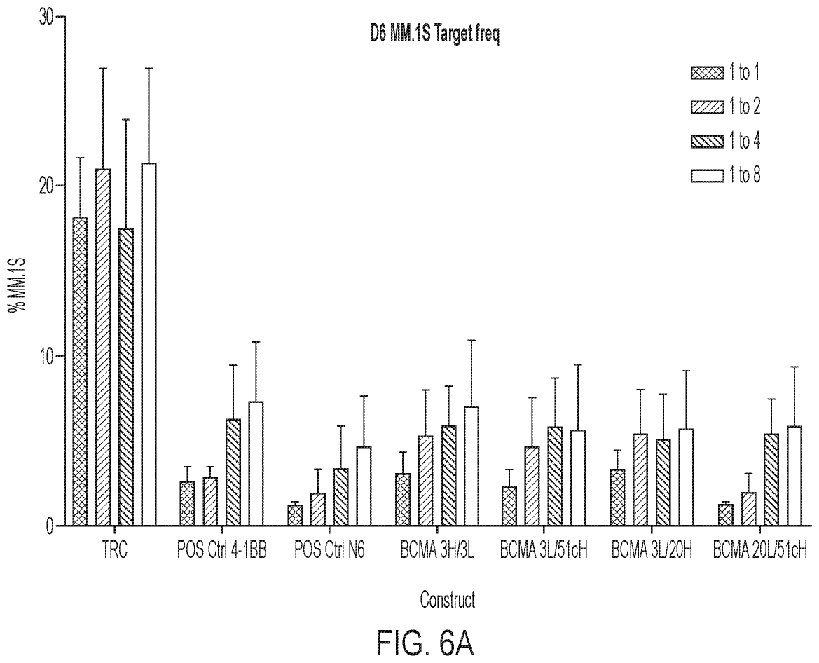

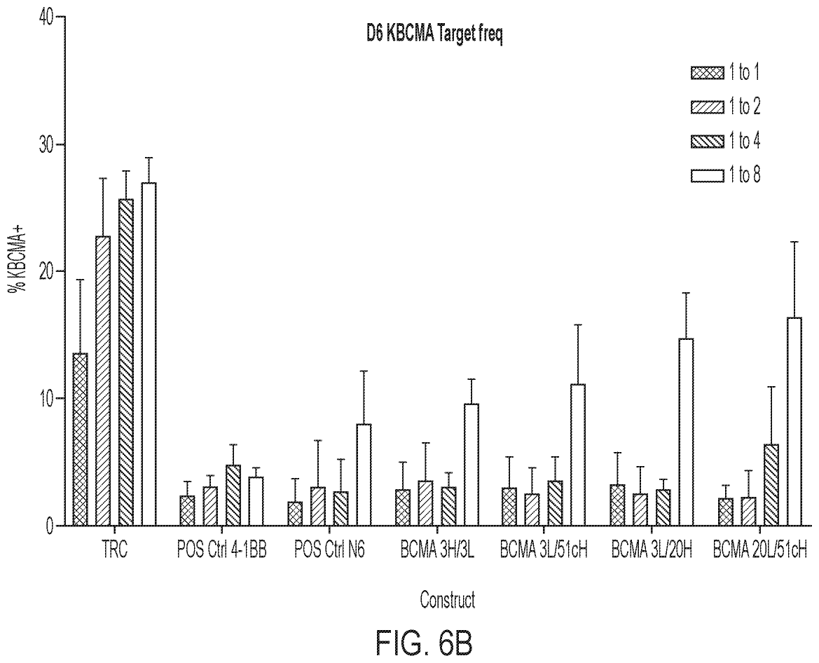

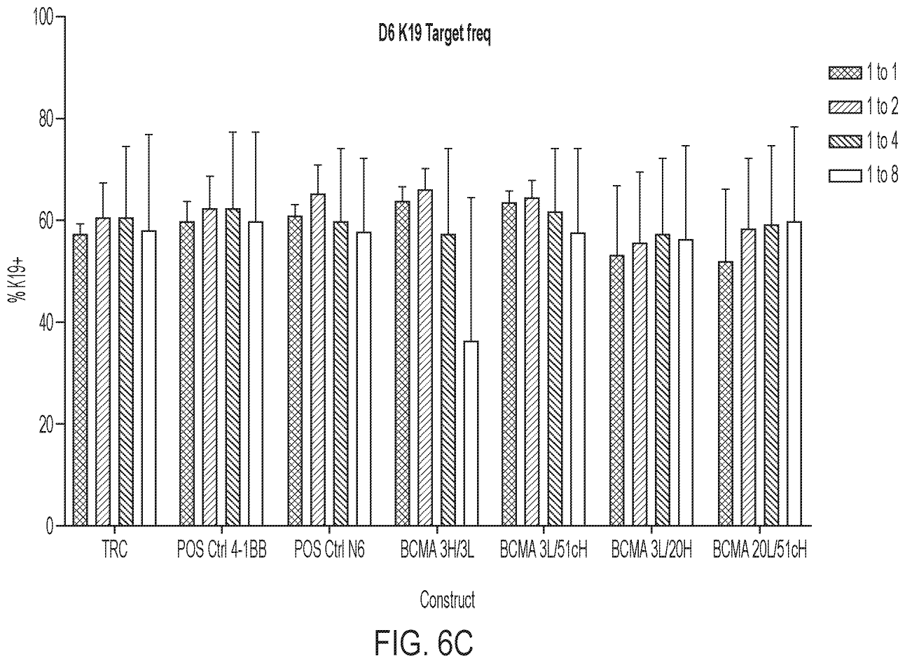

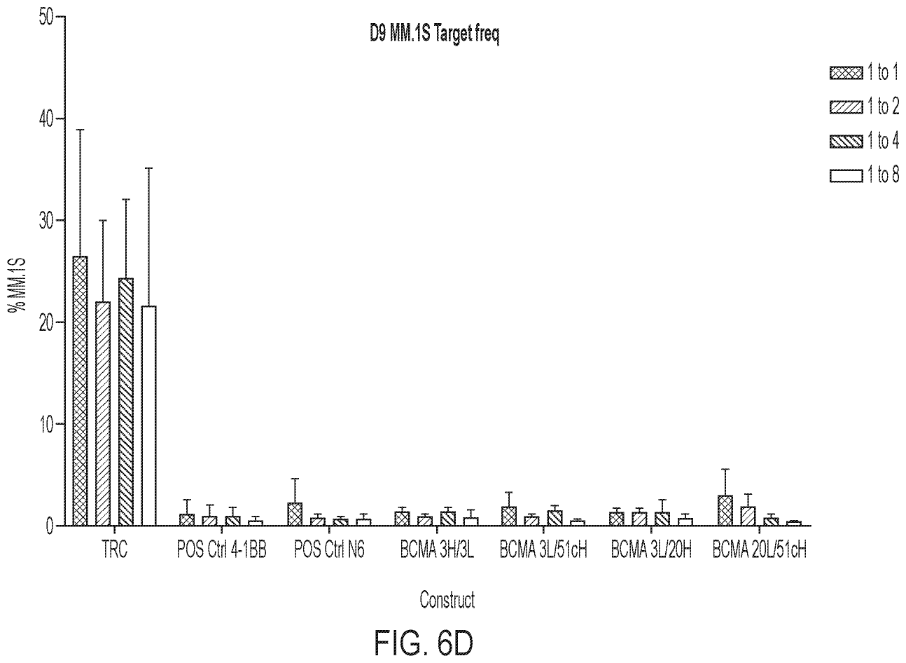

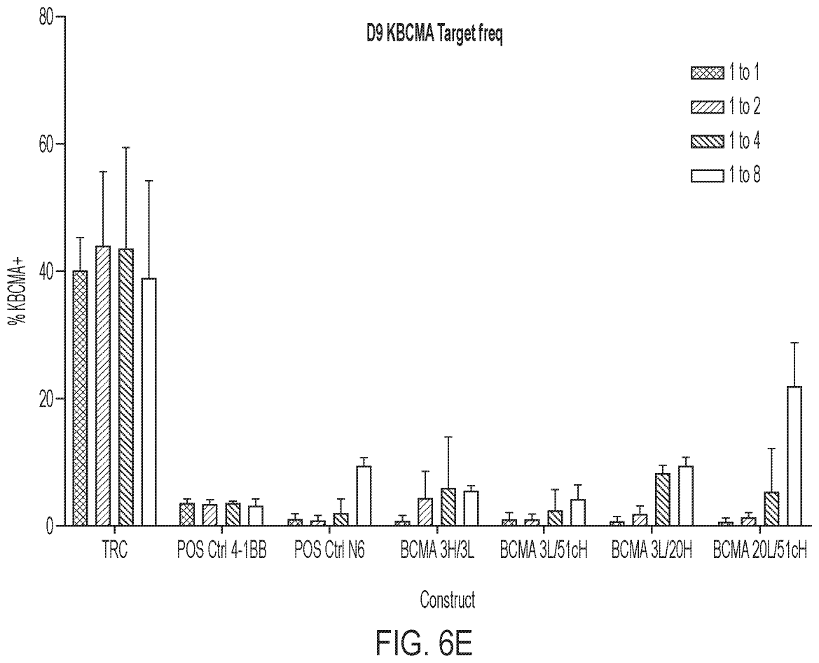

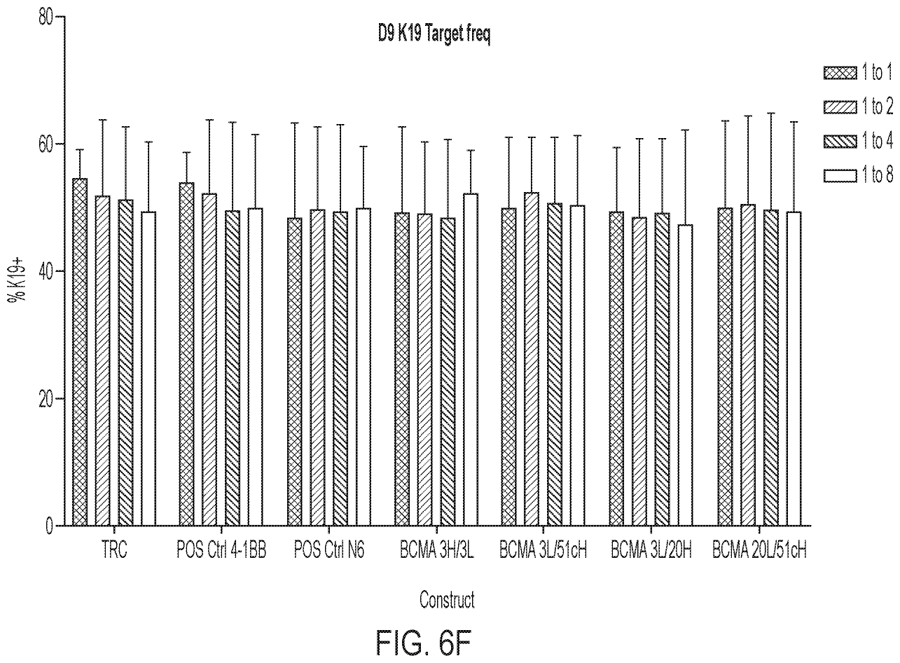

To compare the ability of anti-BCMA CAR T cells to specifically eliminate BCMA+ target cell lines, samples were stained for flow cytometric analysis at set intervals after addition of target lines. In wells seeded with MM.1S targets, CAR T cells expressing the positive control BCMA CAR comprising either a 4-1BB or N6 co-stimulatory domain showed elimination of BCMA+ targets in a dose-dependent fashion (FIG. 6A). For comparison, the experimental anti-BCMA CAR T cells (i.e., BCMA-3L/3H, BCMA-3L/51cH, BCMA-3L/20H, and BCMA-20L/51cH) showed a similar magnitude of MM.1S cell killing, with the highest remaining target frequencies remaining in the 1:8 E:T culture wells. At subsequent time points, residual BCMA+ MM.1S target cell frequencies were low and equivalent in wells containing BCMA-specific CAR T cell populations (FIG. 6D). Of note, TRAC edited only cells failed to show a reduction in MM.1S cell frequency at both experimental time points (FIGS. 6A and 6D).

To determine if elimination of BCMA+ targets differed based on the cell line utilized, residual target frequencies were analyzed in wells seeded with K562-BCMA cells. Interestingly, in day 6 samples, positive control BCMA CAR T cells comprising a 4-1BB co-stimulatory domain showed equivalent frequencies of BCMA+ targets in the all experimental wells regardless of the initial E:T ratio (FIG. 6B). Although the magnitude of BCMA+target cytolysis was similar at lower E:T ratios in wells containing the experimental CAR constructs, notable increases in residual BCMA+ cell frequencies were recovered at the 1:8 E:T ratio compared to the positive control CAR T cells (FIG. 6B). After fresh targets were added, samples were analyzed 72 hours later on day 9 in the experiment. Comparable to day 6, the positive control BCMA CAR T cells comprising a 4-1BB co-stimulatory domain had similar frequencies of detectable K562-BCMA cells regardless of the input E:T ratio (FIG. 6E). In contrast, experimental CAR T cells showed a trending increase in detectable BCMA+ frequency compared to the positive control BCMA CAR T at more stringent E:T ratios, with the BCMA-3L/20H and BCMA-20L/51cH CAR T cells showing the greatest difference (FIG. 6E).

To ensure the specificity of CAR T binding and function, anti-BCMA CAR T cells were cultured with K562-CD19 expressing target cell lines. In both day 6 (FIG. 6C) and day 9 (FIG. 6F) samples, recovered target cell frequencies were equivalent across all conditions including wells cultured with TRAC edited T cells only.

### 3. Conclusions

The elimination of BCMA+MM.1S targets was equivalent between wells cultured with anti-BCMA CAR T cells, and the magnitude of target cell elimination occurred in a dose-dependent fashion. Importantly, TRAC edited residual target cell frequency was significantly higher than all other experimental conditions, supporting the function of the BCMA-targeting moiety in various CAR candidates.

Additionally, K562-BCMA target cells were killed at similar frequencies at lower E:T ratios. It should be noted, however, that substantial differences in recovered BCMA+ target frequencies were seen in conditions cultured at a 1:8 input E:T ratio with the experimental BCMA CAR T cells versus the positive control BCMA CAR T cells comprising a 4-1BB co-stimulatory domain. This suggests a functional difference between this CAR T cell population specifically under conditions of repeated antigen encounter.

Lastly, the K562-CD19 target frequencies were unaffected in conditions co-cultured with anti-BCMA CAR T cells, suggesting that all the BCMA-targeting moieties tested were specific for the intended antigen.

### Example 7

### BCMA Antibody Off-Targeting Testing by Cellular Microarray

### 1. Methods

The CAR T cells expressing the BCMA-targeting moiety are designed to be specifically activated in the presence of BCMA+ target cells that results in CAR T-mediated target cell killing. The BCMA-binding domain is engineered and was selected for specific activity against BCMA+ cells. However, the potential exists for binding to off-target proteins expressed on the surface of human cells. Therefore, the potential for off-target binding to human cell-surface proteins was assessed in a cellular microarray assay.

The cellular microarray assay consists of 4,070 full-length human plasma membrane proteins that are expressed individually in human embryonic kidney (HEK)293 cells. This assay was used to screen for off-target binding activity of the mouse-derived, anti-human BCMA antibody (BCMA-3L/20H) from which the novel single-chain variable fragment (scFv) binding moiety used to construct a BCMA targeting CAR T cell. Additionally, the positive control BCMA antibody described in Example 1 was also tested.

TABLE 2
Materials and equipment

| Material | Mfr. | Catalog No. | Lot No. | Notes |
|---|---|---|---|---|
| Mouse anti-human BCMA (BCMA- | PBI | NA | NA | NA |
| 3L/20H scFv) | | | | |
| Control mouse anti-human BCMA | PBI | NA | NA | NA |
| Expi293F cells | Gibco | A14527 | NA | NA |
| Expi expression medium | Gibco | A14351-01 | NA | NA |
| Opti-MEM I reduced serum media | Gibco | 11058-021 | NA | NA |
| ExpiFectamine 293 transfection kit | Gibco | A14524 | NA | NA |
| MabSelectSuRe | GE | 17-5438-01 | NA | NA |
| | Healthcare | | | |
| HiLoad Superdex-200 | GE | 28-9909-44 | NA | NA |
| 10/300 GL | Healthcare | | | |

Abbreviations: BCMA = B-cell maturation antigen; NA = not applicable; PBI = Precision BioSciences, Inc.; scFv = single-chain variable fragment.

### Mouse Anti-BCMA Antibody Production

The BCMA-3L/20H antibody and the positive control BCMA antibody were expressed in Expi293F cells as full-length recombinant antibodies fused to human immunoglobulin (Ig) G4 fragment crystallizable region (Fc) using pFUSE vectors. Heavy and light chain plasmids for each antibody were cotransfected. Antibodies were purified by affinity and size exclusion chromatography.

### Cellular Microarray Screening

A cellular microarray was conducted by Retrogenix (Whaley Bridge, United Kingdom). Complementary deoxyribonucleic acid (cDNA) encoding 4,070 full-length human plasma membrane proteins were individually spotted onto slides. HEK293 cells were seeded over the cDNA and reverse-transfected, resulting in cell-surface expression of a specific protein from the library. All transfection efficiencies exceeded the minimum threshold. The slides were first incubated with a solution containing the BCMA-3L/20H antibody clone or the BCMA positive control antibody clone (5 μg/mL). An AlexaFluor 647 (AF647) anti-human IgG Fc secondary detection antibody was used to detect binding of the BCMA− 3L/20H clone or the BCMA positive control clone and samples were analyzed by fluorescence. ZsGreenl fluorescence was used to identify transfected HEK293 cells. Primary hits were identified by analyzing fluorescence (cells positive for both AF647 and ZsGreenl fluorescence) on ImageQuant. The intensities (signal to background) ranged from very weak to strong.

### 2. Results

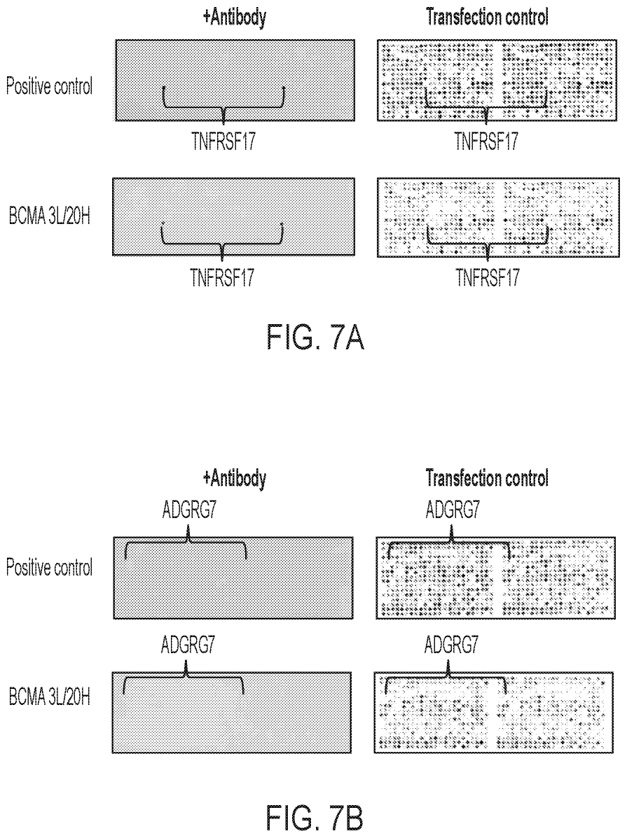

The screening results are summarized in Table 3. BCMA-3L/20H and the BCMA positive control clone showed strong interaction with BCMA, designated by its synonym Tumor Necrosis Factor Receptor Superfamily Member 17 (TNFRSF17). Both BCMA− 3L/20H and the BCMA positive control antibody showed weak to medium interaction with Fc fragment of IgG receptor 1a (FCGR1A). The BCMA positive control antibody demonstrated a strong interaction with Ig heavy constant gamma 3 (IGHG3), whereas the BCMA-3L/20H antibody produced a medium/strong interaction with this protein. These 25 interactions were due to direct interaction of FCGR1A and IGHG3 with the anti-human IgG Fc secondary detection antibody. BCMA-3L/20H showed no interaction with any other human plasma membrane protein in the microarray. A representative immunoblot slide images are provided in FIG. 7. Both the BCMA positive control and BCMA-3L/20H antibodies demonstrated a strong positive staining for TNFRSF17, whereas no staining could be seen for a representative cellular protein ADGRG7 (FIGS. 7A and 7B). The BCMA positive control antibody showed very weak interaction with PLXNA4 whereas the BCMA-3L/20H showed no interaction with this protein.

TABLE 3
Screening results

| | | | BCMA Positive |
|---|---|---|---|
| | | BCMA-3L/20H | control clone |
| Gene ID | Accession No. | (5 μg/mL) | (5 μg/mL) |
| TNFRSF17 | BC058291 | Strong | Strong |
| (BCMA) | | | |
| FCGR1A | BC032634 | Weak/Medium | Weak/Medium |
| IGHG3 | BC089421 | Medium/Strong | Strong |
| PLXNA4 | NM_020911 | — | Very weak |
| FCGR2A | BC020823 | — | — |
| CD72 | BC030227 | — | — |
| NOTCH1 | NM_017617 | — | — |
| PODXL2 | NM_015720 | — | — |
| PANX2 | NM_001160300 | — | — |
| IL20RA | BC113574 | — | — |
| ADGRG7 | NM_032787 | — | — |
| SIGLEC15 | BC029896 | — | — |

Abbreviations: — = no interaction; NA = not applicable; No. = number.

### 3. Conclusions

In this screening test, no off-target interactions were observed with the BCMA-3L/20H anti-human BCMA antibody, from which the scFv used to construct an anti-BCMA CAR of was derived. In contrast, the positive control reacted more strongly with both IGHG3 and PLXNA4, as described above.

### Example 8

### Characterization of the Phenotype, Activity, and Specificity of an Anti-BCMA CAR T Cell

### 1. Methods

### Production of a Demonstration CAR T Batch and Thawing of Cells

BCMA CAR T cells expressing the BCMA-3L/20H CAR (BCMA-3L/20H CAR T cells) were produced as described in Example 4. Cryopreserved CAR T cells were thawed and added to Xuri medium supplemented with 5% fetal bovine serum (FBS). The cell suspension was centrifuged, the supernatant decanted, and the cells resuspended in Xuri medium supplemented with 5% FBS.

### Immunophenotyping

An aliquot of CAR T cells was washed in phosphate buffered saline (PBS), centrifuged, and then stained with an antibody cocktail in PBS for 15 minutes at room temperature. Samples were then washed twice in PBS, resuspended in fresh PBS, and analyzed on a flow cytometer to collect data for frequency of TCR⁻ cells, frequency of CAR⁺ cells, CD4:CD8 ratio, and frequency of T cell memory subpopulations.

### CAR T Cell Coculture

Target BCMA+MM.1S and BK562 cells (human K562 cells engineered to express BCMA) were used to stimulate BCMA-3L/20H CAR T cell responses. BCMA⁻ unmanipulated human K562 cells and TCR⁻ T cells were included as negative controls. MM.1S, BK562, and K562 cells were subjected to flow cytometry to assess the presence of BCMA. The BCMA-3L/20H CAR T cells or negative control TCR⁻ T cells were cocultured with MM.1S in RPMI medium supplemented with 15% FBS at E:T ratios (BCMA-3L/20H CAR T cells or TCR⁻ control T cells:MM.1S cells) of 1:0.5 (1×10⁵:5×10⁴), 1:2 (1×10⁵:2×10⁵), and 1:5 (1×10⁵:5×10⁵). BCMA-3L/20H CAR T cells or negative control TCR⁻ cells were cocultured with BK562 or K562 cells in Xuri medium supplemented with 5% FBS at E:T ratios (BCMA-3L/20H CAR T cells or TCR⁻ control T cells:BK562 or K562 cells) of 1:0.5 (1×10⁵:5×10⁴), 1:2 (1×10⁵:2×10⁵), and 1:5 (1×10⁵:5×10⁵). Cocultured BCMA-3L/20H CAR T cells and target cells were incubated for 48 hours and assessed for cytokine release and incubated for an additional 3 days and then assessed for proliferation and cytotoxicity.

### Assessment of Cytokine Release

After 48 hours of coculture, 50 μL of supernatant was removed from each coculture well and stored at −20° C. until analysis. Coculture supernatants were thawed and diluted at a 1:10 ratio in manufacturer's diluent and a 4-plex cartridge was loaded according to manufacturer's instructions (50 L/well for sample wells, 1 mL/well for wash buffer). Levels of IFNγ, IL-2, IL-6, and TNFα were measured in supernatants on a ProteinSimple Ella plate reader. Activity of BCMA-3L/20H CAR T cells against MM.1S target cells was measured in triplicate coculture wells. Activity of BCMA-3L/20H CAR T cells against K562 control cells was measured in duplicate coculture wells.

### In Vitro Assessment of Proliferation and Cytotoxicity

Samples for proliferation and cytotoxicity assessments were prepared at Day 5. Cocultures were centrifuged and the supernatant was removed. Coculture cells were resuspended in PBS and centrifuged. The PBS was removed and the cocultures were washed once more in PBS and prepared for flow cytometric analyses. Samples were incubated for 15 minutes at 4° C. with PBS containing:
Live/Dead CF-800 (1:1000)
anti-CD29 BV711 (1:200)
anti-BCMA PE (1:200)
anti-CD4 FITC (1:200)
anti-CD8 BV421 (1:200)

After incubation, the cells were washed in PBS, centrifuged, the supernatant decanted, and resuspended in 120 μL of PBS. Sample data were acquired immediately after PBS resuspension on a Beckman-Coulter CytoFLEX-LX flow cytometer. Cell count data were captured and exported for analysis.

### 2. Results

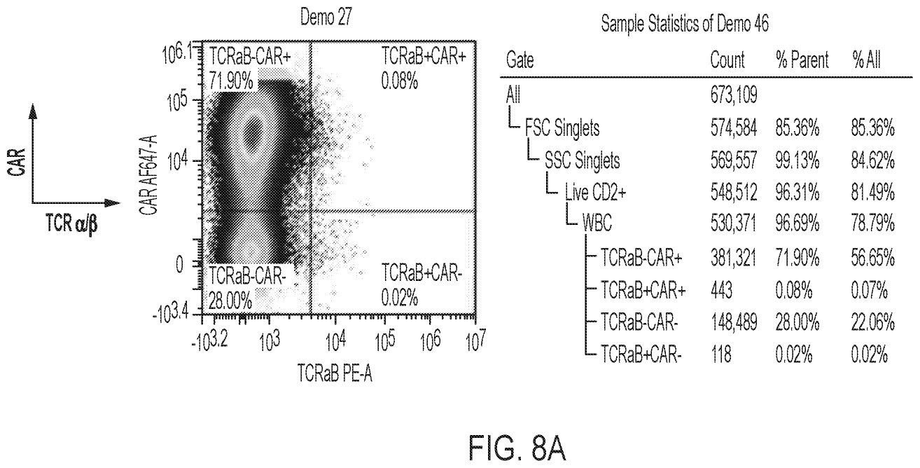

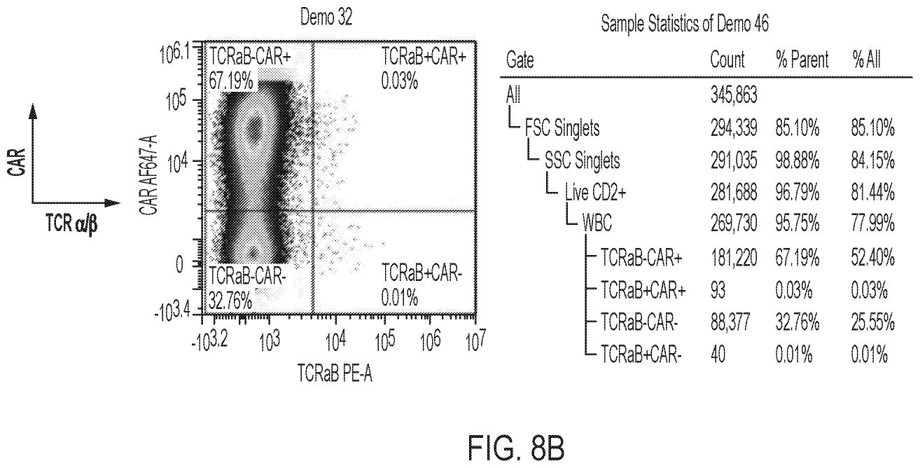

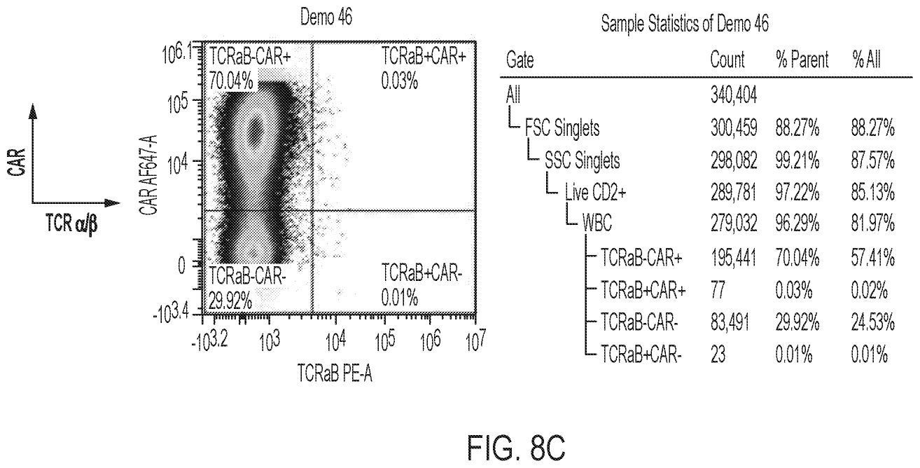

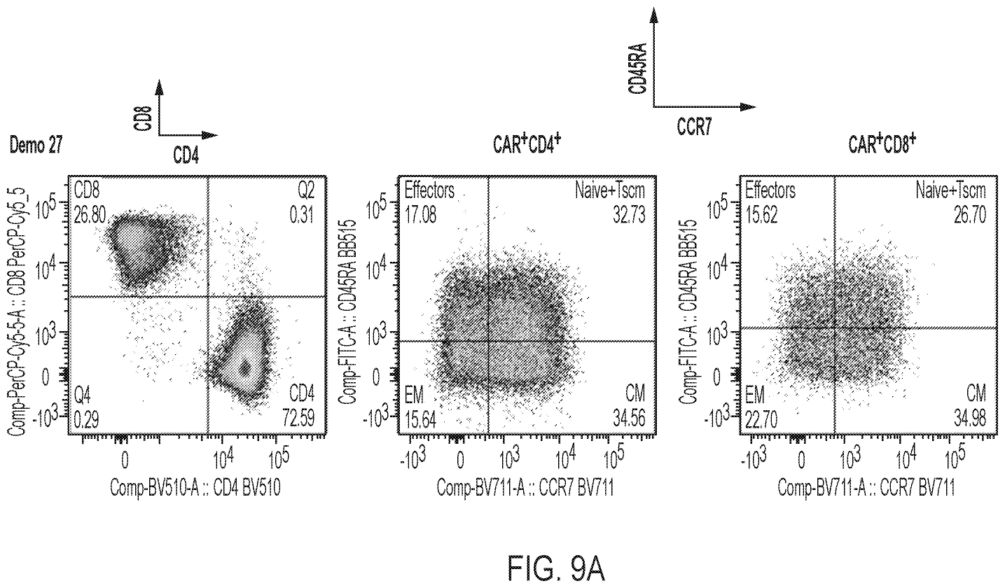

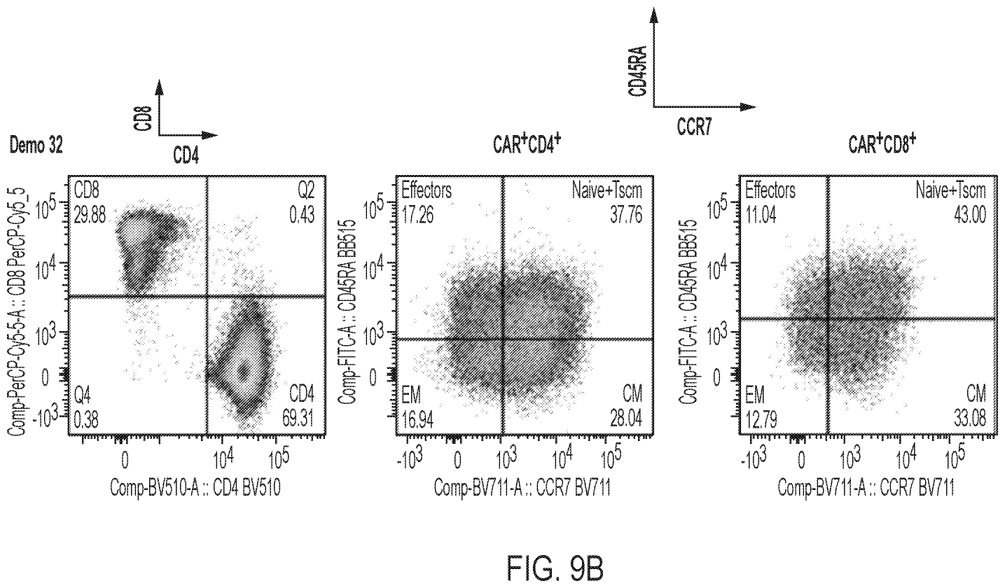

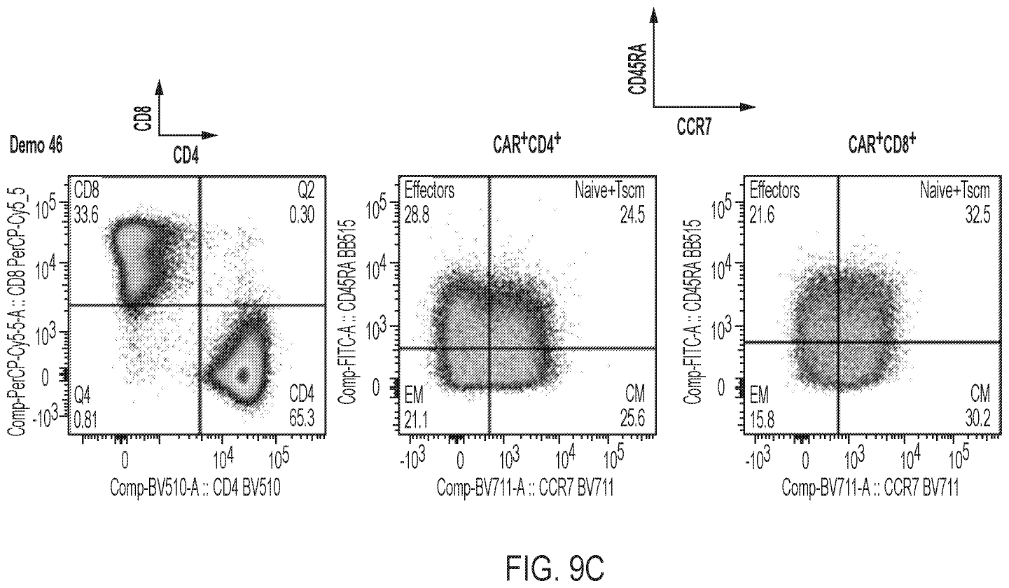

Three Demo batches of BCMA-3L/20H CAR T cells were analyzed by flow cytometry to determine the percentage of T cells that are TCR⁻, CAR⁺, CD4⁺, CD8⁺, Tn, Tcm, and effector memory (Tem) phenotypes. Flow cytometry results for all 3 Demo batches (Demo 27, 32, and 46) of BCMA-3L/20H CAR T cells are summarized in Table 4 and flow cytometry plots are presented in FIG. 8 and FIG. 9. Flow cytometry results demonstrate that >99% of the cells are TCR⁻, of which >50% are TCR⁻CAR⁺ (range: 67.19% to 71.90%). The CD4:CD8 ratios of TCR⁻CAR⁺ cells ranged from 1.94 to 2.71. The majority of CD4⁺CAR⁺ cells are represented by a combination of Tn and Tcm phenotypes. This data profile shows that the process consistently generates an enriched population of TCR⁻CAR⁺ T cells with a desirable composition and phenotype.

TABLE 4
Overview of BCMA-3L/20H CAR T cell characterization

| Parameter | Demo 27 | Demo 32 | Demo 46 |
|---|---|---|---|
| TCRα/β⁻ (%) | 99.90 | 99.95 | 99.96 |
| TCRα/β⁻CAR⁺ (%) | 71.90 | 67.19 | 70.04 |
| KI (% of KO) | 71.97 | 67.22 | 70.07 |
| CD4:CD8 ratio (TCRα/β⁻CAR⁺) | 2.71 | 2.32 | 1.94 |
| CD4⁺ TCRα/β⁻CAR⁺ (%) | 72.59 | 69.31 | 65.30 |
| CD8⁺ TCRα/β⁻CAR⁺ (%) | 26.80 | 29.88 | 33.60 |
| CD4⁺CCR7⁺ (%) | 67.28 | 65.80 | 50.10 |
| CD8⁺CCR7⁺ (%) | 61.68 | 76.20 | 62.70 |
| Viability (%) | 93.50 | 88.00 | 88.9 |

Abbreviations: CAR = chimeric antigen receptor; CCR = C-C chemokine receptor; CD = cluster of differentiation; KI = knock in; KO = knock out; TCR = T cell receptor.

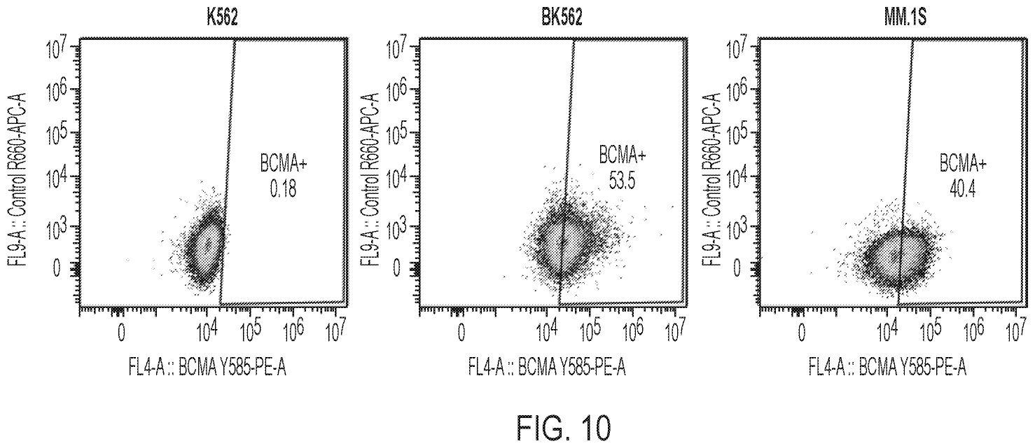

The percentage of K562 cells, BK562 cells, and MM.1S cells expressing BCMA was analyzed by flow cytometry (FIG. 10). Flow cytometry results show that MM.1S cells and BK562 cells express BCMA while negative control K562 cells show no expression of BCMA.

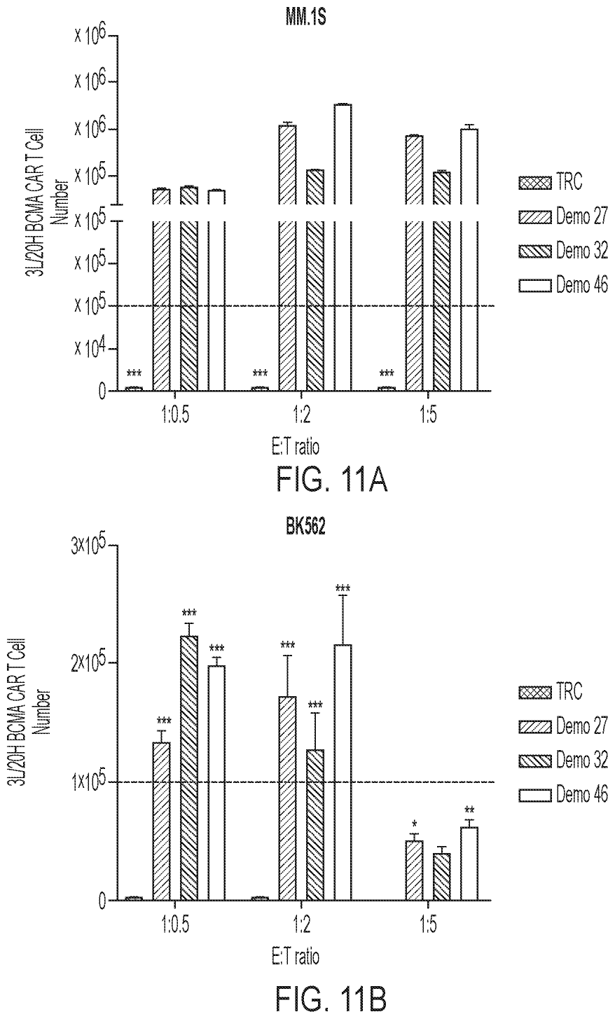

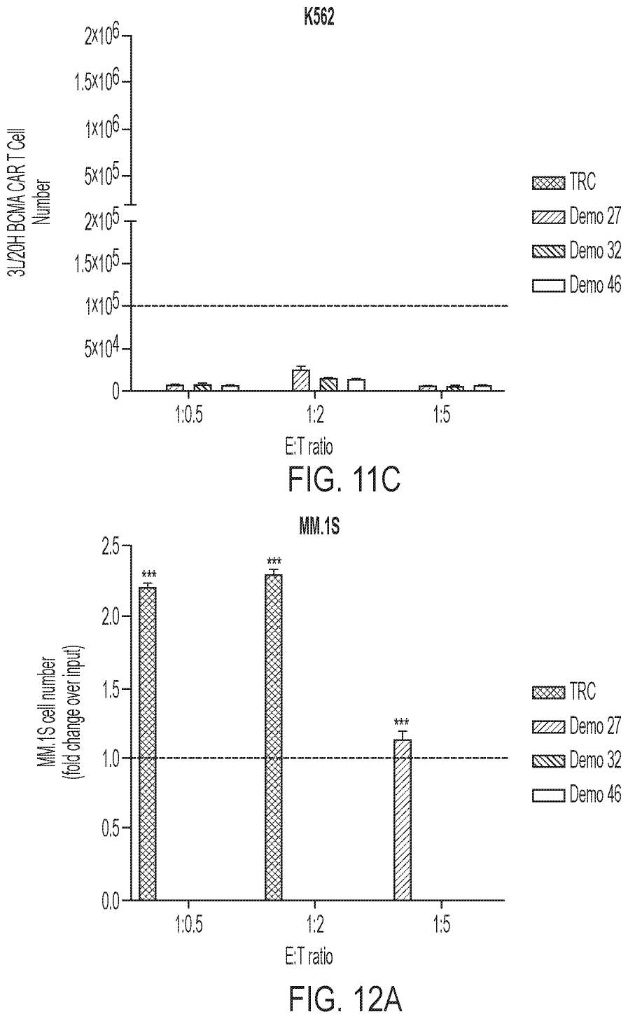

The activity of BCMA-3L/20H cells was assessed by several methods including proliferation, cytotoxicity, and production of effector cytokines in response to coculture with BCMA⁺MM.1S and BK562 cells, and BCMA⁻ K562 negative control target cells. After 5 days of coculture, BCMA-3L/20H cells from 3 different Demo batches (Demo 27, 32, and 46) proliferated in response to stimulation by BCMA⁺MM.1S (FIG. 11A) and BK562 (FIG. 11B) target cells, with higher levels of proliferation observed against the MM.1S multiple myeloma cell line than the engineered BK562 cells. BCMA-3L/20H cells did not proliferate in response to coculture with BCMA⁻ K562 cells (FIG. 11C). Proliferative responses of BCMA-3L/20H cells against BCMA⁺ target cells were similar across all 3 Demo batches.

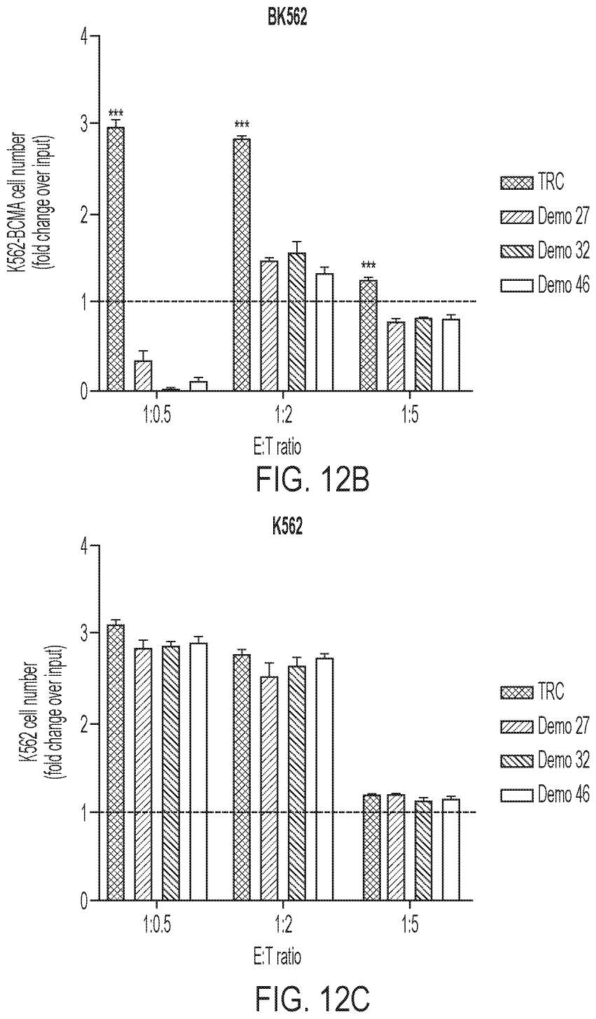

The cytotoxic potential of BCMA-3L/20H cells from 3 different Demo batches was evaluated after 5 days of coculture in the presence of BCMA⁺MM.1S and BK562 target cells and BCMA⁻ K562 negative control cells. Significant BCMA-3L/20H cells-mediated cytotoxic killing was observed against BCMA+MM.1S (FIG. 12A) and BK562 (FIG. 12B) target cells at all E:T ratios across all 3 Demo batches in comparison to TRC control, with higher levels of activity observed against the MM.1S multiple myeloma cell line than the engineered BK562 cells. Target cell killing was not observed when BCMA-3L/20H cells were cocultured with BCMA⁻ K562 negative control target cells (FIG. 12C).

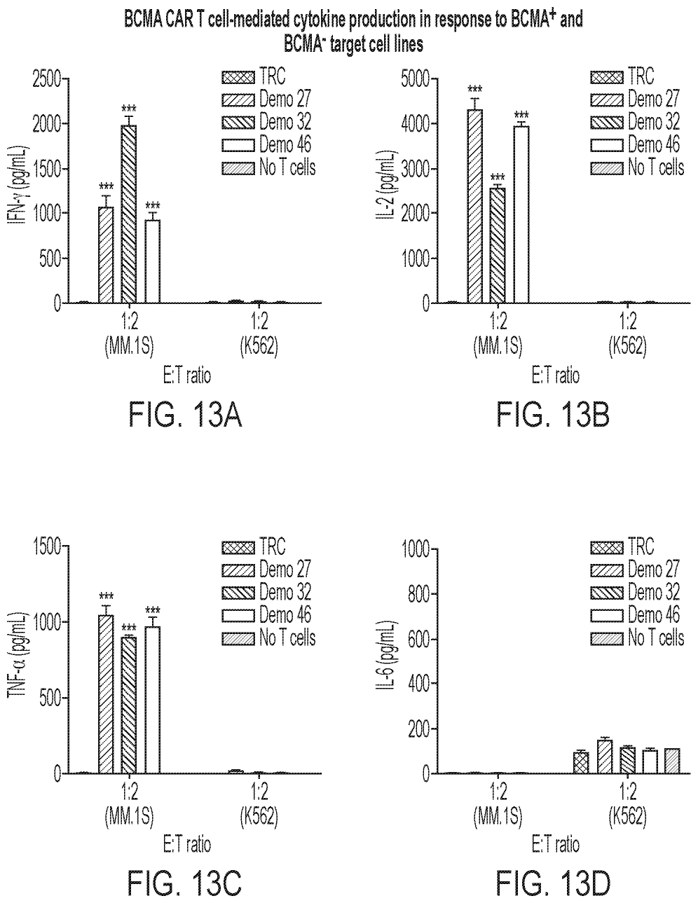

The cytokine production response of BCMA-3L/20H cells from 3 different Demo batches was evaluated after 2 days of coculture in the presence of BCMA⁺MM.1S target cells and BCMA⁻ K562 cells by testing cell culture supernatants by multiplex enzyme-linked immunosorbent assays. In response to coculture with BCMA⁺MM.1S target cells, BCMA-3L/20H cells produce the cytokines IFN7 (FIG. 13A), IL-2 (FIG. 13B), and TNFα (FIG. 13C), but do not produce these cytokines when cocultured with BCMA⁻K562 cells. BCMA-3L/20H cells produced little or no IL-6 in response to coculture with BCMA⁺MM.1S target cells. However, monocytes, which were not included in this in vitro test system, are required for IL-6 production in response to activation of CAR T cells (Singh et al., Cytotherapy. 2017; 19(7):867-880). Low levels of IL-6 were observed in all K562 cell samples (FIG. 13D), and it has been shown that unstimulated K562 cells produce IL-6 (Navarro et al., Exp Hematol. 1991; 19(1):11-7).

### 3. Conclusions

The data provided demonstrates that the BCMA-3L/20H cells process successfully generated T cell products from 2 independent donors that are greatly enriched for TCR⁻ cells with >50% TCR-CAR⁺ T cells and have a desirable composition and phenotype (CD4:CD8 ratio ≥0.5:1, Tn+Tcm ≥50%). These three CAR T cell batches demonstrated activity when cocultured with BCMA⁺ target cells and not in the presence of BCMA⁻ control cells as measured by proliferation, cytotoxicity against target cells, and release of pro-inflammatory cytokines. In summary, these results confirm the specificity and activity of BCMA-3L/20H cells towards BCMA⁺ target cells.

### Example 9

### Efficacy Study of BCMA-3L/20H CAR T Cell Activity in a Disseminated Multiple Myeloma Model

### 1. Methods

To assess the ability of BCMA-3L/20H cells to target BCMA⁺ myeloma tumor cells, a pivotal GLP (with exceptions) study was conducted in NSG mice implanted IV with the luciferase-expressing BCMA⁺MM.1S multiple myeloma cell line (MM.1S-ffLuc). The route of administration in this study was IV, which is the intended route of administration in humans. Doses were selected based on investigative study results that demonstrated tumor size reduction and a survival advantage in tumor-bearing NSG mice administered BCMA-3L/20H cells compared with tumor-bearing NSG mice administered vehicle control or TCR⁻ control T cells.

The objective of this study was to determine the minimum efficacious dose of BCMA-3L/20H cells over a range of doses (1.0×10⁶, 5.0×10⁶, or 1.5×10⁷ cells per animal; 4.0×10⁷, 2.0×10⁸, or 6.0×10⁸ cells/kg, respectively) that results in antitumor efficacy at multiples of approximately 5- to 100-fold over the anticipated maximum dose level (6×10⁶ cells/kg) proposed for administration in the clinic.

BCMA-3L/20H cells were evaluated for efficacy against MM.1S-ffLuc in a mouse xenograft model. Female and male NSG mice were injected IV in the tail with 2.5×10⁶ MM.1S-ffLuc cells. On Day 1 (9 days post-implantation), mice with established disseminated MM.1S-ffLuc tumors were administered IV a single dose of either vehicle control, TCR⁻ control T cells (negative efficacy control) or BCMA-3L/20H cells (test article) (Table 5) to assess efficacy parameters.

TABLE 5
In vivo efficacy study design

| | | | Dose | | Schedule for IV |
|---|---|---|---|---|---|
| Group | n/sex | Treatment | (cells/animal) | Route | Dosing |
| 1 | 6/F | Vehicle^{a)} | NA | IV | QD × 1 |
| 2 | 6/M | Vehicle^{a)} | NA | IV | QD × 1 |
| 3 | 6/F | TCR⁻ control T cells (NP11)^{b)} | 1.5 × 10⁷ | IV | QD × 1 |
| 4 | 6/M | TCR⁻ control T cells (NP11)^{b)} | 1.5 × 10⁷ | IV | QD × 1 |
| 5 | 6/F | BCMA-3L/20H cells low (NP10)^{c)} | 1.0 × 10⁶ | IV | QD × 1 |
| 6 | 6/M | BCMA-3L/20H cells low | 1.0 × 10⁶ | IV | QD × 1 |
| | | (NP10)^{c)} | | | |
| 7 | 6/F | BCMA-3L/20H cells mid | 5.0 × 10⁶ | IV | QD × 1 |
| | | (NP10)^{c)} | | | |
| 8 | 6/M | BCMA-3L/20H cells mid | 5.0 × 10⁶ | IV | QD × 1 |
| | | (NP10)^{c)} | | | |
| 9 | 6/F | BCMA-3L/20H cells high | 1.5 × 10⁷ | IV | QD × 1 |
| | | (NP10)^{c)} | | | |
| 10 | 6/M | BCMA-3L/20H cells high | 1.5 × 10⁷ | IV | QD × 1 |
| | | (NP10)^{c)} | | | |

Abbreviations:; F = female; HSA = human serum albumin; IV = intravenous; M = male; NA = not applicable; QD = once a day.
^{a)}Diluent: Plasma-Lyte A, 2% has.
^{b)}Designation code given for client confidentiality.
^{c)}Designation code given for client confidentiality.
Note:
BCMA-3L/20H cell doses represent CAR T cells per animal.

### Mice

Male and female NSG mice (NOD.Cg-Prkdc^{scid}Il2rg^{tmIWjI}/SzJ, The Jackson Laboratory) were 10 weeks old with body weights ranging from 20.4 to 34.9 grams at the 15 beginning of the study. The test laboratory specifically complies with the recommendations of the Guide for Care and Use of Laboratory Animals with respect to restraint, husbandry, surgical procedures, feed and fluid regulation, and veterinary care.

### Formulation of Test and Control Articles

BCMA-3L/20H CAR T cells (TCR⁻CAR⁺, Batch Demo 32) and TCR⁻ control T cells were generated from the same leukapheresis donor and were produced as described in Example 4. Demo 32 was generated specifically for pivotal, nonclinical studies. The cells were supplied as frozen vials in cryopreservation media and formulated to the appropriate concentration with supplied diluent (Plasma-Lyte A, 2% H). The vehicle for the study was the diluent, which was supplied to in frozen 50 mL conical tube(s).

Pre- and post-injection viability was 89.4% and 87.1%, respectively, for the TCR⁻ control T cells and 92.4% and 90.4% (1.0×10⁶), 91.2% and 89.1% (5.0×10⁶), and 92.7% and 88.6% (1.5×10⁷), respectively, for the BCMA-3L/20H CAR T cell doses.

### Mouse Intravenous Tumor Cell Injection

Frozen vials containing a clone of MM.1S multiple myeloma cells that stably express firefly luciferase (MM.1S-ffLuc) were utilized for this study. Cells were thawed and cultured in RPMI-1640 medium containing 15% fetal bovine serum (FBS), 2 mM glutamine, 100 units/mL penicillin G sodium, 100 μg/mL streptomycin sulfate, and 25 g/mL gentamicin. The tumor cells were cultured in tissue culture flasks in a humidified incubator at 37° C., in an atmosphere of 5% CO₂ and 95% air. Cells were washed, counted, viability determined, and resuspended in sterile phosphate buffered saline (PBS). Each mouse was injected IV in a tail vein with 2.5×10⁶ MM.1S-ffLuc cells in a 0.2 mL suspension. Based on tumor growth characterization results from a model development study, it was determined the optimal time to begin treatment with test agents was 8 to 9 days after tumor implantation. The animals were randomized into 10 groups (n=6/group) based on tumor burden (BLI flux values) and gender.

### Mouse Treatment

Day 1 (9 days post-implantation) of the study designates the day of dosing of the control or test articles (vehicle control, TCR⁻ control T cells, or BCMA-3L/20H CAR T cells) according to the treatment protocol summarized in Table 5. Odd-numbered groups consisted of female animals, and even-numbered groups consisted of male animals. The dosing volume was a fixed 0.2 mL/mouse by IV injection.

### Imaging Methods

In vivo BLI was performed on Day 0 (8 days post-implantation) and once a week thereafter to Day 63. Whole body ventral images were captured 10 minutes after luciferin substrate injection. Luciferase activity was measured in live animals using IVIS® SpectrumCT (Perkin Elmer, MA) equipped with a CCD camera (cooled at −90° C.), mounted on a light-tight specimen chamber. On the day of imaging, animals received intraperitoneal injections with luciferin substrate (total 150 mg/kg) and were placed in anesthesia induction chamber (2.5 to 3.5% isoflurane in oxygen). Upon sedation, animals were positioned in the imaging chamber, equipped with stage heated at physiological temperature, on their dorsal side to acquire images of their ventral side, for image acquisition 10 minutes onwards post-luciferin substrate injection.

Regions of interest were drawn around each mouse image, and flux was quantified and reported as 10⁶ photons per second (p/s). Data was analyzed and exported using Living Image software 4.5.1. (Perkin Elmer, MA). Representative images were defined as animals with whole body flux values closest to the calculated median at the timepoint when >50% of the animals of a group at a given time point remained on study.

When an animal exited the study due to tumor burden or clinical signs of supporting moribundity, the final flux value recorded was carried forward and included with the data to calculate the group mean and median intensities at subsequent time points until <50% of the animals remain. Any animal that died due to nontreatment-related (NTR) deaths attributed to accidental (NTRa) or unknown etiology (NTRu) were excluded from the analysis data set. No NTR deaths were observed in this study.

### Endpoint and Tumor Burden Inhibition Analysis

Treatment efficacy based on tumor burden inhibition (TBI) was determined using data from Day 36, which was the last time point where bioluminescence imaging (BLI) measurements were obtained for the vehicle control group wherein >50% of the animals were alive and able to be measured. The median flux (MF) for the number of animals (n) on this day was determined for each group. Percent TBI was defined as the difference between the MF of the designated vehicle control group and the MF of the drug-treated group, expressed as a percentage of the MF of the control group:

$$\% TBI = [ 1 - ( {MF}_{drug - treated} / {MF}_{control} ) ] \times 100$$

The data set for TBI analysis includes all animals in a group, except those that die due to treatment-related (TR) or NTR causes.

### Endpoint for Survival Study (ILS)

Animals were monitored individually for an endpoint of moribundity due to progression of tumor burden. The time to endpoint (TTE) was recorded for each mouse that died of its disease or was euthanized due to disease progression; these deaths were assigned TTE values equal to the day of death or euthanasia. Median TTE values were calculated for each group. Animals that survived to the end of the study were euthanized and were assigned a TTE value equal to the last day of the study. The median TTE of treated mice is expressed as a percentage of the median TTE of vehicle control mice (% T/C), and the ILS is calculated as:

$$ILS = \% T / C - 100 \% , \\ where T = median {TTE}_{treated} , and \\ C = median {TTE}_{control} . \\ Thus , if T = C , ILS = 0 \% .$$

### Clinical Observations

Animals were weighed daily from Day 1 to Day 5, then 3 times per week until the completion of the study. The mice were observed frequently for overt signs of any TR side effects, and clinical observations were recorded. Individual body weight was monitored as per Study Protocol, and any animal with weight loss exceeding 30% for 1 measurement or exceeding 25% for 3 consecutive measurements was euthanized as a treatment related (TR) death (for treated groups). Group mean body weight loss was also monitored.

Animals presenting with full hindlimb paralysis, ocular proptosis, excessive body weight loss, ruffled fur, hunched posture, or moribundity due to tumor progression were euthanized and classified as death on a survival study (DSS). An animal death was classified as TR if the death was attributable to treatment side effects as evidenced by clinical signs and/or necropsy. A TR classification was also assigned to deaths by unknown causes within 14 days of the administered dose. A death was classified as NTR if there was no evidence that death was related to treatment side effects. No TR or NTR deaths were observed in this study; all animal deaths in this study are classified as death on survival study (DSS).

### Sampling

On the last day of the study (Day 63), a scheduled necropsy of all remaining animals was conducted by a board-certified pathologist to assess gross pathology and ensure sample integrity. If animals reached an unscheduled moribundity, an appropriately-trained technician performed and recorded the necropsy. Necropsies were performed after terminal blood collection but before organ collection. Prior to necropsy, full volume blood of each animal was collected by terminal cardiac puncture under isoflurane anesthesia and was split into 2 parts. Part 1 was collected into tubes without anti-coagulant, processed to serum, and snap frozen for future clinical chemistry assessment. Part 2 (˜200 μL) was collected into tubes containing K₂EDTA anti-coagulant and sent for CBC analysis on the same day of collection. Both CBC and clinical chemistry assessments were performed at Antech Diagnostics (Morrisville, NC).

Immediately after necropsy, organs (brain, bone marrow within both intact femurs, cecum, colon, heart, left kidney, liver, left lung, skin (intrascapular), small intestine, spleen, stomach, and testes or ovaries) were removed, weighed (except femur bone marrow and skin), and fixed in formalin for 48 hours. Samples were then transferred to 70% ethanol and prepped for formalin-fixed paraffin-embedding (FFPE), hematoxylin and eosin (H&E) staining, and hCD45 immunohistochemical (IHC) analysis of bone marrow (a decalcification step was performed on femurs prior to paraffin embedding). Any remaining fixed tissue, FFPE blocks, or stained slides were returned from CRL (Frederick, MD) to CRDS (Morrisville, NC) for storage until archiving. The staining intensity and frequency scales used for the IHC evaluation are listed in Table 6.

TABLE 6
Staining intensity and frequency

| Staining intensity | | Staining frequency | |
|---|---|---|---|
| Score | Result | Score | Result |
| Neg | Negative (no stained cells) | Very rare | <1% stained cells |
| 1+ | Weak | Rare | 1% to 5% stained cells |
| 2+ | Moderate | Rare to occasional | >5% to 25% stained cells |
| 3+ | Strong | Occasional | >25% to 50% stained cells |
| 4+ | Intense | Occasional to frequent | >50% to 75% stained cells |

### Statistical and Graphical Analyses

Prism 8.1.1 (GraphPad) for Windows was used for statistical and graphical analyses. Study groups experiencing clinical endpoints beyond acceptable limits (>20% group mean body weight loss or greater than 10% TR deaths) or having fewer than 5 evaluable observations, were nonevaluable and not included in statistical analyses.

The logrank test, which evaluates overall survival experience, was used to analyze the significance of the differences between the TTE values of two groups. Logrank analysis includes the data for all animals in a group except those assessed as NTR deaths. Statistical analyses of the differences between MF values of control and treated groups on Day 36 was accomplished using the Mann-Whitney U test. Two-tailed statistical analyses were conducted at significance level p=0.05 and not adjusted for multiple comparisons. Prism reports results as non-significant at p>0.05, significant (symbolized by “*”) at 0.01<p≤0.05, very significant (“**”) at 0.001<p≤0.01 and extremely significant (“***”) at p≤0.001. Statistical tests are tests of significance and do not provide an estimate of the magnitude of the difference between groups.

### 2. Results

### Antitumor Efficacy of BCMA-3L/20H CAR T CELLS

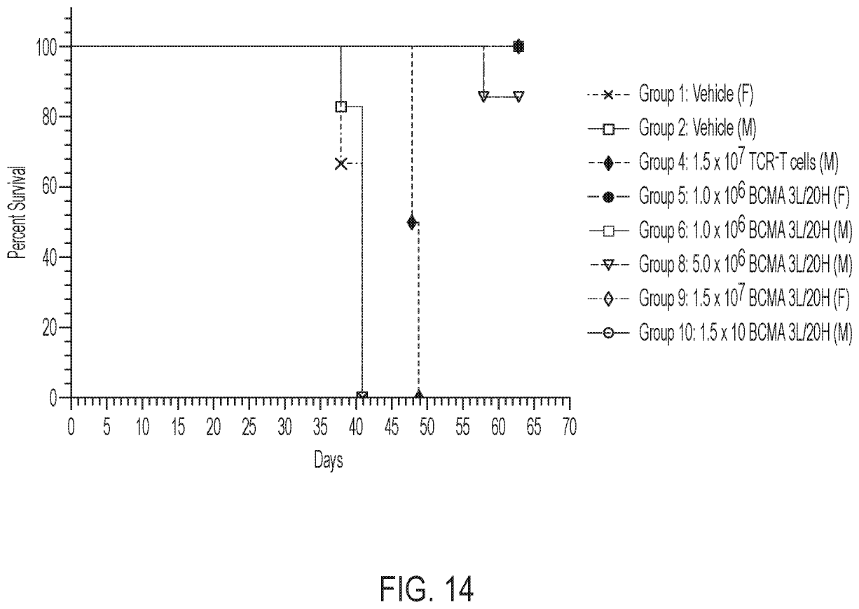

All groups were monitored for survival and bioluminescent tumor signal over time as described in the methods above. BCMA-3L/20H CAR T cells conferred a dose-dependent survival advantage over vehicle control (Group 1, female; Group 2, male) and TCR⁻ control T cell administration (Group 4, male) (FIG. 14). All animals in Groups 5 and 6 (1.0×10⁶ BCMA-3L/20H cells; female and male, respectively) and Groups 9 and 10 (1.5×10⁷ BCMA-3L/20H cells; female and male, respectively) and 5 out of 6 animals in Group 8 (5.0×10⁶ BCMA-3L/20H cells; male) survived until study end (Day 63) (Table 7).

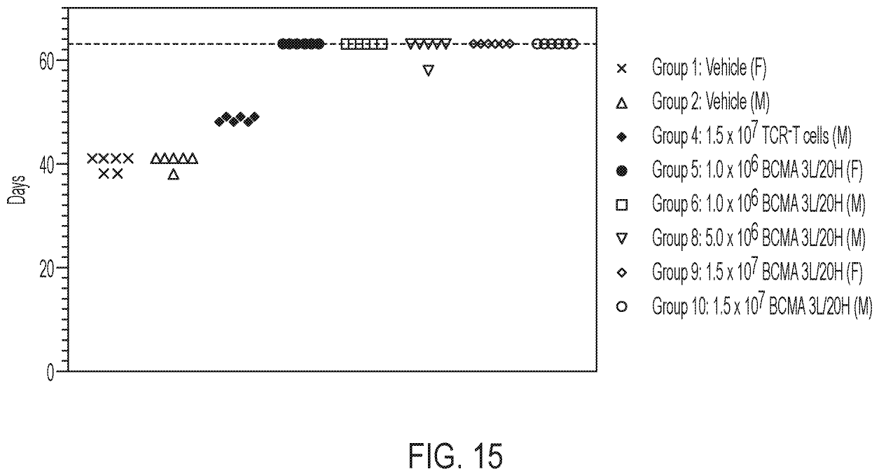

Results show an increase in TTE in BCMA-3L/20H CAR T cell-treated groups compared with vehicle control or TCR⁻ control T cell groups. The TTE for vehicle control groups (Group 1, female; Group 2, male) was 41.0 days, establishing an 18% increased life span (ILS) in TCR-control T cell Group 4 in this 63-day study. In comparison, all BCMA-3L/20H CAR T cell-treated groups demonstrated a maximum ILS of 54% (Table 7). Individual TTEs for all groups are shown in FIG. 15 and median TTEs are summarized in Table 7.

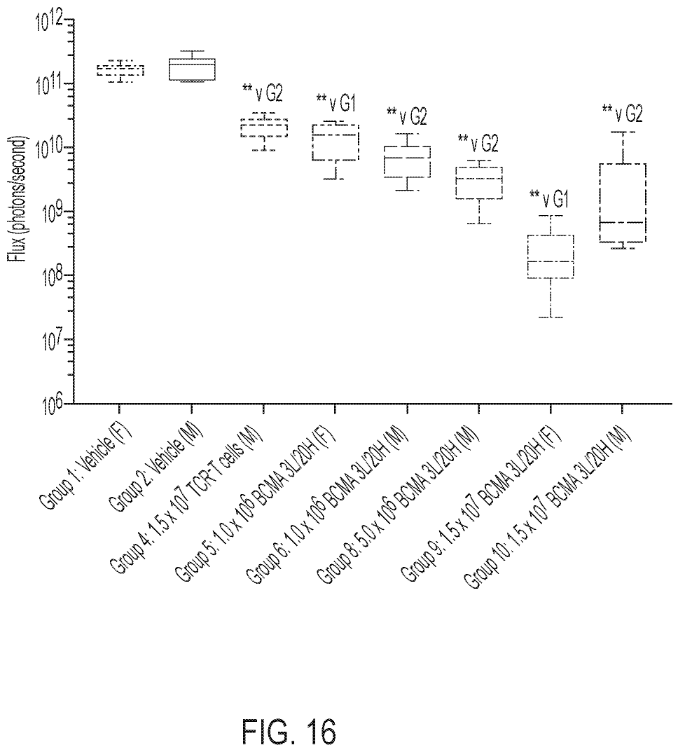

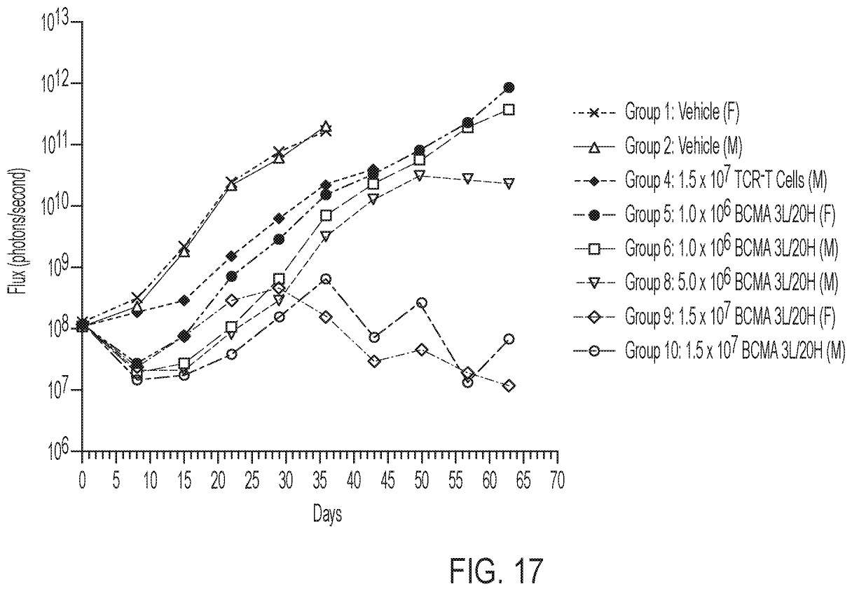

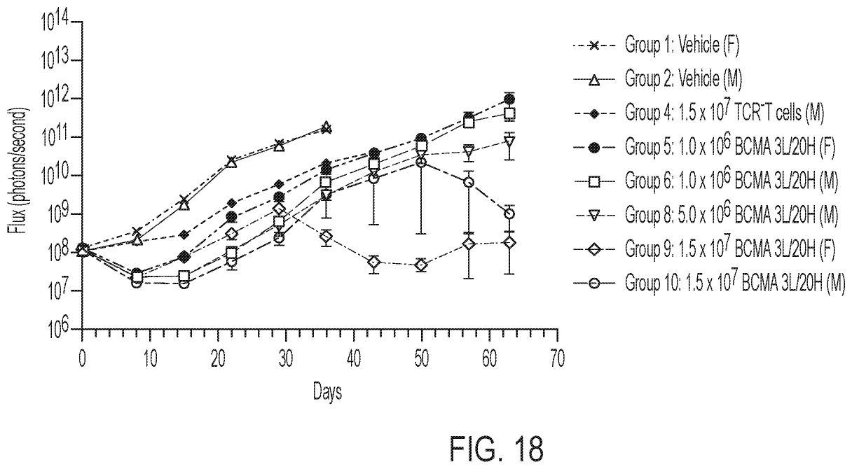

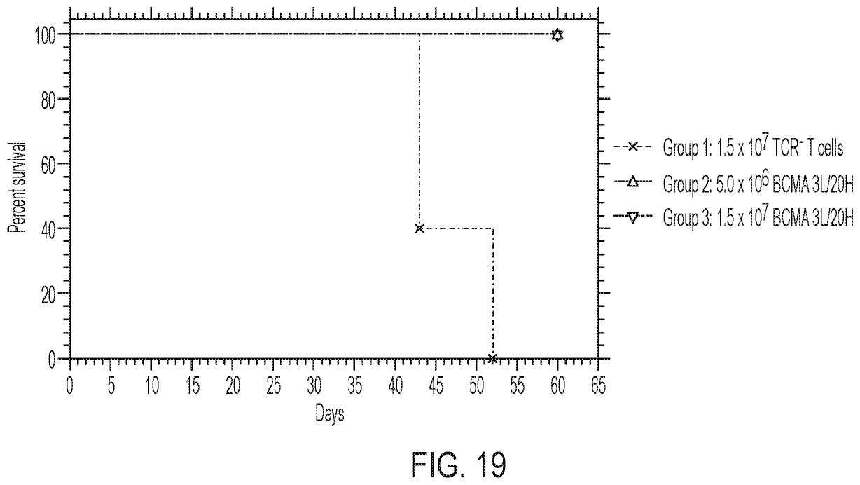

Median flux intensity values at Day 36 were significantly lower in all BCMA-3L/20H CAR T cell-treated groups in comparison to vehicle control-treated groups (Groups 1 and 2) (FIG. 16 and Table 8). Median and mean flux intensity values over time are presented in FIG. 17 and FIG. 18, respectively. All dosed groups with BCMA-3L/20H CAR T cells demonstrated statistically significant increases in TBI in comparison to respective groups administered vehicle control and TCR⁻ control T cells. TBI averaged 91% to 96%, 98%, and 100% in groups administered BCMA-3L/20H CAR T cells at doses of 1.0×10⁶, 5.0×10⁶, or 1.5×10⁷ cells per animal, respectively, in comparison to respective vehicle control groups.

By Day 41 and Day 49 post dose, all animals in the vehicle control groups and all animals in the TCR⁻ control T cell group, respectively, were euthanized after reaching defined humane endpoints (full hind limb paralysis, ruffled fur, excessive body weight loss, and hunched posture). In contrast, mice treated with BCMA-3L/20H cells at all doses demonstrated significant dose-dependent survival advantages compared to controls. This survival advantage directly correlates with reductions in median and mean flux distribution over time following treatment of BCMA-3L/20H CAR T cells (FIG. 17 and FIG. 18).

In summary, animals treated with BCMA-3L/20H CAR T cells showed tumor growth inhibition in a dose-dependent manner coincident with increased survival compared with vehicle control or TCR⁻ control T cell treatment.

TABLE 7
Summary of time to endpoint

| | | | | | | Statistical | | | n | | |
|---|---|---|---|---|---|---|---|---|---|---|---|
| | | | | ILS | | Significance | | | remaining | | |
| Group/ | | Dose | Median | vs | vs | vs | vs | vs | Day | Deaths | |
| Sex | Treatment | (cells/animal) | TTE | G1 | G2 | G1 | G2 | G4 | 63 | TR | NTR |
| 1 (F) | vehicle | NA | 41.0 | — | — | — | — | — | 0 | 0 | 0 |
| 2 (M) | vehicle | NA | 41.0 | — | — | — | — | ** | 0 | 0 | 0 |
| 4 (M) | TCR⁻ control | 1.5 × 10⁷ | 48.5 | — | 18.0 | — | ** | — | 0 | 0 | 0 |
| | T cells | | | | | | | | | | |
| 5 (F) | BCMA- | 1.0 × 10⁶ | 63.0 | 54.0 | — | ** | — | — | 6 | 0 | 0 |
| | 3L/20H CAR | | | | | | | | | | |
| | T cell low | | | | | | | | | | |
| 6 (M) | BCMA- | 1.0 × 10⁶ | 63.0 | — | 54.0 | — | ** | *** | 6 | 0 | 0 |
| | 3L/20H CAR | | | | | | | | | | |
| | T cell low | | | | | | | | | | |
| 8 (M) | BCMA- | 5.0 × 10⁶ | 63.0 | — | 54.0 | — | ** | *** | 5 | 0 | 0 |
| | 3L/20H CAR | | | | | | | | | | |
| | T cell mid | | | | | | | | | | |
| 9 (F) | BCMA- | 1.5 × 10⁷ | 63.0 | 54.0 | — | ** | — | — | 6 | 0 | 0 |
| | 3L/20H CAR | | | | | | | | | | |
| | T cell high | | | | | | | | | | |
| 10 (M) | BCMA- | 1.5 × 10⁷ | 63.0 | — | 54.0 | — | ** | *** | 6 | 0 | 0 |
| | 3L/20H CAR | | | | | | | | | | |
| | T cell high | | | | | | | | | | |

Abbreviations:
F = female;
G1 = Group 1;
G2 = Group 2;
G4 = Group 4;
ILS = increased life span;
M = male;
n = number;
NA = not applicable;
NTR = nontreatment related;
TCR = T cell receptor;
TR = treatment related;
TTE = time to endpoint
Note:
Statistical significance was calculated by Logrank test (* = p < 0.05; ** = p < 0.01; *** = p < 0.001).

TABLE 8
Summary of tumor growth inhibition

| | | | | | Statistical | | | |
|---|---|---|---|---|---|---|---|---|
| | | | Median | | Significance | | | |
| | | Dose | flux Day | | vs | vs | vs | Mean BW |
| Group/Sex | Treatment | (cells/animal) | 36 | % TBI | G1 | G2 | G4 | Nadir |
| 1 (F) | vehicle | NA | 168050 | — | — | — | — | −2.5% Day 36 |
| 2 (M) | vehicle | NA | 205850 | −22 | — | — | ** | −2.0% Day 4 |
| 4 (M) | TCR⁻ control T | 1.5 × 10⁷ | 21820 | 87 | — | ** | — | −1.0% Day 4 |
| | cells | | | | | | | |
| 5 (F) | BCMA-3L/20H | 1.0 × 10⁶ | 15430 | 91 | ** | — | — | — |
| | CAR T cell low | | | | | | | |
| 6 (M) | BCMA-3L/20H | 1.0 × 10⁶ | 6875 | 96 | — | ** | * | — |
| | CAR T cell low | | | | | | | |
| 8 (M) | BCMA-3L/20H | 5.0 × 10⁶ | 3222.5 | 98 | — | ** | ** | — |
| | CAR T cell | | | | | | | |
| | mid | | | | | | | |
| 9 (F) | BCMA-3L/20H | 1.5 × 10⁷ | 162.45 | 100 | ** | — | — | — |
| | CAR T cell | | | | | | | |
| | high | | | | | | | |
| 10 (M) | BCMA-3L/20H | 1.5 × 10⁷ | 662 | 100 | — | ** | ** | — |
| | CAR T cell | | | | | | | |
| | high | | | | | | | |

Abbreviations: BW = body weight; F = female; G1 = Group 1; G2 = Group 2; G4 = Group 4; M = male; NA = not applicable; TCR = T cell receptor; TBI = tumor burden inhibition.
Note:
Statistical significance was calculated by the Mann-Whitney U test (* = 0.01 < p ≤ 0.05; ** = 0.001 < p ≤ 0.01).

### Adverse Events

No TR or NTR deaths were observed in this study; all animal deaths in this study are classified as DSS.

### Gross Pathology

Necropsy findings were identified in all groups. No animals administered vehicle control (Groups 1 and 2) or males administered TCR− control T cells (Group 4) survived to the end of study. All animals from Groups 1, 2, and 4 were euthanized before study end due to reaching defined humane endpoints (body weight loss, hindlimb paralysis, ruffled fur, and hunched posture) due to disease progression. Upon euthanization, gross necropsy was performed by a trained technician. These animals (Groups 1, 2, and 4) were observed with brain swelling and fluid surrounding the brain, a common historical finding in mice engrafted with MM.1S cells. Some mice in Groups 1 and 4 also showed mottled and darkened livers.

With the exception of 1 mouse in Group 8 (5.0×10⁶ BCMA-3L/20H cells, male), all mice treated with BCMA-3L/20H CAR T cells survived to the end of study. Animal No. 6 from Group 8 was euthanized on Day 58 of the study. Upon necropsy by a trained technician it was noted that this animal appeared to have a small amount of fluid in the cranial cavity. For all other mice in the BCMA-3L/20H CAR T cell-treated groups the gross necropsy was performed by a board-certified pathologist on the last day of the study (Day 63). It was noted that mice in Groups 5 and 6 (1.0×10⁶ BCMA-3L/20H cells, female and male, respectively) were observed with increased tissue mass in the abdominal cavity. The increased tissue was found around several organs including the uterine horn and ovaries in females, and kidney, adrenal gland, intestines, and mesentery in both males and females. It was noted that the increased tissue mass did not appear to originate from any of the organs around which it was observed. It is believed the increased tissue mass is due to MM.1S tumor cell metastasis in the abdominal cavity, which is evidenced by the localization of luminescence intensity in the abdominal cavity in these groups of mice. One animal in Group 8 (5.0×10⁶ BCMA-3L/20H cells, male, Animal No. 1) was observed with an increase in tissue mass in the mesentery; however, it was not as frequently observed as that observed in animals from Groups 5 and 6 (1.0×10⁶ BCMA-3L/20H cells, female and male, respectively). Subcutaneous masses in the flank, neck, and shoulder regions were also noted in animals from Groups 5 and 6 (1.0×10⁶ BCMA-3L/20H cells, female and male, respectively). It is believed these masses are due to MM.1S cell metastasis.

Animals in Groups 5 and 6 (1.0×10⁶ BCMA-3L/20H cells, female and male, respectively), Group 8 (5.0×10⁶ BCMA-3L/20H cells, male), and Groups 9 and 10 (1.5×10⁷ BCMA-3L/20H cells, female and male, respectively) were observed to have an enlarged thymic area less than 1 cm that was firm, tan, and contained black foci. The tissue in the thymic region was not considered to be a lesion, but an enhancement of the epithelial or adipose tissue often seen with aging in NSG mice. It is noted that this observation was not identified in Groups 1 and 2 (vehicle control treated mice) or Group 4 (TCR⁻ control T cells). On the final day of the study (Day 63) when BCMA-3L/20H CAR T cell-treated mice were euthanized, a board-certified pathologist performed the gross necropsy. However, when Groups 1 and 2 (vehicle control) or Group 4 (TCR⁻ control T cells) were euthanized due to humane endpoints (body weight loss, hindlimb paralysis, ruffled fur, and hunched posture), a trained technician performed the necropsy. Lack of observation of the enlarged thymic region in Groups 1 and 2 (vehicle treated mice) or Group 4 (TCR⁻ control T cells) is likely due to different observers with different levels of training performing the necropsy and observations. Another possibility is that the increased age of the mice on Day 63 could have caused the differences in the thymic region noted above. Older animals are more likely to have more adipose tissue in the thymic region than their younger counterparts that exited the study earlier and the board-certified pathologist did not consider the enlarged thymic region to be a lesion of an adverse finding.

Animals in Groups 5 and 6 (1.0×10⁶ BCMA-3L/20H cells, female and male, respectively), Group 8 (5.0×10⁶ BCMA-3L/20H cells, male) and Groups 9 and 10 (1.5×10⁷ BCMA-3L/20H cells, female and male, respectively) were observed having dilated small intestines with minimal lumen contents from the duodenum to the proximal jejunum with a tan color. This was not considered an adverse finding by the pathologist. As noted with the enlarged thymic region, this observation was not identified in Groups 1 and 2 (vehicle control) or Group 4 (TCR⁻ control T cells). Again, this is more than likely due to different observers with different levels of training performing the necropsy and observations at different times of the study.

In summary, all mice in the vehicle control-treated groups (Groups 1 and 2) or the TCR⁻ control T cell-treated group (Group 4) were euthanized before study end due to reaching humane endpoints (body weight loss, hindlimb paralysis, ruffled fur, and hunched posture) associated with disease progression. A majority of these mice showed increased brain swelling upon necropsy. In contrast, all mice treated with BCMA-3L/20H CAR T cells, with exception of 1 mouse in Group 8 (5.0×10⁶ BCMA-3L/20H cells, male), survived until the end of the study (Day 63). Mice in the low dose treatment Groups 5 and 6 (1.0×10⁶ BCMA-3L/20H cells, female and male, respectively) were observed having an increase in tissue mass around organs in the abdominal cavity, which included ovary, kidney, adrenal gland, and mesentery. Mice in Groups 5 and 6 also showed an increase in subcutaneous masses in the flank, neck, and shoulder region. The increase in tissue and subcutaneous masses was believed to be derived from metastasis of the engrafted MM.1S cells. It should be noted that all mice in the high dose treatment Groups 9 and 10 (1.5×10⁷ BCMA-3L/20H cells, female and male, respectively) and the majority of mice (5 out of 6) in the mid-dose treated Group 8 (5.0×10⁶ BCMA-3L/20H cells, male) had no increases in tissue mass, suggesting that treatment with BCMA-3L/20H CAR T cells is not only able to increase survival but also decrease tissue enhancement at distal sites. No other observations were deemed adverse or remarkable in this study.

### Histopathological Evaluation and Immunohistochemistry

All male and female NSG mice receiving vehicle control had diffuse myeloma as evidenced by H&E staining and observed throughout the bone marrow, completely replacing the bone marrow and infiltrating throughout the bone. hCD45⁺ mononuclear cells were not detected in the bone marrow from the vehicle control-treated animals, consistent with the lack of human T cell administration in these animals. Vehicle control-treated animals reached terminal endpoint in 41 days, compared to TCR⁻ control T cell animals (48.5 days) and BCMA-3L/20H CAR T cell-treated animals (63 days).

All male NSG mice that received 1.5×10⁷ TCR⁻ control T cells had very rare (<1%) hCD45⁺ mononuclear cells within the bone marrow. The hCD45⁺ mononuclear cells were found individually and randomly scattered throughout the bone marrow. In 2 males (Group 4, Animal No. 5 and 6), rare (1 to 5%) hCD45⁺ mononuclear cells were concentrated in a focus of myeloma in the bone marrow. The bone marrow in the other animals receiving TCR⁻ control T cells was histologically within normal limits for NSG mice. Administration of a large bolus of TCR⁻ T cells (1.5×10⁷ cells) to NSG mice, which are severely immunocompromised, could result in competition for engraftment with MM.1S cells in a limited niche such as the bone marrow. Despite this, animals in the group receiving TCR⁻ T cells showed significantly decreased survival and failed to reach study endpoint due to manifestation of clinical symptoms (body weight loss, hindlimb paralysis, ruffled fur, and hunched posture) compared to mice receiving the same dose of BCMA-3L/20H cells. These results reflect the disseminated nature of the MM.1S model as previously published (Chu et al., Clin Cancer Res. 2014;(15):3989-4000) and aligns with the detectable high systemic BLI signal observed in TCR⁻ T cell-treated animals prior to euthanasia. Collectively, while there does not appear to be evidence of myeloma in the bone marrow of the remaining 4 mice administered TCR⁻ T cells (Animal No. 1 to 4), the MM.1S cells were still able to cause an oncogenic profile that resulted in the early termination of mice treated with TCR⁻ control T cells relative to BCMA-3L/20H CAR T cell treated mice.

All male and female NSG mice administered 1.0×10⁶ BCMA-3L/20H cells showed very rare (<1%) presence of hCD45⁺ mononuclear cells within the bone marrow, except 1 male (Group 6, Animal No. 4) which showed no presence of hCD45⁺. The hCD45⁺ mononuclear cells were found individually and randomly scattered throughout the bone marrow. The bone marrow in animals administered 1.0×10⁶ BCMA-3L/20H cells was histologically within normal limits for NSG mice, except a focal chronic thrombus present in 1 male (Group 6, Animal No. 2).

The bone marrow from all males administered 5.0×10⁶ BCMA-3L/20H cells (Group 8) was histologically within normal limits for NSG mice. hCD45⁺ mononuclear cells were observed very rarely (<1%), individually and randomly scattered throughout the bone marrow from these animals.

All male and female NSG mice administered 1.5×10⁷ BCMA-3L/20H cells showed very rare (<1%) presence of hCD45⁺ mononuclear cells within the bone marrow. hCD45+ mononuclear cells were observed individually and randomly scattered throughout the bone marrow. The bone marrow in all animals administered 1.5×10⁷ BCMA-3L/20H cells was histologically within normal limits for NSG mice.

In summary, myeloma was not evident in the bone marrow from the majority of male and female NSG mice administered 1.5×10⁷ TCR⁻ control T cells or 1.0×10⁶, 5.0×10⁶, or 1.5×10⁷ BCMA-3L/20H cells, in contrast to evidence of myeloma in the bone marrow of animals administered vehicle control. In animals treated with TCR⁻ control T cells or BCMA− 3L/20H CAR T cells, the presence of hCD45+ mononuclear cells was very rare (<1%) to rare (1% to 5%) and observed individually and randomly scattered throughout the bone marrow.

Diffuse myeloma was observed in the bone marrow of all male and female mice administered vehicle control, which may have contributed to the earlier deaths in the vehicle control-treated groups in comparison to animals administered TCR⁻ control T cells or BCMA-3L/20H CAR T cells. Focal myeloma was observed in the bone marrow from 2 males administered 1.5×10⁷ TCR⁻ control T cells.

### 3. Conclusions

This efficacy study evaluated BCMA-3L/20H CAR T cells for antitumor efficacy in NSG mice bearing disseminated BCMA⁺MM.1S multiple myeloma tumors that stably express luciferase. Antitumor efficacy was evaluated by BLI and comparison of survival among vehicle control, TCR⁻ control T cells, and BCMA-3L/20H CAR T cells treatment groups.

BCMA-3L/20H CAR T cell treatment demonstrated a statistically significant and dose-dependent survival advantage over TCR⁻ control T cell or vehicle control treatments. By Day 41 and Day 49 postdose, all animals in the vehicle control groups and all animals in the TCR⁻ control T cell group, respectively, were euthanized after reaching defined humane endpoints (full hind limb paralysis, ruffled fur, hunched posture). In contrast, mice treated with BCMA-3L/20H CAR T cells showed no overt signs of moribundity and demonstrated a statistically significant and dose-dependent inhibition of tumor burden confirmed by BLI measurements. TBI reached statistical significance in each of the BCMA-3L/20H CAR T cell treatment groups compared to respective concurrent controls in the vehicle control and TCR⁻ control T cell groups. TBI averaged 91% to 96%, 98%, and 100% in groups administered BCMA-3L/20H CAR T cells at doses of 1.0×10⁶, 5.0×10⁶, or 1.5×10⁷ CAR T cells per animal, respectively. Median TTE in all BCMA-3L/20H CAR T cell groups was 63.0 days (end of study). By comparison, the median TTE of animals in the vehicle control groups was 41.0 days.

These results show that BCMA-3L/20H CAR T cells mediate clearance of BCMA+tumors in vivo, demonstrating significant and dose-dependent inhibition of tumor growth and conferring significant survival advantages in comparison to controls.

### Example 10

Pharmacokinetic/Pharmacodynamic Study of BCMA-3L/20H CAR T cells In a Disseminated Multiple Myeloma Model

### 1. Methods

To assess the PK and biodistribution of BCMA-3L/20H CAR T cells in a BCMA⁺ myeloma tumor cell model, this study was conducted in NSG mice implanted IV with luciferase-expressing BCMA⁺MM.1S multiple myeloma cells (MM.1S-ffLuc). The route of BCMA-3L/20H CAR T cell administration in this study was IV, which is the intended route of administration in humans. Doses were selected based on investigative study results that demonstrated tumor burden reduction and a survival advantage in tumor-bearing NSG mice administered BCMA-3L/20H CAR T cells compared with tumor-bearing NSG mice administered vehicle control or TCR⁻ control T cells. The objective of this study was to determine the PK of BCMA-3L/20H CAR T cells in a disseminated myeloma xenograft model following intravenous injection.

### Study Design

The PK and efficacy of BCMA-3L/20H CAR T cells against MM.1S-ffLuc in a mouse xenograft model was evaluated. Examples of processes implemented in this study include a prospective protocol, judicious but appropriate numbers of animals per group, test article controls, necropsy conducted by trained personnel, study supervision conducted by a study director and study monitor, and causes of death determined where feasible. Additionally, the animal study groups (e.g., group numbers and dosing preparation solutions) were coded to maintain client confidentiality during study procedures.

Female NSG mice were IV injected in the tail vein with 2.5×10⁶ MM.1S-ffLuc cells. On Day 1 (8 days post-implantation), mice with established MM.1S-ffLuc tumors were dosed IV with either TCR⁻ control T cells (negative efficacy control) or BCMA-3L/20H CAR T cells (test article) (Table 9) to assess efficacy and PK parameters.

TABLE 9
In vivo efficacy study design

| | | | Dose | | Schedule for IV |
|---|---|---|---|---|---|
| Group | n/sex | Treatment | (cells/animal) | Route | Dosing |
| 1 | 10/F | TCR⁻ control T cells (NP11)^{a)} | 1.5 × 10⁷ | IV | QD × 1 |
| 2 | 10/F | BCMA-3L/20H CAR T cell | 5.0 × 10⁶ | IV | QD × 1 |
| | | low (NP10)^{b)} | | | |
| 3 | 10/F | BCMA-3L/20H CAR T cell | 1.5 × 10⁷ | IV | QD × 1 |
| | | high (NP10)^{b)} | | | |

Abbreviations: F = female; IV = intravenous; QD = once a day; TCR = T cell receptor.
^{a)}Designation code given for client confidentiality .
^{b)}Designation code given for client confidentiality.
Note:
BCMA-3L/20H CAR T cell doses represent CAR T cells per animal.

### Mice

Female NSG mice (NOD.Cg-Prkdc^{scid}I2rgtm^{tmIWjI}/SzJ, The Jackson Laboratory) were 10 weeks old with body weights ranging from 20.3 to 26.5 grams at the beginning of the study (Day 1). The study complied with the recommendations of the Guide for Care and Use of Laboratory Animals with respect to restraint, husbandry, surgical procedures, feed and fluid regulation, and veterinary care. The study program was accredited by the Association for Assessment and Accreditation of Laboratory Animal Care International, which assures compliance with accepted standards for the care and use of laboratory animals.

### Formulation of Test and Control Articles

BCMA-3L/20H CAR T cells (TCR⁻CAR⁺, DEMO 32) and TCR⁻ control T cells (TCR⁻CAR⁻, Batch No. 13) were generated from the same leukapheresis donor. The cells were supplied as frozen in cryopreservation media and formulated to the appropriate concentration with supplied diluent.

Pre- and post-injection viability was 92.9% and 91.7%, respectively, for the TCR⁻ control T cells and 91.2% and 90.3% (5×10⁶), and 92.7% and 91.9% (1.5×10⁷), respectively, for the BCMA-3L/20H CAR T cell doses.

TABLE 10
Test articles

| Material | Batch No. |
|---|---|
| BCMA-3L/20H CAR T cells (NP10) | Demo 32 |
| TCR- control T cells (NP11) | Demo 13 |
| Diluent^{a)} | 64 |

Abbreviations: CAR = chimeric antigen receptor; HSA = human serum albumin; NA = not applicable; TCR = T cell receptor.
^{a)}Diluent: Plasma-Lyte A, 2% HSA.

### Intravenous Tumor Cell Injection

Frozen vials containing a clone of MM.1S multiple myeloma cells that stably express firefly luciferase (MM.1S-ffLuc) were utilized for this study. Cells were thawed and cultured in RPMI-1640 medium containing 15% fetal bovine serum (FBS), 2 mM glutamine, 100 units/mL penicillin G sodium, 100 g/mL streptomycin sulfate, and 25 g/mL gentamicin. The tumor cells were cultured in tissue culture flasks in a humidified incubator at 37° C., in an atmosphere of 5% CO₂ and 95% air. Cells were washed, counted, viability determined, and resuspended in sterile phosphate buffered saline (PBS). Each mouse was injected IV in a tail vein with 2.5×10⁶ MM.1S-ffLuc cells in a 0.2 mL suspension. It was determined that the optimal time to begin treatment with test agents was 8 to 9 days after tumor implantation. Eight days after tumor implantation (designated as Day 1 of the study), the animals were randomized into 3 groups (n=10/group) based on tumor burden (BLI flux values).

### Treatment

Day 1 (8 days post-implantation) of the study designates the day of dosing of the control and test articles (TCR⁻ control T cells, or BCMA-3L/20H CAR T cells) according to the treatment protocol summarized in Table 10. The dosing volume was a fixed 0.2 mL/mouse by IV injection.

### Imaging Methods

In vivo BLI was performed on Day 1 (8 days post-implantation) and once a week thereafter to Day 60. Whole body ventral images were captured 10 minutes after substrate injection. Luciferase activity was measured in live animals using IVIS® SpectrumCT (Perkin Elmer, MA) equipped with a CCD camera (cooled at −90° C.), mounted on a light-tight specimen chamber. On the day of imaging, animals received intraperitoneal injections with luciferin substrate (total 150 mg/kg) and were placed in an anesthesia induction chamber (2.5 to 3.5% isoflurane in oxygen). Upon sedation, animals were positioned in the imaging chamber, equipped with a stage heated at physiological temperature, for image acquisition 10 minutes onwards post-luciferin substrate injection.

Regions of interest were drawn around each mouse image, and flux was quantified and reported as 10⁶ photons per second (p/s). Data was analyzed and exported using Living Image software 4.5.1. (Perkin Elmer, MA). Representative images were defined as animals with whole body flux values closest to the calculated median at the timepoint when >50% of the animals of a group at a given time point remained on study.

When an animal exited the study due to tumor burden or clinical signs of supporting moribundity (excessive body weight loss, hind limb paralysis, hunched posture, ruffled fur, ocular proptosis), the final flux value recorded was carried forward and included with the data to calculate the group mean and median intensities at subsequent time points until <50% of the animals remain. Any animal that died due to nontreatment-related (NTR) deaths attributed to accidental (NTRa) or unknown etiology (NTRu) were excluded from the analysis data set.

### Endpoint and Tumor Burden Inhibition Analysis

Treatment efficacy based on tumor burden inhibition (TBI) was determined using data from Day 43. The median flux (MF) for the number of animals (n) on this day was determined for each group. Percent TBI was defined as the difference between the MF of the designated control group and the MF of the drug-treated group, expressed as a percentage of the MF of the control group (TCR⁻ control T cells):

$$\% TBI = [ 1 - ( {MF}_{drug - treated} / {MF}_{control} ) ] \times 100$$

The data set for TBI analysis includes all animals in a group, except those that die due to treatment-related (TR) or NTR causes.

### Endpoint for Survival Study

Animals were monitored individually for an endpoint of moribundity due to progression of tumor burden. The time to endpoint (TTE) was recorded for each mouse that died of its disease or was euthanized due to disease progression; these deaths were assigned TTE values equal to the day of death or euthanasia. Median TTE values were calculated for each group. Animals that survived to the end of the study were euthanized and were assigned a TTE value equal to the last day of the study. The median TTE of treated mice is expressed as a percentage of the median TTE of control mice (% T/C), and the increase in life span (ILS) is calculated as:

$$ILS = \% T / C - 100 \% , \\ where T = median {TTE}_{treated} , and \\ C = median {TTE}_{control} . \\ Thus , if T = C , ILS = 0 \% .$$

### Clinical Observations

Animals were weighed daily from Day 1 to Day 5, then 3 times per week until the completion of the study. The mice were observed frequently for overt signs of any TR side effects, and clinical observations were recorded. Individual body weight was monitored as per protocol, and any animal with weight loss exceeding 30% for 1 measurement or exceeding 25% for 3 consecutive measurements was euthanized as a TR death (for treated groups).

Group mean body weight loss was also monitored. An animal death was classified as death on a survival study (DSS) if animals presented with full hindlimb paralysis, ocular proptosis, excessive body weight loss, or moribundity due to tumor progression. An animal death was classified as TR if the death was attributable to treatment side effects as evidenced by clinical signs and/or necropsy. A TR classification was also assigned to deaths by unknown causes within 14 days of the administered dose. A death was classified as NTR if there was no evidence that death was related to treatment side effects and death was not classified as DSS. No TR or NTR deaths were observed in this study; all animal deaths in this study were classified as DSS.

### Sampling

On Days 3, 10, and 17, whole blood was collected by mandibular bleeds under no anesthesia into K₂EDTA tubes and analyzed by flow cytometry. At study endpoint (either at moribundity or Day 60), animals were sampled for flow cytometry.

An appropriately-trained technician performed and recorded the necropsy for each individual animal just after terminal blood collection but before organ collection. Full volume blood of each animal was collected by terminal cardiac puncture under isoflurane anesthesia and prepared for flow cytometry. Immediately after necropsy, bone marrow and spleen were removed, the spleen was weighed, and both the spleen and bone marrow were prepared for flow cytometry.

### Flow Cytometry Sample Processing and Analysis

Mouse blood and bone marrow samples were processed by adding 10× volume of room temperature ACK lysis buffer to blood samples. Samples were gently mixed and incubated for 3 to 5 minutes at room temperature. Immediately after incubation time, a 10× volume of cold PBS was added to stop the lysis reaction. Samples were centrifuged at 400×g for 5 minutes. An additional PBS wash was performed on samples. Mouse spleen samples were dissociated by gently grinding the tissue across fine mesh with RPMI-1640 media and lysing red blood cells with ACK lysis buffer according to the manufacturer's instructions. Samples were centrifuged and washed twice with PBS.

The final single cell suspensions were prepared in PBS pH 7.4 at 2.0×10⁷ cells/mL and held briefly on ice prior to in-house flow cytometry analysis.

Single cell suspensions were added into 96-well plates and stained for 30 minutes at 4° C. with 100 μL of the reconstituted Live/Dead Aqua (Life Technologies) following manufacturer's instructions. After 2 washes with 150 μL of Staining Buffer, fragment 30 crystallizable region (Fc) receptors were blocked using TruStain FcX (BioLegend) in 100 p L volume for 5 to 10 minutes on ice prior to immunostaining. Cells were stained for 30 minutes at 4° C. with the appropriate antibody panels. Cells were washed twice with 150 μL of Staining Buffer and resuspended in 100 μL of Staining Buffer for analysis. Isotype-control antibodies were used as negative staining controls when deemed necessary. All data were collected on a Fortessa LSR (BD) and analyzed with FlowJo software (version 10.0.7r2, Tree Star, Inc.). Cell populations were defined and the gating strategy was determined by initial gating on singlets (FSC-H vs. FSC-A) and then live cells, based on Live/Dead Aqua viability staining

### Statistical and Graphical Analyses

Prism 8.1.1 (GraphPad) for Windows was employed for statistical and graphical analyses. Study groups experiencing clinical endpoints beyond acceptable limits (>20% group mean body weight loss or greater than 10% treatment-related deaths) or having fewer than 5 evaluable observations, were nonevaluable. The logrank test, which evaluates overall survival experience, was used to analyze the significance of the differences between the TTE values of two groups. Logrank analysis includes the data for all animals in a group except those assessed as NTR deaths. Statistical analyses of the differences between median flux values (MFs) of TCR⁻ control T cell and BCMA-3L/20H CAR T cell-treated groups on Day 43 was accomplished using the Mann-Whitney U test. A two-tailed statistical analysis was conducted at significance level p=0.05 and not adjusted for multiple comparisons. Prism reports results as non-significant at p>0.05, significant (symbolized by “*”) at 0.01<p≤0.05, very significant (“**”) at 0.001<p≤0.01 and extremely significant (“***”) at p≤0.001. Statistical tests are tests of significance and do not provide an estimate of the magnitude of the difference between groups. JMP® 14.3 was used for graphical presentations and statistical analyses of flow cytometry data using ANOVA with a Tukey post-hoc test.

### 2. Results

All groups were monitored for survival, and bioluminescent tumor signal over time. BCMA-3L/20H CAR T cells conferred a survival advantage over TCR⁻ control T cells.

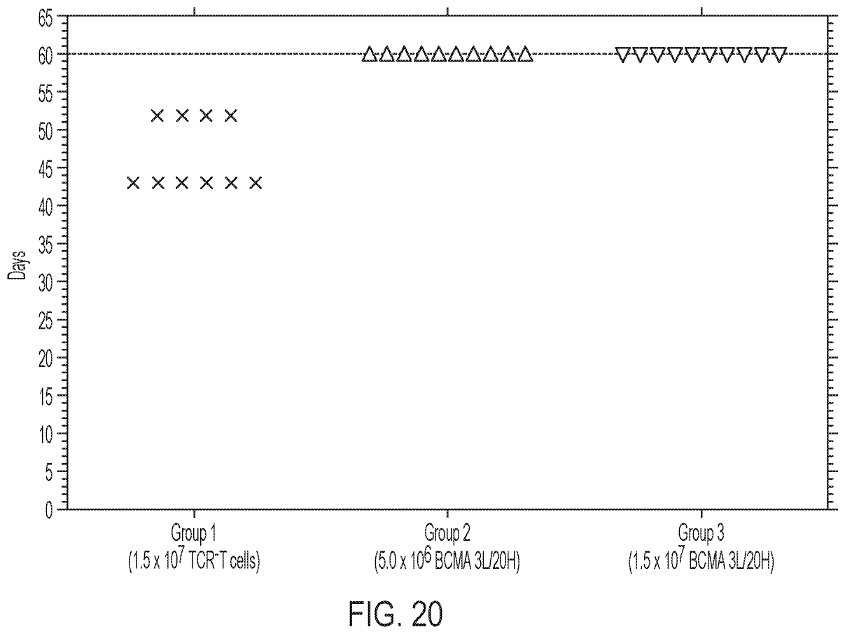

All animals in Group 2 (5×10⁶ BCMA-3L/20H cells) and Group 3 (1.5×10⁷ BCMA-3L/20H cells) survived until study end (Day 60) (Table 11). Results show an increase in TTE in BCMA-3L/20H CAR T cell-treated groups compared with the TCR⁻ control T cell group. The median TTE for TCR⁻ control T cell group was 43.0 days in comparison to a median TTE of 60.0 days (end of study) for animals treated with BCMA-3L/20H CAR T cells, resulting in a maximum ILS of 40% (Table 11). All BCMA-3L/20H CAR T cell-treated animals, regardless of dose, reached the maximum ILS of 40%. Individual TTEs for all groups are shown in FIG. 20 and median TTEs are summarized in Table 11.

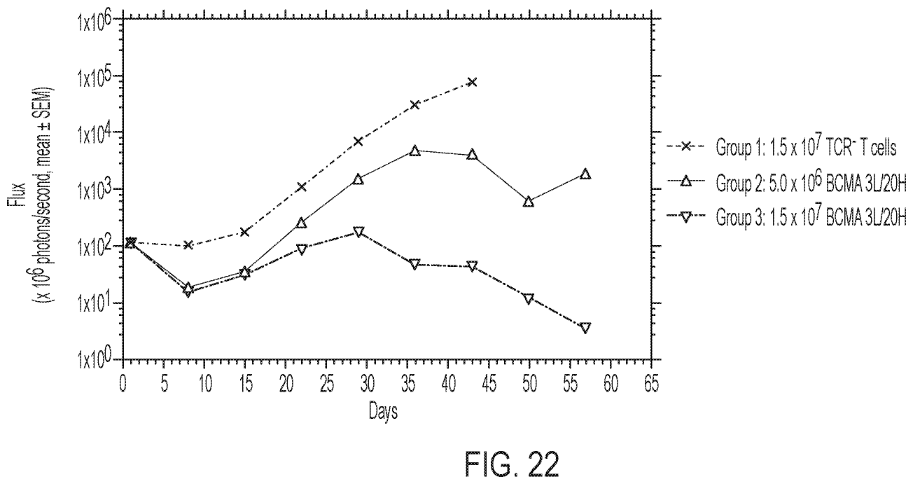

Median flux intensity values at Day 43 were significantly lower in both BCMA-3L/20H CAR T cell-treated groups (Groups 2 and 3) relative to the TCR⁻ control T cell group (Group 1) (FIG. 3 and Table 11). Mean flux intensity values over time are shown in FIG. 4. The mean BLI flux values for Group 2 (5.0×10⁶ BCMA-3L/20H cells) and Group 3 (1.5 ×10⁷ BCMA-3L/20H cells) initially decreased on Day 8, then showed a dose-dependent response whereby Group 2 (5.0×10⁶ BCMA-3L/20H cells) exhibited a delayed, but progressive increase in flux to the study end (FIG. 22). However, the Group 3 (1.5×10⁷ BCMA-3L/20H cells) mean flux remained relatively stable throughout the study (FIG. 22).

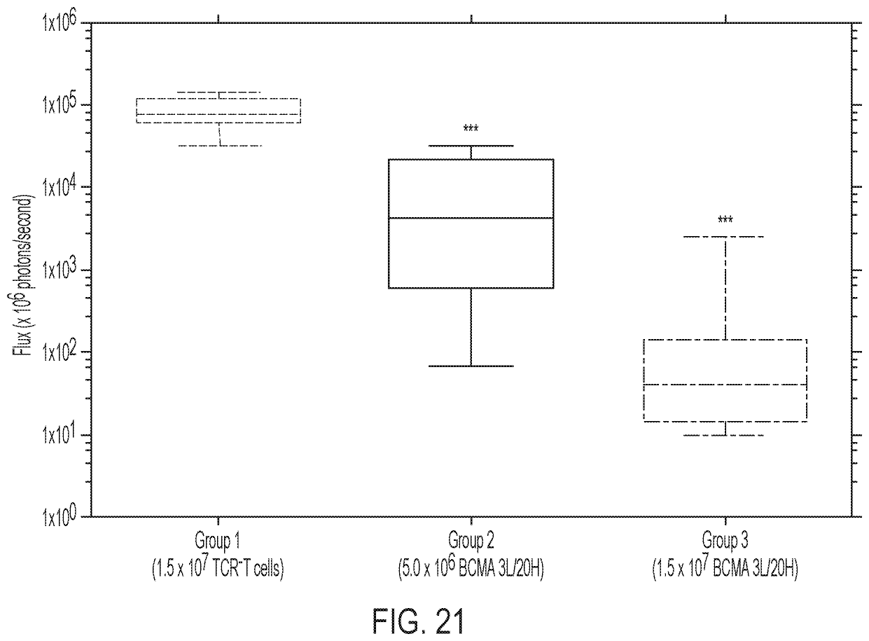

Both BCMA-3L/20H CAR T cell dose groups demonstrated statistically significant increases in TBI in comparison to the control group administered TCR⁻ control T cells (Table 11). Mean TBIs were 94% and 100%, respectively, for mice in Group 2 (5×10⁶ BCMA− 3L/20H cells) and mice in Group 3 (1.5×10⁷ BCMA-3L/20H cells). The Group 2 and 3 TBIs were both significant when compared to the TCR⁻ control T cell group, and the comparison between Groups 2 (5.0×10⁶ BCMA-3L/20H cells) and 3 (1.5×10⁷ BCMA-3L/20H cells) was also statistically significant (Table 11 and FIG. 21, p<0.001).

In summary, animals treated with BCMA-3L/20H CAR T cells at both the low dose (5.0×10⁶ BCMA-3L/20H cells) and the high dose (1.5×10⁷ BCMA-3L/20H cells) showed significant tumor burden inhibition in a dose dependent manner coincident with a significant increase in survival compared with animals administered TCR⁻ control T cells.

TABLE 11
Summary of TTE, ILS, median flux, and TBI

| | | | | | | | | Statistical | | | |
|---|---|---|---|---|---|---|---|---|---|---|---|
| | | | | | | Median | | Significance | | | |
| Group/ | | Dose | Median | | Statistical | flux | Percent | vs | vs | Deaths | |
| Sex | Treatment | (cells/animal) | TTE | ILS | significance | (Day 43) | TBI | G1 | G2 | TR | NTR |
| 1 | TCR⁻ control | 1.5 × 10⁷ | 43.0 | — | — | 77465 | — | — | *** | 0 | 0 |
| (F) | T cells | | | | | | | | | | |
| 2 | BCMA- | 5.0 × 10⁶ | 60.0 | 40 | *** | 4364.5 | 94 | *** | — | 0 | 0 |
| (F) | 3L/20H CAR | | | | | | | | | | |
| | T cells | | | | | | | | | | |
| 3 | BCMA- | 1.5 × 10⁷ | 60.0 | 40 | *** | 42.57 | 100 | *** | *** | 0 | 0 |
| (F) | 3L/20H CAR | | | | | | | | | | |
| | T cells | | | | | | | | | | |

Abbreviations: F = female; G1 = Group 1; G2 = Group 2; ILS = increased lifespan; NTR = nontreatment related; TBI = tumor burden inhibition; TCR = T cell receptor; TR = treatment related; TTE = time to endpoint.
Notes:
Statistical significance was calculated by the Mann-Whitney U test (*** = p < 0.001).
Median flux: Median flux (×10⁶ photons/sec) for the number of animals on the day of TBI analysis.

### Flow Cytometry Analysis

The PK of BCMA-3L/20H CAR T cells was assessed in this study. Flow cytometry analysis of blood, bone marrow, and spleen was performed to evaluate the relative frequencies of T cells (using anti-human CD45, CD4, and CD8 antibodies) and associated expression of exhaustion markers (using anti-PD1, LAG3, and TIM3 antibodies). In addition, 5 frequencies of MM.1S tumor cells (using an anti-human BCMA antibody) was also assessed to correlate with BLI and survival data. Survival blood sampling was conducted on Days 3, 10, and 17 post-BCMA-3L/20H CAR T cell or control T cell injection for all groups; terminal sampling was conducted on Days 43 and 52 for Group 1 (TCR⁻ control T cells) and on Day 60 for Group 2 (5.0×10⁶ BCMA-3L/20H CAR T cells) and Group 3 (1.5×10⁷ BCMA-3L/20H CAR T cells).

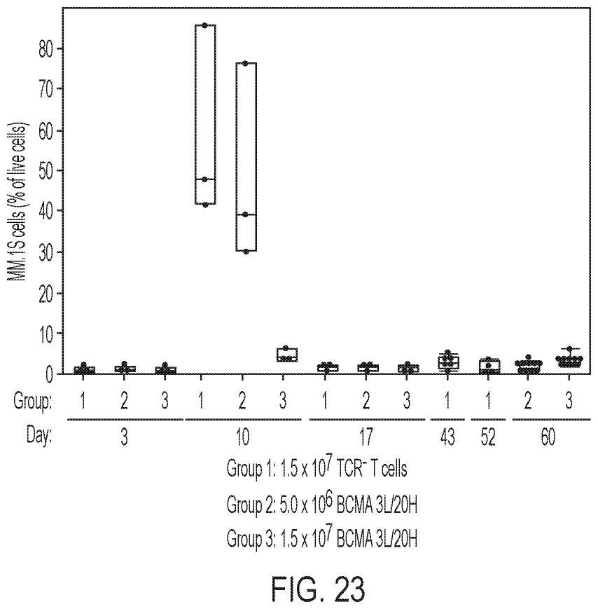

Low frequencies of BCMA⁺MM.1S tumor cells were detected in Day 3 blood samples, with the highest peripheral frequencies detected at Day 10 (FIG. 23). Frequencies of blood resident MM.1S cells correlates with initial engraftment of MM.1S tumor cells in the bone marrow and subsequent dissemination to peripheral sites, as is characteristic of this model (Chu et al., Clin Cancer Res. 2014;(15):3989-4000). Specifically, at Day 10 mean BCMA⁺MM.1S tumor frequencies were approximately 60% of live cells in the blood of animals treated with TCR⁻ control T cells and approximately 50% of live cells in the blood of animals treated with 5.0×10⁶ BCMA-3L/20H cells. Additionally, high dose BCMA-3L/20H CAR T cells (1.5×10⁷ cells) recipients had substantially lower (5%) frequencies of BCMA⁺cells in the blood (FIG. 23), demonstrating both a dose-dependent effect of BCMA-3L/20H CAR T cells and specific in vivo efficacy of BCMA-3L/20H CAR T cells compared to the control T cell population. The observations for peripheral BCMA⁺ tumor cell frequencies at Day 10 correlated with long-term BLI measurements and tumor growth.

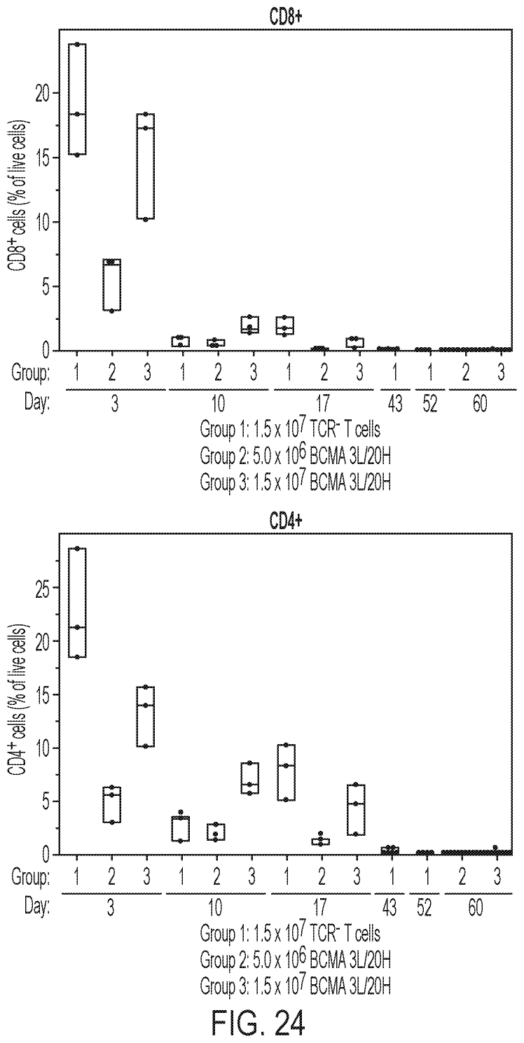

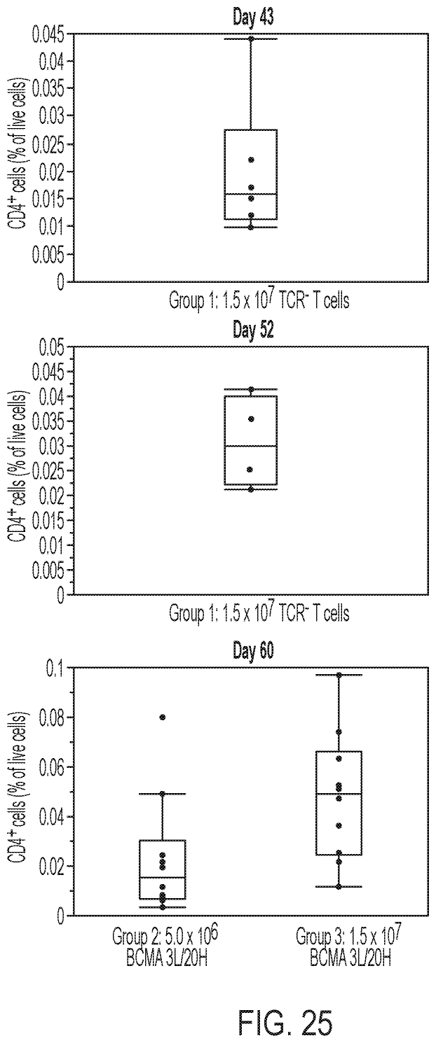

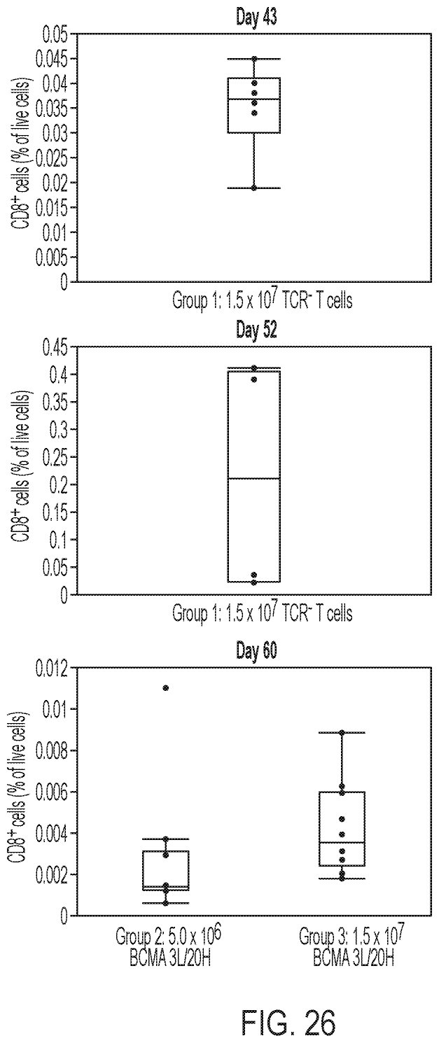

Peak detection of CD4+ and CD8+ T cells in the blood was observed at Day 3, with measurable frequencies declining over the course of the study (FIG. 24). Overall frequencies, however, were low regardless of treatment group but did show an effect that correlated with initial dosing. In relation, detectable CD4⁺ and CD8⁺ T cell frequencies in the bone marrow of BCMA-3L/20H CAR T cell recipient mice also showed a dose-dependent effect in samples taken at study endpoint (FIG. 25 and FIG. 26). Of note, bone marrow resident T cells at study endpoint showed high expression of exhaustion markers, which correlates with an inability to completely clear the tumor based on BLI measurements.

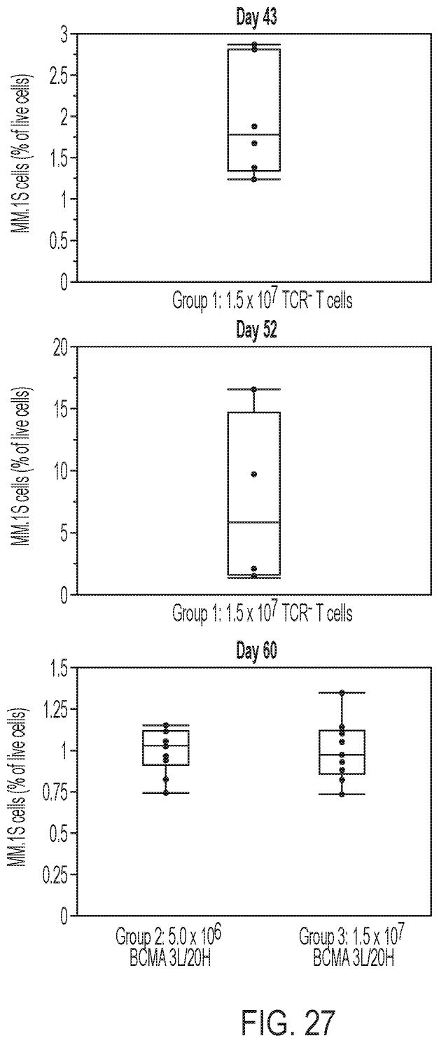

Importantly, end-life bone marrow samples from TCR⁻ control T cell recipients had higher frequencies of BCMA⁺MM.1S cells in the bone marrow compared to animals administered BCMA-3L/20H CAR T cells at either dose (FIG. 27). This observation correlates with reduced flux values in BCMA-3L/20H CAR T cell-treated animals and supports the function of BCMA-3L/20H CAR T cells in clearing BCMA⁺ tumor from engrafted organs (FIG. 21).

### Adverse Events

There were no TR or NTR deaths observed in this study; all animal deaths in this study were classified as DSS.

### 3. Conclusions

This study evaluated the PK, biodistribution, and antitumor efficacy of BCMA-3L/20H CAR T cells in NSG mice bearing 8-day old disseminated BCMA⁺MM.1S multiple myeloma tumors that stably express luciferase. Antitumor efficacy was evaluated by BLI and comparison of survival between TCR⁻ control T cells and BCMA-3L/20H CAR T cells treatment groups. The PK of BCMA-3L/20H CAR T cells was assessed by flow cytometry analysis of blood, bone marrow, and spleen of animals treated with TCR⁻ control T cells and BCMA-3L/20H CAR T cells.

Confirmatory data demonstrated that BCMA-3L/20H CAR T cell treatment conferred a statistically significant survival advantage over TCR⁻ control T cells. All animals in both BCMA-3L/20H CAR T cell groups survived until study end (Day 60). The median TTE for TCR⁻ control T cell group was 43.0 days in comparison to a median TTE of 60.0 days (end of study) for BCMA-3L/20H CAR T cell-treated animals, resulting in a maximum increase in lifespan (ILS) of 40%. Median flux intensity values, as measured by BLI, at Day 43 were significantly lower in both BCMA-3L/20H CAR T cell-treated groups relative to the TCR⁻ control T cell group. Both BCMA-3L/20H CAR T cell dose groups demonstrated statistically significant increases in tumor burden inhibition (TBI) in comparison to the control group administered TCR⁻ control T cells). Mean TBIs were 94% and 100%, respectively, for mice administered 5×10⁶ BCMA-3L/20H CAR T cells and mice administered 1.5×10⁷ BCMA− 3L/20H CAR T cells.

The PK and biodistribution of BCMA-3L/20H CAR T cells was assessed. Flow cytometry analysis of blood, bone marrow, and spleen was performed to evaluate the relative frequencies of T cells and associated expression of exhaustion markers (PD1, TIM3, and LAG3) in addition to the presence of MM.1S tumor cells. Results show that T cells were detected in peripheral blood samples early after administration, with frequencies declining over the course of the study. Relevant exhaustion marker expression was low in temporal blood samples from all groups. At study end, splenic and bone marrow T cell levels were low, but high frequencies of exhaustion marker positive T cells were detected in the bone marrow of animals in all treatment groups. In general, T cell levels detected in the blood, bone marrow, and spleen at the end of study were marginal.

BCMA⁺MM.1S tumor cells in the blood peaked in Day 10 samples. Of note, detectable tumor cell frequencies were suggestive of both a dose-dependent effect of BCMA-3L/20H CAR T cells and specific in vivo efficacy of BCMA-3L/20H CAR T cells compared to the control T cell population. Furthermore, in end-life bone marrow samples TCR⁻ control T cell recipients had higher frequencies of BCMA+ cells remaining compared to BCMA-3L/20H CAR T cell recipients.

In summary, these results demonstrate in vivo activity of BCMA-3L/20H CAR T cells against BCMA⁺ disseminated myeloid tumors, including the ability of IV administered BCMA-3L/20H CAR T cells to traffic to distal tumor sites and mediate antitumor activity.

### Example 11

Evaluation of Graft-Versus-Host Disease in Female and Male NSG Mice Following Intravenous Administration of BCMA-3L/20H CAR T cells

### 1. Methods

### Study Design

Allogenic CAR T cells expressing a diverse array of endogenous TCRs carry the risk of inducing GvHD in human leukocyte antigen (HLA)-mismatched patients (MacLeod et al., Mol Ther. 2017; 25(4):949-961.). BCMA-3L/20H cells have been gene-edited to eliminate TCR expression (TCR⁻) to significantly reduce the possibility of GvHD in HLA-mismatched patients, making it an off-the-shelf treatment for patients who are candidates for CAR T-cell therapy. This study evaluated the incidence of GvHD in immunodeficient mice (King et al., Clin Exp Immunol. 2009; 157(1):104-18; Ali et al., PloS One. 2012;7(8):e44219) treated with gene-edited BCMA-3L/20H cells and unedited TCR⁺ control T cells in a survival study.

Male and female NSG mice were sublethally irradiated on Day 1 to stimulate cell damage and upregulation of major histocompatibility complex molecules in tissue. On Day 2, animals were IV administered vehicle control (Groups 1 and 2), 3×10⁷ unedited TCR⁺ control T cells (Groups 3 and 4), or 3×10⁷ gene-edited BCMA-3L/20H (TCR⁻CAR⁺) test article cells (Groups 5 and 6). Animals were weighed and scored daily for 30 days and then 3 times a week to the end of the study for signs of GvHD. The study endpoint was death or euthanasia due to GvHD progression. Tolerability was determined from the percent ILS, defined as the percent increase in median TTE in treated groups relative to the vehicle treatment group (designated Group 1). Study-specific objectives were to measure GvHD manifestation with appearance of GvHD-like symptoms, body weight loss, mortality, and moribundity. Multiple organ (including gonads) and bone marrow samples were obtained from select animals for histopathological evaluation and analyzed for the presence and persistence of infused human cells.

The incidence of GvHD in immunodeficient mice treated with gene-edited BCMA-3L/20H cells and unedited TCR⁺ control T cells was evaluated. Organ samples were processed and analyzed and for histopathology and immunohistochemistry markers.

The study conducted study-specific, in-phase inspections and critical phase audits. Examples of processes implemented in this study include a prospective protocol, judicious but appropriate numbers of animals of both sexes per group, vehicle control and test article control, organ weights and gross pathology at necropsy under trained personnel and/or a board-certified pathologist, causes of death determined where feasible, and tissues collected at a select timepoint (Day 12) for histopathology and IHC evaluation and at the study end for processing to formalin-fixed paraffin-embedded (FFPE) samples to be archived. The animal study groups (e.g., group numbers and dosing preparation solutions) were coded for client confidentiality and to reduce bias during study procedures.

Male and female immunodeficient mice were submitted to irradiation on Day 1. On Day 2, mice were IV administered the vehicle control, unedited TCR⁺ control T cells, or BCMA-3L/20H cells. IV will be the designated route of administration in humans. Dosing was initiated according to the treatment plan summarized in Table 12. The dose level for BCMA-3L/20H CAR T cells in this study was selected to be higher than the dose range used for the pivotal multiple myeloma MM.1S antitumor efficacy study in mice of Example 8.

Animals were scheduled to be weighed and scored for GvHD moribundity daily up to Day 30 and then 3 times a week to the end of the study. All animals in the unedited TCR⁺ control T cell groups (Groups 3 and 4) met GvHD moribundity criteria by Day 12 and were euthanized along with a subset of animals (5 animals/group) in the vehicle control groups (Groups 1 and 2) and the BCMA-3L/20H CAR T cell groups (Groups 5 and 6) for comparative gross pathology and histopathologic and IHC evaluation. Necropsy was performed on 3 unscheduled deaths to determine cause of death.

TABLE 12
In vivo graft-versus-host disease study design

| | | | Dose | | |
|---|---|---|---|---|---|
| Group^{a)} | n/sex | Treatment^{b)} | (cells/animal) | Route | Schedule for IV Dosing |
| 1 | 10/F | Vehicle^{c)} | 0 | IV | QD × 1 (start on Day 2) |
| 2 | 10/M | Vehicle^{c)} | 0 | IV | QD × 1 (start on Day 2) |
| 3 | 8/F | Unedited TCR⁺ control T cells | 3 × 10⁷ | IV | QD × 1 (start on Day 2) |
| | | (NP9^{d)}) | | | |
| 4 | 8/M | Unedited TCR⁺ control T cells | 3 × 10⁷ | IV | QD × 1 (start on Day 2) |
| | | (NP9^{d)}) | | | |
| 5 | 10/F | BCMA-3L/20H CAR T cells | 3 × 10⁷ | IV | QD × 1 (start on Day 2) |
| | | (NP8^{e)}) | | | |
| 6 | 10/M | BCMA-3L/20H CAR T cells | 3 × 10⁷ | IV | QD × 1 (start on Day 2) |
| | | (NP8^{e)}) | | | |

Abbreviations: F = female; Gy = gray; HSA = human serum albumin; IV = intravenous; M = male; QD = once a day; TCR = T cell receptor.
^{a)}All animals from Groups 3 and 4 were euthanized at the onset of morbidity on Day 12 to preserve sample integrity. A subset of 5 animals from Groups 1, 2, 5, and 6 were also euthanized on Day 12 for direct comparisons to concurrent positive control groups (i.e., Groups 3 and 4).
^{b)}All animals received a single, topical 1.5 Gy radiation dose on Day 1.
^{c)}Diluent: Plasma-Lyte A, 2.0% HSA.
^{d)}Designation code given for client confidentiality and blinding of in-life procedures.
^{e)}Designation code given for client confidentiality and blinding of in-life procedures.

### Mice

Male and female NSG mice (NOD.Cg-Prkdc^{scid} Il2rg^{tmIwjl}/SzJ, The Jackson Laboratory) were 9 weeks old with body weights ranging from 21.0 to 31.1 grams at the beginning of the study. The study complied with the recommendations of the Guide for Care and Use of Laboratory Animals with respect to restraint, husbandry, surgical procedures, feed and fluid regulation, and veterinary care.

### Test and Control Articles

BCMA-3L/20H CAR T cell (TCR CAR⁺, Demo 46) and unedited TCR⁺ control T cells (Batch No. 48 were generated from the same leukapheresis product and were produced. Demo 46 was generated specifically for pivotal IND-enabling studies. The cells were supplied frozen and formulated in cryopreservation media (48% saline, 2% human serum albumin, 47.5% Cryostor CS10, 2.5% dimethyl sulfoxide [DMSO], with the final DMSO concentration at 7.5%). Diluent media served as the vehicle.

TABLE 13
Materials

| Material | Batch No. |
|---|---|
| BCMA-3L/20H CAR T cells (NP8) | Demo 46 |
| Unedited TCR⁺ control T cells (NP9) | 48 |
| Diluent^{a)} | N/A |

Abbreviations: CAR = chimeric antigen receptor; HSA = human serum albumin; TCR = T cell receptor.
^{a)}Diluent: Plasma-Lyte A, 2.0% HSA.

### Whole Body Irradiation

A Faxitron MultiRad 225 X-ray system was used to administer whole body radiation therapy to animals on Day 1, which was 1 day prior to treatment with control or test articles. The following settings were established to deliver 1.5 gray: 225 kV and 17.8 mA for 64 seconds.

### Treatment

On Day 1 of the study, male and female mice were sorted according to body weight and treatment was initiated according to the treatment plan summarized in Table 12. Ten animals were enrolled in each of Groups 1, 2, 5, and 6, whereas 8 animals were enrolled in Groups 3 and 4. Radiation treatment was administered on Day 1, and on Day 2 animals were dosed with vehicle, 3×10⁷ unedited TCR⁺ control T cells (TCR⁺CAR⁻), or 3×10⁷ BCMA-3L/20H CAR T cells (majority are TCR⁻CAR⁺). Doses were administered IV in a fixed volume of 0.2 mL per animal. Pre- and post-injection viability were 89.9% and 81.2%, respectively, for unedited TCR⁺ control T cells and 90.2% and 96.5%, respectively, for the BCMA-3L/20H CAR T cell dosing formulation.

### Survival Endpoints

Animals were monitored individually for an endpoint of moribundity due to progression of GvHD. The TTE was recorded for each mouse that died of its disease or was euthanized due to disease progression; these deaths were assigned TTE values equal to the day of death or euthanasia. Median TTE values were calculated for each group. Animals that survived to the end of the study were euthanized and were assigned a TTE value equal to the last day of the study. The median TTE of treated mice is expressed as a percentage of the median TTE of control mice (% T/C), and the ILS is calculated as:

$$\begin{matrix}ILS = \% T / C - 100 \% , \\ where T = median {TTE}_{treated} , and = C = median {TTE}_{control} \cdot Thus , if T = C , ILS = 0 \% .\end{matrix}$$

### GvHD Scoring

Clinical observations were scored based on the degree of weight loss, activity, posture, fur texture, and skin integrity (Table 14). At each time point, animals were scored in all categories (maximum score of 2 per category across 5 categories), with a maximum possible score of 10 per animal. Individual animal graded scores were summed and averaged across groups then plotted as the median over time. In addition, group mean body weight changes alone were plotted as a function of time.

TABLE 14
GvHD scoring categories

| Criteria | Grade 0 | Grade 1 | Grade 2 |
|---|---|---|---|
| Weight Loss | <10% | ≥10%, <25% | ≥25% |
| Activity | Normal | Mild to moderately decreased | Stationary until stimulated |
| Posture | Normal | Hunching only at rest | Severe gait, impaired movement |
| Fur Texture | Normal | Mild to moderate ruffling | Severe ruffling/poor grooming |
| Skin Integrity | Normal | Scaling of paws and/or tail | Obvious areas of denuded skin |

### Toxicity

Animals were weighed daily for 30 days, then 3 times a week until the completion of the study on Day 47. The mice were observed frequently for overt signs of any adverse, treatment-related (TR) side effects, and clinical signs were recorded when observed in addition to the GvHD scoring already described. Individual body weight loss was monitored and any animal that exceeded the limits for acceptable body weight loss (>30% body weight loss or 3 consecutive measurements of >25% body weight loss) was euthanized; group mean body weight loss was also monitored.

A death was classified as a GvHD death if attributable to GvHD as evidenced by signs of GvHD moribundity. One female mouse (Group 3) and 2 male mice (Group 4) in the unedited TCR⁺ control T cell treatment groups were found dead on Day 12 and underwent necropsy for cause of death, which were classified as GvHD deaths.

A death was classified as TR if attributable to treatment side effects as evidenced by clinical signs and/or necropsy.

Deaths were classified as nontreatment-related (NTR) if there was no evidence that they were related to treatment side effects. NTR deaths were further characterized based on the cause of death. A death was classified as NTRa if it resulted from an accident or human error. A death was classified as NTRu if the cause of death was unknown and there was no available evidence of death related to treatment side effects, accident or human error; however, death due to these etiologies cannot be excluded. There were no NTR deaths in in this study.

### Sampling

Samples were collected for further analysis. On Day 12, all animals treated with TCR⁺ control T cells (Groups 3 and 4) demonstrated severe GvHD symptoms and reached the study endpoint of moribundity and were euthanized (except for 1 GvHD death in Group 3 and 2 GvHD deaths in Group 4) and sampled for immunopathological comparisons across all groups on the same day. Necropsies were conducted for each animal by a board-certified pathologist. Additionally, 5 animals each (odd-numbered animals) from vehicle control groups (Groups 1 and 2) and BCMA-3L/20H CAR T cell-treated groups (Groups 5 and 6) were euthanized on Day 12 and underwent necropsy and organ collection to allow for direct comparisons with Groups 3 and 4.

At the Day 12 and Day 47 necropsies, all organs (brain, bone marrow within both femurs, cecum, colon, heart, left kidney, liver, left lung, skin [intrascapular], small intestines, spleen, stomach, and testes or ovaries) were removed, weighed (except femur bone marrow and skin), examined macroscopically for abnormalities, and fixed in 10% neutral-buffered formalin for 48 hours at the testing facility. Samples were then transferred to 70% ethanol where they were subsequently processed to FFPE preparation. The Day 12 samples were processed to slides and underwent hematoxylin and eosin (H&E) staining, hCD45 and hCD3 immunohistochemical analysis and histopathological evaluation by a board-certified Study Pathologist. Per Study Protocol, because there were no macroscopic findings at the Day 47 necropsy of the vehicle control and BCMA-3L/20H CAR T cell-treated animals, the organs from these animals were not examined microscopically but were archived as FFPE blocks.

### Histopathology and Immunohistochemistry (Day 12)

The IHC and histopathology staining and evaluation were conducted and the experimental procedures applicable to IHC investigations are summarized in Table 15. FFPE tissues from Day 12 were sectioned at approximately 5 m, and adjacent sections were stained with H&E for the histopathology evaluation and IHC staining to detect the presence of hCD45⁺ and hCD3⁺ cells (indicative of human T cells) in the tissues. IHC staining was performed in accordance with test site standard operating procedures, including positive and negative control materials in each staining run and using additional slides for the negative control antibody to ensure stain specificity.

After H&E and IHC staining, slides were visualized under light microscopy by the study pathologist. Each IHC slide (detection antibody or negative control antibody) was examined for the presence of stained cells. The staining intensity and frequency scales used for the IHC evaluation are listed Table 15.

TABLE 15
Staining intensity and frequency

| Staining intensity | | Staining frequency | |
|---|---|---|---|
| Score | Result | Score | Result |
| Neg | Negative (no stained cells) | Very rare | <10% stained cells |
| <1 | Trace/very minimal | Rare | 10% to 20% |
| | | | stained cells |
| 1+ | Minimal (light stain) | Occasional | 20% to 50% |
| | | | stained cells |
| 2+ | Slight (light-medium stain) | Frequent | >50% stained cells |
| 3+ | Moderate (medium stain) | | |
| 4+ | Marked (intense/dark stain) | | |

### Statistical and Graphical Analyses (In-Life)

Prism 8.1.1 (GraphPad) for Windows was employed for statistical and graphical analyses. Survival was analyzed by the Kaplan-Meier method, based on TTE values. Individual and group median GvHD scores were plotted as a function of time. Kaplan-Meier plots show the percentage of animals in each group remaining in the study versus time. Group mean body weight changes over the course of the study were graphed as percent change, ±standard error of the mean, from Day 1.

Survival data was evaluated by Logrank. Two-tailed statistical analyses were conducted at significance level p=0.05 and were not corrected for multiple comparisons.

### 2. Results

### In-Life Findings

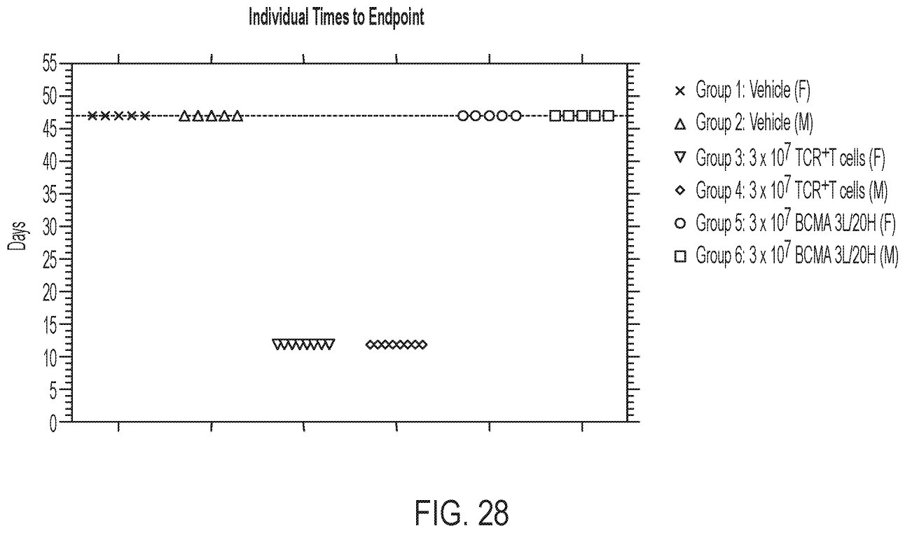

All animals injected with unedited TCR⁺ control T cells (Groups 3 and 4, female and male, respectively) developed severe weight loss and showed clinical signs consistent with GvHD, requiring euthanasia on Day 12 (TTE=12 days; FIG. 28).

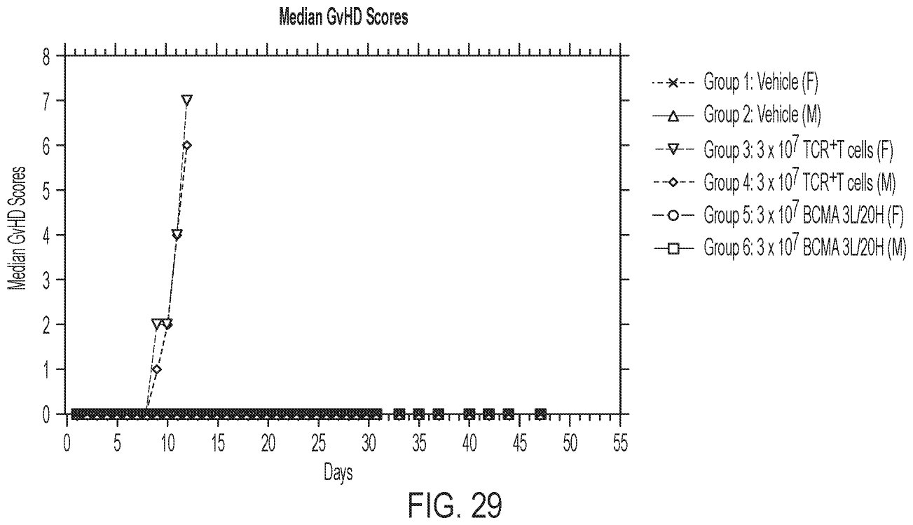

One female mouse (Group 3, Animal No. 3) and 2 male mice (Group 4, Animal No. 2 and 3) in the unedited TCR⁺ control T cell treatment groups were found dead on Day 12 and underwent necropsy for cause of death, which were classified as a GvHD death. Evidence of GvHD was first noted on Day 9 in all animals administered unedited TCR⁺ control T cells (Groups 3 and 4). Mean Day 12 GvHD scores were 6.4 and 5.8 for Groups 3 and 4, respectively (FIG. 29). Animals administered vehicle control (Groups 1 and 2) showed initial median GvHD scores starting on Day 3 that did not exceed a median score of 1 during the study (FIG. 29). All animals administered BCMA-3L/20H CAR T cells (Groups 5 and 6) exhibited low GvHD scores of 0 or <1, respectively (FIG. 29).

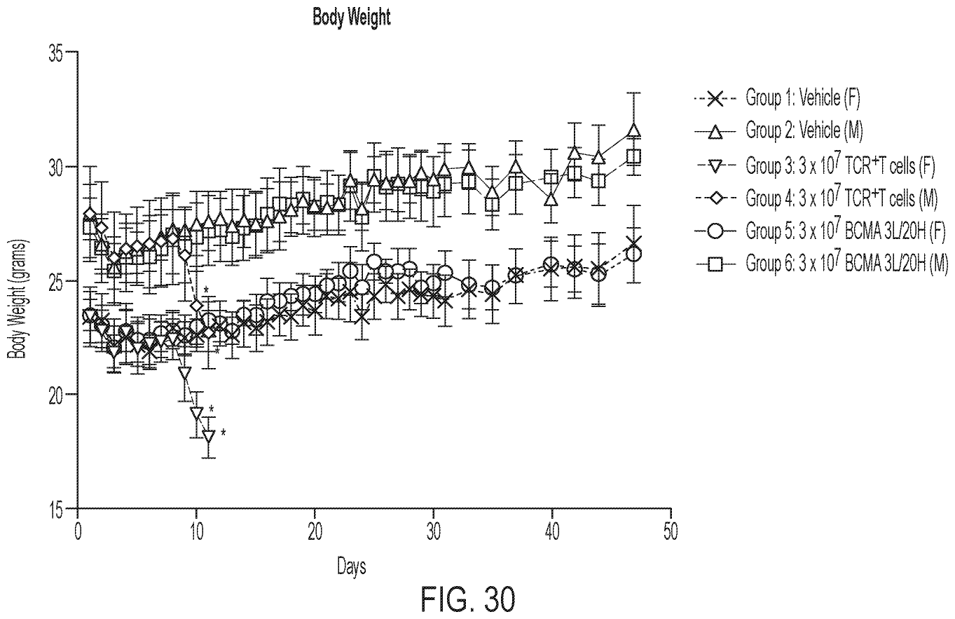

On Day 12, considerable group mean body weight loss (compared to body weight on Day 1) was observed with reductions of 25.5% and 19.6% in Groups 3 and 4 (unedited TCR⁺ control T cells), respectively (FIG. 30).

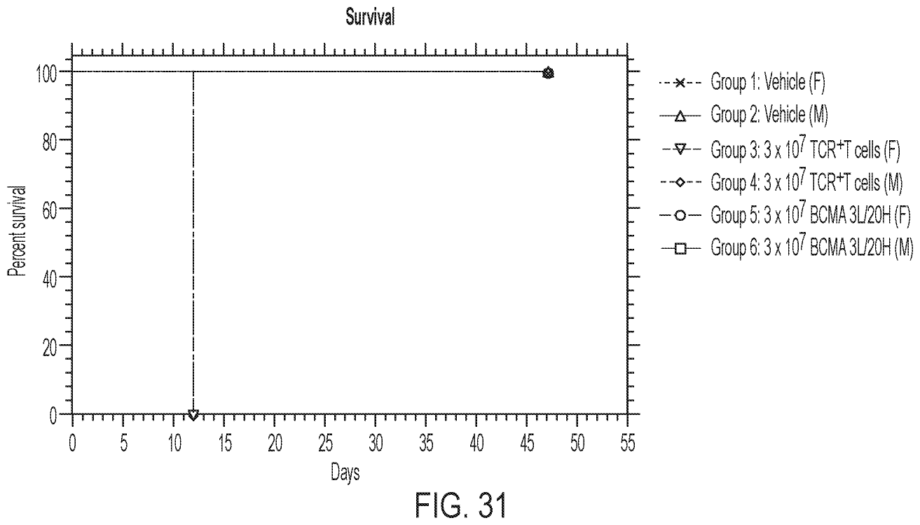

Due to these presentations, all remaining animals in the unedited TCR⁺ control T cell groups (Groups 3 and 4) were euthanized on Day 12 (FIG. 31) to preserve sampling and the data was processed as if all remaining animals progressed to survival endpoint or moribundity on Day 12. One female mouse (Group 3) and 2 male mice (Group 4) in the unedited TCR⁺ control T cell treatment groups were found dead on Day 12 and underwent necropsy for cause of death, which were classified as a GvHD deaths. These animals were excluded from Day 12 graph and statistical analysis. With the exception of a subset of animals from the vehicle control and BCMA-3L/20H CAR T cell groups that underwent scheduled necropsy at Day 12 for comparison with unedited TCR⁺ control T cell groups (Groups 3 and 4), all animals treated with BCMA-3L/20H CAR T cell or vehicle control remained alive through Day 47 (FIG. 31).

In summary, female and male NSG mice treated with BCMA-3L/20H CAR T cells or the vehicle control had minimal mean GvHD scores ranging from 0.1 to 0.2 (FIG. 29), showed normal increases in body weight over the duration of the study (FIG. 30), and had a median TTE of 47 days, the duration of the study (FIG. 28). Female and male NSG mice treated with unedited TCR⁺ control T cells exhibited signs of GvHD (Day 12 mean GvHD scores of 6.4 and 5.8, respectively), severe body weight loss, and mortality and moribundity by Day 12 of the study.

### Gross Pathology

On Day 12, necropsy findings were observed in all groups, but were considered non-adverse. Male and female mice in the vehicle control and BCMA-3L/20H CAR T cell groups had foci (<8 mm in diameter) present on the spleen, which was not observed in the TCR⁺ control T cell animals, with the exception of 1 mouse in the male group. Observations in the spleen is a common occurrence observed in NSG mice, indicative of their immunocompromised profile. Animals administered unedited TCR⁺ control T cells (male and female) had findings in the brain with a focal cortical color change (<5 mm in diameter) that was attributed to the method of euthanization. One female administered unedited TCR⁺ control T cells (Group 3, Animal No. 3) was found dead on Day 12 with observations of dark purple, gelatinous cecum, colon, small intestines, and spleen. Dark lobes of both the liver and lung were noted with a purple color. These findings are consistent with autolysis of the internal organs for mice found dead. One male mouse administered TCR⁺ control T cells (Group 4, Animal No. 3) was found dead on Day 12 and observed with purple discoloration of the lung.

At the Day 47 necropsy 1 female mouse administered vehicle control (Group 1, Animal No. 8) was observed with a single focus in the spleen (<5 mm). One female mouse in the BCMA-3L/20H CAR T cell-treated group (Group 5, Animal No. 2) was found to have a red discoloration of the lung; the study pathologist deemed this finding was not attributed to BCMA-3L/20H CAR T cells, but rather due to euthanization.

### Organ Weights

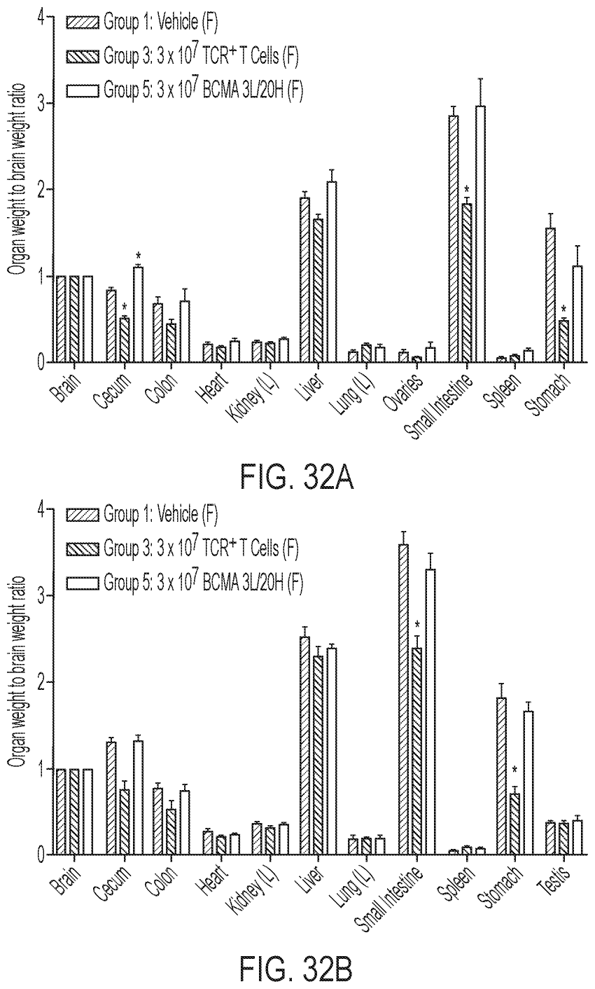

Day 12 organ weight results (normalized to brain) show that in females, the mice treated with unedited TCR⁺ control T cells had significantly lower cecum, small intestine, and stomach weights compared with the vehicle control group (FIG. 32A). In males on Day 12, the small intestine and stomach weights were statistically lower in unedited TCR⁺ control T cell groups compared with the vehicle control (FIG. 32B), which may be related to the rapid body weight loss and deteriorating clinical condition observed in these animals prior to euthanasia due to moribundity. These observations were not seen in male mice treated with BCMA-3L/20H CAR T cells.

A statistically significant increase in cecum weight was observed in female mice administered BCMA-3L/20H CAR T cells compared to mice treated with unedited TCR⁺ control T cells or vehicle control (FIG. 32A); however, this finding is not considered to be biologically significant since these animals appeared healthy and there was no histologic correlate, nor was there a significant difference in small intestine or stomach weight in these female mice compared to vehicle control-treated mice. No other statistically significant organ weight differences were observed in female mice treated with BCMA-3L/20H CAR T cells.

No significant organ weight differences were evident when comparing BCMA-3L/20H CAR T cell treated males to vehicle control males or males administered unedited TCR⁺ control T cells (FIG. 32B).

### Histopathological Evaluation and Immunohistochemistry

Treatment-related findings are summarized in Table 16. Diffuse lymphoid depletion with absence of lymphoid follicles was noted in the spleen from all animals in all groups, which were NSG mouse strain-specific findings.

### Vehicle Control (Group 1 Female; Group 2 Male)

No significant microscopic findings were evident in the vehicle controls. Additionally, hCD45⁺ and hCD3⁺ cells were not detected (no positive staining) in any of the tissues from the vehicle control animals which is consistent with a lack of human T-cell administration in these animals.

### Unedited TCR⁺ Control T Cells (Group 3 Female; Group 4 Male)

In mice administered unedited TCR⁺ control T cells, microscopic findings were identified in the brain, lung, spleen, liver, kidney, stomach, colon, and bone marrow. In the lung, liver, kidney, stomach, colon, and bone marrow of mice administered unedited TCR⁺ control T cells, microscopic findings included variably severe, often perivascular mononuclear cell inflammation characterized by infiltrates of large mononuclear cells with high nuclear to cytoplasmic ratios and prominent nucleoli admixed with pyknotic and karyorrhectic debris. Similar cells filled the red and white pulp of the spleen, expanded the pulmonary alveolar septa, and were multifocally present throughout the hepatic sinusoids of mice administered unedited TCR⁺ control T cells. These cells were positive for hCD45 and often also hCD3. Extramedullary hematopoiesis was not observed in the spleens of mice administered unedited TCR⁺ control T cells.

Necrosis was also observed in multiple organs of mice administered unedited TCR⁺ control T cells, including the lung, liver, spleen, kidney, and bone marrow. Throughout the lung, segments of alveolar septa were hypereosinophilic and lacked cellular detail, indicating acute necrosis. In the liver, clusters of bridging hepatocytes in all areas of the liver were hypereosinophilic with shrunken, fragmented, or absent nuclei (hepatocellular necrosis). Multifocally, small segments of renal glomerular tufts were necrotic, characterized by a lack of cellular detail and fragmented nuclear debris. Pyknotic debris was scattered diffusely throughout the spleen (single cell necrosis), and the bone marrow was severely hypocellular and largely replaced by necrotic nuclear debris and hemorrhage.

Other microscopic findings observed in mice administered unedited TCR⁺ control T cells were considered incidental, of the nature commonly observed in this strain and age of mice, and/or were of similar incidence and severity in animals administered vehicle control and BCMA-3L/20H CAR T cells, and therefore were considered unrelated to administration of unedited TCR⁺ control T cells.

In mice administered unedited TCR⁺ control T cells, hCD45⁺ and hCD3⁺ cells were frequently observed in the lung, spleen, liver, and bone marrow. In the lung, hCD45⁺ and hCD3⁺ cells expanded the alveolar septa, frequently surrounded blood vessels, and were present intravascularly. In the liver, hCD45⁺ and hCD3⁺ cells were present within portal triads, blood vessels, and sinusoids. hCD45⁺ and hCD3⁺ cells filled the red and white pulp of the spleen and were diffusely present throughout the bone marrow. hCD45⁺ and hCD3⁺ cells were observed occasionally or less often in the brain, skin, heart, kidney, stomach, small intestine, cecum, colon, and ovary. hCD3⁺ cells were observed at a similar or slightly less frequent incidence than hCD45⁺ cells in these organs. In all of these organs, hCD45⁺ and hCD3⁺ cells were frequently seen within or surrounding blood vessels. In the brain, hCD45⁺ and hCD3⁺ cells were mainly located intravascularly, in the meninges, in the stroma of the choroid plexus, free within ventricles, within foci of acute hemorrhage, or scattered individually in the neuropil (most likely within capillaries). hCD45⁺ and hCD3⁺ cells were present intravascularly or individually within the dermis or subcutis of the skin and were very rare in some animals. In the heart, hCD45⁺ and hCD3⁺ cells were present intravascularly (includes heart chambers) or scattered throughout the myocardium. hCD45⁺ and hCD3⁺ cells were present throughout the renal cortex and medulla but were most frequent in the cortex. hCD45⁺ and hCD3⁺ cells in the kidney were located intravascularly, perivascularly, interstitially, or within glomerular tufts. Within segments of the gastrointestinal tract, hCD45⁺ and hCD3⁺ cells were primarily within the mucosal lamina propria, often adjacent to the muscularis mucosa. hCD45⁺ and hCD3⁺ cells were scattered throughout the ovarian stroma, occasionally within corpora lutea.

hCD45⁺ and hCD3⁺ cells were very rare in the testes and were only found as individual cells within blood vessels, immediately adjacent to blood vessels, or within the interstitium.

### BCMA-3L/20H CAR T Cell (Groups 5 and 6)

The examined tissues from mice administered BCMA-3L/20H CAR T cells were largely unremarkable. Minimal to mild acute hemorrhage was observed microscopically in the lung of mice administered BCMA-3L/20H CAR T cells but was at a slightly lower frequency and lower severity than that seen in mice administered unedited TCR⁺ control T cells. Minimal to mild acute pulmonary hemorrhage is a common incidental finding in euthanized mice. As such, the acute pulmonary hemorrhage observed in mice administered BCMA-3L/20H CAR T cells in this study may be an incidental finding; however, because acute pulmonary hemorrhage was not observed in vehicle control mice, a relationship to BCMA-3L/20H CAR T cell administration cannot be fully ruled out.

Other microscopic findings observed were considered incidental, of the nature commonly observed in this strain and age of mice, and/or were of similar incidence and severity in animals administered vehicle control and, therefore, were considered unrelated to administration of BCMA-3L/20H CAR T cells.

In mice administered BCMA-3L/20H CAR T cells, hCD45⁺ cells were most frequently observed (“rare” to “occasional” stained cells) in the lung and spleen. Only “very rare” or “rare” positive cells (or sometimes no positive cells) were identified in the other tissues examined (brain, skin, heart, liver, kidney, stomach, small intestine, cecum, colon, ovary, testis, and bone marrow). hCD3⁺ cells were generally not detected, and when present were “rare” to “very rare,” with the exception of occasional hCD3⁺ cells in the spleen of 2 females and 1 male administered BCMA-3L/20H CAR T cells.

In animals administered BCMA-3L/20H CAR T cells, hCD45⁺ cells were observed in anatomically similar locations as observed in animals administered unedited TCR⁺ control T cells. Although there were rare occurrences of hCD3⁺staining in animals administered BCMA-3L/20H CAR T cells, these cells were also observed in similar locations as in animals administered unedited TCR⁺ control T cells. hCD45⁺ and hCD3⁺ cells were often within or around blood vessels but were also found throughout alveolar septa in the lung, scattered within the myocardium, within hepatic portal triads, within the renal interstitium or glomerular tufts, and within the mucosal lamina propria of various segments of the gastrointestinal tract. In the spleen, hCD45⁺, and more rarely, hCD3⁺ cells were scattered throughout the red pulp, but were more densely aggregated in the periarteriolar lymphoid sheaths. hCD45⁺, and in only 3 mice, hCD3⁺ cells were scattered throughout the bone marrow individually or in small aggregates.

TABLE 16
Summary of microscopic findings related to positive control treatment and
comparisons with vehicle control and BCMA-3L/20H CAR T cells (Day 12)

| | | | Unedited TCR⁺ | | BCMA-3L/20H | |
|---|---|---|---|---|---|---|
| | Vehicle | | control T Cells | | CAR T cells | |
| | Group | | | | | |
| | 1 | 2 | 3 | 4 | 5 | 6 |
| | Sex (No. Animals Examined) | | | | | |
| | F (5) | M (5) | F (7) | M (6) | F (5) | M (5) |
| Brain (No. Examined) | 5 | 5 | 7 | 6 | 5 | 5 |
| Hemorrhage, acute | (0) | (0) | (5) | (5) | (0) | (0) |
| Minimal | 0 | 0 | 4 | 3 | 0 | 0 |
| Mild | 0 | 0 | 1 | 2 | 0 | 0 |
| Lung (No. Examined) | 5 | 5 | 7 | 6 | 5 | 5 |
| Necrosis, septal/vascular | (0) | (0) | (7) | (6) | (0) | (0) |
| Minimal | 0 | 0 | 0 | 0 | 0 | 0 |
| Mild | 0 | 0 | 4 | 0 | 0 | 0 |
| Moderate | 0 | 0 | 3 | 6 | 0 | 0 |
| Inflammation, mononuclear cell | (0) | (0) | (7) | (6) | (0) | (0) |
| Minimal | 0 | 0 | 1 | 1 | 0 | 0 |
| Mild | 0 | 0 | 6 | 4 | 0 | 0 |
| Moderate | 0 | 0 | 0 | 1 | 0 | 0 |
| Hypercellularity, septal (septal | (0) | (0) | (7) | (6 | (1) | (0) |
| thickening) | | | | | | |
| Minimal | 0 | 0 | 0 | 0 | 1 | 0 |
| Mild | 0 | 0 | 7 | 5 | 0 | 0 |
| Moderate | 0 | 0 | 0 | 1 | 0 | 0 |
| Hemorrhage, acute | (0) | (0) | (5) | (3) | (4) | (1) |
| Minimal | 0 | 0 | 0 | 2 | 3 | 1 |
| Mild | 0 | 0 | 4 | 1 | 1 | 0 |
| Moderate | 0 | 0 | 1 | 0 | 0 | 0 |
| Spleen (No. Examined) | 5 | 5 | 7 | 6 | 5 | 5 |
| Single cell necrosis | (0) | (0) | (7) | (6 | (0) | (0) |
| Mild | 0 | 0 | 4 | 5 | 0 | 0 |
| Moderate | 0 | 0 | 3 | 1 | 0 | 0 |
| Infiltration, mononuclear cells, | (0) | (0) | (7) | (6) | (0) | (0) |
| suspected injected human T cells | | | | | | |
| Moderate | 0 | 0 | 5 | 6 | 0 | 0 |
| Marked | 0 | 0 | 2 | 0 | 0 | 0 |
| Infiltration, mononuclear cells, typical | (5) | (5) | (0) | (0) | (5) | (5) |
| of extramedullary hematopoiesis | | | | | | |
| Minimal | 0 | 2 | 0 | 0 | 0 | 0 |
| Mild | 1 | 1 | 0 | 0 | 0 | 1 |
| Moderate | 2 | 1 | 0 | 0 | 2 | 2 |
| Marked | 2 | 1 | 0 | 0 | 3 | 2 |
| Liver (No. Examined) | 5 | 5 | 7 | 6 | 5 | 5 |
| Necrosis, hepatocellular | (0) | (0) | (7) | (6) | (0) | (0) |
| Moderate | 0 | 0 | 7 | 6 | 0 | 0 |
| Hypercellularity, sinusoids | (0) | (0) | (7) | (6) | (0) | (0) |
| Minimal | 0 | 0 | 3 | 5 | 0 | 0 |
| Mild | 0 | 0 | 4 | 1 | 0 | 0 |
| Fibroplasia, portal triads | (0) | (0) | (7) | (6) | (0) | (0) |
| Mild | 0 | 0 | 7 | 6 | 0 | 0 |
| Inflammation, mononuclear cell | (0) | (0) | (4) | (4) | (0) | (0) |
| Minimal | 0 | 0 | 4 | 4 | 0 | 0 |
| Kidney (No. Examined) | 5 | 5 | 7 | 6 | 5 | 5 |
| Inflammation, mononuclear cell | (0) | (0) | (7) | (6) | (0) | (0) |
| Minimal | 0 | 0 | 5 | 4 | 0 | 0 |
| Mild | 0 | 0 | 2 | 2 | 0 | 0 |
| Hypercellularity, glomerular tuft | (0) | (0) | (7) | (6) | (0) | (0) |
| Minimal | 0 | 0 | 7 | 6 | 0 | 0 |
| Necrosis, glomerular tuft | (0) | (0) | (6) | (1) | (0) | (0) |
| Minimal | 0 | 0 | 6 | 1 | 0 | 0 |
| Stomach (No. Examined) | 5 | 5 | 7 | 6 | 5 | 5 |
| Dilation, glandular | (1) | (2) | (6) | (6) | (0) | (1) |
| Minimal | 1 | 2 | 4 | 1 | 0 | 1 |
| Mild | 0 | 0 | 2 | 5 | 0 | 0 |
| Inflammation, mononuclear cell | (0) | (0) | (4) | (2) | (0) | (0) |
| Minimal | 0 | 0 | 4 | 2 | 0 | 0 |
| Colon (No. Examined) | 5 | 5 | 7 | 6 | 5 | 5 |
| Inflammation, mononuclear cell | (0) | (0) | (0) | (1) | (0) | (0) |
| Minimal | 0 | 0 | 0 | 1 | 0 | 0 |
| Bone Marrow, femur (No. Examined) | 4 | 5 | 7 | 6 | 5 | 5 |
| Fibroplasia | (0) | (0) | (3) | (0) | (0) | (0) |
| Minimal | 0 | 0 | 1 | 0 | 0 | 0 |
| Mild | 0 | 0 | 2 | 0 | 0 | 0 |
| Necrosis | (0) | (0) | (7) | (6) | (0) | (0) |
| Severe | 0 | 0 | 7 | 6 | 0 | 0 |
| Inflammation, mononuclear cell, | (0) | (0) | (1) | (0) | (0) | (0) |
| perivascular | | | | | | |
| Minimal | 0 | 0 | 1 | 0 | 0 | 0 |

Abbreviations: F = female; M = male; No. = number; TCR = T cell receptor.
Notes:
Numbers in parenthesis represent the total number of animals with the finding.

### Histopathology and Immunohistochemistry Conclusions

Administration of unedited TCR⁺ control T cells resulted in multiple microscopic findings across most organs examined. In the lung of unedited TCR⁺ control T cell-treated mice, microscopic findings included variably severe, often perivascular mononuclear cell inflammation and expansion of the alveolar septa, and acute necrosis of segments of the alveolar septa. In the spleen, scattered single cell necrosis was observed and mononuclear cell infiltration was observed in the red and white pulp. Hypocellularity and hemorrhage was observed in the bone marrow. Multifocally, small segments of renal glomerular tufts were necrotic. Hepatocellular necrosis was observed in the liver of mice administered unedited TCR⁺ control T cells, and mononuclear cell infiltration was observed throughout the hepatic sinusoids.

In mice administered unedited TCR+ control T cells, hCD45+ and hCD3+ cells were frequently observed in the lung, spleen, liver, and bone marrow. hCD45⁺ and hCD3⁺ cells were observed occasionally or less often in the brain, skin, heart, kidney, stomach, small intestine, cecum, colon, and ovary. hCD3⁺ cells were observed at a similar or slightly less frequent incidence than hCD45⁺ cells in these organs.

The examined tissues from mice administered BCMA-3L/20H CAR T cells were largely unremarkable. Minimal to mild acute hemorrhage was observed microscopically in the lung of mice administered BCMA-3L/20H CAR T cells but was at a slightly lower frequency and lower severity than that seen in mice administered unedited TCR⁺ control T cells.

In mice administered BCMA-3L/20H CAR T cells, hCD45⁺ cells were most frequently observed (“rare” to “occasional” stained cells) in the lung and spleen. Only “very rare” or “rare” hCD45⁺positive cells (or sometimes no positive cells) were identified in the other tissues examined (brain, skin, heart, liver, kidney, stomach, small intestine, cecum, colon, ovary, testis, and bone marrow). hCD3⁺ cells were generally not detected, and when present were “rare” to “very rare,” with the exception of occasional hCD3⁺ cells in the spleen of 2 females and 1 male administered BCMA-3L/20H CAR T cells.

### 3. Conclusions

All mice treated with unedited TCR⁺ control T cells (i.e., TCR-expressing) demonstrated an acute onset of GvHD characteristics and associated moribundity by Day 12, requiring sacrifice. Additionally, treatment with unedited TCR+ control T cells resulted in multiple microscopic findings across most organs examined consistent with GvHD. In contrast, administration of a high dose of BCMA-3L/20H CAR T cells, which is almost entirely composed of gene-edited TCR⁻ T cells (i.e., TCR knock out), did not cause any significant clinical signs or body weight changes in mice that would be consistent with the development of GvHD.

BCMA-3L/20H CAR T cell treatment did not result in any microscopic findings (Day 12) except for minimal to mild acute hemorrhage that was observed microscopically in the lung of mice administered BCMA-3L/20H CAR T cells but was at a slightly lower frequency and lower severity than that seen in mice administered unedited TCR⁺ control T cells. Mice administered a high dose of BCMA-3L/20H CAR T cells remained on study through Day 47 (last day of study) with the exception of a subset of animals that underwent scheduled necropsy at Day 12 for comparison with the positive control, unedited TCR⁺ control T cell groups. This study demonstrates that BCMA-3L/20H CAR T cells do not induce GvHD in an immune-deficient mouse model of GvHD.

Based on the results of this study, a dose of 3×10⁷ BCMA-3L/20H CAR T cells/animal did not induce GvHD and did not produce any adverse findings; this dose equates to a dosage of 1.2×10⁹ BCMA-3L/20H CAR T cells/kg (average body weight of mice, 25 g), which is 125-fold higher than the highest dosage (6×10⁶ BCMA-3L/20H CAR T cells/kg) proposed for administration to humans in clinical studies.

1. An isolated antibody, or antigen-binding fragment thereof, comprising a variable heavy (VH) region that comprises a complementarity-determining region heavy 1 (CDRH1) domain, a complementarity-determining region heavy 2 (CDRH2) domain, and a complementarity-determining region heavy 3 (CDRH3) domain; and a variable light (VL) region that comprises a complementarity-determining region light 1 (CDRL1) domain, a complementarity-determining region light 2 (CDRL2) domain, and a complementarity-determining region light 3 (CDRL3) domain, wherein said CDRH1 domain, said CDRH2 domain, and said CDRH3 domain are from any VH region set forth in any one of SEQ ID NOs: 2, 6, and 10; and wherein said CDRL1 domain, said CDRL2 domain, and said CDRL3 domain are from any VL region set forth in any one of SEQ ID NOs: 4, 8, and 12, wherein said isolated antibody, or antigen-binding fragment thereof, specifically binds to human BCMA.

2. The isolated antibody, or antigen-binding fragment thereof, of claim 1, wherein said CDRH1 domain comprises an amino acid sequence set forth in any one of SEQ ID NOs: 14, 20, and 26.

3. The isolated antibody, or antigen-binding fragment thereof, of claim 1 or 2, wherein said CDRH2 domain comprises an amino acid sequence set forth in any one of SEQ ID NOs: 15, 21, and 27.

4. The isolated antibody, or antigen-binding fragment thereof, of any one of claims 1-3, wherein said CDRH3 domain comprises an amino acid sequence set forth in any one of SEQ ID NOs: 16, 22, and 28.

5. The isolated antibody, or antigen-binding fragment thereof, of any one of claims 1-4, wherein said CDRL1 domain comprises an amino acid sequence set forth in any one of SEQ ID NOs: 17, 23, and 29.

6. The isolated antibody, or antigen-binding fragment thereof, of any one of claims 1-5, wherein said CDRL2 domain comprises an amino acid sequence set forth in any one of SEQ ID NOs: 18, 24, and 30.

7. The isolated antibody, or antigen-binding fragment thereof, of any one of claims 1-6, wherein said CDRL3 domain comprises an amino acid sequence set forth in any one of SEQ ID NOs: 19, 25, and 31.

8. The isolated antibody, or antigen-binding fragment thereof, of any one of claims 1-7, wherein:
(a) said CDRH1 domain comprises an amino acid sequence set forth in SEQ ID NO: 14; said CDRH2 domain comprises an amino acid sequence set forth in SEQ ID NO: 15;
and said CDRH3 domain comprises an amino acid sequence set forth in SEQ ID NO: 16;
(b) said CDRH1 domain comprises an amino acid sequence set forth in SEQ ID NO: 20; said CDRH2 domain comprises an amino acid sequence set forth in SEQ ID NO: 21; and said CDRH3 domain comprises an amino acid sequence set forth in SEQ ID NO: 22; or
(c) said CDRH1 domain comprises an amino acid sequence set forth in SEQ ID NO: 26; said CDRH2 domain comprises an amino acid sequence set forth in SEQ ID NO: 27; and said CDRH3 domain comprises an amino acid sequence set forth in SEQ ID NO: 28.

9. The isolated antibody, or antigen-binding fragment thereof, of any one of claims 1-8, wherein:
(a) said CDRL1 domain comprises an amino acid sequence set forth in SEQ ID NO: 17; said CDRL2 domain comprises an amino acid sequence set forth in SEQ ID NO: 18; and said CDRL3 domain comprises an amino acid sequence set forth in SEQ ID NO: 19;
(b) said CDRL1 domain comprises an amino acid sequence set forth in SEQ ID NO: 23; said CDRL2 domain comprises an amino acid sequence set forth in SEQ ID NO: 24; and said CDRL3 domain comprises an amino acid sequence set forth in SEQ ID NO: 25; or
(c) said CDRL1 domain comprises an amino acid sequence set forth in SEQ ID NO: 29; said CDRL2 domain comprises an amino acid sequence set forth in SEQ ID NO: 30; and said CDRL3 domain comprises an amino acid sequence set forth in SEQ ID NO: 31.

10. The isolated antibody, or antigen-binding fragment thereof, of any one of claims 1-9, wherein:
(a) said CDRH1 domain comprises an amino acid sequence set forth in SEQ ID NO: 14; said CDRH2 domain comprises an amino acid sequence set forth in SEQ ID NO: 15; said CDRH3 domain comprises an amino acid sequence set forth in SEQ ID NO: 16; said CDRL1 domain comprises an amino acid sequence set forth in SEQ ID NO: 17; said CDRL2 domain comprises an amino acid sequence set forth in SEQ ID NO: 18; and said CDRL3 domain comprises an amino acid sequence set forth in SEQ ID NO: 19;
(b) said CDRH1 domain comprises an amino acid sequence set forth in SEQ ID NO: 20; said CDRH2 domain comprises an amino acid sequence set forth in SEQ ID NO: 21; said CDRH3 domain comprises an amino acid sequence set forth in SEQ ID NO: 22; said CDRL1 domain comprises an amino acid sequence set forth in SEQ ID NO: 23; said CDRL2 domain comprises an amino acid sequence set forth in SEQ ID NO: 24; and said CDRL3 domain comprises an amino acid sequence set forth in SEQ ID NO: 25;
(c) said CDRH1 domain comprises an amino acid sequence set forth in SEQ ID NO: 26; said CDRH2 domain comprises an amino acid sequence set forth in SEQ ID NO: 27; said CDRH3 domain comprises an amino acid sequence set forth in SEQ ID NO: 28; said CDRL1 domain comprises an amino acid sequence set forth in SEQ ID NO: 29; said CDRL2 domain comprises an amino acid sequence set forth in SEQ ID NO: 30; and said CDRL3 domain comprises an amino acid sequence set forth in SEQ ID NO: 31;
(d) said CDRH1 domain comprises an amino acid sequence set forth in SEQ ID NO: 14; said CDRH2 domain comprises an amino acid sequence set forth in SEQ ID NO: 15; said CDRH3 domain comprises an amino acid sequence set forth in SEQ ID NO: 16; said CDRL1 domain comprises an amino acid sequence set forth in SEQ ID NO: 23; said CDRL2 domain comprises an amino acid sequence set forth in SEQ ID NO: 24; and said CDRL3 domain comprises an amino acid sequence set forth in SEQ ID NO: 25;
(e) said CDRH1 domain comprises an amino acid sequence set forth in SEQ ID NO: 14; said CDRH2 domain comprises an amino acid sequence set forth in SEQ ID NO: 15; said CDRH3 domain comprises an amino acid sequence set forth in SEQ ID NO: 16; said CDRL1 domain comprises an amino acid sequence set forth in SEQ ID NO: 29; said CDRL2 domain comprises an amino acid sequence set forth in SEQ ID NO: 30; and said CDRL3 domain comprises an amino acid sequence set forth in SEQ ID NO: 31;
(f) said CDRH1 domain comprises an amino acid sequence set forth in SEQ ID NO: 20; said CDRH2 domain comprises an amino acid sequence set forth in SEQ ID NO: 21; said CDRH3 domain comprises an amino acid sequence set forth in SEQ ID NO: 22; said CDRL1 domain comprises an amino acid sequence set forth in SEQ ID NO: 17; said CDRL2 domain comprises an amino acid sequence set forth in SEQ ID NO: 18; and said CDRL3 domain comprises an amino acid sequence set forth in SEQ ID NO: 19;
(g) said CDRH1 domain comprises an amino acid sequence set forth in SEQ ID NO: 20; said CDRH2 domain comprises an amino acid sequence set forth in SEQ ID NO: 21; said CDRH3 domain comprises an amino acid sequence set forth in SEQ ID NO: 22; said CDRL1 domain comprises an amino acid sequence set forth in SEQ ID NO: 29; said CDRL2 domain comprises an amino acid sequence set forth in SEQ ID NO: 30; and said CDRL3 domain comprises an amino acid sequence set forth in SEQ ID NO: 31;
(h) said CDRH1 domain comprises an amino acid sequence set forth in SEQ ID NO: 26; said CDRH2 domain comprises an amino acid sequence set forth in SEQ ID NO: 27; said CDRH3 domain comprises an amino acid sequence set forth in SEQ ID NO: 28; said CDRL1 domain comprises an amino acid sequence set forth in SEQ ID NO: 17; said CDRL2 domain comprises an amino acid sequence set forth in SEQ ID NO: 18; and said CDRL3 domain comprises an amino acid sequence set forth in SEQ ID NO: 19; or
(i) said CDRH1 domain comprises an amino acid sequence set forth in SEQ ID NO: 26; said CDRH2 domain comprises an amino acid sequence set forth in SEQ ID NO: 27; said CDRH3 domain comprises an amino acid sequence set forth in SEQ ID NO: 28; said CDRL1 domain comprises an amino acid sequence set forth in SEQ ID NO: 23; said CDRL2 domain comprises an amino acid sequence set forth in SEQ ID NO: 24; and said CDRL3 domain comprises an amino acid sequence set forth in SEQ ID NO: 25.

11. The isolated antibody, or antigen-binding fragment thereof, of any one of claims 1-10, wherein said VH region comprises an amino acid sequence having at least about 80% sequence identity to a sequence set forth in any one of SEQ ID NOs: 2, 6, and 10.

12. The isolated antibody, or antigen-binding fragment thereof, of any one of claims 1-11, wherein said VH region is encoded by a nucleic acid sequence having at least about 80% sequence identity to a sequence set forth in any one of SEQ ID NOs: 3, 7, and 11.

13. The isolated antibody, or antigen-binding fragment thereof, of any one of claims 1-12, wherein said VL region comprises an amino acid sequence having at least about 80% sequence identity to a sequence set forth in any one of SEQ ID NOs: 4, 8, and 12.

14. The isolated antibody, or antigen-binding fragment thereof, of any one of claims 1-13, wherein said VL region is encoded by a nucleic acid sequence having at least about 80% sequence identity to a sequence set forth in any one of SEQ ID NOs: 5, 9, and 13.

15. The isolated antibody, or antigen-binding fragment thereof, of any one of claims 1-14, wherein:
(a) said VH region comprises an amino acid sequence having at least about 80% sequence identity to a sequence set forth in any one of SEQ ID NOs: 2, 6, and 10; and
(b) said VL region comprises an amino acid sequence having at least about 80% sequence identity to a sequence set forth in any one of SEQ ID NOs: 4, 8, and 12.

16. The isolated antibody, or antigen-binding fragment thereof, of any one of claims 1-15, wherein:
(a) said VH region is encoded by a nucleic acid sequence having at least about 80% sequence identity to a sequence set forth in any one of SEQ ID NOs: 3, 7, and 11; and
(b) said VL region is encoded by a nucleic acid sequence having at least about 80% sequence identity to a sequence set forth in any one of SEQ ID NOs: 5, 9, and 13.

17. The isolated antibody, or antigen-binding fragment thereof, of any one of claims 1-16, wherein:
(a) said VH region comprises an amino acid sequence having at least about 80% sequence identity to the sequence set forth in SEQ ID NO: 2, and said VL region comprises an amino acid sequence having at least about 80% sequence identity to the sequence set forth in SEQ ID NO: 4;
(b) said VH region comprises an amino acid sequence having at least about 80% sequence identity to the sequence set forth in SEQ ID NO: 6, and said VL region comprises an amino acid sequence having at least about 80% sequence identity to the sequence set forth in SEQ ID NO: 8;
(c) said VH region comprises an amino acid sequence having at least about 80% sequence identity to the sequence set forth in SEQ ID NO: 10, and said VL region comprises an amino acid sequence having at least about 80% sequence identity to the sequence set forth in SEQ ID NO: 12;
(d) said VH region comprises an amino acid sequence having at least about 80% sequence identity to the sequence set forth in SEQ ID NO: 2, and said VL region comprises an amino acid sequence having at least about 80% sequence identity to the sequence set forth in SEQ ID NO: 8;
(e) said VH region comprises an amino acid sequence having at least about 80% sequence identity to the sequence set forth in SEQ ID NO: 2, and said VL region comprises an amino acid sequence having at least about 80% sequence identity to the sequence set forth in SEQ ID NO: 12;
(f) said VH region comprises an amino acid sequence having at least about 80% sequence identity to the sequence set forth in SEQ ID NO: 6, and said VL region comprises an amino acid sequence having at least about 80% sequence identity to the sequence set forth in SEQ ID NO: 4;
(g) said VH region comprises an amino acid sequence having at least about 80% sequence identity to the sequence set forth in SEQ ID NO: 6, and said VL region comprises an amino acid sequence having at least about 80% sequence identity to the sequence set forth in SEQ ID NO: 12;
(h) said VH region comprises an amino acid sequence having at least about 80% sequence identity to the sequence set forth in SEQ ID NO: 10, and said VL region comprises an amino acid sequence having at least about 80% sequence identity to the sequence set forth in SEQ ID NO: 4; or
(i) said VH region comprises an amino acid sequence having at least about 80% sequence identity to the sequence set forth in SEQ ID NO: 10, and said VL region comprises an amino acid sequence having at least about 80% sequence identity to the sequence set forth in SEQ ID NO: 8.

18. The isolated antibody, or antigen-binding fragment thereof, of any one of claims 1-17, wherein:
(a) said VH region is encoded by a nucleic acid sequence having at least about 80% sequence identity to the sequence set forth in SEQ ID NO: 3, and said VL region is encoded by a nucleic acid sequence having at least about 80% sequence identity to the sequence set forth in SEQ ID NO: 5;
(b) said VH region is encoded by a nucleic acid sequence having at least about 80% sequence identity to the sequence set forth in SEQ ID NO: 7, and said VL region is encoded by a nucleic acid sequence having at least about 80% sequence identity to the sequence set forth in SEQ ID NO: 9;
(c) said VH region is encoded by a nucleic acid sequence having at least about 80% sequence identity to the sequence set forth in SEQ ID NO: 11, and said VL region is encoded by a nucleic acid sequence having at least about 80% sequence identity to the sequence set forth in SEQ ID NO: 13;
(d) said VH region is encoded by a nucleic acid sequence having at least about 80% sequence identity to the sequence set forth in SEQ ID NO: 3, and said VL region is encoded by a nucleic acid sequence having at least about 80% sequence identity to the sequence set forth in SEQ ID NO: 9;
(e) said VH region is encoded by a nucleic acid sequence having at least about 80% sequence identity to the sequence set forth in SEQ ID NO: 3, and said VL region is encoded by a nucleic acid sequence having at least about 80% sequence identity to the sequence set forth in SEQ ID NO: 13;
(f) said VH region is encoded by a nucleic acid sequence having at least about 80% sequence identity to the sequence set forth in SEQ ID NO: 7, and said VL region is encoded by a nucleic acid sequence having at least about 80% sequence identity to the sequence set forth in SEQ ID NO: 5;
(g) said VH region is encoded by a nucleic acid sequence having at least about 80% sequence identity to the sequence set forth in SEQ ID NO: 7, and said VL region is encoded by a nucleic acid sequence having at least about 80% sequence identity to the sequence set forth in SEQ ID NO: 13;
(h) said VH region is encoded by a nucleic acid sequence having at least about 80% sequence identity to the sequence set forth in SEQ ID NO: 11, and said VL region is encoded by a nucleic acid sequence having at least about 80% sequence identity to the sequence set forth in SEQ ID NO: 5; or
(i) said VH region is encoded by a nucleic acid sequence having at least about 80% sequence identity to the sequence set forth in SEQ ID NO: 11, and said VL region is encoded by a nucleic acid sequence having at least about 80% sequence identity to the sequence set forth in SEQ ID NO: 9.

19. The isolated antibody, or antigen-binding fragment thereof, of any one of claims 1-18, wherein said VH region comprises an amino acid sequence set forth in any one of SEQ ID NOs: 2, 6, and 10.

20. The isolated antibody, or antigen-binding fragment thereof, of any one of claims 1-19, wherein said VH region is encoded by a nucleic acid sequence set forth in any one of SEQ ID NOs: 3, 7, and 11.

21. The isolated antibody, or antigen-binding fragment thereof, of any one of claims 1-20, wherein said VL region comprises an amino acid sequence set forth in any one of SEQ ID NOs: 4, 8, and 12.

22. The isolated antibody, or antigen-binding fragment thereof, of any one of claims 1-21, wherein said VL region is encoded by a nucleic acid sequence set forth in any one of SEQ ID NOs: 5, 9, and 13.

23. The isolated antibody, or antigen-binding fragment thereof, of any one of claims 1-22, wherein:
(a) said VH region comprises an amino acid sequence set forth in any one of SEQ ID NOs: 2, 6, and 10; and
(b) said VL region comprises an amino acid sequence set forth in any one of SEQ ID NOs: 4, 8, and 12.

24. The isolated antibody, or antigen-binding fragment thereof, of any one of claims 1-23, wherein:
(a) said VH region is encoded by a nucleic acid sequence set forth in any one of SEQ ID NOs: 3, 7, and 11; and
(b) said VL region is encoded by a nucleic acid sequence set forth in any one of SEQ ID NOs: 5, 9, and 13.

25. The isolated antibody, or antigen-binding fragment thereof, of any one of claims 1-24, wherein:
(a) said VH region comprises an amino acid sequence set forth in SEQ ID NO: 2, and said VL region comprises an amino acid sequence set forth in SEQ ID NO: 4;
(b) said VH region comprises an amino acid sequence set forth in SEQ ID NO: 6, and said VL region comprises an amino acid sequence set forth in SEQ ID NO: 8;
(c) said VH region comprises an amino acid sequence set forth in SEQ ID NO: 10, and said VL region comprises an amino acid sequence set forth in SEQ ID NO: 12;
(d) said VH region comprises an amino acid sequence set forth in SEQ ID NO: 2, and said VL region comprises an amino acid sequence set forth in SEQ ID NO: 8;
(e) said VH region comprises an amino acid sequence set forth in SEQ ID NO: 2, and said VL region comprises an amino acid sequence set forth in SEQ ID NO: 12;
(f) said VH region comprises an amino acid sequence set forth in SEQ ID NO: 6, and said VL region comprises an amino acid sequence set forth in SEQ ID NO: 4;
(g) said VH region comprises an amino acid sequence set forth in SEQ ID NO: 6, and said VL region comprises an amino acid sequence set forth in SEQ ID NO: 12;
(h) said VH region comprises an amino acid sequence set forth in SEQ ID NO: 10, and said VL region comprises an amino acid sequence set forth in SEQ ID NO: 4; or
(i) said VH region comprises an amino acid sequence set forth in SEQ ID NO: 10, and said VL region comprises an amino acid sequence set forth in SEQ ID NO: 8.

26. The isolated antibody, or antigen-binding fragment thereof, of any one of claims 1-25, wherein:
(a) said VH region is encoded by a nucleic acid sequence set forth in SEQ ID NO: 3, and said VL region is encoded by a nucleic acid sequence set forth in SEQ ID NO: 5;
(b) said VH region is encoded by a nucleic acid sequence set forth in SEQ ID NO: 7, and said VL region is encoded by a nucleic acid sequence set forth in SEQ ID NO: 9;
(c) said VH region is encoded by a nucleic acid sequence set forth in SEQ ID NO: 11, and said VL region is encoded by a nucleic acid sequence set forth in SEQ ID NO: 13;
(d) said VH region is encoded by a nucleic acid sequence set forth in SEQ ID NO: 3, and said VL region is encoded by a nucleic acid sequence set forth in SEQ ID NO: 9;
(e) said VH region is encoded by a nucleic acid sequence set forth in SEQ ID NO: 3, and said VL region is encoded by a nucleic acid sequence set forth in SEQ ID NO: 13;
(f) said VH region is encoded by a nucleic acid sequence set forth in SEQ ID NO: 7, and said VL region is encoded by a nucleic acid sequence set forth in SEQ ID NO: 5;
(g) said VH region is encoded by a nucleic acid sequence set forth in SEQ ID NO: 7, and said VL region is encoded by a nucleic acid sequence set forth in SEQ ID NO: 13;
(h) said VH region is encoded by a nucleic acid sequence set forth in SEQ ID NO: 11, and said VL region is encoded by a nucleic acid sequence set forth in SEQ ID NO: 5; or
(i) said VH region is encoded by a nucleic acid sequence set forth in SEQ ID NO: 11, and said VL region is encoded by a nucleic acid sequence set forth in SEQ ID NO: 9.

27. The isolated antibody, or antigen-binding fragment thereof, of any one of claims 1-26, wherein said isolated antibody, or antigen binding fragment thereof, comprises a heavy chain constant (CH) region, wherein said CH region comprises an amino acid sequence having at least about 80% sequence identity to a sequence set forth in SEQ ID NO: 77.

28. The isolated antibody, or antigen-binding fragment thereof, of any one of claims 1-27, wherein said CH region is encoded by a nucleic acid sequence having at least about 80% sequence identity to a sequence set forth in SEQ ID NO: 78.

29. The isolated antibody, or antigen-binding fragment thereof, of any one of claims 1-28, wherein said CH region comprises an amino acid sequence set forth in SEQ ID NO: 77.

30. The isolated antibody, or antigen-binding fragment thereof, of any one of claims 1-29, wherein said CH region is encoded by a nucleic acid sequence set forth in SEQ ID NO: 78.

31. The isolated antibody, or antigen-binding fragment thereof, of any one of claims 1-30, wherein said isolated antibody, or antigen binding fragment thereof, comprises a light chain constant (CL) region, wherein said LC region comprises an amino acid sequence having at least about 80% sequence identity to a sequence set forth in SEQ ID NO: 79.

32. The isolated antibody, or antigen-binding fragment thereof, of any one of claims 1-31, wherein said CL region is encoded by a nucleic acid sequence having at least about 80% sequence identity to a sequence set forth in SEQ ID NO: 80.

33. The isolated antibody, or antigen-binding fragment thereof, of any one of claims 1-32, wherein said CL region comprises an amino acid sequence set forth in SEQ ID NO: 79.

34. The isolated antibody, or antigen-binding fragment thereof, of any one of claims 1-33, wherein said CL region is encoded by a nucleic acid sequence set forth in SEQ ID NO: 80.

35. The isolated antibody, or antigen-binding fragment thereof, of any one of claims 1-34, wherein said antigen-binding fragment of said antibody is an Fab, Fab′, F(ab′)2, Fv or single chain Fv (scFv).

36. The isolated antibody, or antigen-binding fragment thereof, of any one of claims 1-35, wherein said antigen-binding fragment of said antibody is an scFv.

37. The isolated antibody, or antigen-binding fragment thereof, of claim 35 or 36, wherein said scFv comprises a linker connecting said VH region and said VL region.

38. The isolated antibody, or antigen-binding fragment thereof, of claim 37, wherein said VH region, said VL region, and said linker have a 5′ to 3′ orientation of VH region-linker-VL region or VL region-linker-VH region.

39. The isolated antibody, or antigen-binding fragment thereof, of claim 37 or 38, wherein said linker comprises an amino acid sequence having at least about 80% sequence identity to a sequence set forth in any one of SEQ ID NOs: 34-51.

40. The isolated antibody, or antigen-binding fragment thereof, of any one of claims 37-39, wherein said linker comprises an amino acid sequence set forth in any one of SEQ ID NOs: 34-51.

41. The isolated antibody, or antigen-binding fragment thereof, of any one of claims 35-40, wherein said scFv comprises an amino acid sequence having at least about 80% sequence identity to a sequence set forth in any one of SEQ ID NOs: 81-98.

42. The isolated antibody, or antigen-binding fragment thereof, of any one of claims 35-41, wherein said scFv is encoded by a nucleic acid sequence having at least about 80% sequence identity to a sequence set forth in any one of SEQ ID NOs: 99-116.

43. The isolated antibody, or antigen-binding fragment thereof, of any one of claims 35-42, wherein said scFv comprises an amino acid sequence set forth in any one of SEQ ID NOs: 81-98.

44. The isolated antibody, or antigen-binding fragment thereof, of any one of claims 35-43, wherein said scFv is encoded by a nucleic acid sequence set forth in any one of SEQ ID NOs: 99-116.

45. The isolated antibody, or antigen-binding fragment thereof, of any one of claims 1-44, wherein said isolated antibody, or antigen-binding fragment thereof, binds to a human BCMA comprising the amino acid sequence set forth in SEQ ID NO: 1.

46. The isolated antibody, or antigen-binding fragment thereof, of any one of claims 1-45, wherein said isolated antibody, or antigen-binding fragment thereof, binds to human BCMA with a binding affinity (KD) of from about 1×10⁻⁹ M to about 1×10⁻⁸ M.

47. The isolated antibody, or antigen-binding fragment thereof, of any one of claims 1-18 or 27-46, wherein said isolated antibody, or antigen-binding fragment thereof, comprises a human variable region framework region.

48. The isolated antibody, or antigen-binding fragment thereof, of any one of claims 1-46, which is a fully murine antibody, or antigen-binding fragment thereof.

49. The isolated antibody, or antigen-binding fragment thereof, of any one of claims 1-18 or 27-46, which is a chimeric antibody, or antigen-binding fragment thereof.

50. The isolated antibody, or antigen-binding fragment thereof, of any one of claims 1-18 or 27-46, which is a humanized antibody, or antigen-binding fragment thereof.

51. An isolated antibody, or antigen-binding fragment thereof, comprising a VH region that comprises a CDRH1 domain, a CDRH2 domain, and a CDRH3 domain of any VH region set forth in any one of SEQ ID NOs: 2, 6, and 10, wherein said isolated antibody, or antigen-binding fragment thereof, specifically binds to human BCMA.

52. The isolated antibody, or antigen-binding fragment thereof, of claim 51, wherein said isolated antibody, or antigen-binding fragment thereof, is a single domain antibody (sdAb).

53. The isolated antibody, or antigen-binding fragment thereof, of claim 51 or 52, wherein said CDRH1 domain, said CDRH2 domain, and said CDRH3 domain are identified by the Kabat numbering scheme or by the Chothia numbering scheme.

54. The isolated antibody, or antigen-binding fragment thereof, of any one of claims 51-53, wherein said CDRH1 domain comprises an amino acid sequence set forth in any one of SEQ ID NOs: 14, 20, and 26.

55. The isolated antibody, or antigen-binding fragment thereof, of any one of claims 51-54, wherein said CDRH2 domain comprises an amino acid sequence set forth in any one of SEQ ID NOs: 15, 21, and 27.

56. The isolated antibody, or antigen-binding fragment thereof, of any one of claims 51-55, wherein said CDRH3 domain comprises an amino acid sequence set forth in any one of SEQ ID NOs: 16, 22, and 28.

57. The isolated antibody, or antigen-binding fragment thereof, of any one of claims 51-56, wherein:
(a) said CDRH1 domain comprises an amino acid sequence set forth in SEQ ID NO: 14; said CDRH2 domain comprises an amino acid sequence set forth in SEQ ID NO: 15; and said CDRH3 domain comprises an amino acid sequence set forth in SEQ ID NO: 16;
(b) said CDRH1 domain comprises an amino acid sequence set forth in SEQ ID NO: 20; said CDRH2 domain comprises an amino acid sequence set forth in SEQ ID NO: 21; and said CDRH3 domain comprises an amino acid sequence set forth in SEQ ID NO: 22; or
(c) said CDRH1 domain comprises an amino acid sequence set forth in SEQ ID NO: 26; said CDRH2 domain comprises an amino acid sequence set forth in SEQ ID NO: 27; and said CDRH3 domain comprises an amino acid sequence set forth in SEQ ID NO: 28.

58. The isolated antibody, or antigen-binding fragment thereof, of any one of claims 51-57, wherein said VH region comprises an amino acid sequence having at least about 80% sequence identity to a sequence set forth in any one of SEQ ID NOs: 2, 6, and 10.

59. The isolated antibody, or antigen-binding fragment thereof, of any one of claims 51-58, wherein said VH region is encoded by a nucleic acid sequence having at least about 80% sequence identity to a sequence set forth in any one of SEQ ID NOs: 3, 7, and 11.

60. The isolated antibody, or antigen-binding fragment thereof, of any one of claims 51-59, wherein said VH region comprises an amino acid sequence set forth in any one of SEQ ID NOs: 2, 6, and 10.

61. The isolated antibody, or antigen-binding fragment thereof, of any one of claims 51-60, wherein said VH region is encoded by a nucleic acid sequence set forth in any one of SEQ ID NOs: 3, 7, and 11.

62. The isolated antibody, or antigen-binding fragment thereof, of any one of claims 51-61, wherein said isolated antibody, or antigen-binding fragment thereof, binds to a human BCMA comprising the amino acid sequence set forth in SEQ ID NO: 1.

63. The isolated antibody, or antigen-binding fragment thereof, of any one of claims 51-62, wherein said isolated antibody, or antigen-binding fragment thereof, binds to human BCMA with a binding affinity (KD) of from about 1×10⁻⁹ M to about 1×10⁻⁸ M.

64. The isolated antibody, or antigen-binding fragment thereof, of any one of claims 51-59, 62, or 63, wherein said isolated antibody, or antibody fragment thereof, comprises a human variable region framework region.

65. The isolated antibody, or antigen-binding fragment thereof, of any one of claims 51-63, which is a fully murine antibody, or antigen-binding fragment thereof.

66. The isolated antibody, or antigen-binding fragment thereof, of any one of claims 51-59, 62, or 63, which is a chimeric antibody, or antigen-binding fragment thereof.

67. The isolated antibody, or antigen-binding fragment thereof, of any one of claims 51-59, 62, or 63, which is a humanized antibody, or antigen-binding fragment thereof.

68. An isolated antibody, or antigen-binding fragment thereof, which cross-competes for binding to human BCMA with an isolated antibody, or an antigen-binding fragment thereof, of any one of claims 1-67.

69. An isolated antibody, or antigen-binding fragment thereof, which binds to the same epitope on human BCMA as said isolated antibody, or antigen-binding fragment thereof, of any one of claims 1-67.

70. A pharmaceutical composition comprising said isolated antibody, or antigen-binding fragment thereof, of any one of claims 1-69, and a pharmaceutically acceptable carrier.

71. An immunoconjugate comprising said isolated antibody, or antigen-binding fragment thereof, of any one of claims 1-69, linked to a therapeutic agent.

72. The immunoconjugate of claim 71, wherein said therapeutic agent is a drug, a cytotoxin, or a radioactive isotope.

73. A pharmaceutical composition comprising said immunoconjugate of claim 71 or 72 and a pharmaceutically acceptable carrier.

74. A bispecific molecule comprising said isolated antibody, or antigen-binding fragment thereof, of any one of claims 1-69, linked to a second functional moiety.

75. The bispecific molecule of claim 74, wherein said second functional moiety has a different binding specificity than said isolated antibody, or antigen binding fragment thereof.

76. A pharmaceutical composition comprising said bispecific molecule of claim 74 or 75 and a pharmaceutically acceptable carrier.

77. A polynucleotide comprising a nucleic acid sequence encoding said isolated antibody, or antigen-binding fragment thereof, of any one of claims 1-69.

78. An expression vector comprising said polynucleotide of claim 77.

79. A host cell comprising said expression vector of claim 78.

80. A method for detecting BCMA in a whole cell or tissue, comprising:
(a) contacting a cell or tissue with said isolated antibody, or antigen-binding fragment thereof, of any one of claims 1-69, wherein said isolated antibody, or antigen-binding fragment thereof, comprises a detectable label; and
(b) determining the amount of said labeled isolated antibody, or antigen-binding fragment thereof, bound to said cell or tissue by measuring the amount of detectable label associated with said cell or tissue, wherein the amount of bound isolated antibody, or antigen-binding fragment thereof, indicates the amount of BCMA in said cell or tissue.

81. A method of treating a cancer in a subject, comprising administering an effective amount of said isolated antibody, or antigen-binding fragment thereof, of any one of claims 1-69 to said subject, thereby inducing death of a cancer cell in said subject.

82. The method of claim 81, wherein said method reduces the number of said cancer cells.

83. The method of claim 81 or 82, wherein said method reduces the size of said cancer.

84. The method of any one of claims 81-83, wherein said method eradicates said cancer in said subject.

85. The method of any one of claims 81-84, wherein said cancer is selected from the group consisting of multiple myeloma, Non-Hodgkin Lymphoma, Hodgkin Lymphoma, Chronic Lymphocytic Leukemia (CLL), glioblastoma, and Waldenstrom's Macroglobulinemia.

86. The method of any one of claims 81-85, wherein said cancer is multiple myeloma.

87. The method of any one of claims 81-86, wherein said subject is a human.

88. Use of said isolated antibody, or antigen-binding fragment thereof, of any one of claims 1-69 for the treatment of a cancer.

89. The use of claim 88, wherein said cancer is selected from the group consisting of multiple myeloma, Non-Hodgkin Lymphoma, Hodgkin Lymphoma, Chronic Lymphocytic Leukemia (CLL), glioblastoma, and Waldenstrom's Macroglobulinemia.

90. The use of claim 88 or claim 89, wherein said cancer is multiple myeloma.

91. The isolated antibody, or antigen-binding fragment thereof, of any one of claims 1-69 for use in treating a cancer in a subject.

92. The isolated antibody, or antigen-binding fragment thereof, of claim 91, wherein said cancer is selected from the group consisting of multiple myeloma, Non-Hodgkin Lymphoma, Hodgkin Lymphoma, Chronic Lymphocytic Leukemia (CLL), glioblastoma, and Waldenstrom's Macroglobulinemia.

93. The isolated antibody, or antigen-binding fragment thereof, of claim 91 or 92, wherein said cancer is multiple myeloma.

94. A kit for treating a cancer, comprising said isolated antibody, or antigen-binding fragment thereof, of any one of claims 1-69.

95. The kit of claim 94, wherein said kit further comprises written instructions for using said isolated antibody, or antigen-binding fragment thereof, for treating a subject having said cancer.

96. The kit of claim 94 or 95, wherein said cancer is multiple myeloma.

97. A polynucleotide comprising a nucleic acid sequence encoding a chimeric antigen receptor (CAR), wherein said CAR comprises a human anti-BCMA binding domain, a transmembrane domain, and an intracellular domain, and wherein said anti-BCMA binding domain comprises said isolated antibody, or antigen-binding fragment thereof, of any one of claims 1-69.

98. The polynucleotide of claim 97, wherein said anti-BCMA binding domain comprises said scFv of any one of claims 35-44.

99. The polynucleotide of claim 97, wherein said anti-BCMA binding domain comprises said sdAb of claim 52.

100. The polynucleotide of any one of claims 97-99, wherein said anti-BCMA binding domain binds to a human BCMA comprising an amino acid sequence set forth in SEQ ID NO: 1.

101. The polynucleotide of any one of claims 97-100, wherein said transmembrane domain comprises a transmembrane domain of a protein selected from the group consisting of the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137 and CD154.

102. The polynucleotide of any one of claims 97-101, wherein said transmembrane domain comprises an amino acid sequence having at least about 80% sequence identity to a sequence set forth in SEQ ID NO: 56.

103. The polynucleotide of any one of claims 97-102, wherein said transmembrane domain comprises an amino acid sequence set forth in SEQ ID NO: 56.

104. The polynucleotide of any one of claims 97-103, wherein said CAR comprises a hinge domain connecting said anti-BCMA binding domain and said transmembrane domain.

105. The polynucleotide of claim 104, wherein said hinge domain comprises an amino acid sequence having at least about 80% sequence identity to the sequence set forth in SEQ ID NO: 54.

106. The polynucleotide of claim 104 or 105, wherein said hinge domain comprises an amino acid sequence set forth in SEQ ID NO: 54.

107. The polynucleotide of any one of claims 97-106, wherein said intracellular signaling domain comprises a co-stimulatory domain.

108. The polynucleotide of claim 107, wherein said co-stimulatory domain comprises a Novel 6 (N6) domain, a Novel 1 (N1) domain, a 4-1BB domain, a CD28 domain, or a functional signaling domain obtained from a protein including an MHC class I molecule, TNF receptor proteins, Immunoglobulin-like proteins, cytokine receptors, integrins, signaling lymphocytic activation molecules (SLAM proteins), activating NK cell receptors, BTLA, a Toll ligand receptor, OX40, CD2, CD7, CD27, CD30, CD40, CDS, ICAM-1, LFA-1 (CD1 1a/CD18), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD1 1d, ITGAE, CD103, ITGAL, CD1 1a, LFA-1, ITGAM, CD1 1b, ITGAX, CD1 c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRT AM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83.

109. The polynucleotide of claim 107 or 108, wherein said co-stimulatory domain comprises an amino acid sequence having at least about 80% sequence identity to a sequence set forth in any one of SEQ ID NOs: 58, 60, 62, and 64.

110. The polynucleotide of any one of claims 107-109, wherein said co-stimulatory domain comprises an amino acid sequence set forth in any one of SEQ ID NOs: 58, 60, 62, and 64.

111. The polynucleotide of any one of claims 97-110, wherein said intracellular domain comprises a signaling domain.

112. The polynucleotide of claim 111, wherein said signaling domain is a CD3 zeta signaling domain.

113. The polynucleotide of claim 111 or 112, wherein said signaling domain comprises an amino acid sequence having at least about 80% sequence identity to a sequence set forth in SEQ ID NO: 66.

114. The polynucleotide of any one of claims 111-113, wherein said signaling domain comprises an amino acid sequence set forth in SEQ ID NO: 66.

115. The polynucleotide of any one of claims 111-114, wherein the sequences encoding said co-stimulatory domain and said signaling domain are expressed in the same frame and as a single polypeptide chain.

116. The polynucleotide of any one of claims 104-106, wherein said CAR comprises a spacer connecting said hinge domain to said anti-BCMA binding domain.

117. The polynucleotide of claim 116, wherein said spacer comprises an amino acid sequence having at least about 80% sequence identity to a sequence set forth in SEQ ID NO: 52.

118. The polynucleotide of claim 116 or 117, wherein said spacer comprises an amino acid sequence set forth in SEQ ID NO: 52.

119. The polynucleotide of any one of claims 97-118, wherein said CAR comprises a signal peptide.

120. The polynucleotide of claim 119, wherein said signal peptide comprises an amino acid sequence having at least about 80% sequence identity to a sequence set forth in SEQ ID NO: 68, 70, or 189.

121. The polynucleotide of claim 119 or claim 120, wherein said signal peptide comprises an amino acid sequence set forth in SEQ ID NO: 68, 70, or 189.

122. The polynucleotide of any one of claims 97-121, wherein said CAR comprises an amino acid sequence having at least about 80% sequence identity to a sequence set forth in any one of SEQ ID NOs: 117-134.

123. The polynucleotide of any one of claims 97-122, wherein said CAR is encoded by a nucleic acid sequence having at least 80% sequence identity to a sequence set forth in any one of SEQ ID NOs: 135-152.

124. The polynucleotide of any one of claims 97-123, wherein said CAR comprises an amino acid sequence set forth in any one of SEQ ID NOs: 117-134.

125. The polynucleotide of any one of claims 97-124, wherein said CAR is encoded by a nucleic acid sequence set forth in any one of SEQ ID NOs: 135-152.

126. The polynucleotide of any one of claims 97-121, wherein said CAR comprises an amino acid sequence having at least about 80% sequence identity to a sequence set forth in any one of SEQ ID NOs: 153-170.

127. The polynucleotide of any one of claims 97-121 and 126, wherein said CAR is encoded by a nucleic acid sequence having at least 80% sequence identity to a sequence set forth in any one of SEQ ID NOs: 171-188.

128. The polynucleotide of any one of claims 97-121, 126, and 127, wherein said CAR comprises an amino acid sequence set forth in any one of SEQ ID NOs: 153-170.

129. The polynucleotide of any one of claims 97-121 and 126-128, wherein said CAR is encoded by a nucleic acid sequence set forth in any one of SEQ ID NOs: 171-188.

130. The polynucleotide of any one of claims 97-129, wherein said polynucleotide comprises a promoter that is operably linked to said nucleic acid sequence encoding said CAR.

131. The polynucleotide of claim 130, wherein said promoter comprises a nucleic acid sequence having at least about 80% sequence identity to a sequence set forth in SEQ ID NO: 72 or 73.

132. The polynucleotide of claim 130 or 131, wherein said promoter comprises a nucleic acid sequence set forth in SEQ ID NO: 72 or 73.

133. A CAR polypeptide encoded by said polynucleotide of any one of claims 97-132.

134. A recombinant DNA construct comprising said polynucleotide of any one of claims 97-132.

135. A recombinant virus comprising said polynucleotide of any one of claims 97-132, wherein said recombinant virus is a recombinant adeno-associated virus (AAV), a recombinant lentivirus, a recombinant adenovirus, or a recombinant retrovirus.

136. The recombinant virus of claim 135, wherein said recombinant virus is a recombinant AAV.

137. A genetically-modified eukaryotic cell comprising in its genome said polynucleotide of any one of claims 97-132, wherein said CAR is expressed by said genetically-modified eukaryotic cell.

138. The genetically-modified eukaryotic cell of claim 137, wherein said genetically-modified eukaryotic cell comprises an inactivated T cell receptor (TCR) alpha gene, an inactivated TCR alpha constant region (TRAC) gene, and/or an inactivated TCR beta gene.

139. The genetically-modified eukaryotic cell of claim 137 or 138, wherein said polynucleotide is randomly integrated within the genome of said genetically-modified eukaryotic cell.

140. The genetically-modified eukaryotic cell of claim 137 or 138, wherein said polynucleotide is positioned within the genome of said genetically-modified eukaryotic cell within a target gene, wherein expression of a polypeptide encoded by said target gene is disrupted.

141. The genetically-modified eukaryotic cell of claim 140, wherein said target gene is a TCR alpha gene, a TRAC gene, or a TCR beta gene.

142. The genetically-modified eukaryotic cell of claim 140 or 141, wherein said target gene is a TRAC gene.

143. The genetically-modified eukaryotic cell of any one of claims 140-142, wherein said polynucleotide is positioned within a sequence set forth in SEQ ID NO: 74.

144. The genetically-modified eukaryotic cell of any one of claims 140-143, wherein said polynucleotide is positioned between nucleotide positions 13 and 14 of a sequence set forth in SEQ ID NO: 74.

145. The genetically-modified eukaryotic cell of any one of claims 137-144, wherein said genetically-modified eukaryotic cell is a genetically-modified immune cell.

146. The genetically-modified eukaryotic cell of claim 145, wherein said genetically-modified immune cell is a genetically-modified T cell, a genetically-modified natural killer (NK) cell, a genetically-modified B cell, or a genetically-modified macrophage.

147. The genetically-modified eukaryotic cell of claim 145 or 146, wherein said genetically-modified immune cell is a genetically-modified T cell.

148. The genetically-modified eukaryotic cell of any one of claims 137-144, wherein said genetically-modified eukaryotic cell is a genetically-modified induced pluripotent stem cell (iPSC).

149. The genetically-modified eukaryotic cell of any one of claims 137-148, wherein said genetically-modified eukaryotic cell is a genetically-modified human cell.

150. A method of producing a genetically-modified eukaryotic cell, said method comprising introducing into a eukaryotic cell a template nucleic acid comprising said polynucleotide of any one of claims 97-132, wherein said polynucleotide is integrated into the genome of said eukaryotic cell, and wherein said CAR is expressed by said genetically-modified eukaryotic cell.

151. The method of claim 150, wherein said polynucleotide is introduced by a recombinant lentivirus, and wherein said polynucleotide is inserted into the genome of said eukaryotic cell by random integration.

152. The method of claim 150 or 151, wherein said eukaryotic cell comprises an inactivated TCR alpha gene, an inactivated TRAC gene, and/or an inactivated TCR beta gene.

153. The method of claim 150, wherein said method comprises introducing into said eukaryotic cell:
(a) a nucleic acid encoding an engineered nuclease having specificity for a recognition sequence in the genome of said eukaryotic cell, wherein said engineered nuclease is expressed in said eukaryotic cell; and
(b) said template nucleic acid comprising said polynucleotide;
wherein said engineered nuclease generates a cleavage site at said recognition sequence, and wherein said polynucleotide is inserted into the genome of said eukaryotic cell at said cleavage site.

154. The method of claim 153, wherein said template nucleic acid is introduced into said eukaryotic cell using a recombinant virus.

155. The method of claim 154, wherein said recombinant virus is a recombinant AAV.

156. The method of claim 155, wherein said recombinant AAV has a serotype of AAV6.

157. The method of any one of claims 153-156, wherein said nucleic acid encoding said engineered nuclease is an mRNA.

158. The method of any one of claims 153-157, wherein said template nucleic acid comprises a 5′ homology arm and a 3′ homology arm which have homology to sequences 5′ upstream and 3′ downstream, respectively, of said cleavage site, and wherein said polynucleotide is inserted into said cleavage site by homologous recombination.

159. The method of any one of claims 153-158, wherein said engineered nuclease is an engineered meganuclease, a zinc finger nuclease, a TALEN, a compact TALEN, a CRISPR system nuclease, or a megaTAL.

160. The method of any one of claims 153-159, wherein said engineered nuclease is an engineered meganuclease.

161. The method of claim 159 or 160, wherein said engineered meganuclease comprises an amino acid sequence set forth in SEQ ID NO: 76.

162. The method of any one of claims 153-161, wherein said recognition sequence is positioned within a target gene, and wherein insertion of said polynucleotide at said cleavage site disrupts expression of a polypeptide encoded by said target gene.

163. The method of claim 162, wherein said target gene is a TCR alpha gene, a TRAC gene, or a TCR beta gene.

164. The method of claim 162 or 163, wherein said target gene is a TRAC gene.

165. The method of any one of claims 162-164, wherein said polynucleotide is inserted within a sequence set forth in SEQ ID NO: 74.

166. The method of any one of claims 162-165, wherein said polynucleotide is inserted between nucleotide positions 13 and 14 of a sequence set forth in SEQ ID NO: 74.

167. The method of any one of claims 150-166, wherein said genetically-modified eukaryotic cell is a genetically-modified immune cell.

168. The method of claim 167, wherein said genetically-modified immune cell is a genetically-modified T cell, a genetically-modified natural killer (NK) cell, a genetically-modified B cell, or a genetically-modified macrophage.

169. The method of claim 167 or 168, wherein said genetically-modified immune cell is a genetically-modified T cell.

170. The method of any one of claims 150-167, wherein said genetically-modified eukaryotic cell is a genetically-modified induced pluripotent stem cell (iPSC).

171. The method of any one of claims 150-170, wherein said genetically-modified eukaryotic cell is a genetically-modified human cell.

172. A genetically-modified eukaryotic cell produced by the method of any one of claims 150-171.

173. A population of eukaryotic cells comprising a plurality of said genetically-modified eukaryotic cells of any one of claims 137-149 and 172.

174. The population of claim 173, wherein at least about 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96, 97%, 98%, 99%, or 100% of said eukaryotic cells in said population are said genetically-modified eukaryotic cells.

175. The population of claim 173 or 174, wherein said genetically-modified eukaryotic cells in said population express said CAR and comprise an inactivated TCR alpha gene, an inactivated TRAC gene, and/or an inactivated TCR beta gene.

176. A pharmaceutical composition comprising a plurality of said genetically-modified eukaryotic cells of any one of claims 137-149 and 172, or said population of eukaryotic cells of any one of claims 173-175, and a pharmaceutically-acceptable carrier.

177. A method of treating a cancer in a subject, comprising administering to said subject an effective amount of said pharmaceutical composition of claim 176 to said subject, thereby inducing death of a cancer cell in said subject.

178. The method of claim 177, wherein said method reduces the number of said cancer cells.

179. The method of claim 177 or 178, wherein said method reduces the size of said cancer.

180. The method of any one of claims 177-179, wherein said method eradicates said cancer in said subject.

181. The method of any one of claims 177-180, wherein said cancer is selected from the group consisting of multiple myeloma, Non-Hodgkin Lymphoma, Hodgkin Lymphoma, Chronic Lymphocytic Leukemia (CLL), glioblastoma, and Waldenstrom's Macroglobulinemia.

182. The method of any one of claims 177-181, wherein said cancer is multiple myeloma.

183. The method of any one of claims 177-182, wherein said pharmaceutical composition is administered in combination with a cancer therapy selected from the group consisting of chemotherapy, surgery, radiation, and gene therapy.

184. The method of any one of claims 177-183, wherein said subject is a human.

185. Use of said genetically-modified eukaryotic cell of any one of claims 137-149 and 172 for the treatment of a cancer.

186. The use of claim 185, wherein said cancer is selected from the group consisting of multiple myeloma, Non-Hodgkin Lymphoma, Hodgkin Lymphoma, Chronic Lymphocytic Leukemia (CLL), glioblastoma, and Waldenstrom's Macroglobulinemia.

187. The use of claim 185 or 186, wherein said cancer is multiple myeloma.

188. The genetically-modified eukaryotic cell of any one of claims 137-149 and 172 for use in treating a cancer in a subject.

189. The genetically-modified eukaryotic cell of claim 188, wherein said cancer is selected from the group consisting of multiple myeloma, Non-Hodgkin Lymphoma, Hodgkin Lymphoma, Chronic Lymphocytic Leukemia (CLL), glioblastoma, and Waldenstrom's Macroglobulinemia.

190. The genetically-modified eukaryotic cell of claim 188 or claim 189, wherein said cancer is multiple myeloma.

191. A kit for treating a cancer, comprising said genetically-modified eukaryotic cell of any one of claims 137-149 and 172.

192. The kit of claim 191, wherein said kit further comprises written instructions for using said genetically-modified eukaryotic cell for treating a subject having said cancer.

193. The kit of claim 191 or claim 192, wherein said cancer is multiple myeloma.

194. The genetically-modified eukaryotic cell of any one of claims 137-149 and 172 for use as a medicament.
